(19) United States
(12) Patent Application Publication
(10) Pub. No.: US 2025/0213713 A1
(43) Pub. Date: Jul. 3, 2025

(54) Antibody-Drug Conjugates Targeting uPARAP Comprising Exatecan Derivatives

(71) Applicant: ADCENDO APS, Frederiksberg (DK)

(72) Inventors: Christoffer Fagernæs NIELSEN, Frederiksberg (DK); Yu ZHANG, Shanghai (CN); Zhongyuan ZHU, Shanghai (CN); Carmel LYNCH, Frederiksberg (DK); Dominik MUMBERG, Frederiksberg (DK); Feng WANG, Frederiksberg (DK)

(21) Appl. No.: 19/046,030

(22) Filed: Feb. 5, 2025

Related U.S. Application Data
(63) Continuation of application No. PCT/EP2023/087885, filed on Dec. 28, 2023.

(30) Foreign Application Priority Data
Dec. 28, 2022 (WO) PCT/CN2022/142620
Sep. 26, 2023 (EP) 23199767.7

(51) Int. Cl. A61K 47/68; A61P 35/00; C07K 16/28

(52) U.S. Cl. CPC A61K 47/68037; A61K 47/6849; A61K 47/6889; A61P 35/00; C07K 16/2851

(57) ABSTRACT

The present invention relates to antibody-drug conjugates targeting the receptor uPARAP, in particular antibody-drug conjugates (ADCs) comprising humanized antibodies directed against uPARAP and exatecan derivatives and their use in delivery of active agents to cells and tissues expressing uPARAP. The invention further relates to the use of said ADCs in the treatment of diseases involving uPARAP expressing cells, such as certain cancers.

### CROSS-REFERENCE TO RELATED APPLICATIONS

This application is a continuation of PCT/EP2023/087885, filed Dec. 28, 2023, which claims priority to Chinese Application No. PCT/CN2022/142620, filed Dec. 28, 2022, and EP Application number 23199767.7, filed Sep. 26, 2023, the entire contents of which are incorporated herein by reference.

### SEQUENCE LISTING

The instant application contains a Sequence Listing which is being submitted herewith electronically in XML format and is hereby incorporated by reference in its entirety. Said XML copy, created on Dec. 22, 2023, is named 105728_000123_Sequence_listing.xml and is 24 KB in size.

### FIELD OF INVENTION

The present invention relates to antibody-drug conjugates targeting the receptor uPARAP, in particular antibody-drug conjugates (ADCs) comprising humanized antibodies directed against uPARAP and exatecan derivatives and their use in delivery of the active agents to cells and tissues expressing uPARAP. The invention further relates to the use of said ADCs in the treatment of diseases involving uPARAP expressing cells, such as certain cancers.

### BACKGROUND

Urokinase-type Plasminogen Activator Receptor Associated Protein (uPARAP), also known as CD280, Endo180 and mannose receptor C type 2, is a member of the macrophage mannose receptor family of endocytic transmembrane glycoproteins. uPARAP is a membrane protein involved in matrix turnover during tissue remodelling, particularly the uptake and intracellular degradation of collagen. The uPARAP receptor consists of an N-terminal cysteine-rich domain (CysR), a fibronectin type II (FN-II) domain, and eight C-type lectin-like domains (CTLDs 1-8).

The receptor uPARAP is upregulated in the tumour cells of specific cancers, including sarcomas and late-stage glioblastoma. Additionally, the receptor is most often upregulated in stromal cells surrounding solid tumours and some literature suggests a high expression of uPARAP in bone metastasis from prostate cancer (Caley et al., 2012, J. Pathol 5: 775-783). In healthy adult individuals, the receptor displays a restricted expression pattern (Melander et al., 2015, Int J Oncol 47: 1177-1188).

Antibody-drug conjugates (ADCs) are a class of highly potent biopharmaceutical drugs designed as a targeted therapy, in particular for the treatment of cancer. ADCs are complex molecules composed of an antibody (a whole mAb or an antibody fragment) linked, via a stable, chemical, linker that may possess labile bonds, to an active agent, such as a biologically active drug or cytotoxic compound. By combining the unique targeting capabilities of antibodies with the cell-killing ability of cytotoxic drugs, antibody-drug conjugates allow sensitive discrimination between healthy and diseased tissue, based on expression of the antibody antigen. This means that, in contrast to traditional chemotherapeutic agents, antibody-drug conjugates actively target and attack cancer cells, so that healthy cells with little or no antigen expression are less severely affected.

To date, more than 10 ADCs have received market approval and several ADCs are currently in clinical trials.

WO 2010/111198 discloses conjugates comprising an anti-uPARAP antibody and suggests use of such conjugates in the delivery of therapeutic agents to cells that express uPARAP.

WO 2017/133745 discloses ADCs directed against uPARAP.

WO 2022/068878 discloses exatecan derivatives and ADC's comprising same.

Treatment methods currently exist for most cancer types. However, in many cases with unsatisfactory efficiency or with adverse effects due to high dosing of the therapeutic agent. Thus, there is a need for more efficient treatments with increased potency.

### SUMMARY

Provided herein is an antibody drug conjugate comprising a humanized version of the murine 9b7 antibody targeting the uPARAP receptor and exatecan derivatives. The murine 9b7 antibody was originally described in WO 2017/133745. The antibody-drug conjugates as described herein are capable of specifically targeting cells and tissues expressing uPARAP. ADCs comprising the herein described humanized 9b7 antibody display enhanced efficacy compared to ADCs comprising the murine 9b7 antibody as well as enhanced efficacy compared to ADCs comprising other humanized versions of the murine 9b7 antibody.

In particular, the present disclosure relates to an antibody-drug conjugate comprising an antibody which binds to uPARAP comprising:
a. an immunoglobulin light chain variable region comprising or consisting of the amino acid sequence of SEQ ID NO: 3; and/or
b. an immunoglobulin heavy chain variable region comprising or consisting of the amino acid sequence of SEQ ID NO: 6;
wherein the antibody-drug conjugate comprises an active agent with a structure according to one selected from the group consisting of: formula (I-A), formula (II-A) and formula (III-A) as described herein below; and
wherein the antibody-drug conjugate optionally comprises a linker that links the antibody with the active agent.

Furthermore, the present disclosure provides for a method of manufacture of an antibody-drug conjugate as described herein, the method comprising use of a compound according to one selected from the group consisting of: formula (I-A), formula (II-Fx), formula (II-Fy) and formula (III-F) as described herein.

Furthermore, the present disclosure relates to a method for treatment of a disease characterised by cells expressing uPARAP, said method comprising administering to a subject the antibody-drug conjugate as defined herein, or a pharmaceutical composition comprising the antibody-drug conjugate as defined herein.

An even further aspect of the present disclosure is a kit comprising the antibody-drug conjugate as defined herein, or a pharmaceutical composition comprising he antibody-drug conjugate as defined herein, optionally further comprising means for administering the antibody-drug conjugate to a subject and/or instructions for use

### DESCRIPTION OF DRAWINGS

FIG. 1: In vitro cell viability assays of U937 cancer cell lines exposed to MMAE-based ADCs comprising either the LC0HC0 antibody (comprising the variable domains of the original murine 9b7 antibody fused to human IgG constant regions), or the humanized LC4HC3 antibody. But for the antibody, the two ADCs are identical and were produced by identical methods. Cells were incubated for 96 hours, before being analyzed by colorimetric viability assay. The assay for the U937 cell line shows that ADCs based on LC4HC3 have a significantly greater reduction in overall cell viability compared to the LC0HC0 ADCs.

FIG. 2: Internalization of humanized antibodies LC4HC3 and LC3HC3 in SAOS-2 osteosarcoma cells. Detailed protocols are presented in Example 2. The data shows that LC4HC3 is internalized not only faster than LC3HC3 but also to a greater extent in SAOS-2 osteosarcoma cells.

FIG. 3: In vivo efficacy of Vedotin-type ADCs based on LC4HC3 (LC4HC3-vc-MMAE, FIG. 3a) and LC3HC3 (LC3HC3-vc-MMAE, FIG. 3b). CB17 mice were inoculated with U937 cells to induce tumor growth. Tumor size was closely monitored and treatment initiated once a size of approximately 80-150 mm³ was reached. But for the antibody, the two ADCs are identical and were produced by identical methods. Each line in FIGS. 3a and 3b represents tumor size in a mouse administered a 4 mg/kg dose of the referenced ADC for 7 days, twice daily. The data shows that ADCs based on humanized 9b7 antibody LC4HC3 are superior antitumor agents compared to ADCs based on a different humanized 9b7 antibody, LC3HC3.

FIG. 4: Calculated tumour size from calliper measurements using the formula tumour size={(length*width²)/2}. 4a: dosing at 3 mg/kg. 4b: dosing at 10 mg/kg. The data shown are mean±standard deviation (SD) when 50% or more of the mice are alive, n=7. The mice were treated IV in the tail vein weekly for 3 times as indicated by the triangles on the x-axis. Employed cell line is SK-LMS-1, employed ADCs are ADCE-D01 (squares) and control ADCE-D51 (circles). Both ADCs are also compared to vehicle treatment with PBS (triangles).

FIG. 5: Calculated tumour size from calliper measurements using the formula tumour size={(length*width²)/2}. 5a: dosing at 3 mg/kg. 5b: dosing at 6 mg/kg. The data shown are mean±SD when 50% or more of the mice are alive. The mice were treated IV in the tail vein weekly for 3 times as indicated by the triangles on the x-axis. Employed cell line is RD, employed ADCs are ADCE-D01 (squares) and control ADCE-D51 (circles). Both ADCs are also compared to vehicle treatment with PBS (triangles).

### DETAILED DESCRIPTION

The ADCs of the present disclosure are internalised upon binding to uPARAP receptors at the cell surface, thus allowing for intracellular actions of the active agent of the antibody-drug conjugate complex.

### Anti-uPARAP Humanised Antibodies

Methods of generating antibodies are well known in the art. For example, antibodies may be generated via any one of several methods which employ induction of in vivo production of antibody molecules, screening of immunoglobulin libraries, or generation of monoclonal antibody molecules by cell lines in culture. These include, but are not limited to, the hybridoma technique, the human B-cell hybridoma technique, and the Epstein-Barr virus (EBV)-hybridoma technique.

Humanised antibodies are generally preferred in medicines intended for humans and methods for humanising antibodies are well known in the art. Although humanisation techniques are known, it can be a challenge to achieve humanised antibodies that retain the binding properties of the initial antibody and even more challenging to achieve humanised antibodies with improved characteristics, such as improved ligand affinity and efficacy compared to the initial antibody.

The inventors herein provide an ADC comprising an improved anti-uPARAP antibody, which is a humanised version of the 9b7 murine antibody and which displays improved ligand affinity and efficacy compared to the 9b7 murine antibody as well as improved internalization and in vivo efficacy compared to other humanized versions of the 9b7 antibody.

The ADCs of the present disclosure comprising the anti-uPARAP antibody as described herein may be of any immunoglobulin class including IgG, IgM, IgD, IgE, IgA, and any subclass thereof. IgG subclasses are also well known to those in the art and include but are not limited to human IgG1, IgG2, IgG3 and IgG4. In one embodiment the antibody is an IgG monoclonal antibody. In one embodiment the antibody is IgG1K.

The anti-uPARAP antibody of the present disclosure is a humanised 9b7 antibody, which binds to the uPARAP receptor, more specifically, the humanized 9b7 antibody disclosed herein binds at least to the fibronectin type II (FN-II) domain of the uPARAP receptor.

The humanized 9b7 antibody, also referred to herein as 980.2 LC4HC3, comprises a light chain variable region of amino acids comprising SEQ ID NO: 3, which is the variable region of LC4, and a heavy chain variable region of amino acids comprising SEQ ID NO:6, which is the variable region of HC3.

The humanized 9b7 antibody, also referred to herein as 980.2 LC4HC3, may comprise a light chain of amino acids comprising or consisting of SEQ ID NO: 1, which is LC4, and a heavy chain of amino acids comprising or consisting of SEQ ID NO:4, which is HC3.

In one embodiment of the present disclosure, the anti-uPARAP antibody as defined herein comprises:
a. an immunoglobulin light chain variable region comprising or consisting of the amino acid sequence of SEQ ID NO: 3; and
b. an immunoglobulin heavy chain variable region comprising or consisting of the amino acid sequence of SEQ ID NO: 6.

In one embodiment of the present disclosure, the antibody which binds to uPARAP as defined herein comprises:
a. an immunoglobulin light chain comprising the amino acid sequence of SEQ ID NO: 1 (LC4); and
b. an immunoglobulin heavy chain comprising the amino acid sequence of SEQ ID NO: 4 (HC3).

### Antibody-Drug Conjugates (ADCs) Comprising Anti-uPARAP Humanised Antibodies

The data of the inventors surprisingly shows that ADCs comprising the humanized LC4HC3 antibody result in a significantly greater reduction in overall cell viability compared to ADCs based LC0HC0 (having variable domains of the 9b7 murine antibody fused to human IgG constant regions). ADCs based on LC4HC3 also exhibit improved internalization and in vivo efficacy compared to ADCs based on LC3HC3 (another humanized 9b7 antibody).

The antibody drug conjugates described herein comprise a particular humanized version of the murine 9b7 antibody targeting the uPARAP receptor, more particularly LC4HC3, and exatecan derivatives. In some embodiments, the ADCs described herein also comprise a linker connecting the exatecan derivatives to LC4HC3. The exatecan derivatives disclosed herein, methods for their preparation and conjugation to antibodies are described in WO 2022/068878; the entire teachings of which are incorporated by reference.

One embodiment of the present disclosure is an antibody-drug conjugate (ADC) comprising an antibody which binds to uPARAP comprising:
i. an immunoglobulin light chain variable region comprising or consisting of the amino acid sequence of SEQ ID NO: 3; and
ii. an immunoglobulin heavy chain variable region comprising or consisting of the amino acid sequence of SEQ ID NO: 6;
   wherein the antibody-drug conjugate comprises an active agent with a structure according to one selected from the group consisting of:
a. formula (I-A) or a tautomer, a mesomer, a racemate, an enantiomer or a diastereoisomer thereof, or a mixture thereof, or a pharmaceutically acceptable salt thereof,

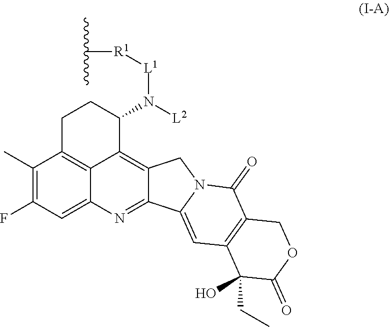

wherein, R¹ is selected from the group consisting of: —O—, —(R²)N—, —P(═O)(R²)—, —P(R²)— and —S—;
L² is —(C(R^{3a})(R^{3b}))ₘ—R,
wherein 0 or no less than 1 methylene unit of L² is independently replaced by —Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
L¹ is —(C(R^{5a})(R^{5b}))ₙ—,
wherein 0 or no less than 1 methylene unit of L¹ is independently replaced by —Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
-Cy- is selected from the group consisting of: 6-10 membered arylene, 5-8 membered heteroarylene, 3-10 membered heterocyclylene, and 3-10 membered saturated or partially unsaturated carbocyclylene, wherein -Cy- is unsubstituted or independently substituted with no less than 1 substituent R⁷;
wherein each R^{3a}, each R^{3b}, each R⁴, each R^{5a}, each R^{5b} and each R⁶ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R; or, R^{3a} and R^{5a}, R⁴ and R^{5a}, R^{3a} and R⁶ or R⁴ and R⁶ each independently optionally form a ring B together with an atom therebetween, wherein the ring B is selected from the group consisting of: 5-8 membered heteroarylene and 3-10 membered saturated or partially unsaturated heterocyclylene, and the ring B is unsubstituted or independently substituted with no less than 1 substituent R⁸;
wherein each R², each R⁷ and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n are each independently selected from the group consisting of integers≥1; and wherein R¹ links the structure shown in formula (I-A) to the antibody either directly or optionally through a linker;
b. Formula (II-A) or a tautomer, a mesomer, a racemate, an enantiomer or a diastereoisomer thereof, or a mixture thereof, or a pharmaceutically acceptable salt thereof

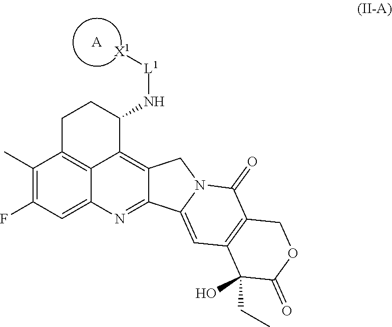

wherein, X¹ is selected from the group consisting of: N, P, and saturated or unsaturated C; when X¹ is saturated C, X¹ is substituted with Rⁿ;
wherein ring A links the structure according to formula II-A to the antibody either directly or optionally through a linker;
when X¹ is saturated C, ring A is selected from the group consisting of: 3-10 membered saturated or partially unsaturated heterocyclyl, and 3-10 membered saturated or partially unsaturated carbocyclyl, wherein ring A is substituted with 0 or no less than 1 substituent R^{1a};
or, when X¹ is unsaturated C, ring A is selected from the group consisting of: 6-10 membered aryl, 5-8 membered heteroaryl, 3-10 membered partially unsaturated heterocyclyl, and 3-10 membered partially unsaturated carbocyclyl, wherein ring A is substituted with 0 or no less than 1 substituent R^{1b};
or, when X¹ is N or P, ring A is selected from the group consisting of: 5-8 membered heteroaryl and 3-10 membered saturated or partially unsaturated heterocyclyl, wherein ring A is substituted with 0 or no less than 1 substituent R^{1c}; when ring A is selected from the group consisting of: 6-10 membered aryl, 5-8 membered heteroaryl, and 3-10 membered saturated or partially unsaturated carbocyclyl, ring A is substituted with p L², wherein L² is not Rⁿ;
or, when ring A is 3-10 membered saturated or partially unsaturated heterocyclyl, ring A is substituted with p L², or ring A comprises q ring-forming heteroatom X² and X² is used for linking formula (II-A) to the antibody either directly or optionally through a linker;
X² is selected from the group consisting of: N and P;
L² is —R²-L³-, and R² is used for linking formula (II-A) to the antibody either directly or optionally through a linker;
L³ is —(C(R^{3a})(R^{3b}))ₘ—, wherein when L³ comprises a methylene unit, 0 or no less than 1 methylene unit of L³ is independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² is selected from the group consisting of: —O—, —(R^{2a})N—, —S— and —P(═O)(R^{2a})—; L¹ is —(C(R^{5a})(R^{5b}))ₙ—, wherein when L¹ comprises a methylene unit, 0 or no less than 1 methylene unit of L¹ is independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1a}, each R^{1b}, each R^{1c}, each R^{2a}, each R^{3a}, each R^{3b}, each R⁴, each R^{5a}, each R^{5b}, each R⁶ and each Rⁿ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n are each independently selected from the group consisting of integers≥0, and p and q are each independently selected from the group consisting of integers≥1;
c. Formula (III-A) or a tautomer, a mesomer, a racemate, an enantiomer or a diastereoisomer thereof, or a mixture thereof, or a pharmaceutically acceptable salt thereof

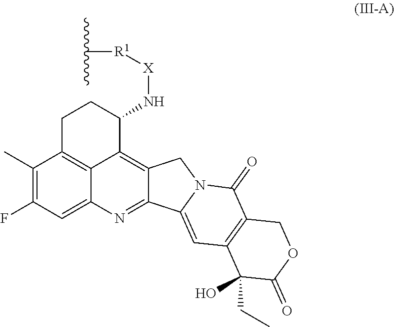

wherein R¹ is selected from the group consisting of: —O—, —(R²)N—, —P(═O)(R²)— and —S—; wherein R¹ links the structure according to formula (III-A) to the antibody either directly or optionally through a linker;
X is selected from the group consisting of: -L¹-C(R^{1a})(R^{1b})—C(O)—, -L¹-C(R^{1a})(R^{1b})—C(S)—, -L¹-L⁰- and -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
   wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
   m is selected from the group consisting of integers≥0, and n is selected from the group consisting of integers≥1;
   when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
   when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
   when R¹ is —O—, X is -L³-C(O)—, and 1 methylene unit of L³ is replaced by —NR⁸, R⁸ is not —CH₂—CH₂—NH₂;
   when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—;
and
wherein the antibody-drug conjugate optionally comprises a linker that links the antibody with the active agent.

### I-A Embodiments

In one embodiment, the antibody-drug conjugate comprises an active agent with a structure according to formula (I-A) or a tautomer, a mesomer, a racemate, an enantiomer or a diastereoisomer thereof, or a mixture thereof, or a pharmaceutically acceptable salt thereof:

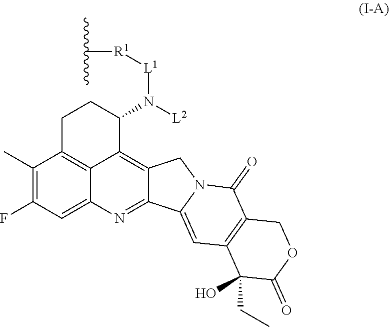

wherein, R¹ may be selected from the group consisting of: —O—, —(R²)N—, —P(═O)(R²)— and —S—;
L² may be —(C(R^{3a})(R^{3b}))ₘ—R, and m may be selected from the group consisting of integers≥1;
wherein 0 or no less than 1 methylene unit of L² may be independently replaced by -Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
L¹ may be —(C(R^{5a})(R^{5b}))ₙ—, and n may be selected from the group consisting of integers≥1;
wherein 0 or no less than 1 methylene unit of L¹ may be independently replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
Cy- may be selected from the group consisting of: 6-10 membered arylene, 5-8 membered heteroarylene, 3-10 membered heterocyclylene, and 3-10 membered saturated or partially unsaturated carbocyclylene, wherein -Cy- is unsubstituted or may be independently substituted with no less than 1 substituent R⁷;
wherein each R^{3a}, each R^{3b}, each R⁴, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R², each R⁷ and each R^{a} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (I-A) or a tautomer, a mesomer, a racemate, an enantiomer or a diastereoisomer thereof, or a mixture thereof, or a pharmaceutically acceptable salt thereof:

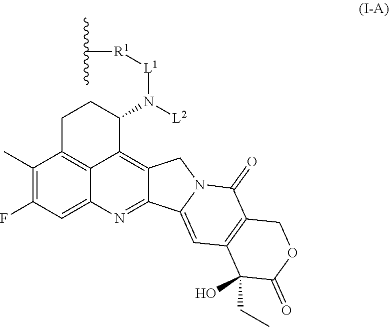

wherein, R¹ may be selected from the group consisting of: —O—, —(R²)N—, —P(═O)(R²)— and —S—;
L² may be —(C(R^{3a})(R^{3b}))ₘ—R, and m may be selected from the group consisting of integers≥1;
wherein 0 or no less than 1 methylene unit of L² may be independently replaced by -Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
L¹ may be —(C(R^{5a})(R^{5b}))ₙ—, and n may be selected from the group consisting of integers≥1;
wherein 0 or no less than 1 methylene unit of L¹ may be independently replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
Cy- may be selected from the group consisting of: 6-10 membered arylene, 5-8 membered heteroarylene, 3-10 membered heterocyclylene, and 3-10 membered saturated or partially unsaturated carbocyclylene, wherein -Cy- is unsubstituted or may be independently substituted with no less than 1 substituent R⁷;
wherein each R^{3a}, each R^{3b}, each R⁴, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R², each R⁷ and each R^{a} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (I-A) or a tautomer, a mesomer, a racemate, an enantiomer or a diastereoisomer thereof, or a mixture thereof, or a pharmaceutically acceptable salt thereof:

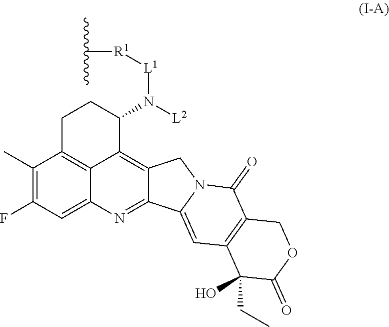

for example, wherein R^{3a} and R^{5a}, R⁴ and R^{5a}, R^{3a} and R⁶ or R⁴ and R⁶ may each independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸; each R^{3a}, each R^{3b}, each R⁴, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R; or R^{3a} and R^{5a}, R⁴ and R^{5a}, R^{3a} and R⁶ or R⁴ and R⁶ may each independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸;
for example, wherein R^{3a} and R^{5a} may form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸; each R^{3b}, each R⁴, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R; or R^{3a} and R^{5a}, R⁴ and R^{5a}, R^{3a} and R⁶ or R⁴ and R⁶ may each independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸;
for example, wherein R⁴ and R^{5a} may form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸; each R^{3a}, each R^{3b}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R; or R^{3a} and R^{5a}, R⁴ and R^{5a}, R^{3a} and R⁶ or R⁴ and R⁶ may each independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸;
for example, wherein R^{3a} and R⁶ may form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸; each R^{3b}, each R⁴, each R^{5a} and each R^{5b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R; or R^{3a} and R^{5a}, R⁴ and R^{5a}, R^{3a} and R⁶ or R⁴ and R⁶ may each independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸;
for example, wherein R⁴ and R⁶ may independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸; each R^{3a}, each R^{3b}, each R^{5a} and each R^{5b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R; or R^{3a} and R^{5a}, R⁴ and R^{5a}, R^{3a} and R⁶ or R⁴ and R⁶ may each independently optionally form a ring B together with an atom therebetween, wherein the ring B may be selected from the group consisting of: 5-8 membered heteroarylene and 3-10 membered saturated or partially unsaturated heterocyclylene, and the ring B is unsubstituted or may be substituted with no less than 1 substituent R⁸.

In one embodiment, the antibody-drug conjugate comprises an active agent with a structure according to formula (I-A) or a tautomer, a mesomer, a racemate, an enantiomer or a diastereoisomer thereof, or a mixture thereof, or a pharmaceutically acceptable salt thereof:

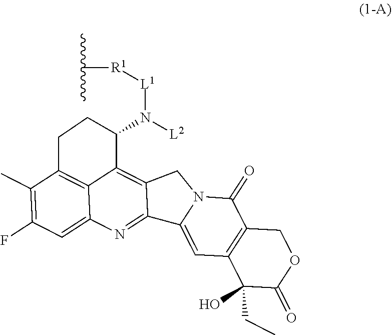

wherein, R¹ may be —O—;
L² may be —(C(R^{3a})(R^{3b}))ₘ—R, and m may be selected from the group consisting of integers from 1 to 3;
wherein 0 methylene units of L² may be replaced;
L¹ may be —(C(R^{5a})(R^{5b}))ₙ—, and n may be selected from the group consisting of integers from 2 to 4;
wherein 0, 1 or 2 methylene units of L¹ may be replaced by —N(R⁶)C(O)—, —C(O)—, —OC(O)—, —NR⁶—, —O— or —C(═S)—;
wherein each R^{3a}, each R^{3b}, each R^{5a}, each R^{5b} and each R⁶ may each independently be hydrogen, halogen, or a C₁₋₆ aliphatic group which may be optionally substituted with R; or R^{3a} and R^{5a} may form a ring B together with an atom therebetween, wherein the ring B may be selected from 5 membered saturated heterocyclylene, and the ring B is unsubstituted;
R may be hydrogen or halogen.

In one embodiment, the antibody-drug conjugate comprises an active agent with a structure according to formula (I-A) or a tautomer, a mesomer, a racemate, an enantiomer or a diastereoisomer thereof, or a mixture thereof, or a pharmaceutically acceptable salt thereof:

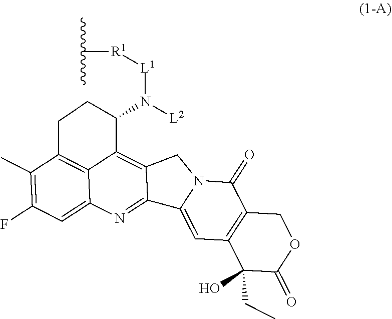

wherein, R¹ may be —O—;
L² may be —(C(R^{3a})(R^{3b}))ₘ—R, and m may be selected from the group consisting of integers 1 and 2;
wherein 0 methylene units of L² may be replaced;
L¹ may be —(C(R^{5a})(R^{5b}))ₙ—, and n may be selected from the group consisting of integers 2 and 3;
wherein 0 or 1 methylene unit of L¹ may be replaced by —C(O)—;
wherein each R^{3a}, each R^{3b}, each R^{5a} and each R^{5b} may each independently be hydrogen; or R^{3a} and R^{5a} may form a ring B together with an atom therebetween, wherein the ring B may be selected from 5 membered saturated heterocyclylene having 1 nitrogen heteroatom, and the ring B is unsubstituted;
R may be hydrogen.

In one embodiment, the antibody-drug conjugate comprises an active agent with a structure according to formula (I-A) or a tautomer, a mesomer, a racemate, an enantiomer or a diastereoisomer thereof, or a mixture thereof, or a pharmaceutically acceptable salt thereof:

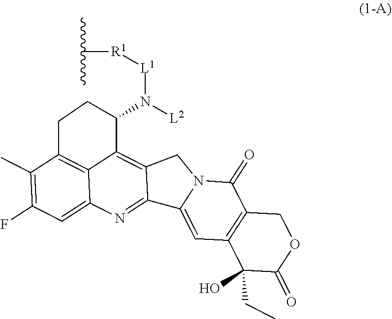

wherein, R¹ may be —O—;
L² may be —(C(R^{3a})(R^{3b}))ₘ—R, and m may be selected from the group consisting of integers 1 and 2;
wherein 0 methylene units of L² may be replaced;
L¹ may be —(C(R^{5a})(R^{5b}))₂—;
wherein 1 methylene unit of L¹ may be replaced by —C(O)—;
wherein each R^{3a}, each R^{3b}, each R^{5a} and each R^{5b} may each independently be hydrogen; or R^{3a} and R^{5a} may form a ring B together with an atom therebetween, wherein the ring B may be selected from 5 membered saturated heterocyclylene having 1 nitrogen heteroatom, and the ring B is unsubstituted;
R may be hydrogen.

In one embodiment, the antibody-drug conjugate comprises an active agent with a structure according to any one of the structures according to formula (I-A-1) to (I-A-17) or a tautomer, a mesomer, a racemate, an enantiomer or a diastereoisomer thereof, or a mixture thereof, or a pharmaceutically acceptable salt thereof:

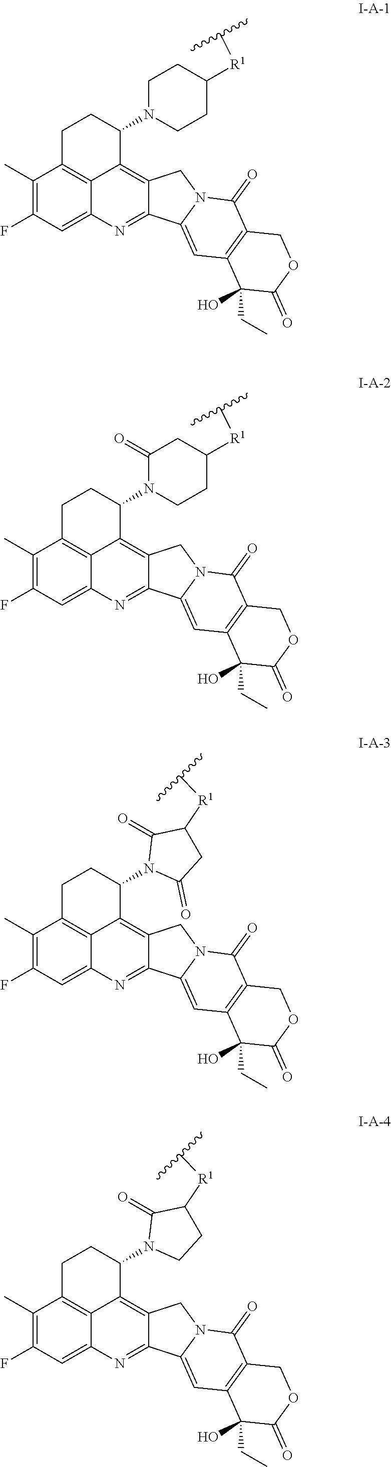

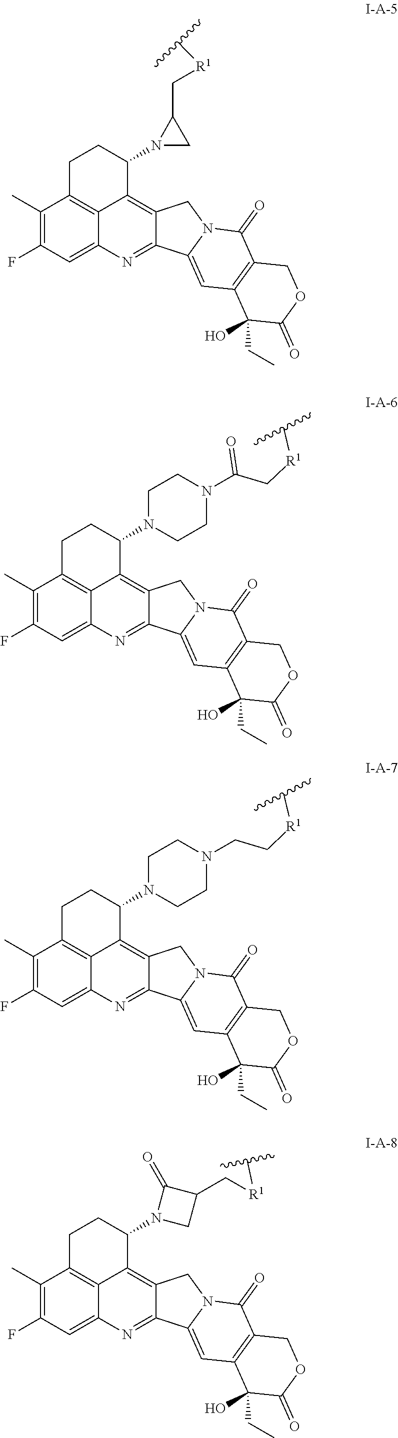

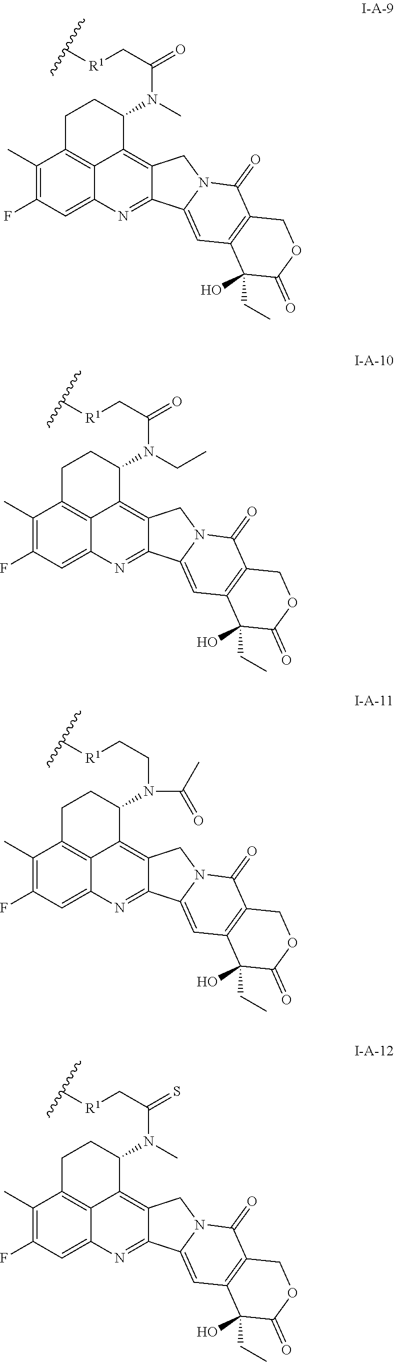

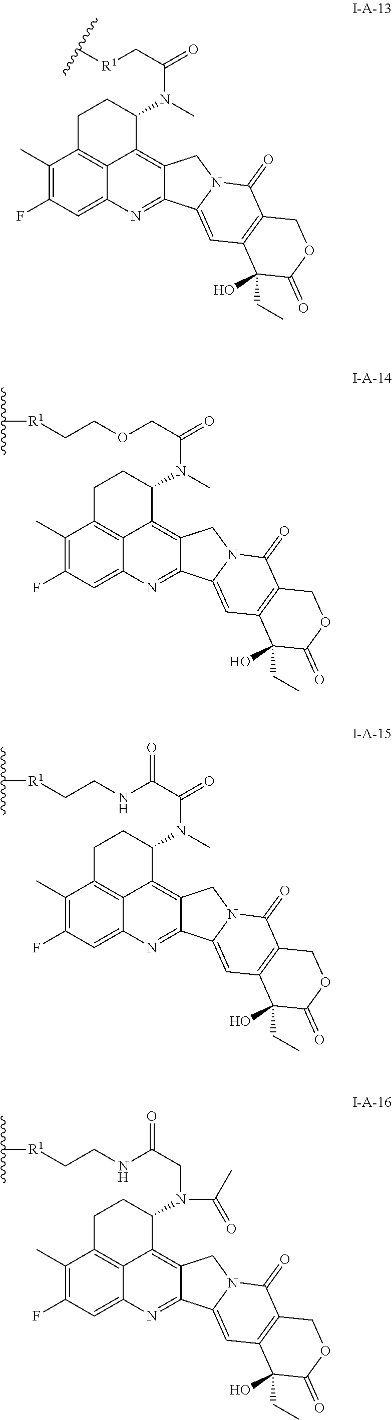

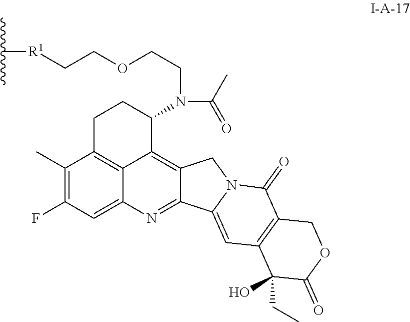

wherein, R¹ may be selected from the group consisting of: —O—, —HN—, —P(═O)H— and —S—, and wherein R¹ links the structure shown in any one of formulas (I-A-1) to (I-A-17) to the antibody as described herein either directly or through a linker.

### II-A Embodiments

In one embodiment, the antibody-drug conjugate comprises an active agent with a structure according to formula (II-A) or a tautomer, a mesomer, a racemate, an enantiomer or a diastereoisomer thereof, or a mixture thereof, or a pharmaceutically acceptable salt thereof:

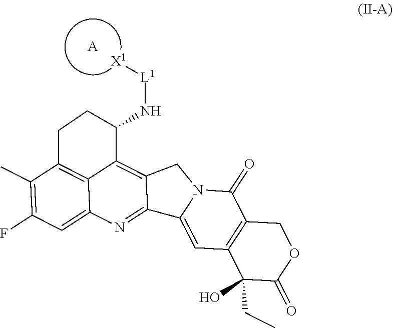

wherein, X¹ may be selected from the group consisting of: n, P, and saturated or unsaturated C; when X¹ may be saturated C, X¹ may be substituted with Rⁿ; ring A optionally links the structure shown as formula (II-A) to other molecular moieties;
when X¹ may be saturated C, ring A may be selected from the group consisting of: 3-10 membered saturated or partially unsaturated heterocyclyl, and 3-10 membered saturated or partially unsaturated carbocyclyl, wherein ring A may be substituted with 0 or no less than 1 substituent R^{1a};
or, when X¹ may be unsaturated C, ring A may be selected from the group consisting of: 6-10 membered aryl, 5-8 membered heteroaryl, 3-10 membered partially unsaturated heterocyclyl, and 3-10 membered partially unsaturated carbocyclyl, wherein ring A may be substituted with 0 or no less than 1 substituent R^{1b};
or, when X¹ may be N or P, ring A may be selected from the group consisting of: 5-8 membered heteroaryl and 3-10 membered saturated or partially unsaturated heterocyclyl, wherein ring A may be substituted with 0 or no less than 1 substituent R^{1c};
when ring A may be selected from the group consisting of: 6-10 membered aryl, 5-8 membered heteroaryl, and 3-10 membered saturated or partially unsaturated carbocyclyl, ring A may be substituted with p L², wherein L² cannot be Rⁿ;
or, when ring A may be 3-10 membered saturated or partially unsaturated heterocyclyl, ring A may be substituted with p L², or ring A may comprise q ring-forming heteroatom X², and X² is used to link the structure shown as formula (II-A) to other molecular moieties;
X² may be selected from the group consisting of: N and P;
L² may be —R²-L³-, and R² is used to link the structure shown as formula (II-A) to other molecular moieties;
L³ may be —(C(R^{3a})(R^{3b}))ₘ—, wherein when L³ may comprise a methylene unit, 0 or no less than 1 methylene unit of L³ may be independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—; R² may be selected from the group consisting of: —O—, —(R^{2a})N—, —S— and —P(═O)(R^{2a})—;
L¹ may be —(C(R^{5a})(R^{5b}))ₙ—, wherein when L¹ may comprise a methylene unit, 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1a}, each R^{1b}, each R^{1c}, each R^{2a}, each R^{3a}, each R^{3b}, each R⁴, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n may each independently be selected from the group consisting of integers≥0, and p and q may each independently be selected from the group consisting of integers≥1.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

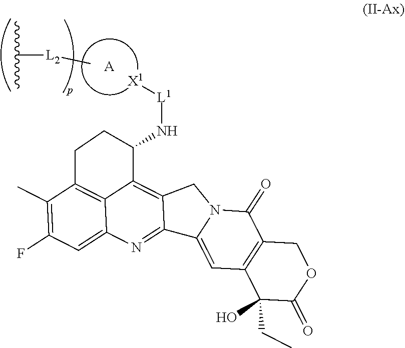

wherein, X¹ may be saturated C, and X¹ may be substituted with Rⁿ;
ring A may be selected from the group consisting of: 3-10 membered saturated or partially unsaturated heterocyclyl and 3-10 membered saturated or partially unsaturated carbocyclyl, wherein ring A may be substituted with 0 or no less than 1 substituent R^{1a};
ring A may be substituted with p L², wherein p may be selected from the group consisting of integers 1, and L² can not be Rⁿ;
L² may be —R²-L³-;
L³ may be —(C(R^{3a})(R^{3b}))ₘ, and m may be selected from the group consisting of integers≥0;
wherein when L³ may comprise a methylene unit, 0 or no less than 1 methylene unit of L³ may be independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² may be selected from the group consisting of: —O—, —(R^{2a})N—, —S— and —P(═O)(R^{2a})—;
L¹ may be —(C(R^{5a})(R^{5b}))ₙ—, and n may be selected from the group consisting of integers≥0;
wherein when L¹ may comprise a methylene unit, 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1a}, each R^{2a}, each R^{3a}, each R^{3b}, each R⁴, each R^{5a}, each R^{5b}, each R⁶ and each Rⁿ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

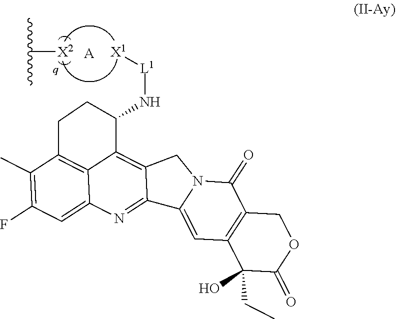

wherein, X¹ may be saturated C, and X¹ may be substituted with Rⁿ;
ring A may be 3-10 membered saturated or partially unsaturated heterocyclyl, and ring A can not be substituted or may be substituted with no less than 1 substituent R^{1a}; ring A may comprise q ring-forming heteroatom X², and X² is used for linking to the antibody, optionally via a linker; q may be selected from the group consisting of integers 1, and X² may be selected from the group consisting of: N and P;
L¹ may be —(C(R^{5a})(R^{5b}))ₙ—, and n may be selected from the group consisting of integers≥0;
wherein 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1a}, each R^{5a}, each R^{5b}, each R⁶ and each Rⁿ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

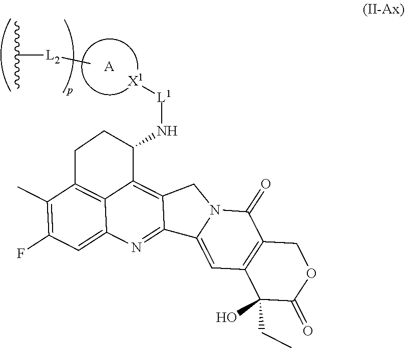

wherein, X¹ may be unsaturated C;
ring A may be selected from the group consisting of: 6-10 membered aryl, 5-8 membered heteroaryl, 3-10 membered partially unsaturated heterocyclyl, and 3-10 membered partially unsaturated carbocyclyl, and ring A can not be substituted or may be substituted with no less than 1 substituent R^{1b};
ring A may be substituted with p L², wherein p may be selected from the group consisting of integers≥1;
L² may be —R²-L³-, and R² is used for linking to the antibody, optionally via a linker;
L³ may be —(C(R^{3a})(R^{3b}))ₘ, and m may be selected from the group consisting of integers≥0;
wherein 0 or no less than 1 methylene unit of L³ may be independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² may be selected from the group consisting of: —O—, —(R^{2a})N—, —S— and —P(═O)(R^{2a})—;
L¹ may be —(C(R^{5a})(R^{5b}))ₙ—, and n may be selected from the group consisting of integers≥0;
wherein 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1b}, each R^{2a}, each R^{3a}, each R^{3b}, each R⁴, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

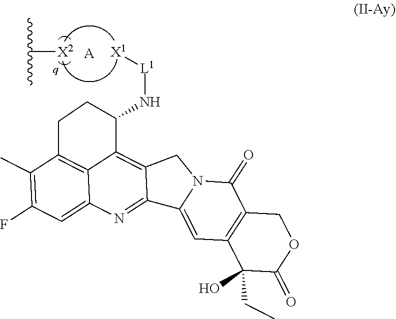

wherein, X¹ may be unsaturated C;
ring A may be 3-10 membered partially unsaturated heterocyclyl, and ring A can not be substituted or may be substituted with no less than 1 substituent R^{1b};
ring A may comprise q ring-forming heteroatom X², and X² is used for linking to the antibody either directly or through a linker; q may be selected from the group consisting of integers≥1, and X² may be selected from the group consisting of: N and P;
L¹ may be —(C(R^{5a})(R^{5b}))ₙ—, and n may be selected from the group consisting of integers≥0;
wherein 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1b}, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

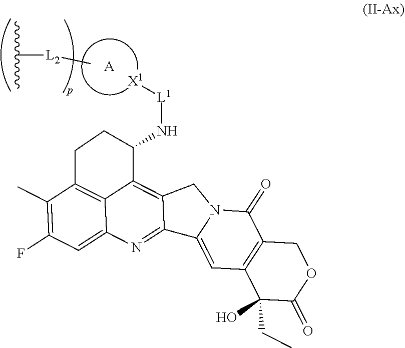

wherein, X¹ may be N or P;
ring A may be selected from the group consisting of: 5-8 membered heteroaryl and 3-10 membered saturated or partially unsaturated heterocyclyl, and ring A can not be substituted or may be substituted with no less than 1 substituent R^{1c};
ring A may be substituted with p L², wherein p may be selected from the group consisting of integers≥1;
L² may be —R²-L³-, and R² is used for linking to the antibody either directly or through a linker;
L³ may be —(C(R^{3a})(R^{3b}))ₘ, and m may be selected from the group consisting of integers≥0;
wherein 0 or no less than 1 methylene unit of L³ may be independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² may be selected from the group consisting of: —O—, —(R^{2a})N—, —S— and —P(═O)(R^{2a})—;
L¹ may be —(C(R^{5a})(R^{5b}))ₙ—, and n may be selected from the group consisting of integers≥0;
wherein 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1c}, each R^{2a}, each R^{3a}, each R^{3b}, each R⁴, each R^{5a}, each R^{5b} and each
R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

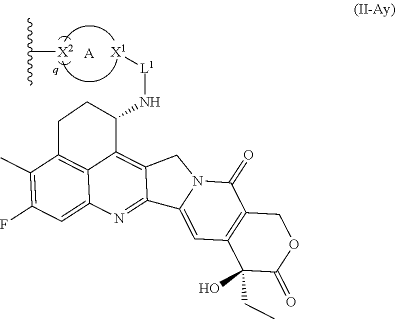

wherein, X¹ may be N or P;
ring A may be 3-10 membered saturated or partially unsaturated heterocyclyl, and ring A cannot be substituted or may be substituted with no less than 1 substituent R^{1c};
ring A may comprise q ring-forming heteroatom X², and X² is used for linking to the antibody either directly or through a linker; q may be selected from the group consisting of integers≥1, and X² may be selected from the group consisting of: N and P;
L¹ may be —(C(R^{5a})(R^{5b}))ₙ—, and n may be selected from the group consisting of integers≥0;
wherein 0 or no less than 1 methylene unit of L¹ may be independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1c}, each R^{5a}, each R^{5b} and each R⁶ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

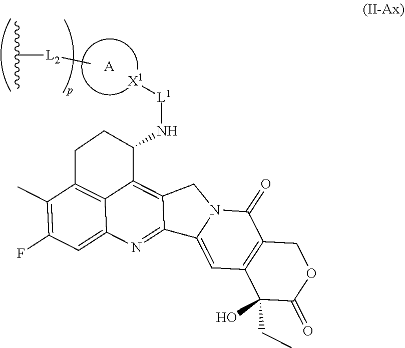

wherein, X¹ may be saturated C, and X¹ may be substituted with Rⁿ;
ring A may be selected from the group consisting of: 3-6 membered saturated heterocyclyl and 3-6 membered saturated or partially unsaturated carbocyclyl;
p may be 1, and L² can not be Rⁿ;
L² may be —R²-L³-;
L³ may be —(C(R^{3a})(R^{3b}))ₘ—, and m may be selected from the group consisting of integers from 0 to 2, wherein when L³ may comprise a methylene unit, 0 or 1 methylene unit of L³ may be replaced by —C(O)— or —C(═S)—;
R² may be selected from —O—;
L¹ may be —(C(R^{5a})(R^{5b}))ₙ—, and n may be selected from the group consisting of 0 and 1;
wherein when L¹ may comprise a methylene unit, 0 or 1 methylene unit of L¹ may be replaced by —C(O)— or —C(═S)—;
wherein each R^{3a}, each R^{3b}, each R^{5a}, each R^{5b} and each Rⁿ may each independently be hydrogen, halogen, or a C₁₋₆ aliphatic group which may be optionally substituted with R;
wherein each R may independently be hydrogen or halogen.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

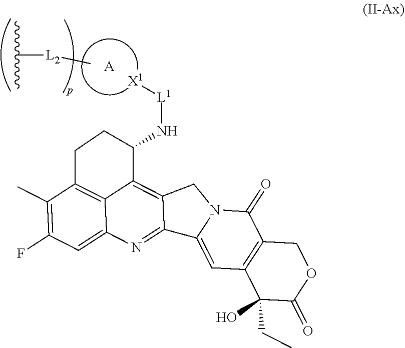

wherein, X¹ may be saturated C, and X¹ is linked to Rⁿ, wherein Rⁿ may be H;
ring A may be selected from the group consisting of: 5 membered saturated heterocyclyl having 1 nitrogen heteroatom, and 4-6 membered saturated carbocyclyl;
P may be 1;
L² may be —R²-L³-, and L³ is directly linked to ring A;
L³ may be —(C(R^{3a})(R^{3b}))ₘ—, and m may be 0 or 2;
R² may be —O—;
L¹ may be —C(O)—;
wherein each R^{3a} and each R^{3b} may each independently be hydrogen.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

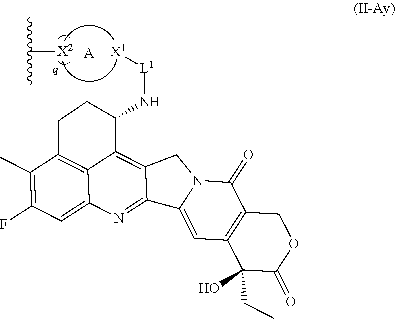

wherein, X¹ may be saturated C, and X¹ may be substituted with Rⁿ;
ring A may be 3-6 membered saturated heterocyclyl;
ring A may comprise 1 ring-forming heteroatom N, and N is used for linking to the antibody either directly or through a linker;
L¹ may be —(C(R^{5a})(R^{5b}))ₙ—, and n may be selected from the group consisting of 0 and 1;
wherein 0 or 1 methylene unit of L¹ may be replaced by —C(O)— or —C(═S)—;
wherein each R^{5a}, each R^{5b} and each Rⁿ may each independently be hydrogen, halogen, or a C₁₋₆ aliphatic group which may be optionally substituted with R;
wherein each R may independently be hydrogen or halogen.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

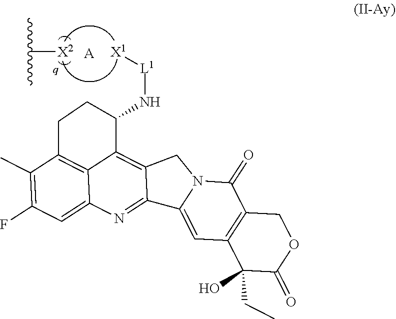

wherein, X¹ may be saturated C, and X¹ may be substituted with H;
ring A may be 5 membered saturated heterocyclyl having 1 heteroatom N;
ring A may comprise 1 ring-forming heteroatom N, and N is used for linking to the antibody either directly or through a linker;
L¹ may be —C(O)—.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

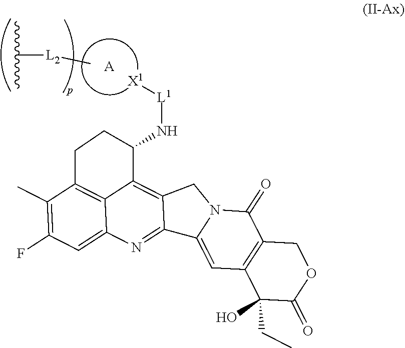

wherein, X¹ may be unsaturated C;
ring A may be selected from the group consisting of: 6 membered aryl, 5-8 membered heteroaryl, 3-10 membered partially unsaturated heterocyclyl, and 3-10 membered partially unsaturated carbocyclyl, and ring A can not be substituted or may be independently substituted with 1 substituent R^{1b};
P may be 1;
L² may be —R²-L³-, and R² is used for linking to the antibody either directly or through a linker;
L³ may be —C(R^{3a})(R^{3b})—;
R² may be selected from the group consisting of: —O—, —(R^{2a})N— and —S—;
L¹ may be —C(R^{5a})(R^{5b})—;
wherein 0 or 1 methylene unit of L¹ may be replaced by —C(O)— or —C(═S)—;
wherein each R^{1b}, each R^{2a}, each R^{3a}, each R^{3b}, each R^{5a} and each R^{5b} may each independently be hydrogen, halogen, or a C₁₋₆ aliphatic group which may be optionally substituted with R;
wherein each R may independently be hydrogen or halogen.

For example, the compound may comprise a structure shown as formula (II-Ax):
wherein, X¹ may be unsaturated C;
ring A may be selected from the group consisting of: 6 membered aryl and 5-8 membered heteroaryl;
P may be 1;
L² may be —R²-L³-, and is used for linking to the antibody either directly or through a linker;
L³ may be —C(R^{3a})(R^{3b})—;
R² may be selected from the group consisting of: —O—, —(R^{2a})N— and —S—;
L¹ may be —C(R^{5a})(R^{5b})—;
wherein 0 or 1 methylene unit of L¹ may be replaced by —C(O)— or —C(═S)—;
wherein each R^{2a}, each R^{3a}, each R^{3b}, each R^{5a} and each R^{5b} may each independently be hydrogen, halogen, or a C₁₋₆ aliphatic group which may be optionally substituted with R;
wherein each R may independently be hydrogen or halogen.

For example, the compound may comprise a structure shown as formula (II-Ax):
wherein, X¹ may be unsaturated C;
ring A may be 6 membered aryl;
P may be 1;
L² may be —R²-L³-, and R² is used for linking to the antibody either directly or through a linker;
L³ may be —C(R^{3a})(R^{3b})—;
R² may be —O—;
L¹ may be —C(O)—;
wherein each R^{3a}, each R^{3b}, each R^{5a} and each R^{5b} may each independently be hydrogen or a C₁₋₆ aliphatic group.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

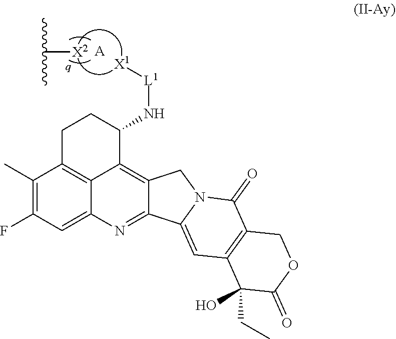

wherein, X¹ may be unsaturated C;
ring A may be 5 membered partially unsaturated heterocyclyl;
ring A may comprise 1 ring-forming heteroatom N, and N is used for linking to the antibody either directly or through a linker;
L¹ may be —C(R^{5a})(R^{5b})
wherein 0 or 1 methylene unit of L¹ may be replaced by —C(O)— or —C(═S)—;
wherein each R^{5a} and each R^{5b} may each independently be hydrogen, halogen, or a C₁₋₆ aliphatic group which may be optionally substituted with R;
wherein each R may independently be hydrogen or halogen.

In one embodiment, the antibody-drug conjugate comprises an active agent with a structure according to formula (II-Ax) or a tautomer, a mesomer, a racemate, an enantiomer or a diastereoisomer thereof, or a mixture thereof, or a pharmaceutically acceptable salt thereof:

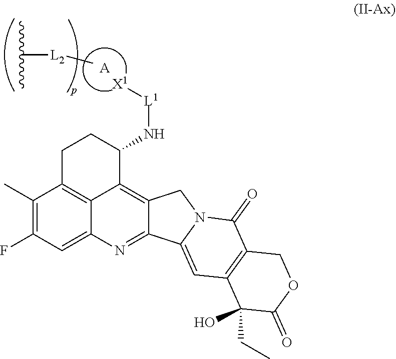

wherein, X¹ may be N;
ring A may be 6 membered saturated heterocyclyl;
P may be 1;
L² may be —R²-L³-, and R² is used for linking to the antibody either directly or through a linker;
L³ may be —(C(R^{3a})(R^{3b}))ₘ—, and m may be 1 or 2,
wherein 0 or 1 methylene unit of L³ may be replaced by —C(O)— or —C(═S)—;
R² may be selected from the group consisting of: —O—, —(R^{2a})N— and —S—;
L¹ may be —C(R^{5a})(R^{5b})—,
wherein 1 methylene unit of L¹ may be replaced by —C(O)— or —C(═S)—;
wherein each R^{2a}, each R^{3a}, each R^{3b}, each R^{5a} and each R^{5b} may each independently be hydrogen, halogen, or a C₁₋₆ aliphatic group which may be optionally substituted with R;
wherein each R may independently be hydrogen or halogen.

In one embodiment, the antibody-drug conjugate comprises an active agent with a structure according to formula (II-Ay) or a tautomer, a mesomer, a racemate, an enantiomer or a diastereoisomer thereof, or a mixture thereof, or a pharmaceutically acceptable salt thereof:

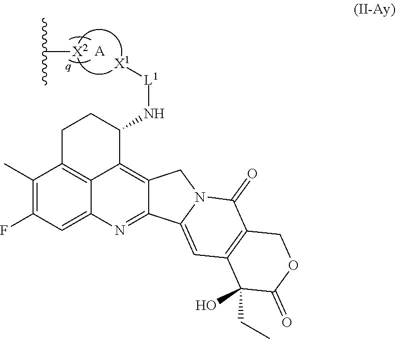

wherein, X¹ may be N;
ring A may be 5 membered partially unsaturated heterocyclyl;
ring A may comprise 1 ring-forming heteroatom N, and N is used for linking to the antibody either directly or through a linker;
L¹ may be —C(R^{5a})(R^{5b})
wherein no less than 1 methylene unit of L¹ may be replaced by —C(O)— or —C(═S)—;
wherein each R^{5a} and each R^{5b} may each independently be hydrogen, halogen, or a C₁₋₆ aliphatic group which may be optionally substituted with R;
wherein each R may independently be hydrogen or halogen.

In one embodiment, the antibody-drug conjugate comprises an active agent with a structure according to any one of formulas (II-A-1) to (II-A-12), or a tautomer, a mesomer, a racemate, an enantiomer or a diastereoisomer thereof, or a mixture thereof, or a pharmaceutically acceptable salt thereof:

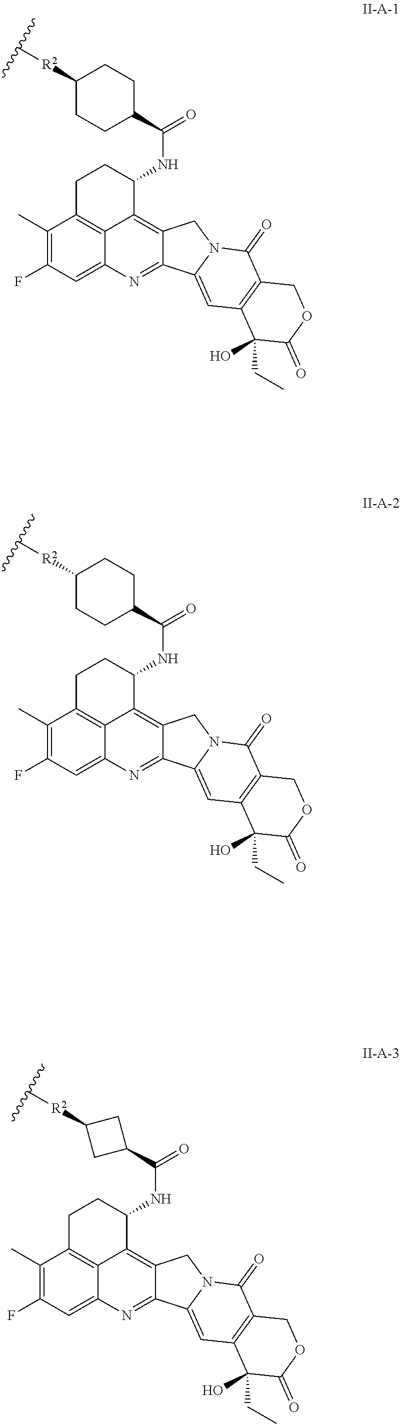

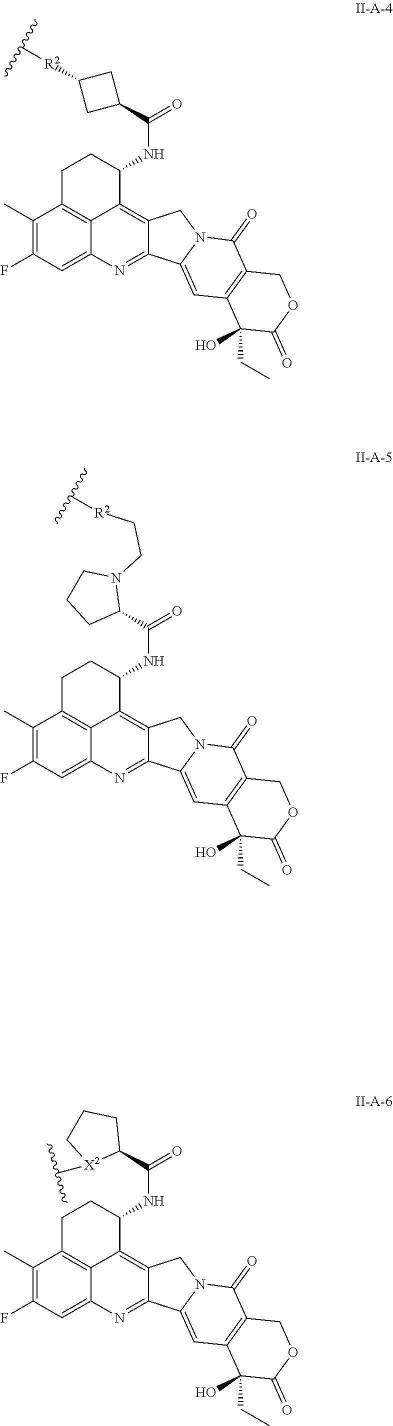

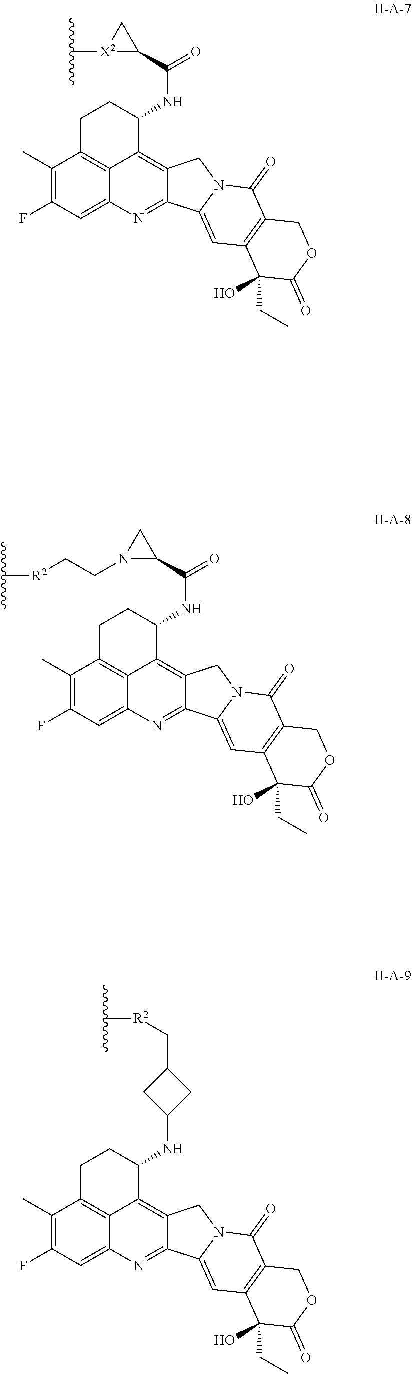

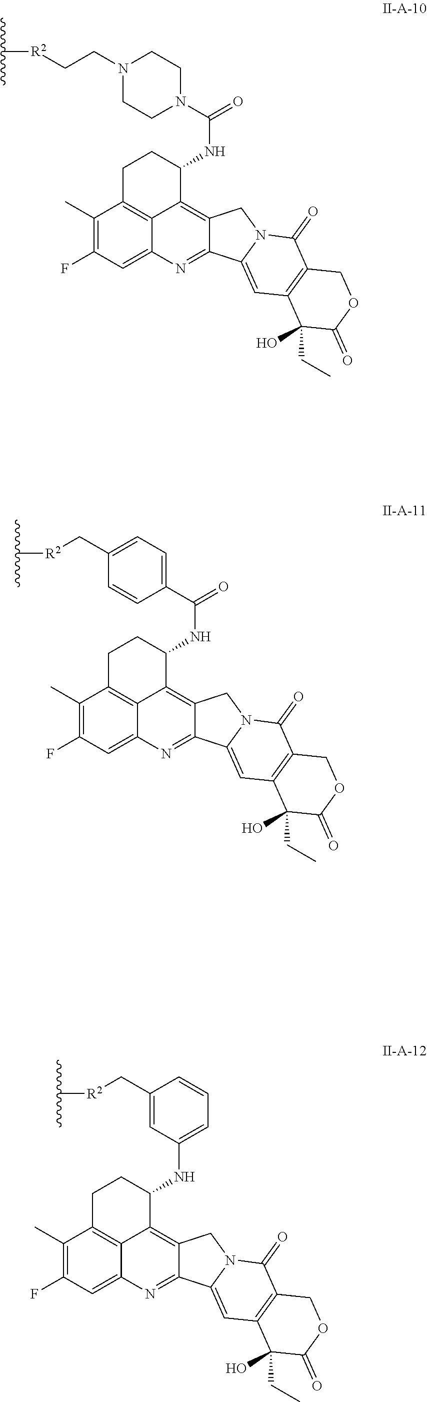

wherein, R² may be selected from the group consisting of: —O—, —HN—, —P(═O)H— and —S—; X² may be selected from the group consisting of N and P; wherein R² or X² links any one the structures shown in formulas (II-A-1) to (II-A-12) to the antibody as described herein either directly or through a linker.

### III-A Embodiments

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

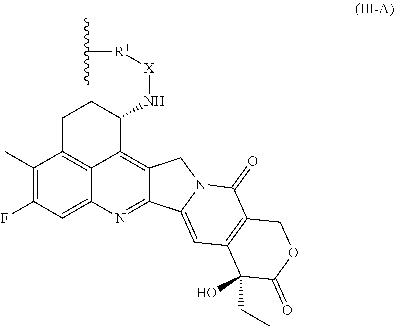

wherein, R¹ may be selected from the group consisting of: —O—, —(R²)N—, —P(═O)(R²)— and —S—;
X may be selected from the group consisting of: -L¹-C(R^{1a})(R^{1b})—C(O)—, -L¹-C(R^{1a})(R^{1b})—C(S)—, -L¹-L⁰- and -L³-L²-;
L¹ may be —(C(R^{3a})(R^{3b}))ₘ—, wherein when L¹ may comprise a methylene unit, 0 or no less than 1 methylene unit of L¹ may be independently replaced by —C(O)—, —C(═S)—, —C(═NR^{4b})— or —C(═N₂)—;
L⁰ may be —C(R^{2a})(R^{2b})—, or L⁰ may be —C(═S)—, —C(═NR^{4a})— or —C(═N₂)—;
L² may be —C(R^{5a})(R^{5b})—, wherein 0 or 1 methylene unit of L² may be replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ may be —(C(R^{7a})(R^{7b}))ₙ—, wherein no less than 1 methylene unit of L³ may be independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ may also independently be replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R^{a} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m may be selected from the group consisting of integers≥0, and n may be selected from the group consisting of integers≥1;
when R¹ may be —O— or —HN—, and X may be -L¹-CH₂—C(O)—, and when L¹ may comprise a methylene unit, no less than 1 methylene unit of L¹ may be independently replaced by —C(O)—, —C(═S)—, —C(═NR^{4b})— or —C(═N₂)—, or R^{3a} and R^{3b} can not be both hydrogen in each —C(R^{3a})(R^{3b})— of L¹;
when R¹ may be —HN—, X may be -L¹-L⁰-, and L⁰ may be —CH₂—, no less than 1 methylene unit of L¹ may be independently replaced by —C(O)—, —C(═S)—, —C(═NR^{4b})— or —C(═N₂)—, or each R^{3a} and each R^{3b} can not be both hydrogen;
when R¹ may be —O—, X may be -L³-C(O)—, and 1 methylene unit of L³ may be replaced by —NR⁸, R⁸ can not be —CH₂—CH₂—NH₂;
when R¹ may be —NH—, and X may be -L³-C(O)—, no less than 1 methylene unit of L³ may be replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

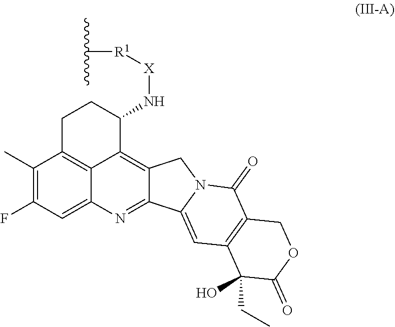

wherein, R¹ may be selected from the group consisting of: —O—, —(R²)N— and —S—;
X may be -L¹-C(R^{1a})(R^{1b})—C(S)—;
L¹ may be —(C(R^{3a})(R^{3b}))ₘ, and m may be selected from the group consisting of integers≥0;
wherein 0 or no less than 1 methylene unit of L¹ may be independently replaced by —C(O)—, —C(═S)—, —C(═NR^{4b})— or —C(═N₂)—;
R² may be halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R^{1a}, each R^{1b}, each R^{3a}, each R^{3b} and each R^{4b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

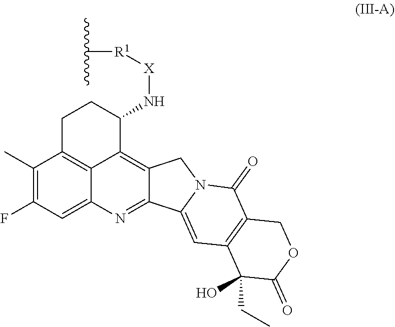

wherein, R¹ may be —S— or —(R²)N—;
X may be -L¹-C(R^{1a})(R^{1b})—C(O)—;
L¹ may be —(C(R^{3a})(R^{3b}))ₘ, and m may be selected from the group consisting of integers≥0;
wherein 0 or no less than 1 methylene unit of L¹ may be independently replaced by —C(O)—, —C(═S)—, —C(═NR^{4b})— or —C(═N₂)—;
R² may be halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R^{1a}, each R^{1b}, each R^{3a}, each R^{3b} and each R^{4b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

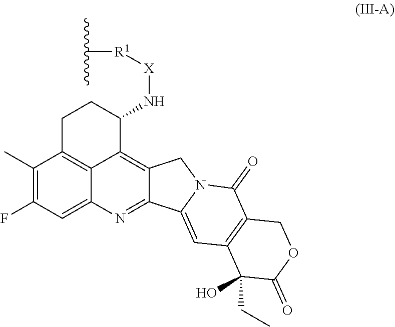

wherein, R¹ may be —O— or —HN—;
X may be -L¹-CH₂—C(O)—;
L¹ may be —(C(R^{3a})(R^{3b}))ₘ, and m may be selected from the group consisting of integers≥1;
wherein no less than 1 methylene unit of L¹ may be independently replaced by —C(O)—, —C(═S)—, —C(═NR^{4b})— or —C(═N₂)—;
wherein each R^{3a}, each R^{3b} and each R^{4b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

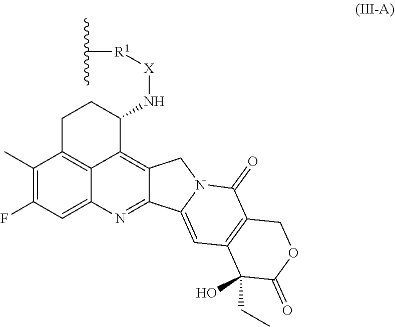

wherein, R¹ may be —O— or —HN—;
X may be -L¹-CH₂—C(O)—;
L¹ may be —(C(R^{3a})(R^{3b}))ₘ, and m may be selected from the group consisting of integers≥0;
R^{3a} and R^{3b} can not be both hydrogen in each —C(R^{3a})(R^{3b})—, or no less than 1 methylene unit of L¹ may be independently replaced by —C(O)—, —C(═S)—, —C(═NR^{4b})— or —C(═N₂)—;
wherein each R^{3a}, each R^{3b} and each R^{4b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

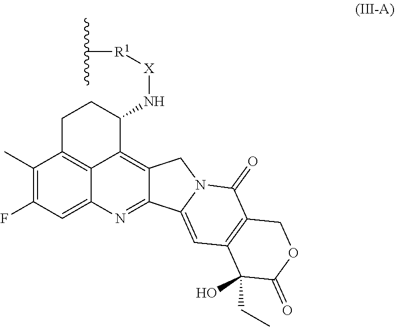

wherein, R¹ may be —O—, —S— or —(R²)N—;
X may be -L¹-L⁰-;
L⁰ may be —C(R^{2a})(R^{2b})—, or L⁰ may be —C(═S)—, —C(═NR^{4a})— or —C(═N₂)—;
L¹ may be —(C(R^{3a})(R^{3b}))ₘ, and m may be selected from the group consisting of integers≥0;
wherein 0 or no less than 1 methylene unit of L¹ may be independently replaced by —C(O)—, —C(═S)—, —C(═NR^{4b})— or —C(═N₂)—;
R² may be halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R^{2a}, each R^{2b}, R^{3a}, each R^{3b}, each R^{4a} and each R^{4b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

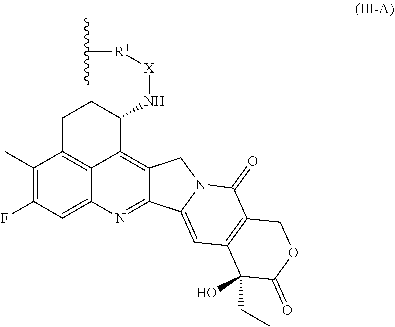

wherein, R¹ may be —HN—;
X may be -L¹-L⁰-;
L⁰ may be —C(R^{2a})(R^{2b})—, or L⁰ may be —C(═S)—, —C(═NR^{4a})— or —C(═N₂)—;
L¹ may be —(C(R^{3a})(R^{3b}))ₘ—, m may be selected from the group consisting of integers≥0, and each R^{3a} and each R^{3b} can not be both hydrogen;
wherein 0 or no less than 1 methylene unit of L¹ may be independently replaced by —C(O)—, —C(═S)—, —C(═NR^{4b})— or —C(═N₂)—;
wherein each R^{2a}, each R^{2b}, R^{3a}, each R^{3b}, each R^{4a} and each R^{4b} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

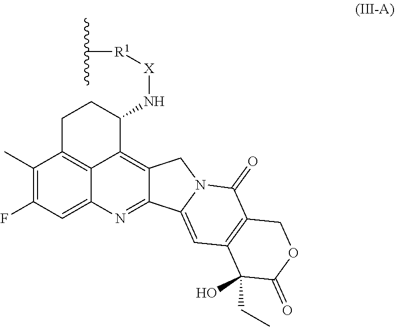

wherein, X may be -L³-L²-;
L² may be —C(R^{5a})(R^{5b})—, wherein 0 or 1 methylene unit of L² may be replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
R¹ may be —S— or —(R²)N—; or R¹ may be —O— and L² can not be —C(O)—; or R¹ may be —NH— and L² can not be —C(O)—; L³ may be —(C(R^{7a})(R^{7b}))ₙ—, and n may be selected from the group consisting of integers≥1;
wherein no less than 1 methylene unit of L³ may be independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ may also be independently replaced by —C(O)—, —C(═S)—, —C(═NR⁸)— or —C(═N₂)—;
R² may be halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R^{5a}, each R^{5b}, each R⁶, each R^{7a}, each R^{7b} and each R⁸ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

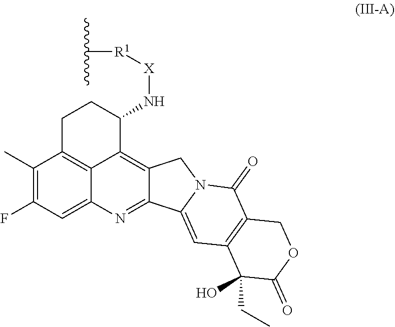

wherein, R¹ may be —O—;
X may be -L³-L²-;
wherein L² may be —C(O)—;
L³ may be —(C(R^{7a})(R^{7b}))ₙ—, and n may be selected from the group consisting of integers≥0;
wherein no less than 1 methylene unit of L³ may be independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ may also be independently replaced by —C(O)—, —C(═S)—, —C(═NR⁸)— or —C(═N₂)—;
wherein each R^{7a}, each R^{7b} and each R⁸ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R or a C₁₋₆ aliphatic group which may be optionally substituted with R;
wherein R, R^{a} and R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group; when 1 methylene unit of L³ may be replaced by —NR⁸, R⁸ can not be a C₁₋₆ aliphatic group which may be substituted with —NH₂.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

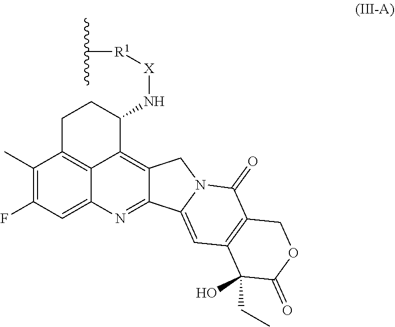

wherein, R¹ may be —HN—;
X may be -L³-L²-;
wherein L² may be —C(O)—;
L³ may be —(C(R^{7a})(R^{7b}))ₙ—, and n may be selected from the group consisting of integers≥1;
no less than 1 methylene unit of L³ may be replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ may also be independently replaced by —C(O)N(R⁸)—, —NR⁸— or —O—, and 0 or no less than 1 methylene unit of L³ may also be independently replaced by —C(O)—, —C(═S)—, —C(═NR⁸)— or —C(═N₂)—;
wherein each R^{7a}, each R^{7b} and each R⁸ may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R or a C₁₋₆ aliphatic group which may be optionally substituted with R;
wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

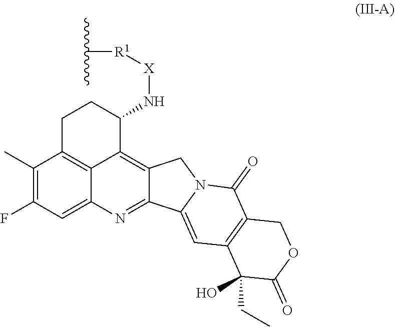

wherein, R¹ may be —O—, —S— or —(R²)N—;
X may be -L¹-C(R^{1a})(R^{1b})—C(S)—;
L¹ may be —(C(R^{3a})(R^{3b}))ₘ—, and m may be 0, 1 or 2;
wherein 0 or 1 methylene unit of L¹ may be replaced by —C(O)—;
wherein each R^{1a}, each R^{1b}, each R², each R^{3a} and each R^{3b} may each independently be hydrogen, or a C₁₋₆ aliphatic group which may be optionally substituted with R;
wherein each R may be hydrogen.

For example, the compound may comprise a structure shown as formula (III-A):
wherein, R¹ may be —O—;
X may be -L¹-C(R^{1a})(R^{1b})—C(S)—;
L¹ may be —(CH₂)ₘ—, and m may be 1 or 2;
wherein 0 or 1 methylene unit of L¹ may be replaced by —C(O)—;
wherein each R^{1a} and each R^{1b} may each independently be hydrogen, or a C₁₋₆ aliphatic group which may be optionally substituted with R;
wherein each R may be hydrogen.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

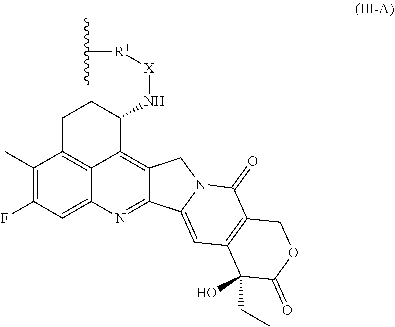

wherein, R¹ may be —S— or —(R²)N—;
X may be -L¹-C(R^{1a})(R^{1b})—C(O)—;
L¹ may be —(C(R^{3a})(R^{3b}))ₘ—, and m may be 0, 1 or 2;
wherein 0 or 1 methylene unit of L¹ may be replaced by —C(O)—;
R² may be a C₁₋₆ aliphatic group;
wherein each R^{1a}, each R^{1b}, each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

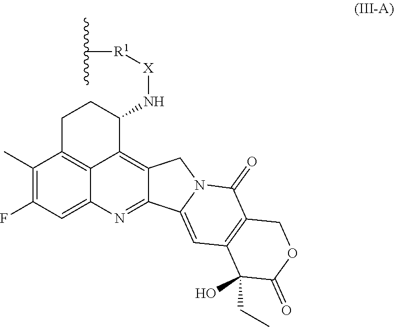

wherein, R¹ may be —S— or —(R²)N—;
X may be -L¹-C(R^{1a})(R^{1b})—C(O)—;
L¹ may be —(C(R^{3a})(R^{3b}))ₘ—, and m may be 1 or 2;
wherein 0 or 1 methylene unit of L¹ may be replaced by —C(O)—;
R² may be a C₁₋₆ aliphatic group;
wherein each R^{1a}, each R^{1b}, each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

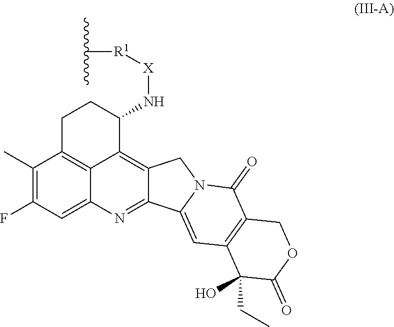

wherein, when R¹ may be —S— or —(R²)N—,
X may be -L¹-C(R^{1a})(R^{1b})—C(O)—,
L¹ may be —(C(R^{3a})(R^{3b}))ₘ—, and m may be 0, 1 or 2,
0 or 1 methylene unit of L¹ may be replaced by —C(O)—;
R² may be a C₁₋₆ aliphatic group;
wherein each R^{1a}, each R^{1b}, each R^{3a} and each R^{3b} may each independently be hydrogen or a C₁₋₆ aliphatic group;
or, when R¹ may be —O—,
X may be -L¹-CH₂—C(O)—, and
L¹ may be —(C(R^{3a})(R^{3b}))₂—,
0 or 1 methylene unit of L¹ may be replaced by —C(O)—;
wherein each R^{3a} and each R^{3b} may each independently be hydrogen or a C₁₋₆ aliphatic group;
each R^{3a} and each R^{3b} can not be both hydrogen, or 1 methylene unit of L¹ may be replaced by —C(O)—;
wherein each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

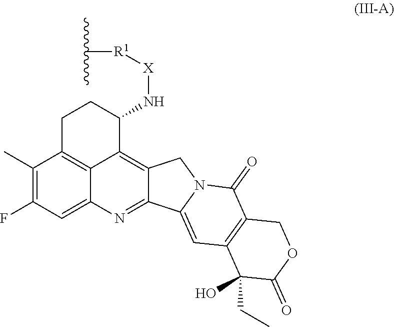

wherein, R¹ may be —O—;
X may be -L¹-CH₂—C(O)—;
L¹ may be —(C(R^{3a})(R^{3b}))₂—;
wherein 0 or 1 methylene unit of L¹ may be replaced by —C(O)—;
wherein each R^{3a} and each R^{3b} may each independently be hydrogen or a C₁₋₆ aliphatic group;
each R^{3a} and each R^{3b} can not be both hydrogen, or 1 methylene unit of L¹ may be replaced by —C(O)—;
wherein each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

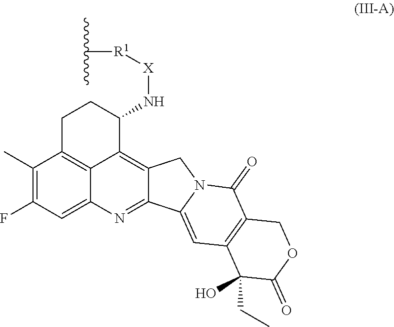

wherein, when R¹ may be —S— or —(R²)N—,
X may be -L¹-C(R^{1a})(R^{1b})—C(O)—,
L¹ may be —(C(R^{3a})(R^{3b}))ₘ—, and m may be 0, 1 or 2,
0 or 1 methylene unit of L¹ may be replaced by —C(O)—;
R² may be a C₁₋₆ aliphatic group;
wherein each R^{1a}, each R^{1b}, each R^{3a} and each R^{3b} may each independently be hydrogen or a C₁₋₆ aliphatic group;
or, when R¹ may be —O—, and
X may be -L¹-CH₂—C(O)—,
L¹ may be —C(R^{3a})(R^{3b})—, and R^{3a} and R^{3b} can not be both hydrogen in each —C(R^{3a})(R^{3b})—;
wherein each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

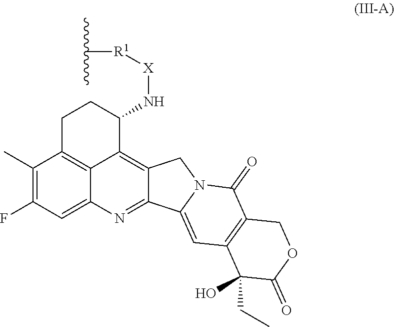

wherein, R¹ may be —O—;
X may be -L¹-CH₂—C(O)—;
L¹ may be —(C(R^{3a})(R^{3b}))₂—;
wherein 0 or 1 methylene unit of L¹ may be replaced by —C(O)—;
wherein each R^{3a} and each R^{3b} may each independently be hydrogen or a C₁₋₆ aliphatic group;
R^{3a} and R^{3b} can not be both hydrogen in each —C(R^{3a})(R^{3b}), or 1 methylene unit of L¹ may be replaced by —C(O)—;
wherein each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

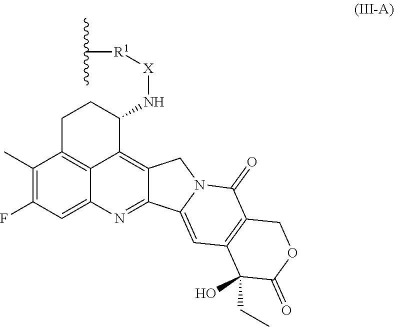

wherein, R¹ may be —O—;
X may be -L¹-CH₂—C(O)—;
L¹ may be —(CH₂)₂—;
wherein 1 methylene unit of L¹ may be replaced by —C(O)—.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

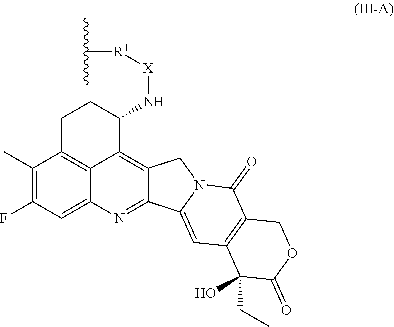

wherein, R¹ may be —O—;
X may be -L¹-CH₂—C(O)—;
L¹ may be —C(R^{3a})(R^{3b})—ₘ-, m may be selected from the group consisting of integers from 1 to 5, and R^{3a} and R^{3b} can not be both hydrogen in each —C(R^{3a})(R^{3b})—;
wherein each R^{3a} and each R^{3b} may each independently be hydrogen, halogen, or a C₁₋₆ aliphatic group which may be optionally substituted with R;
wherein each R may be hydrogen or halogen.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

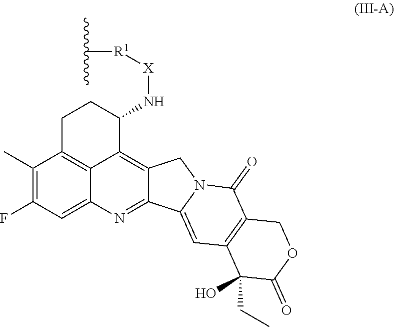

wherein, R¹ may be —O—;
X may be -L¹-CH₂—C(O)—;
L¹ may be —C(R^{3a})(R^{3b})—, and R^{3a} and R^{3b} can not be both hydrogen;
wherein each R^{3a} and each R^{3b} may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

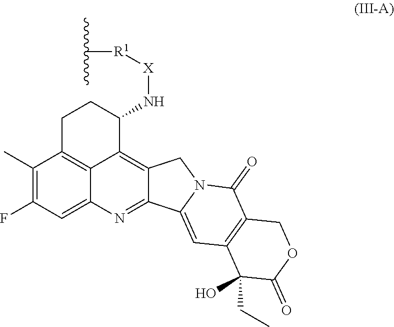

wherein, R¹ may be —O— or —(R²)N—;
X may be -L¹-L⁰-;
L⁰ may be —CH₂—, or L⁰ may be —C(═S)—;
L¹ may be —(CH₂)ₘ—, and m may be selected from the group consisting of integers from 0 to 2;
wherein 0 or 1 methylene unit of L¹ may be replaced by —C(O)— or —C(═S)—;
R² may be a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

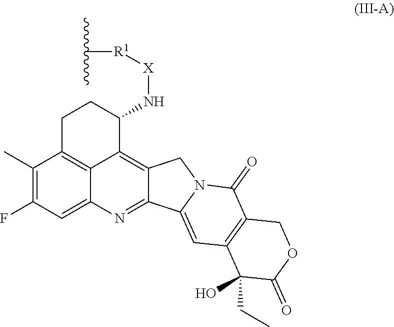

wherein, R¹ may be —NH—;
X may be -L¹-L⁰-;
L⁰ may be —CH₂—, or L⁰ may be —C(═S)—;
L¹ may be —(CH₂)ₘ—, and m may be selected from the group consisting of integers from 0 to 2;
wherein 0 or 1 methylene unit of L¹ may be replaced by —C(O)— or —C(═S)—.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

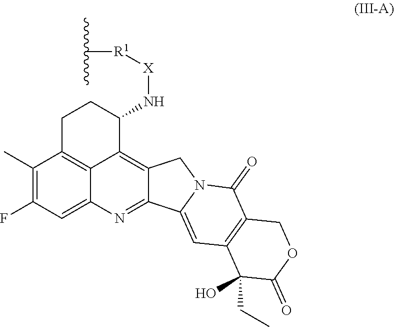

wherein, R¹ may be —S— or —(R²)N—;
X may be -L³-L²-;
wherein L² may be —C(O)—;
L³ may be —(CH₂)ₙ—, and n may be 4 or 5;
wherein 1 methylene unit of L³ may be replaced by —NR⁸—, —O—, —S— or —SO—;
R² may be a C₁₋₆ aliphatic group.

For example, the compound may comprise a structure shown as formula (III-A):
wherein, R¹ may be —S— or —(R²)N—;
X may be -L³-L²-;
wherein L² may be —C(O)—;
L³ may be —(CH₂)ₙ—, and n may be 4 or 5;
wherein 1 methylene unit of L³ may be replaced by —O—;
R² may be a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

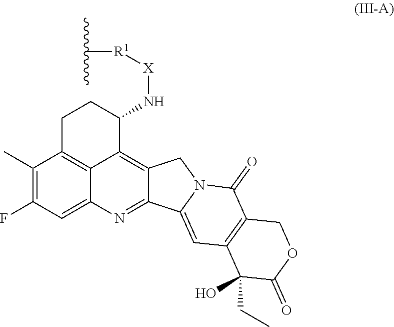

wherein, R¹ may be —O—;
X may be -L³-L²-;
wherein L² may be —C(O)—;
L³ may be —(C(R^{7a})(R^{7b}))ₙ—, and n may be 4 or 5;
wherein 1 methylene unit of L³ may be replaced by —NR⁸— or —O—, and 0 or 1 methylene unit of L³ may also be independently replaced by —C(O)— or —C(═S)—;
wherein each R^{7a}, each R^{7b} and each R⁸ may each independently be hydrogen or a C₁₋₆ aliphatic group.

For example, the compound may comprise a structure shown as formula (III-A):
wherein, R¹ may be —O—;
X may be -L³-L²-;
wherein L² may be —C(O)—;
L³ may be —(C(R^{7a})(R^{7b}))₄—;
wherein 1 methylene unit of L³ may be replaced by —NR⁸— or —O—;
wherein each R^{7a}, each R^{7b} and each R⁸ may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

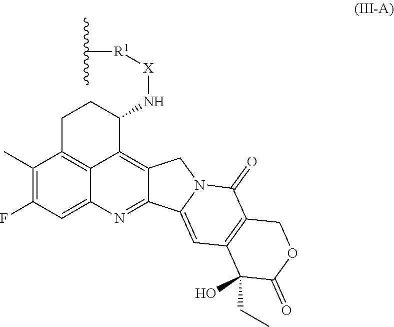

wherein, R¹ may be —O—;
X may be -L³-L²-;
wherein L² may be —C(O)—;
L³ may be —(C(R^{7a})(R^{7b}))₄—;
wherein 1 methylene unit of L³ may be replaced by —NR⁸—;
wherein each R^{7a}, each R^{7b} and each R⁸ may each independently be hydrogen or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

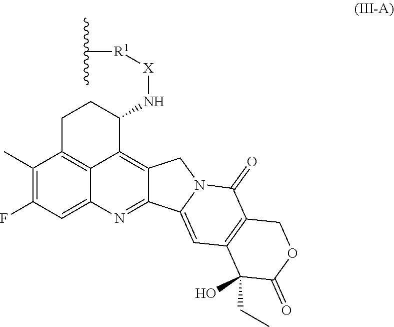

wherein, R¹ may be —NH—;
X may be -L³-L²-;
wherein L² may be —C(O)—;
L³ may be —(CH₂)ₙ—, and n may be 4 or 5;
wherein 1 methylene unit of L³ may be replaced by —S—.

In one embodiment, the antibody-drug conjugate comprises an active agent with a structure according to any one of formulas (III-A-1) to (III-A-17), or a tautomer, a mesomer, a racemate, an enantiomer or a diastereoisomer thereof, or a mixture thereof, or a pharmaceutically acceptable salt thereof:

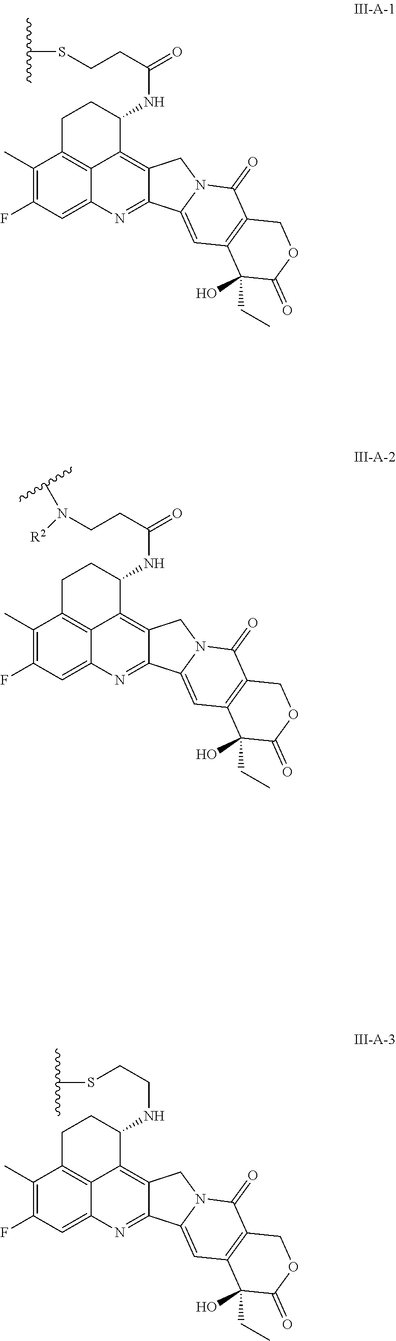

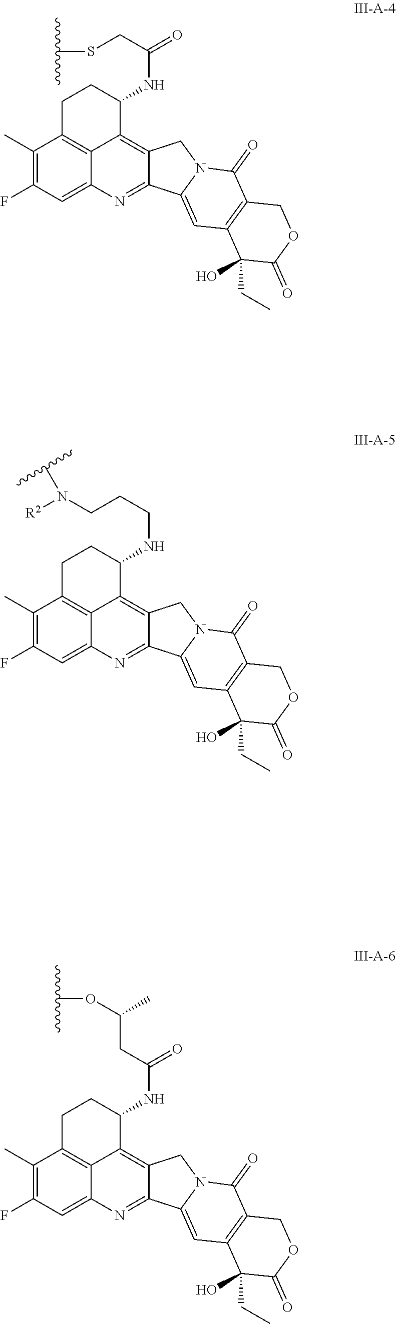

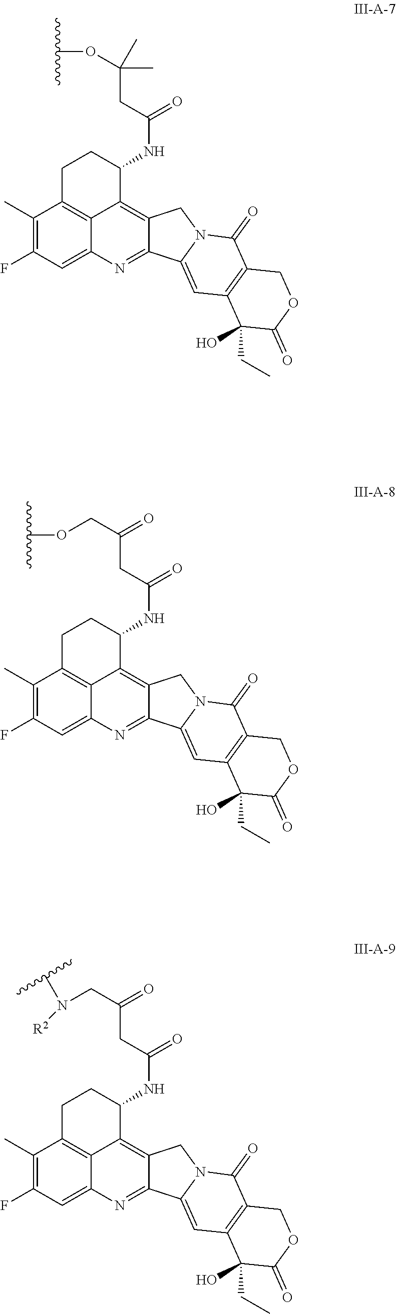

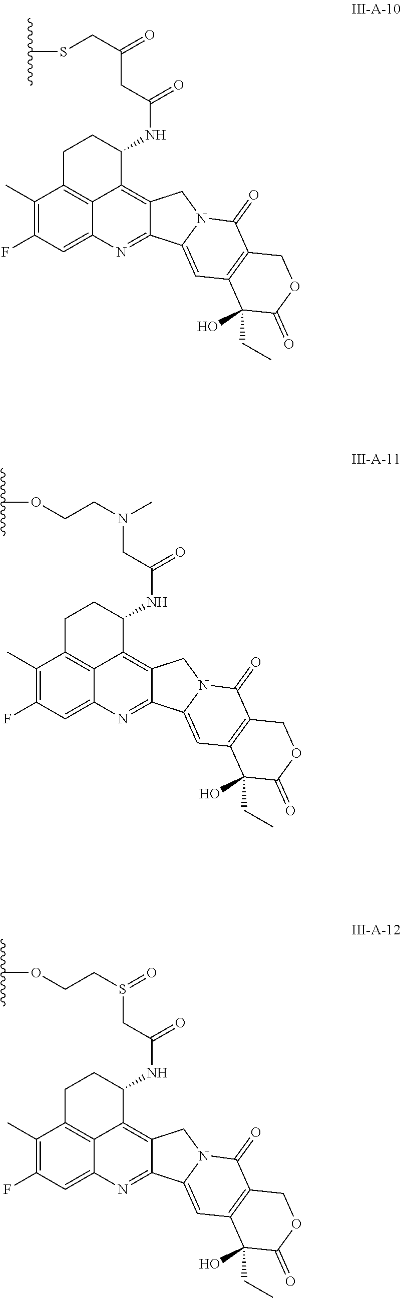 [SMILES: *SCC(=O)CC(=O)N[C@H]1CCc2c(C)c(F)cc3nc4c(c1c23)Cn1c-4cc2c(c1=O)COC(=O)[C@]2(O)CC ; *OCCN(C)CC(=O)N[C@H]1CCc2c(C)c(F)cc3nc4c(c1c23)Cn1c-4cc2c(c1=O)COC(=O)[C@]2(O)CC ; *OCCS(=O)CC(=O)N[C@H]1CCc2c(C)c(F)cc3nc4c(c1c23)Cn1c-4cc2c(c1=O)COC(=O)[C@]2(O)CC]

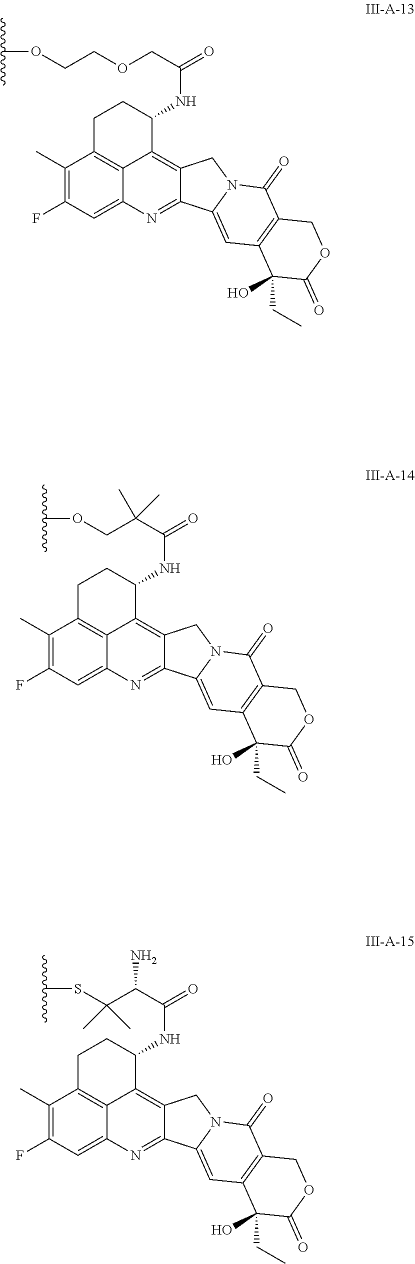 [SMILES: *OCCOCC(=O)N[C@H]1CCc2c(C)c(F)cc3nc4c(c1c23)Cn1c-4cc2c(c1=O)COC(=O)[C@]2(O)CC ; *OCC(C)(C)C(=O)N[C@H]1CCc2c(C)c(F)cc3nc4c(c1c23)Cn1c-4cc2c(c1=O)COC(=O)[C@]2(O)CC ; *SC(C)(C)[C@H](N)C(=O)N[C@H]1CCc2c(C)c(F)cc3nc4c(c1c23)Cn1c-4cc2c(c1=O)COC(=O)[C@]2(O)CC]

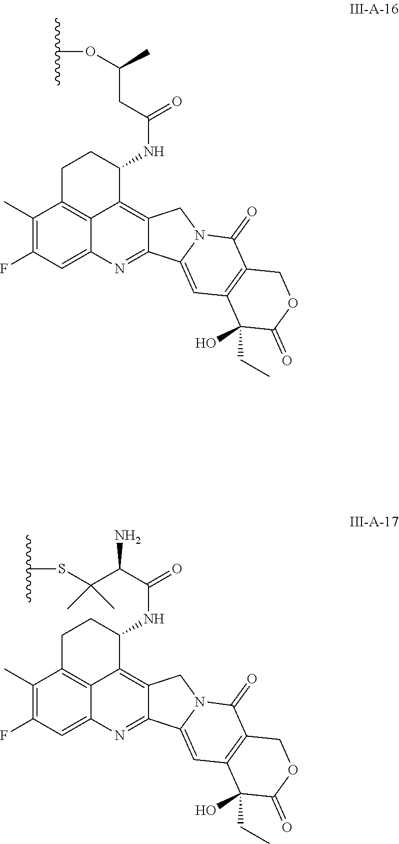 [SMILES: *O[C@@H](C)CC(=O)N[C@H]1CCc2c(C)c(F)cc3nc4c(c1c23)Cn1c-4cc2c(c1=O)COC(=O)[C@]2(O)CC ; *SC(C)(C)[C@@H](N)C(=O)N[C@H]1CCc2c(C)c(F)cc3nc4c(c1c23)Cn1c-4cc2c(c1=O)COC(=O)[C@]2(O)CC]

wherein R² may be a C₁₋₆ aliphatic group which may be optionally replaced by R, wherein R may be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H, or a C₁₋₆ aliphatic group, or wherein, R² may be halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group; wherein the wavy line denotes the link of the structure shown in any one of formulas (III-A-1) to (III-A-17) to the antibody as defined herein either directly or through a linker.

For example, R² may be methyl which may be optionally substituted with one or more hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or C₁₋₆ aliphatic groups. For example, R² may be ethyl which may be optionally substituted with hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group. For example, R² may be propyl which may be optionally substituted with hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure selected from any one of the structures in Table 1, or a tautomer, a mesomer, a racemate, an enantiomer or a diastereoisomer thereof, or a mixture thereof, or a pharmaceutically acceptable salt thereof.

TABLE 1

| No. | Structure |
|---|---|
| P-I-1 | 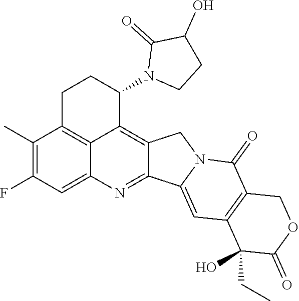 [SMILES: CC[C@@]1(O)C(=O)OCc2c1cc1n(c2=O)Cc2c-1nc1cc(F)c(C)c3c1c2[C@@H](N1CCC(O)C1=O)CC3] |
| P-I-2 | 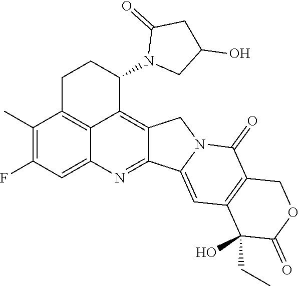 [SMILES: CC[C@@]1(O)C(=O)OCc2c1cc1n(c2=O)Cc2c-1nc1cc(F)c(C)c3c1c2[C@@H](N1CC(O)CC1=O)CC3] |
| P-I-3 | 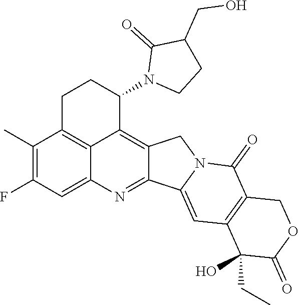 [SMILES: CC[C@@]1(O)C(=O)OCc2c1cc1n(c2=O)Cc2c-1nc1cc(F)c(C)c3c1c2[C@@H](N1CCC(CO)C1=O)CC3] |
| P-I-4 | 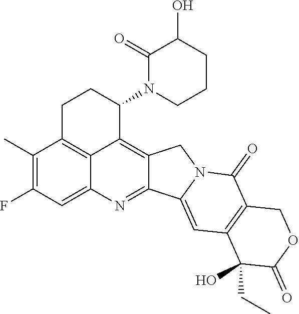 [SMILES: CC[C@@]1(O)C(=O)OCc2c1cc1n(c2=O)Cc2c-1nc1cc(F)c(C)c3c1c2[C@@H](N1CCCC(O)C1=O)CC3] |
| P-I-5 | 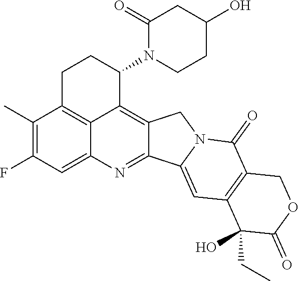 [SMILES: CC[C@@]1(O)C(=O)OCc2c1cc1n(c2=O)Cc2c-1nc1cc(F)c(C)c3c1c2[C@@H](N1CCC(O)CC1=O)CC3] |
| P-I-6 | 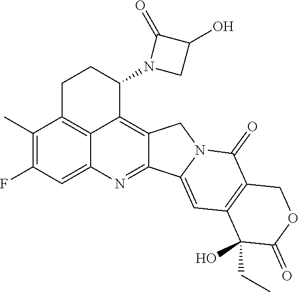 [SMILES: CC[C@@]1(O)C(=O)OCc2c1cc1n(c2=O)Cc2c-1nc1cc(F)c(C)c3c1c2[C@@H](N1CC(O)C1=O)CC3] |
| P-I-7 | 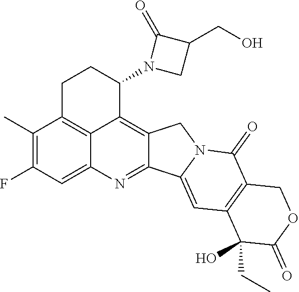 [SMILES: CC[C@@]1(O)C(=O)OCc2c1cc1n(c2=O)Cc2c-1nc1cc(F)c(C)c3c1c2[C@@H](N1CC(CO)C1=O)CC3] |
| P-I-8 | 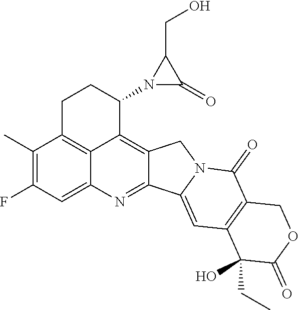 [SMILES: CC[C@@]1(O)C(=O)OCc2c1cc1n(c2=O)Cc2c-1nc1cc(F)c(C)c3c1c2[C@@H](N1C(=O)C1CO)CC3] |
| P-I-9 | 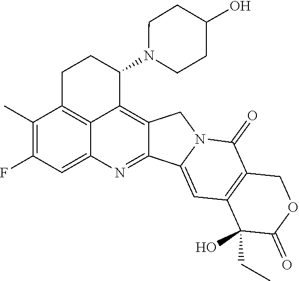 [SMILES: CC[C@@]1(O)C(=O)OCc2c1cc1n(c2=O)Cc2c-1nc1cc(F)c(C)c3c1c2[C@@H](N1CCC(O)CC1)CC3] |
| P-I-10 | 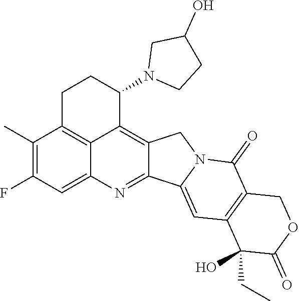 [SMILES: CC[C@@]1(O)C(=O)OCc2c1cc1n(c2=O)Cc2c-1nc1cc(F)c(C)c3c1c2[C@@H](N1CCC(O)C1)CC3] |
| P-I-11 | 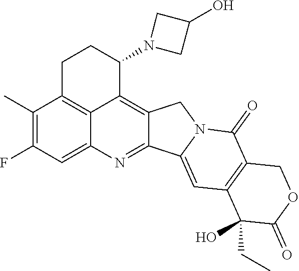 [SMILES: CC[C@@]1(O)C(=O)OCc2c1cc1n(c2=O)Cc2c-1nc1cc(F)c(C)c3c1c2[C@@H](N1CC(O)C1)CC3] |
| P-I-12 | 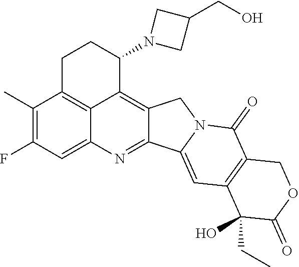 [SMILES: CC[C@@]1(O)C(=O)OCc2c1cc1n(c2=O)Cc2c-1nc1cc(F)c(C)c3c1c2[C@@H](N1CC(CO)C1)CC3] |
| P-I-13 | 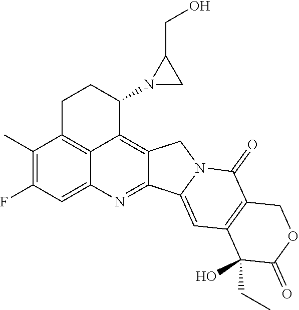 [SMILES: CC[C@@]1(O)C(=O)OCc2c1cc1n(c2=O)Cc2c-1nc1cc(F)c(C)c3c1c2[C@@H](N1CC1CO)CC3] |
| P-I-14 | 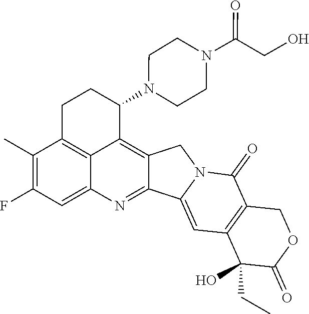 [SMILES: CC[C@@]1(O)C(=O)OCc2c1cc1n(c2=O)Cc2c-1nc1cc(F)c(C)c3c1c2[C@@H](N1CCN(C(=O)CO)CC1)CC3] |
| P-I-15 | 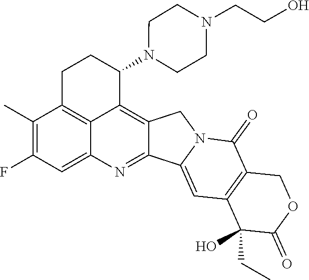 [SMILES: CC[C@@]1(O)C(=O)OCc2c1cc1n(c2=O)Cc2c-1nc1cc(F)c(C)c3c1c2[C@@H](N1CCN(CCO)CC1)CC3] |
| P-I-16 | 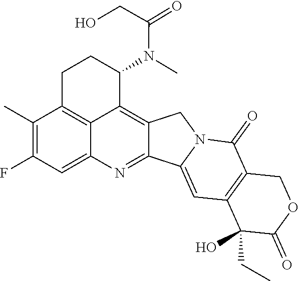 [SMILES: CC[C@@]1(O)C(=O)OCc2c1cc1n(c2=O)Cc2c-1nc1cc(F)c(C)c3c1c2[C@@H](N(C)C(=O)CO)CC3] |
| P-I-17 | 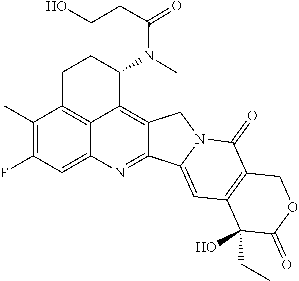 [SMILES: CC[C@@]1(O)C(=O)OCc2c1cc1n(c2=O)Cc2c-1nc1cc(F)c(C)c3c1c2[C@@H](N(C)C(=O)CCO)CC3] |
| P-I-18 | 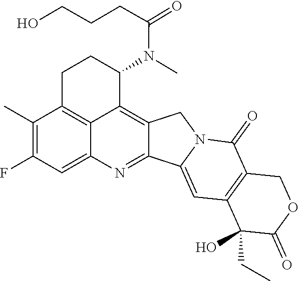 [SMILES: CC[C@@]1(O)C(=O)OCc2c1cc1n(c2=O)Cc2c-1nc1cc(F)c(C)c3c1c2[C@@H](N(C)C(=O)CCCO)CC3] |
| P-I-19 | 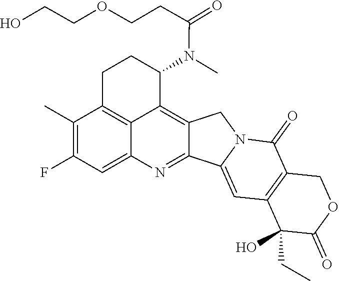 [SMILES: CC[C@@]1(O)C(=O)OCc2c1cc1n(c2=O)Cc2c-1nc1cc(F)c(C)c3c1c2[C@@H](N(C)C(=O)CCOCCO)CC3] |
| P-I-20 | 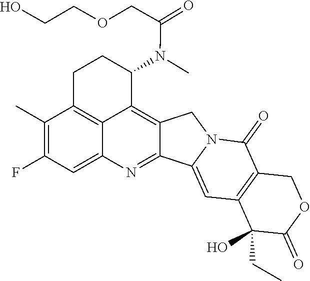 [SMILES: CC[C@@]1(O)C(=O)OCc2c1cc1n(c2=O)Cc2c-1nc1cc(F)c(C)c3c1c2[C@@H](N(C)C(=O)COCCO)CC3] |
| P-I-21 | 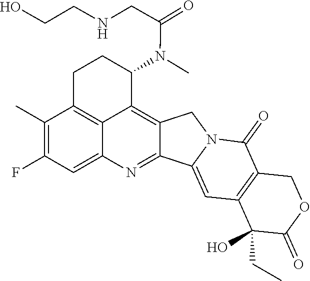 [SMILES: CC[C@@]1(O)C(=O)OCc2c1cc1n(c2=O)Cc2c-1nc1cc(F)c(C)c3c1c2[C@@H](N(C)C(=O)CNCCO)CC3] |
| P-I-22 | 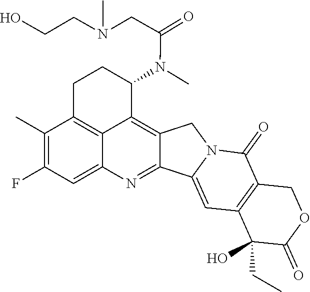 [SMILES: CC[C@@]1(O)C(=O)OCc2c1cc1n(c2=O)Cc2c-1nc1cc(F)c(C)c3c1c2[C@@H](N(C)C(=O)CN(C)CCO)CC3] |
| P-I-23 | 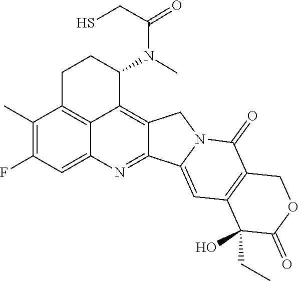 [SMILES: CC[C@@]1(O)C(=O)OCc2c1cc1n(c2=O)Cc2c-1nc1cc(F)c(C)c3c1c2[C@@H](N(C)C(=O)CS)CC3] |
| P-I-24 | 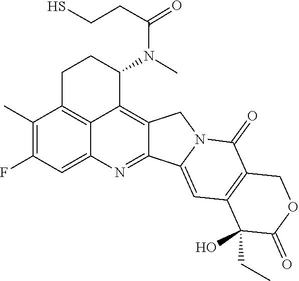 [SMILES: CC[C@@]1(O)C(=O)OCc2c1cc1n(c2=O)Cc2c-1nc1cc(F)c(C)c3c1c2[C@@H](N(C)C(=O)CCS)CC3] |
| P-I-25 | 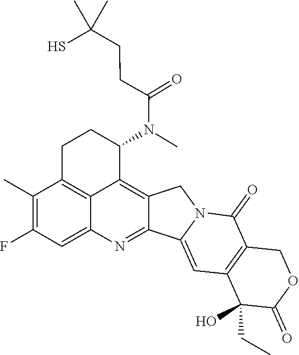 [SMILES: CC[C@@]1(O)C(=O)OCc2c1cc1n(c2=O)Cc2c-1nc1cc(F)c(C)c3c1c2[C@@H](N(C)C(=O)CCC(C)(C)S)CC3] |
| P-I-26 | 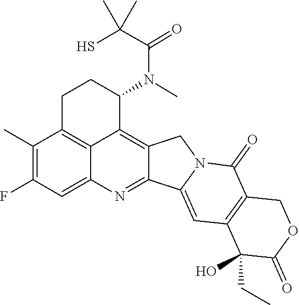 [SMILES: CC[C@@]1(O)C(=O)OCc2c1cc1n(c2=O)Cc2c-1nc1cc(F)c(C)c3c1c2[C@@H](N(C)C(=O)C(C)(C)S)CC3] |
| P-I-27 | 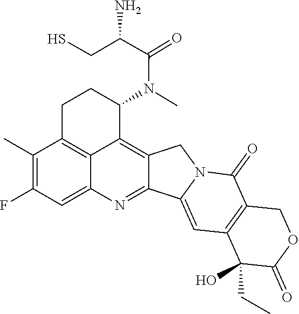 [SMILES: CC[C@@]1(O)C(=O)OCc2c1cc1n(c2=O)Cc2c-1nc1cc(F)c(C)c3c1c2[C@@H](N(C)C(=O)[C@@H](N)CS)CC3] |
| P-I-28 | 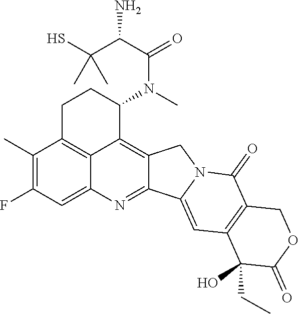 [SMILES: CC[C@@]1(O)C(=O)OCc2c1cc1n(c2=O)Cc2c-1nc1cc(F)c(C)c3c1c2[C@@H](N(C)C(=O)[C@@H](N)C(C)(C)S)CC3] |
| P-I-29 | 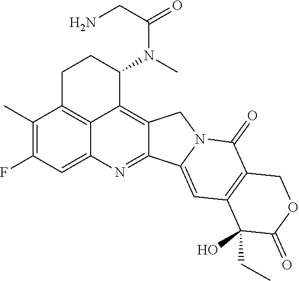 [SMILES: CC[C@@]1(O)C(=O)OCc2c1cc1n(c2=O)Cc2c-1nc1cc(F)c(C)c3c1c2[C@@H](N(C)C(=O)CN)CC3] |
| P-I-30 | 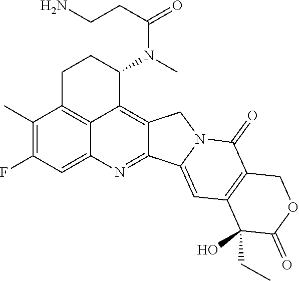 [SMILES: CC[C@@]1(O)C(=O)OCc2c1cc1n(c2=O)Cc2c-1nc1cc(F)c(C)c3c1c2[C@@H](N(C)C(=O)CCN)CC3] |
| P-I-31 | 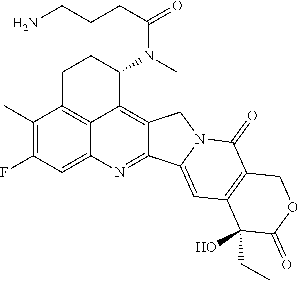 [SMILES: CC[C@@]1(O)C(=O)OCc2c1cc1n(c2=O)Cc2c-1nc1cc(F)c(C)c3c1c2[C@@H](N(C)C(=O)CCCN)CC3] |
| P-I-32 | 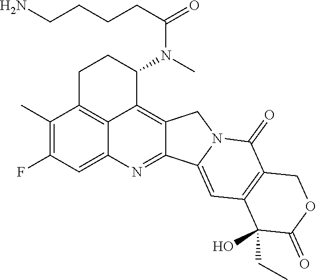 [SMILES: CC[C@@]1(O)C(=O)OCc2c1cc1n(c2=O)Cc2c-1nc1cc(F)c(C)c3c1c2[C@@H](N(C)C(=O)CCCCN)CC3] |
| P-I-33 | 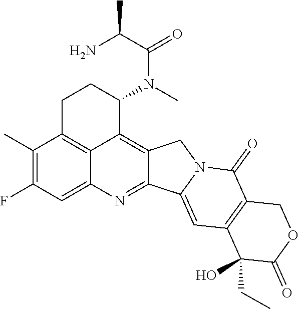 [SMILES: CC[C@@]1(O)C(=O)OCc2c1cc1n(c2=O)Cc2c-1nc1cc(F)c(C)c3c1c2[C@@H](N(C)C(=O)[C@H](C)N)CC3] |
| P-I-34 | 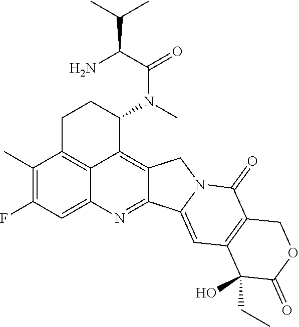 [SMILES: CC[C@@]1(O)C(=O)OCc2c1cc1n(c2=O)Cc2c-1nc1cc(F)c(C)c3c1c2[C@@H](N(C)C(=O)[C@@H](N)C(C)C)CC3] |
| P-I-35 | 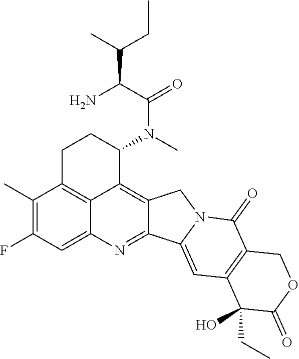 [SMILES: CCC(C)[C@H](N)C(=O)N(C)[C@H]1CCc2c(C)c(F)cc3nc4c(c1c23)Cn1c-4cc2c(c1=O)COC(=O)[C@]2(O)CC] |
| P-I-36 | 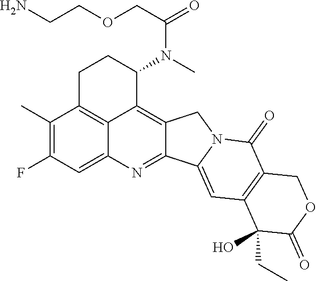 [SMILES: CC[C@@]1(O)C(=O)OCc2c1cc1n(c2=O)Cc2c-1nc1cc(F)c(C)c3c1c2[C@@H](N(C)C(=O)COCCN)CC3] |
| P-I-37 | 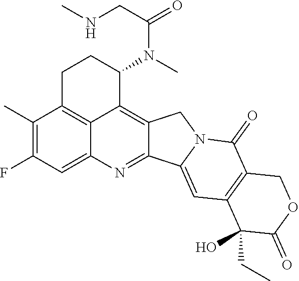 [SMILES: CC[C@@]1(O)C(=O)OCc2c1cc1n(c2=O)Cc2c-1nc1cc(F)c(C)c3c1c2[C@@H](N(C)C(=O)CNC)CC3] |
| P-I-38 | 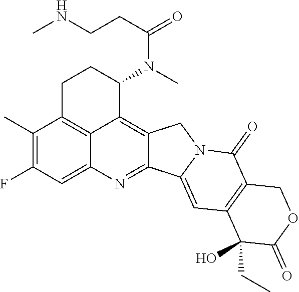 [SMILES: CC[C@@]1(O)C(=O)OCc2c1cc1n(c2=O)Cc2c-1nc1cc(F)c(C)c3c1c2[C@@H](N(C)C(=O)CCNC)CC3] |
| P-I-39 | 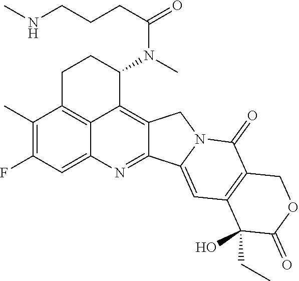 [SMILES: CC[C@@]1(O)C(=O)OCc2c1cc1n(c2=O)Cc2c-1nc1cc(F)c(C)c3c1c2[C@@H](N(C)C(=O)CCCNC)CC3] |
| P-I-40 | 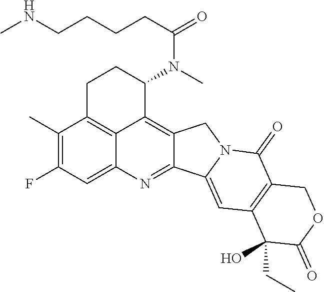 [SMILES: CC[C@@]1(O)C(=O)OCc2c1cc1n(c2=O)Cc2c-1nc1cc(F)c(C)c3c1c2[C@@H](N(C)C(=O)CCCCNC)CC3] |
| P-I-41 | 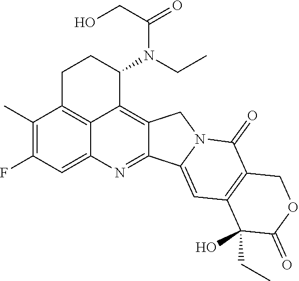 [SMILES: CCN(C(=O)CO)[C@H]1CCc2c(C)c(F)cc3nc4c(c1c23)Cn1c-4cc2c(c1=O)COC(=O)[C@]2(O)CC] |
| P-I-42 | 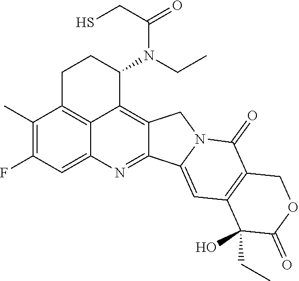 [SMILES: CCN(C(=O)CS)[C@H]1CCc2c(C)c(F)cc3nc4c(c1c23)Cn1c-4cc2c(c1=O)COC(=O)[C@]2(O)CC] |
| P-I-43 | 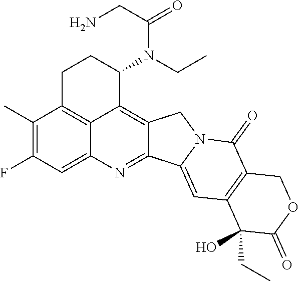 [SMILES: CCN(C(=O)CN)[C@H]1CCc2c(C)c(F)cc3nc4c(c1c23)Cn1c-4cc2c(c1=O)COC(=O)[C@]2(O)CC] |
| P-I-44 | 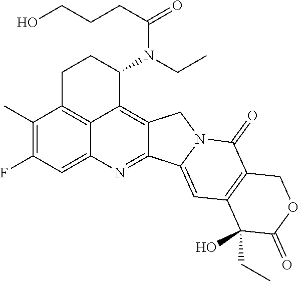 [SMILES: CCN(C(=O)CCCO)[C@H]1CCc2c(C)c(F)cc3nc4c(c1c23)Cn1c-4cc2c(c1=O)COC(=O)[C@]2(O)CC] |
| P-I-45 | 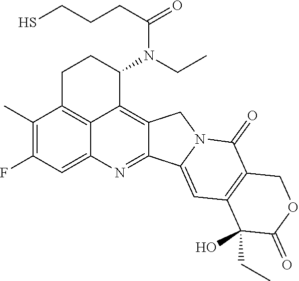 [SMILES: CCN(C(=O)CCCS)[C@H]1CCc2c(C)c(F)cc3nc4c(c1c23)Cn1c-4cc2c(c1=O)COC(=O)[C@]2(O)CC] |
| P-I-46 | 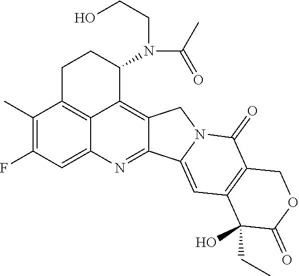 [SMILES: CC[C@@]1(O)C(=O)OCc2c1cc1n(c2=O)Cc2c-1nc1cc(F)c(C)c3c1c2[C@@H](N(CCO)C(C)=O)CC3] |
| P-I-47 | 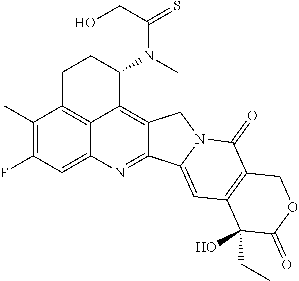 [SMILES: CC[C@@]1(O)C(=O)OCc2c1cc1n(c2=O)Cc2c-1nc1cc(F)c(C)c3c1c2[C@@H](N(C)C(=S)CO)CC3] |
| P-II-1 | 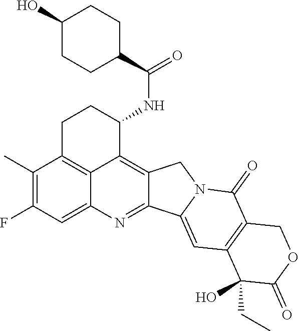 |
| P-II-2 | 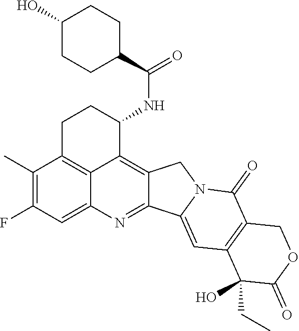 |
| P-II-3 | 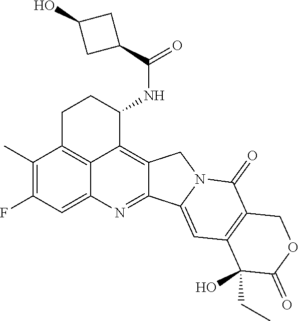 [SMILES: CC[C@@]1(O)C(=O)OCc2c1cc1n(c2=O)Cc2c-1nc1cc(F)c(C)c3c1c2[C@@H](NC(=O)[C@H]1C[C@@H](O)C1)CC3] |
| P-II-4 | 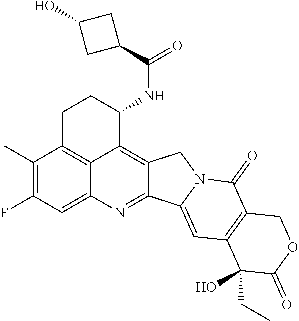 [SMILES: CC[C@@]1(O)C(=O)OCc2c1cc1n(c2=O)Cc2c-1nc1cc(F)c(C)c3c1c2[C@@H](NC(=O)[C@H]1C[C@H](O)C1)CC3] |
| P-II-5 | 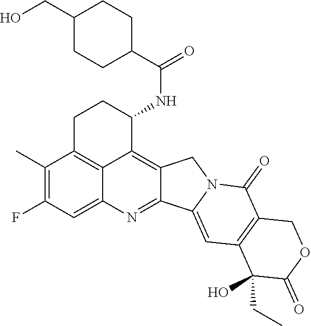 [SMILES: CC[C@@]1(O)C(=O)OCc2c1cc1n(c2=O)Cc2c-1nc1cc(F)c(C)c3c1c2[C@@H](NC(=O)C1CCC(CO)CC1)CC3] |
| P-II-6 | 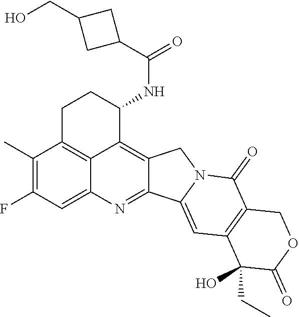 [SMILES: CC[C@@]1(O)C(=O)OCc2c1cc1n(c2=O)Cc2c-1nc1cc(F)c(C)c3c1c2[C@@H](NC(=O)C1CC(CO)C1)CC3] |
| P-II-7 | 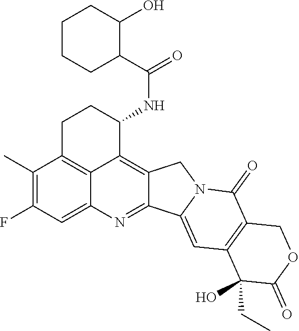 [SMILES: CC[C@@]1(O)C(=O)OCc2c1cc1n(c2=O)Cc2c-1nc1cc(F)c(C)c3c1c2[C@@H](NC(=O)C1CCCCC1O)CC3] |
| P-II-8 | 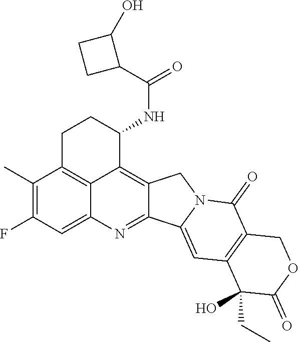 [SMILES: CC[C@@]1(O)C(=O)OCc2c1cc1n(c2=O)Cc2c-1nc1cc(F)c(C)c3c1c2[C@@H](NC(=O)C1CCC1O)CC3] |
| P-II-9 | 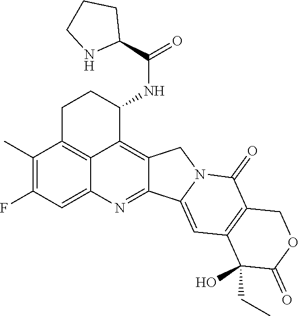 [SMILES: CC[C@@]1(O)C(=O)OCc2c1cc1n(c2=O)Cc2c-1nc1cc(F)c(C)c3c1c2[C@@H](NC(=O)[C@@H]1CCCN1)CC3] |
| P-II-10 | 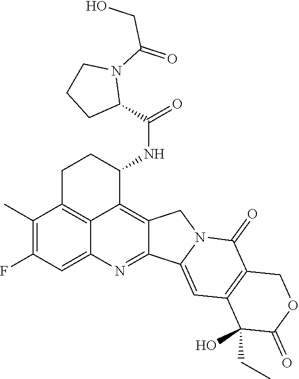 [SMILES: CC[C@@]1(O)C(=O)OCc2c1cc1n(c2=O)Cc2c-1nc1cc(F)c(C)c3c1c2[C@@H](NC(=O)[C@@H]1CCCN1C(=O)CO)CC3] |
| P-II-11 | 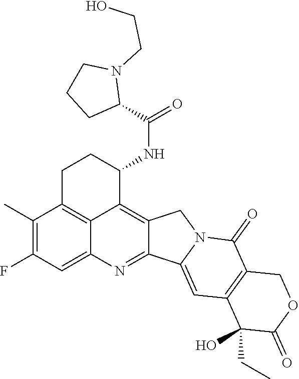 [SMILES: CC[C@@]1(O)C(=O)OCc2c1cc1n(c2=O)Cc2c-1nc1cc(F)c(C)c3c1c2[C@@H](NC(=O)[C@@H]1CCCN1CCO)CC3] |
| P-II-12 | 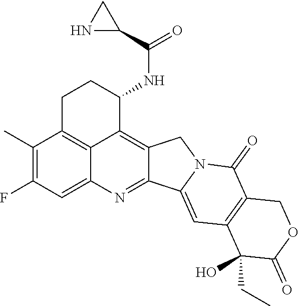 [SMILES: CC[C@@]1(O)C(=O)OCc2c1cc1n(c2=O)Cc2c-1nc1cc(F)c(C)c3c1c2[C@@H](NC(=O)[C@@H]1CN1)CC3] |
| P-II-13 | 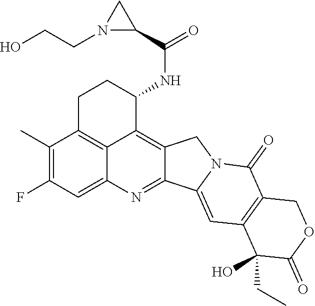 [SMILES: CC[C@@]1(O)C(=O)OCc2c1cc1n(c2=O)Cc2c-1nc1cc(F)c(C)c3c1c2[C@@H](NC(=O)[C@@H]1CN1CCO)CC3] |
| P-II-14 | 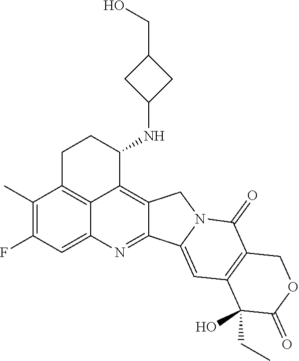 [SMILES: CC[C@@]1(O)C(=O)OCc2c1cc1n(c2=O)Cc2c-1nc1cc(F)c(C)c3c1c2[C@@H](NC1CC(CO)C1)CC3] |
| P-II-15 | 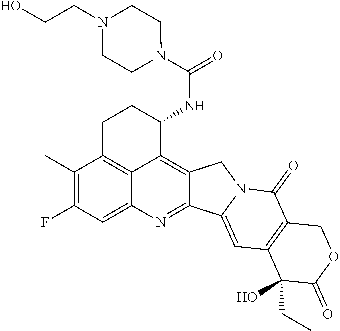 [SMILES: CC[C@@]1(O)C(=O)OCc2c1cc1n(c2=O)Cc2c-1nc1cc(F)c(C)c3c1c2[C@@H](NC(=O)N1CCN(CCO)CC1)CC3] |
| P-II-16 | 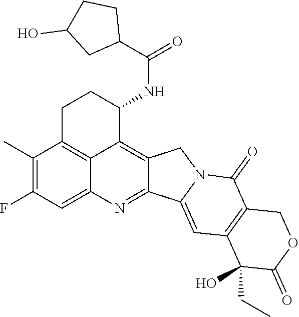 [SMILES: CC[C@@]1(O)C(=O)OCc2c1cc1n(c2=O)Cc2c-1nc1cc(F)c(C)c3c1c2[C@@H](NC(=O)C1CCC(O)C1)CC3] |
| P-II-17 | 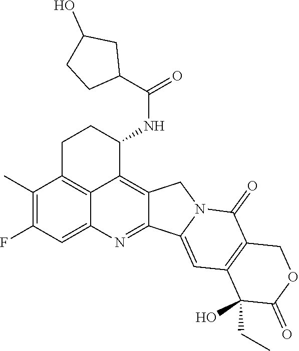 [SMILES: CC[C@@]1(O)C(=O)OCc2c1cc1n(c2=O)Cc2c-1nc1cc(F)c(C)c3c1c2[C@@H](NC(=O)C1CCC(O)C1)CC3] |
| P-II-18 | 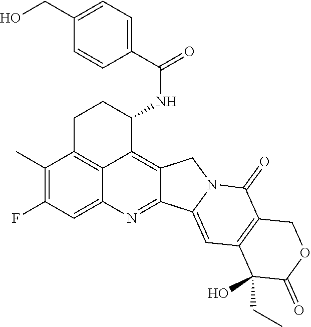 [SMILES: CC[C@@]1(O)C(=O)OCc2c1cc1n(c2=O)Cc2c-1nc1cc(F)c(C)c3c1c2[C@@H](NC(=O)c1ccc(CO)cc1)CC3] |
| P-II-19 | 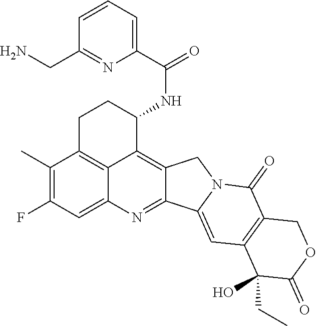 [SMILES: CC[C@@]1(O)C(=O)OCc2c1cc1n(c2=O)Cc2c-1nc1cc(F)c(C)c3c1c2[C@@H](NC(=O)c1cccc(CN)n1)CC3] |
| P-II-20 | 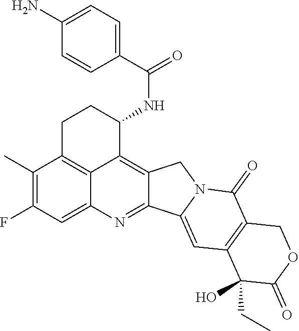 [SMILES: CC[C@@]1(O)C(=O)OCc2c1cc1n(c2=O)Cc2c-1nc1cc(F)c(C)c3c1c2[C@@H](NC(=O)c1ccc(N)cc1)CC3] |
| P-II-21 | 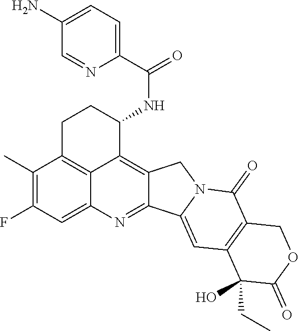 [SMILES: CC[C@@]1(O)C(=O)OCc2c1cc1n(c2=O)Cc2c-1nc1cc(F)c(C)c3c1c2[C@@H](NC(=O)c1ccc(N)cn1)CC3] |
| P-II-22 | 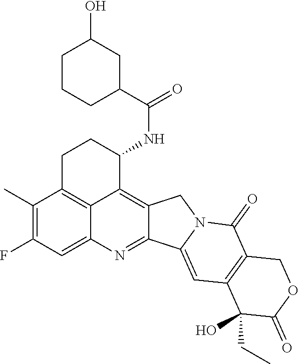 [SMILES: CC[C@@]1(O)C(=O)OCc2c1cc1n(c2=O)Cc2c-1nc1cc(F)c(C)c3c1c2[C@@H](NC(=O)C1CCCC(O)C1)CC3] |
| P-II-23 | 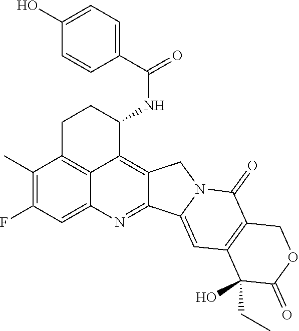 [SMILES: CC[C@@]1(O)C(=O)OCc2c1cc1n(c2=O)Cc2c-1nc1cc(F)c(C)c3c1c2[C@@H](NC(=O)c1ccc(O)cc1)CC3] |
| P-II-24 | 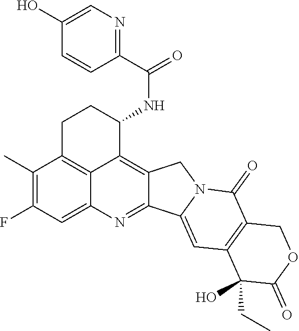 [SMILES: CC[C@@]1(O)C(=O)OCc2c1cc1n(c2=O)Cc2c-1nc1cc(F)c(C)c3c1c2[C@@H](NC(=O)c1ccc(O)cn1)CC3] |
| P-II-25 | 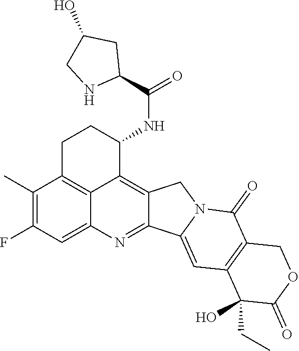 [SMILES: CC[C@@]1(O)C(=O)OCc2c1cc1n(c2=O)Cc2c-1nc1cc(F)c(C)c3c1c2[C@@H](NC(=O)[C@@H]1C[C@@H](O)CN1)CC3] |
| P-III-1 | 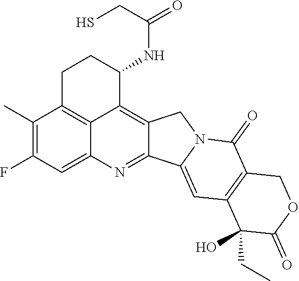 [SMILES: CC[C@@]1(O)C(=O)OCc2c1cc1n(c2=O)Cc2c-1nc1cc(F)c(C)c3c1c2[C@@H](NC(=O)CS)CC3] |
| P-III-2 | 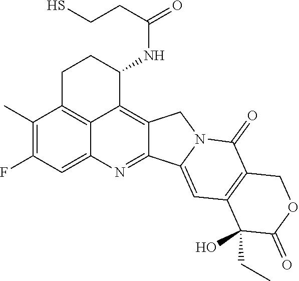 [SMILES: CC[C@@]1(O)C(=O)OCc2c1cc1n(c2=O)Cc2c-1nc1cc(F)c(C)c3c1c2[C@@H](NC(=O)CCS)CC3] |
| P-III-3 | 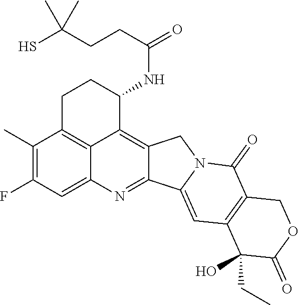 [SMILES: CC[C@@]1(O)C(=O)OCc2c1cc1n(c2=O)Cc2c-1nc1cc(F)c(C)c3c1c2[C@@H](NC(=O)CCC(C)(C)S)CC3] |
| P-III-4 | 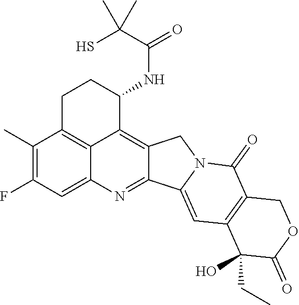 [SMILES: CC[C@@]1(O)C(=O)OCc2c1cc1n(c2=O)Cc2c-1nc1cc(F)c(C)c3c1c2[C@@H](NC(=O)C(C)(C)S)CC3] |
| P-III-5 | 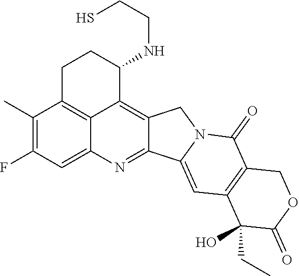 [SMILES: CC[C@@]1(O)C(=O)OCc2c1cc1n(c2=O)Cc2c-1nc1cc(F)c(C)c3c1c2[C@@H](NCCS)CC3] |
| P-III-6 | 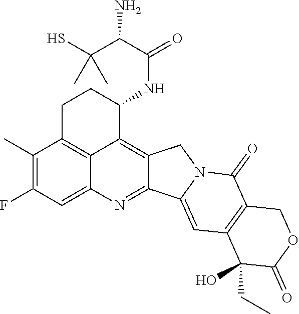 [SMILES: CC[C@@]1(O)C(=O)OCc2c1cc1n(c2=O)Cc2c-1nc1cc(F)c(C)c3c1c2[C@@H](NC(=O)[C@@H](N)C(C)(C)S)CC3] |
| P-III-7 | 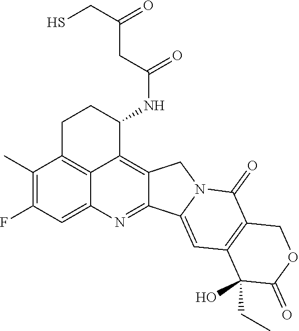 [SMILES: CC[C@@]1(O)C(=O)OCc2c1cc1n(c2=O)Cc2c-1nc1cc(F)c(C)c3c1c2[C@@H](NC(=O)CC(=O)CS)CC3] |
| P-III-8 | 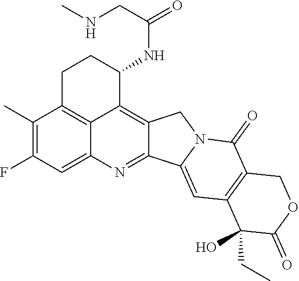 [SMILES: CC[C@@]1(O)C(=O)OCc2c1cc1n(c2=O)Cc2c-1nc1cc(F)c(C)c3c1c2[C@@H](NC(=O)CNC)CC3] |
| P-III-9 | 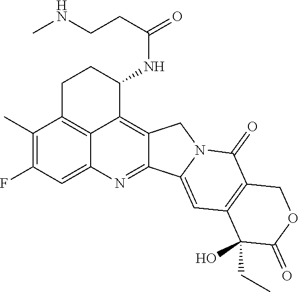 [SMILES: CC[C@@]1(O)C(=O)OCc2c1cc1n(c2=O)Cc2c-1nc1cc(F)c(C)c3c1c2[C@@H](NC(=O)CCNC)CC3] |
| P-III-10 | 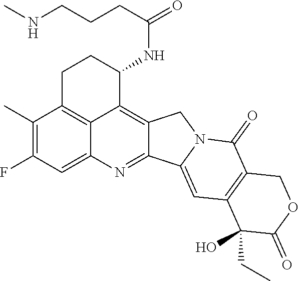 [SMILES: CC[C@@]1(O)C(=O)OCc2c1cc1n(c2=O)Cc2c-1nc1cc(F)c(C)c3c1c2[C@@H](NC(=O)CCCNC)CC3] |
| P-III-11 | 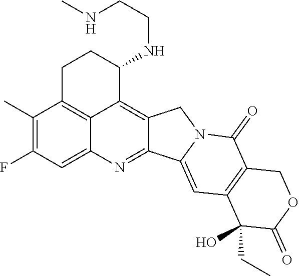 [SMILES: CC[C@@]1(O)C(=O)OCc2c1cc1n(c2=O)Cc2c-1nc1cc(F)c(C)c3c1c2[C@@H](NCCNC)CC3] |
| P-III-12 | 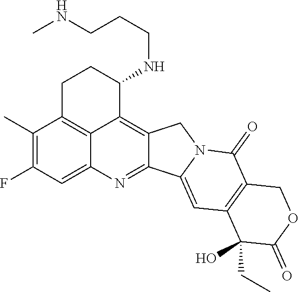 [SMILES: CC[C@@]1(O)C(=O)OCc2c1cc1n(c2=O)Cc2c-1nc1cc(F)c(C)c3c1c2[C@@H](NCCCNC)CC3] |
| P-III-13 | 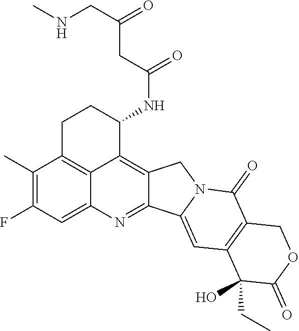 [SMILES: CC[C@@]1(O)C(=O)OCc2c1cc1n(c2=O)Cc2c-1nc1cc(F)c(C)c3c1c2[C@@H](NC(=O)CC(=O)CNC)CC3] |
| P-III-14 | 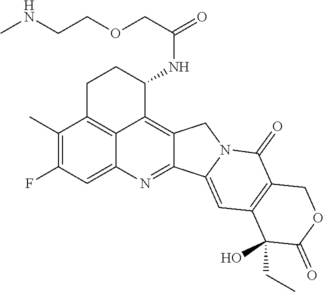 [SMILES: CC[C@@]1(O)C(=O)OCc2c1cc1n(c2=O)Cc2c-1nc1cc(F)c(C)c3c1c2[C@@H](NC(=O)COCCNC)CC3] |
| P-III-15 | 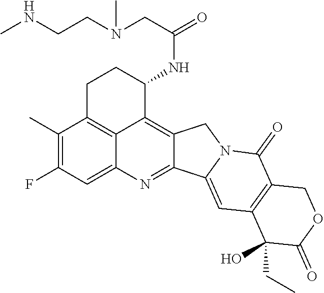 [SMILES: CC[C@@]1(O)C(=O)OCc2c1cc1n(c2=O)Cc2c-1nc1cc(F)c(C)c3c1c2[C@@H](NC(=O)CN(C)CCNC)CC3] |
| P-III-16 | 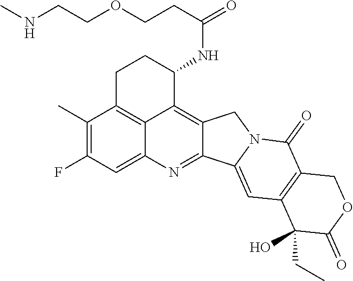 [SMILES: CC[C@@]1(O)C(=O)OCc2c1cc1n(c2=O)Cc2c-1nc1cc(F)c(C)c3c1c2[C@@H](NC(=O)CCOCCNC)CC3] |
| P-III-17 | 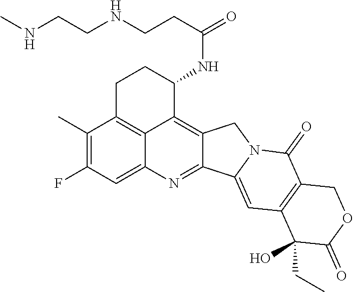 [SMILES: CC[C@@]1(O)C(=O)OCc2c1cc1n(c2=O)Cc2c-1nc1cc(F)c(C)c3c1c2[C@@H](NC(=O)CCNCCNC)CC3] |
| P-III-18 | 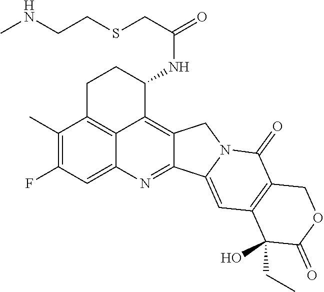 [SMILES: CC[C@@]1(O)C(=O)OCc2c1cc1n(c2=O)Cc2c-1nc1cc(F)c(C)c3c1c2[C@@H](NC(=O)CSCCNC)CC3] |
| P-III-19 | 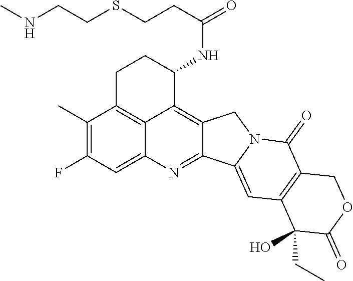 [SMILES: CC[C@@]1(O)C(=O)OCc2c1cc1n(c2=O)Cc2c-1nc1cc(F)c(C)c3c1c2[C@@H](NC(=O)CCSCCNC)CC3] |
| P-III-20 | 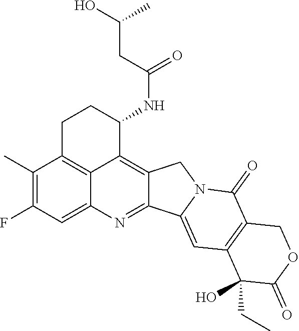 [SMILES: CC[C@@]1(O)C(=O)OCc2c1cc1n(c2=O)Cc2c-1nc1cc(F)c(C)c3c1c2[C@@H](NC(=O)C[C@@H](C)O)CC3] |
| P-III-21 | 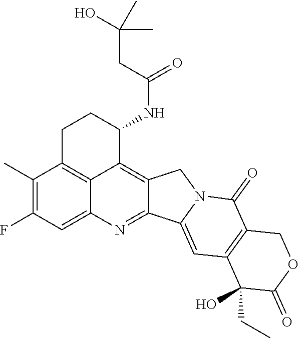 [SMILES: CC[C@@]1(O)C(=O)OCc2c1cc1n(c2=O)Cc2c-1nc1cc(F)c(C)c3c1c2[C@@H](NC(=O)CC(C)(C)O)CC3] |
| P-III-22 | 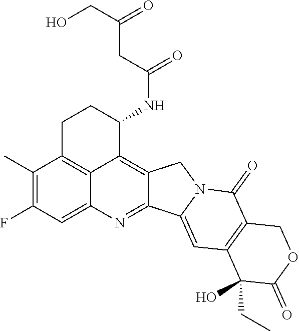 [SMILES: CC[C@@]1(O)C(=O)OCc2c1cc1n(c2=O)Cc2c-1nc1cc(F)c(C)c3c1c2[C@@H](NC(=O)CC(=O)CO)CC3] |
| P-III-23 | 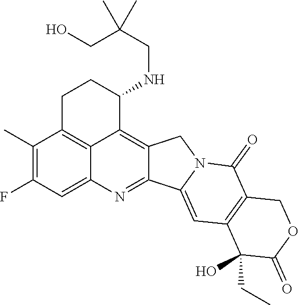 [SMILES: CC[C@@]1(O)C(=O)OCc2c1cc1n(c2=O)Cc2c-1nc1cc(F)c(C)c3c1c2[C@@H](NCC(C)(C)CO)CC3] |
| P-III-24 | 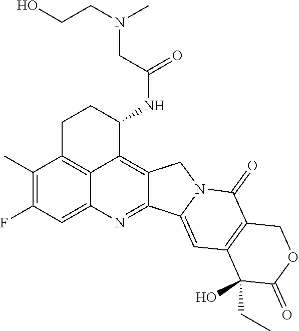 [SMILES: CC[C@@]1(O)C(=O)OCc2c1cc1n(c2=O)Cc2c-1nc1cc(F)c(C)c3c1c2[C@@H](NC(=O)CN(C)CCO)CC3] |
| P-III-25 | 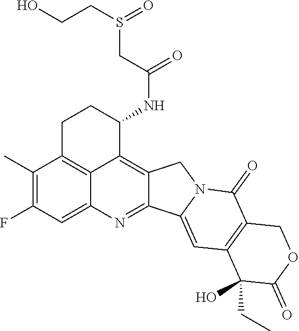 [SMILES: CC[C@@]1(O)C(=O)OCc2c1cc1n(c2=O)Cc2c-1nc1cc(F)c(C)c3c1c2[C@@H](NC(=O)CS(=O)CCO)CC3] |
| P-III-26 | 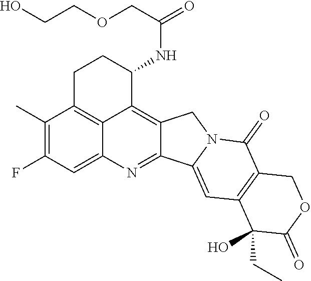 [SMILES: CC[C@@]1(O)C(=O)OCc2c1cc1n(c2=O)Cc2c-1nc1cc(F)c(C)c3c1c2[C@@H](NC(=O)COCCO)CC3] |
| P-III-27 | 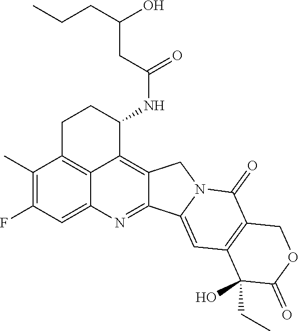 [SMILES: CCCC(O)CC(=O)N[C@H]1CCc2c(C)c(F)cc3nc4c(c1c23)Cn1c-4cc2c(c1=O)COC(=O)[C@]2(O)CC] |
| P-III-28 | 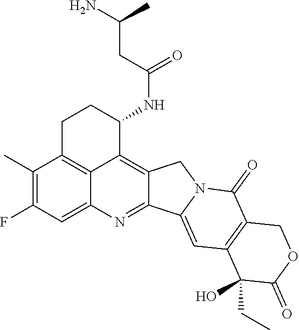 [SMILES: CC[C@@]1(O)C(=O)OCc2c1cc1n(c2=O)Cc2c-1nc1cc(F)c(C)c3c1c2[C@@H](NC(=O)C[C@H](C)N)CC3] |
| P-III-29 | 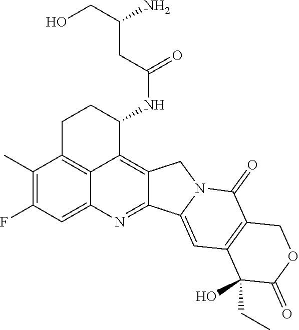 [SMILES: CC[C@@]1(O)C(=O)OCc2c1cc1n(c2=O)Cc2c-1nc1cc(F)c(C)c3c1c2[C@@H](NC(=O)C[C@@H](N)CO)CC3] |
| P-III-30 | 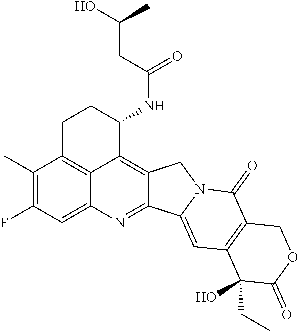 [SMILES: CC[C@@]1(O)C(=O)OCc2c1cc1n(c2=O)Cc2c-1nc1cc(F)c(C)c3c1c2[C@@H](NC(=O)C[C@H](C)O)CC3] |
| P-III-31 | |

There may be one or more units of drug per antibody molecule. The ratio between the number of drug molecules per antibody is denoted the drug-to-antibody ratio (DAR). In one embodiment, the DAR is between 1 and 10, such as between 2 and 8, for example between 2 and 6, such as 2 or 4.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

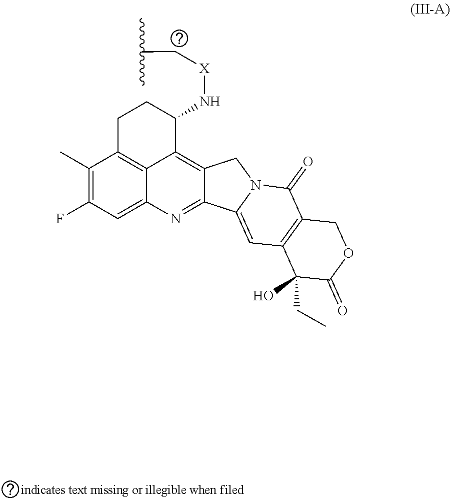

wherein, R¹ may be —O— or —(R²)N— or —S—; and
X may be -L¹-CH₂—C(O)—, or may be -A-C(O)—;
wherein, when X is -L¹-CH₂—C(O)—, L¹ may be —(C(R^{3a})(R^{3b}))ₘ, wherein m may be selected from the group consisting of 0, 1 and 2, wherein 0 or 1 methylene unit of L¹ may be independently replaced by —C(O)— or —C(S)—;
wherein when X is -A-C(O)—, A may be a ring structure moiety, selected from the group consisting of phenyl, pyridyl, cyclohexyl, cyclopentyl, and cyclobutyl, wherein A may be un-substituted or may be substituted with one or more substituents R^{1c};
wherein each of R², R^{3a}, R^{3b} and R^{1c} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R or a C₁₋₆ aliphatic group, such as methyl, which may be optionally substituted with R, wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

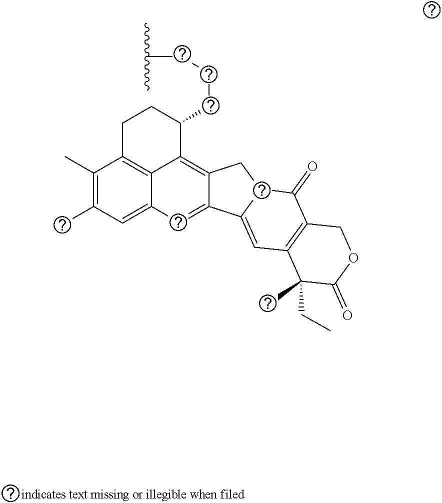

wherein, R¹ may be —O— or —HN—; and
X may be -L¹-CH₂—C(O)—, or may be -A-C(O)—;
wherein, when X is -L¹-CH₂—C(O)—, L¹ may be —(C(R^{3a})(R^{3b}))ₘ, wherein m may be selected from the group consisting of 0, 1 and 2, wherein 0 or 1 methylene unit of L¹ may be independently replaced by —C(O)—;
wherein when X is -A-C(O)—, A may be a ring moiety, selected from the group consisting of phenyl, pyridyl, cyclohexyl, cyclopentyl, and cyclobutyl, wherein A may be un-substituted or may be substituted with one or more substituents R^{1c};
wherein each of R^{3a}, R^{3b} and R^{1c} may each independently be hydrogen, protium, deuterium, tritium, halogen, —NO₂, —CN, —OR, —SR, —N(R^{a})(R^{b}), —C(O)R or a C₁₋₆ aliphatic group, such as methyl, which may be optionally substituted with R, wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

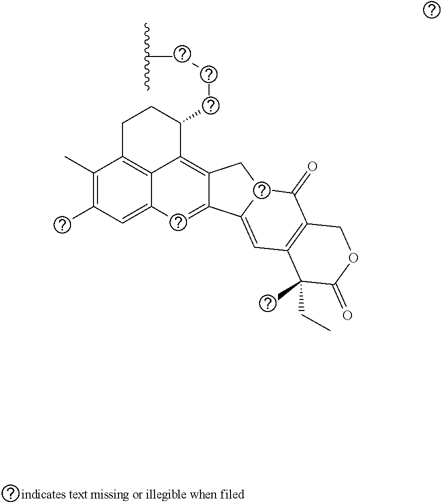

wherein, R¹ may be —O— or —HN—; and
X may be -L¹-CH₂—C(O)—, or may be -A-C(O)—;
wherein, when X is -L¹-CH₂—C(O)—, L¹ may be —(C(R^{3a})(R^{3b}))ₘ, wherein m may be selected from the group consisting of 0, 1 and 2, wherein 0 or 1 methylene unit of L¹ may be independently replaced by —C(O)—;
wherein when X is -A-C(O)—, A may be a ring moiety, selected from the group consisting of phenyl, pyridyl, cyclohexyl, cyclopentyl, and cyclobutyl, wherein A may be un-substituted or may be substituted with one or more substituents R^{1c};
wherein each of R^{3a}, R^{3b} and R^{1c} may each independently be hydrogen or a C₁₋₆ aliphatic group, such as methyl, which may be optionally substituted with R, wherein R may be hydrogen, halogen, —NO₂, —CN, —OH, —NH₂ or a C₁₋₆ aliphatic group.

In one embodiment, the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof:

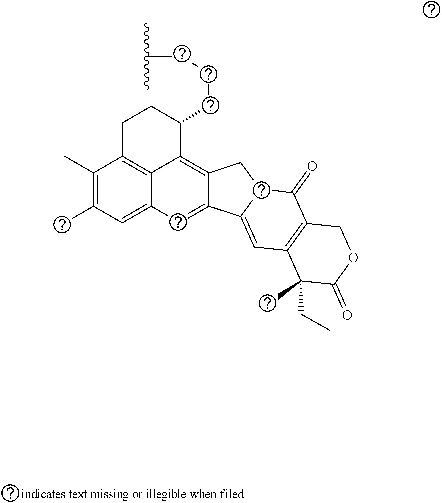

wherein, R¹ may be —O— or —HN—; and
X may be -L¹-CH₂—C(O)—;
wherein, L¹ may be —(C(R^{3a})(R^{3b}))ₘ, wherein m may be selected from the group consisting of 0, 1 and 2, wherein 0 or 1 methylene unit of L¹ may be independently replaced by —C(O)—;
wherein each of R^{3a} and R^{3b} may each independently be hydrogen or a C₁₋₆ aliphatic group, such as methyl, which may be optionally substituted with R, wherein R may be hydrogen, halogen, —NO₂, —CN, —OH, —NH₂ or a C₁₋₆ aliphatic group.

In one embodiment of the above described structures of Formula (III-A), R¹ is chemically bonded to X described above via the L¹ moiety of X.

In one embodiment, the antibody-drug conjugate comprises an active agent with a structure according to formula (IV-A) or a tautomer, a mesomer, a racemate, an enantiomer or a diastereoisomer thereof, or a mixture thereof, or a pharmaceutically acceptable salt thereof:

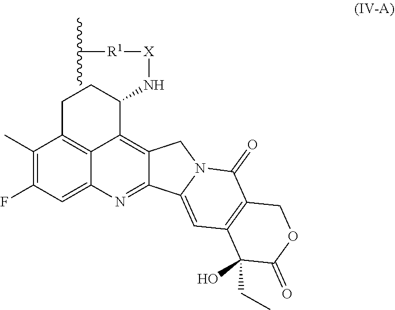

wherein in formula (IV-A), R¹ may be —O— or —(R²)N— or —S—; and X may be -L¹-CH₂—C(O)—, or may be -A-C(O)—;
wherein, when X is -L¹-CH₂—C(O)—, L¹ may be —(C(R^{3a})(R^{3b}))ₘ, wherein m may be selected from the group consisting of 0, 1 and 2, wherein 0 or 1 methylene unit of L¹ may be independently replaced by —C(O)— or —C(S)—;
wherein when X is -A-C(O)—, A may be a ring structure moiety, selected from the group consisting of phenyl, pyridyl, cyclohexyl, cyclopentyl, and cyclobutyl, wherein A may be un-substituted or may be substituted with one or more substituents R^{1c};
wherein each of R², R^{3a}, R^{3b} and R^{1c} may each independently be hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R or a C₁₋₆ aliphatic group, such as methyl, which may be optionally substituted with R, wherein each R, each R^{a} and each R^{b} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group; or
wherein in formula (IV-A), R¹ may be —O— or —HN—; and
X may be -L¹-CH₂—C(O)—, or may be -A-C(O)—;
wherein, when X is -L¹-CH₂—C(O)—, L¹ may be —(C(R^{3a})(R^{3b}))ₘ, wherein m may be selected from the group consisting of 0, 1 and 2, wherein 0 or 1 methylene unit of L¹ may be independently replaced by —C(O)—;
wherein when X is -A-C(O)—, A may be a ring moiety, selected from the group consisting of phenyl, pyridyl, cyclohexyl, cyclopentyl, and cyclobutyl, wherein A may be un-substituted or may be substituted with one or more substituents R^{1c}; wherein each of R^{3a}, R^{3b} and R^{1c} may each independently be hydrogen or a C₁₋₆ aliphatic group, such as methyl, which may be optionally substituted with R, wherein R may be hydrogen, halogen, —NO₂, —CN, —OH, —NH₂ or a C₁₋₆ aliphatic group; or wherein in formula (IV-A), R¹ may be —O— or —HN—; and
X may be -L¹-CH₂—C(O)—;
wherein, L¹ may be —(C(R^{3a})(R^{3b}))ₘ, wherein m may be selected from the group consisting of 0, 1 and 2, wherein 0 or 1 methylene unit of L¹ may be independently replaced by —C(O)—;
wherein each of R^{3a} and R^{3b} may each independently be hydrogen or a C₁₋₆ aliphatic group, such as methyl, which may be optionally substituted with R, wherein R may be hydrogen, halogen, —NO₂, —CN, —OH, —NH₂ or a C₁₋₆ aliphatic group.

In one embodiment of the above described structures of Formula (III-A), R¹ is chemically bonded to X described above via the L¹ moiety of X.

In one embodiment of formula (IV-A), R¹ may be —O—; and X may be -L¹-CH₂—C(O)—. In one further embodiment, L¹ may be —(C(R^{3a})(R^{3b}))ₘ, wherein m may be selected from the group consisting of 0, 1 and 2, wherein 0 or 1 methylene unit of L¹ may be independently replaced by —C(O)—. Preferably m is 1 such that L¹ may be —(C(R^{3a})(R^{3b})), wherein each of R^{3a} and R^{3b} may each independently be hydrogen or a C₁₋₆ aliphatic group, such as methyl, which may be optionally substituted with R, wherein R may be hydrogen, halogen, —NO₂, —CN, —OH, —NH₂ or a C₁₋₆ aliphatic group.

### Linker

A stable link between the antibody and the active agent is an important aspect of ADC technology. Linkers may e.g. be based on chemical motifs including disulfides, hydrazones or peptides (cleavable), or thioethers (noncleavable), and control the distribution and delivery of the active agent to the target cell. Cleavable and noncleavable types of linkers have been proven to be safe in preclinical and clinical trials. For example, Brentuximab Vedotin includes an enzyme-sensitive cleavable linker that delivers the potent and highly toxic antimicrotubule agent monomethyl auristatin E (MMAE), a synthetic antineoplastic agent, to cells.

Trastuzumab Emtansine, another approved ADC, is a combination of the microtubule-formation inhibitor mertansine (DM-1), a derivative of the Maytansine, and antibody Trastuzumab (Herceptin™, Genentech/Roche), attached by a stable, non-cleavable linker.

The type of linker, cleavable or non-cleavable, lends specific properties to the delivered active agent. For example, cleavable linkers can e.g. be cleaved by enzymes in the target cell, leading to efficient intracellular release of the active agent, for example a cytotoxic agent. In contrast, an ADC containing a non-cleavable linker has no mechanism for drug release, and must rely on mechanisms such as degradation of the targeting antibody, for drug release. Furthermore, as is appreciated by those skilled in the art, the linker composition may influence critical factors such as solubility and pharmacokinetic properties of the ADC as a whole.

For both types of linker, drug release is crucial for obtaining a cellular effect. Drugs which are able to freely diffuse across cell membranes may escape from the targeted cell and, in a process called “bystander killing,” also attack neighbouring cells, such as cancer cells in the vicinity of the uPARAP expressing target cell.

In a preferred embodiment of the present disclosure, the ADC targeting uPARAP as disclosed herein comprises a linker that links the antibody to the active agent.

In one embodiment of the present disclosure, the linker may be cleavable or non-cleavable.

Cleavable groups include a disulfide bond, an amide bond, a substituted amide bond in the form of a peptide bond, a thioamide, bond, an ester bond, a thioester bond, a vicinal diol bond, or a hemiacetal. These, or other cleavable bonds, may include enzymatically-cleavable bonds, such as peptide bonds (cleaved by peptidases), phosphate bonds (cleaved by phosphatases), nucleic acid bonds (cleaved by endonucleases), and sugar bonds (cleaved by glycosidases).

In a further embodiment of the present disclosure, the linker is a cleavable linker allowing for intracellular release of the active agent inside the target cells.

In a further embodiment the linker is a peptide linker. The choice of peptide sequence is critical to the success of the conjugate. In some embodiments the linker is stable to serum proteases, yet is cleaved by lysosomal enzymes in the target cell.

In a further embodiment the linker is an enzyme-cleavable peptide-containing linker, such as a cathepsin cleavable peptide-containing linker. Cathepsin can be one of several cathepsin types, being one of a group of lysosomal proteases.

In a further embodiment of the present disclosure, the linker comprises or consists of a dipeptide, such as valine-citrulline (VC) or valine-alanine (VA).

In one embodiment the linker comprises or consists of a dipeptide, such as valine-citrulline (VC) or valine-alanine (VA), which may be further connected through an amide linkage to other structural elements. Valine-citrulline-based linkers, in which the citrulline carboxyl function is modified to a substituted amide, can be cleaved by lysosomal cathepsins, whereas valine-alanine-based linkers, in which the alanine carboxyl function is modified to a substituted amide, can be cleaved by other lysosomal proteases, including other cathepsins.

In a further embodiment of the present disclosure, the antibody-drug conjugate as defined herein further comprises a spacer, such as a spacer comprising p-aminobenzoic acid (PAB), p-aminobenzylcarbamate (PABC), p-aminobenzoyloxycabonyl, or polyethyleneglycol (PEG).

In one embodiment of the present disclosure, the antibody-drug conjugate as defined herein comprises p-aminobenzylcarbamate (PABC).

In a further embodiment of the present disclosure, the antibody-drug conjugate as defined herein further comprises an attachment group, such as an attachment group comprising or consisting of maleimide and caproic acid (MC), N-hydroxysuccinimide, reactive attachment groups directed to modified or unmodified protein-bound carbohydrate, peptide sequences that are required for enzymatic reactions, azides or alkynes or being derived from these by reaction with the antibody or a chemically or enzymatically generated derivative thereof.

In one embodiment of the present disclosure, the ADC of the present disclosure further comprises an attachment entity. The attachment entity may for example connect the antibody and the cleavable linker, where the attachment entity is the reaction product between an antibody amino acid side chain and a reactive attachment group in the linker precursor. In one embodiment, this reactive attachment group comprises or consists of maleimide and caproic acid (MC), where maleimide reacts preferably with cysteine thiols during coupling. In other embodiments, the attachment group comprises or consists of N-hydroxysuccinimide, reactive attachment groups directed to modified or unmodified protein-bound carbohydrate, peptide sequences that are required for enzymatic reactions, azides or alkynes or being derived from these by reaction with the antibody or a chemically or enzymatically generated derivative thereof.

### I-B

In one embodiment, antibody-drug conjugate comprises a structure according to formula (I-B) or a tautomer, a mesomer, a racemate, an enantiomer or a diastereoisomer thereof, or a mixture thereof, or a pharmaceutically acceptable salt thereof:

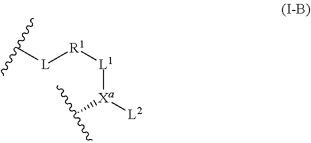

wherein, X^{a} may be a nitrogen generated by removal of two hydrogen atoms from an amino group of exatecan (EXA);
L may be -Lₐ-L_{b}-L_{c}-; wherein L links the structure shown in formula (I-B) to the antibody as defined herein;
-Lₐ- may be selected from the group consisting of:

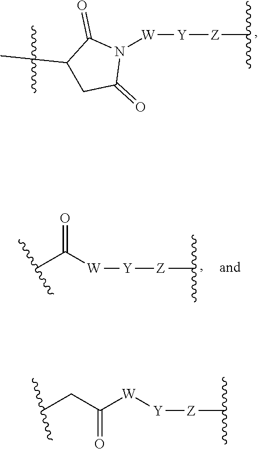

wherein W may be —(C(R^{wa})(R^{wb}))_{wn}—, Y may be —(OCH₂CH₂)_{yn}—O_{yp}—, and Z may be —(C(R^{za})(R^{zb}))_{zn};
wherein Z connects —La— to -Lb-;
wherein wn may be selected from the group consisting of integers≥0, and
0 or no less than 1 methylene unit of W may be independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn may be selected from the group consisting of integers≥0, and yp may be 0 or 1;
wherein zn may be selected from the group consisting of integers≥0, and
0 or no less than 1 methylene unit of Z may be independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- may be selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or may be independently substituted with no less than 1 substituent R^{cx};
wherein each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r} or a C₁₋₆ aliphatic group which may be optionally substituted with R^{r};
wherein each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- may be selected from the group consisting of:

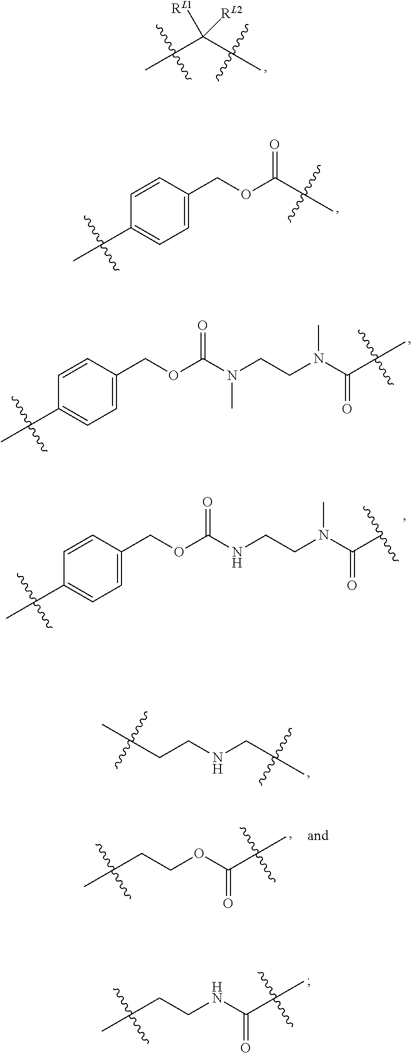

wherein R^{L1} and R^{L2} may each independently be selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group;
R¹, L¹ and L² are defined as in any formula (I-A) in embodiments described herein above.

### II-Bx, II-By

In one embodiment, antibody-drug conjugate comprises a structure according to formula (II-Bx) or formula (II-By) or a tautomer, a mesomer, a racemate, an enantiomer or a diastereoisomer thereof, or a mixture thereof, or a pharmaceutically acceptable salt thereof:

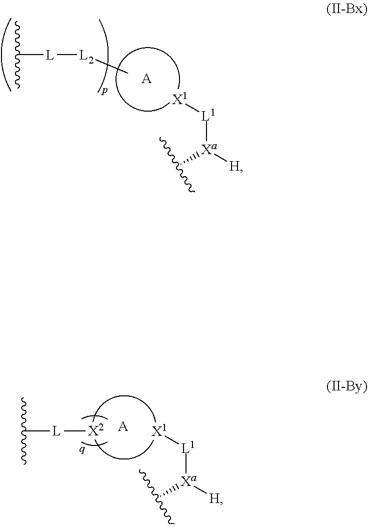

wherein, X^{a} may be nitrogen generated by removal of two hydrogen atoms from an amino group of exatecan (EXA);
L may be -Lₐ-L_{b}-L_{c}-; wherein L links the structure shown in formula (II-Bx) or formula (II-By) to the antibody as defined herein;
-Lₐ- may be selected from the group consisting of:

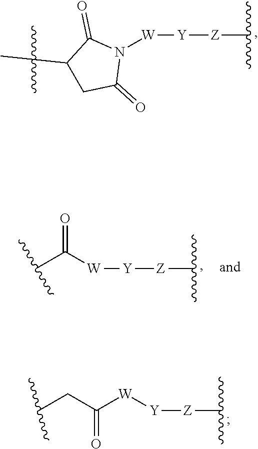

wherein W may be —(C(R^{wa})(R^{wb}))_{wn}—, Y may be —(OCH₂CH₂)_{yn}—O_{yp}—, and Z may be —(C(R^{za})(R^{zb}))_{zn};
wherein Z connects —La— to -Lb-;
wherein wn may be selected from the group consisting of integers≥0, and
0 or no less than 1 methylene unit of W may be independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn may be selected from the group consisting of integers≥0, and yp may be 0 or 1;
wherein zn may be selected from the group consisting of integers≥0, and 0 or no less than 1 methylene unit of Z may be independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- may be selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or may be independently substituted with no less than 1 substituent R^{cx};
wherein each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r} or a C₁₋₆ aliphatic group which may be optionally substituted with R^{r};
wherein each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- may be selected from the group consisting of:

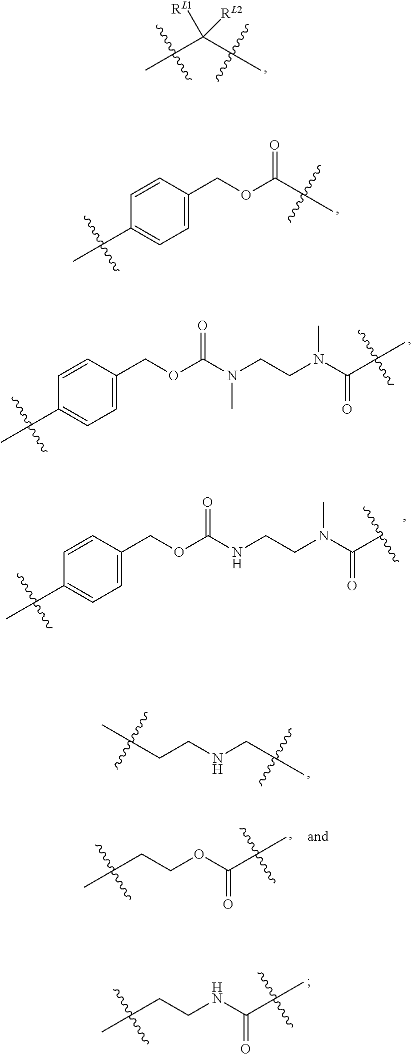

wherein R^{L1} and R^{L2} may each independently be selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group;
L², p, ring A, X¹ and L¹ are defined as in any formula (II-Ax) in embodiments described herein above;
or X², q, ring A, X¹ and L¹ are defined as in any formula (II-Ay) in embodiments described herein above.

### III-B

In one embodiment, antibody-drug conjugate comprises a structure according to formula (III-B) or a tautomer, a mesomer, a racemate, an enantiomer or a diastereoisomer thereof, or a mixture thereof, or a pharmaceutically acceptable salt thereof:

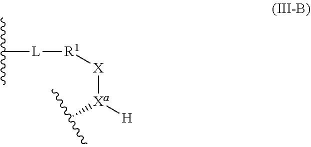

wherein, X^{a} may be nitrogen generated by removal of two hydrogen atoms from an amino group of exatecan (EXA);
L may be -Lₐ-L_{b}-L_{c}-; wherein L links the structure shown in formula (II-Bx) or formula (II-By) to the antibody as defined herein;
-Lₐ- may be selected from the group consisting of:

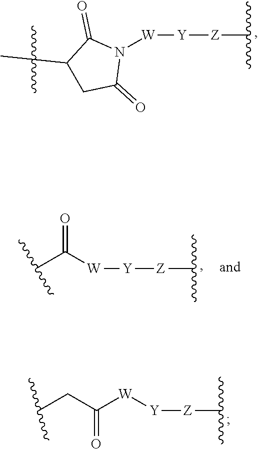

wherein W may be —(C(R^{wa})(R^{wb}))_{wn}—, Y may be —(OCH₂CH₂)_{yn}—O_{yp}, and Z may be —(C(R^{za})(R^{zb}))_{zn};
wherein Z connects —La— to -Lb-;
wherein wn may be selected from the group consisting of integers≥0, and
0 or no less than 1 methylene unit of W may be independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn may be selected from the group consisting of integers≥0, and yp may be 0 or 1;
wherein zn may be selected from the group consisting of integers≥0, and 0 or no less than 1 methylene unit of Z may be independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- may be selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or may be independently substituted with no less than 1 substituent R^{cx};
wherein each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r} or a C₁₋₆ aliphatic group which may be optionally substituted with R^{r};
wherein each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- may be selected from the group consisting of:

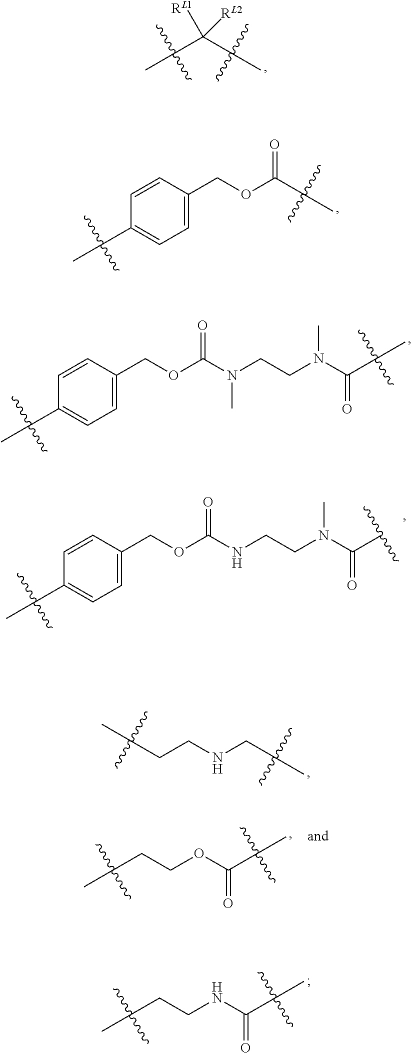

wherein R^{L1} and R^{L2} may each independently be selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group;
wherein R¹ and X are defined as in any formula (III-A) in embodiments as described herein above.

In another embodiment, wn may be selected from the group consisting of integers from 2 to 6, and 0 or 1 methylene unit of W may be independently replaced by -Cyr-, —N(R^{wx})C(O)—, —C(O)N(R^{wx})—, —C(O)—, —NR^{wx}— or —O—.

For example, wn may be 1, 2, 3 or 6, and 1 methylene unit of W may be independently replaced by -Cyr-, —N(R^{wx})C(O)—, —C(O)N(R^{wx})— or —C(O)—.

In another embodiment, yn may be selected from the group consisting of integers from 0 to 12, and yp may be 0 or 1.

For example, yn may be 0, 4 or 8, and yp may be 0 or 1.

In another embodiment, zn may be selected from the group consisting of integers from 0 to 10, and 0 or 1 methylene unit of Z may be independently replaced by -Cyr-, —N(R^{zx})C(O)—, —C(O)N(R^{zx})— or —C(O)—.

For example, zn may be 1, 2 or 3, and 1 methylene unit of Z may be independently replaced by -Cyr-, —N(R^{zx})C(O)—, —C(O)N(R^{zx})— or —C(O)—.

In another embodiment, —Cyr- may be selected from the group consisting of: 6-10 membered arylene and 3-10 membered saturated or partially unsaturated carbocyclylene, wherein -Cyr- is unsubstituted or may be independently substituted with 1 to 3 substituent R^{cx}.

For example, —Cyr- may be 3-10 membered saturated carbocyclylene, wherein -Cyr- is unsubstituted or may be independently substituted with 1 to 3 substituent R^{cx}.

In another embodiment, each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group which may be optionally substituted with R^{r}; each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

For example, each R^{wa}, each R^{wb}, each R^{za}, each R^{zb}, each R^{wx}, each R^{zx} and each R^{cx} may each independently be hydrogen, halogen, —OR^{r}, or a C₁₋₆ aliphatic group which may be optionally substituted with R^{r}; each R^{r} may independently be hydrogen, halogen or a C₁₋₆ aliphatic group.

In another embodiment, -L_{b}- represents a peptide residue consisting of 2 to 7 amino acids, and the peptide residue of -L_{b}- may be a peptide residue which may be formed of amino acids which may be selected from the group consisting of: phenylalanine, glycine, alanine, valine, citrulline, lysine, serine, glutamic acid and aspartic acid.

For example, -L_{b}- represents a peptide residue consisting of 2 to 4 amino acids, and the peptide residue of -L_{b}- may be a peptide residue which may be formed of amino acids which may be selected from the group consisting of: phenylalanine, glycine, alanine, valine, citrulline and lysine. In one embodiment of the present disclosure -L_{b}- may comprise or consist of SEQ ID NO 14 or 15.

For example, -L_{b}- may be selected from the group consisting of:

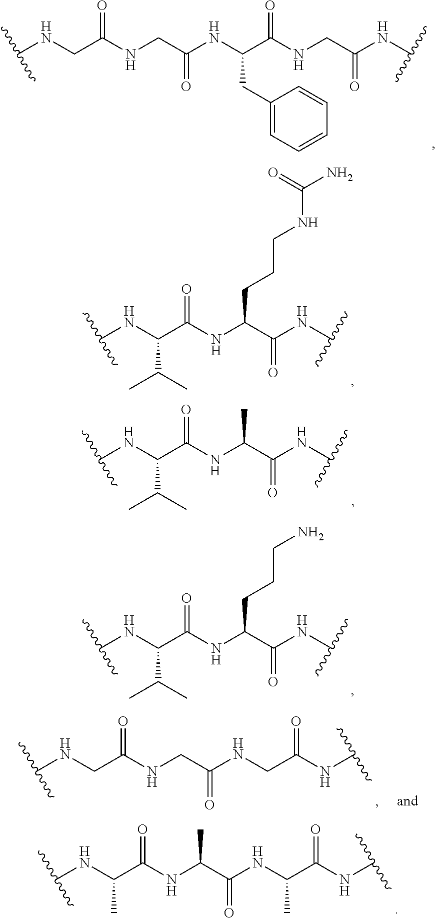

For example, -L_{b}- may be

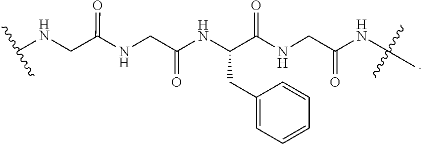 [SMILES: *NCC(=O)NCC(=O)N[C@@H](Cc1ccccc1)C(=O)NCC(=O)N*]

In another embodiment, -L_{c}- may be selected from the group consisting of:

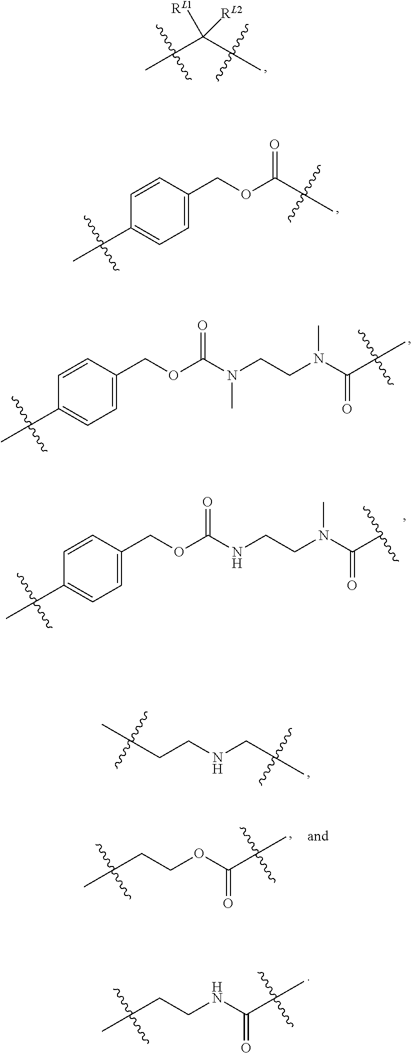

For example, -L_{c}- may be selected from the group consisting of:

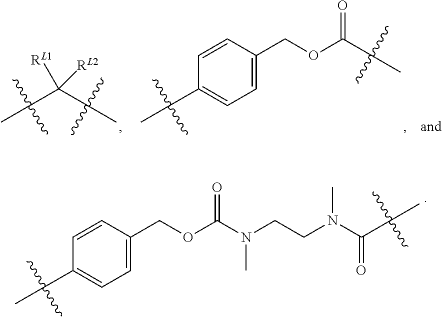

-L_{c}- may be

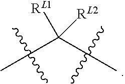

In another embodiment, R^{L1} and R^{L2} may each independently be selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group.

For example, R^{L1} and R^{L2} may each independently be selected from the group consisting of: hydrogen, halogen, —OH and a C₁₋₆ aliphatic group.

In another embodiment, -Lₐ- may be

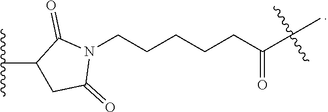 [SMILES: *C(=O)CCCCCN1C(=O)CC(*)C1=O]

In another embodiment, -L_{b}- may be selected from the group consisting of:

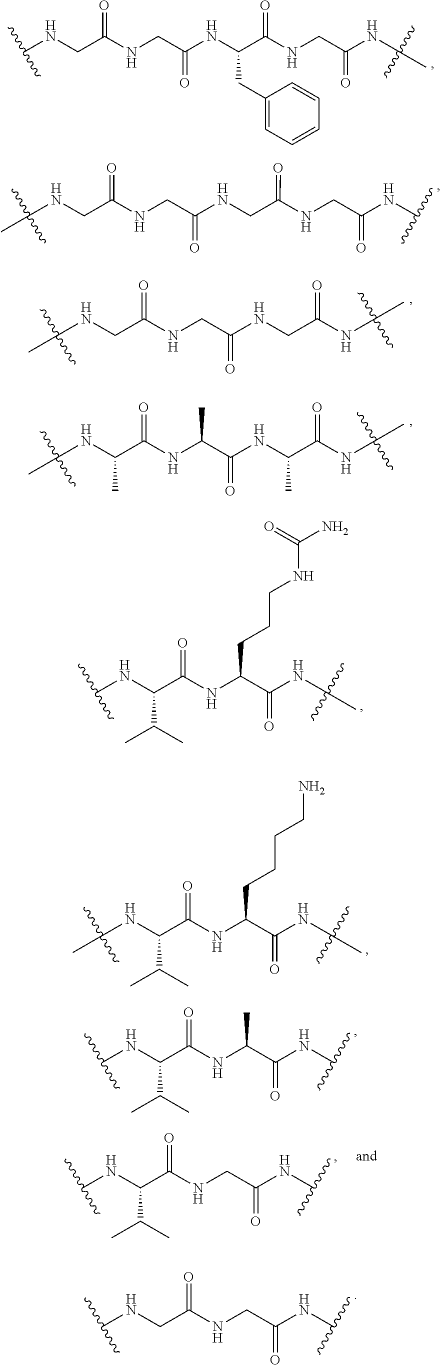

For example, -L_{b}- may be selected from the group consisting of:

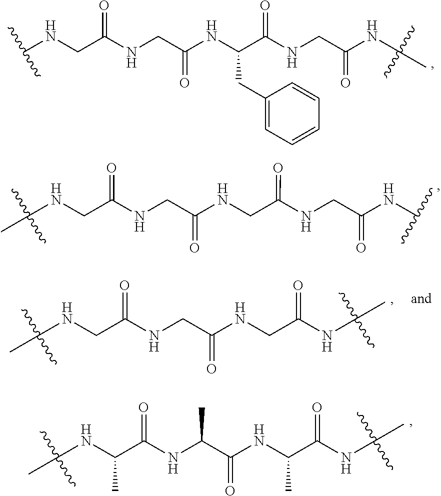

In one embodiment, -L_{c}- may be

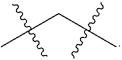 [SMILES: *C*]

In one embodiment, -Lₐ-L_{b}-L_{c}- may be selected from the group consisting of:

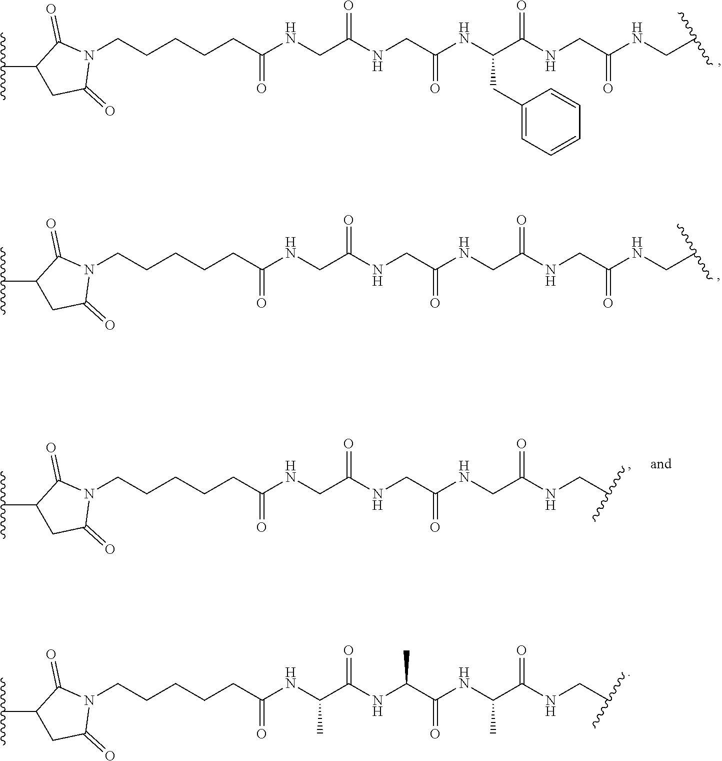 [SMILES: *CNC(=O)CNC(=O)[C@H](Cc1ccccc1)NC(=O)CNC(=O)CNC(=O)CCCCCN1C(=O)CC(*)C1=O ; *CNC(=O)CNC(=O)CNC(=O)CNC(=O)CNC(=O)CCCCCN1C(=O)CC(*)C1=O ; *CNC(=O)CNC(=O)CNC(=O)CNC(=O)CCCCCN1C(=O)CC(*)C1=O ; *CNC(=O)[C@H](C)NC(=O)[C@H](C)NC(=O)[C@H](C)NC(=O)CCCCCN1C(=O)CC(*)C1=O]

As used herein, the formula referred to as exatecan (EXA) is

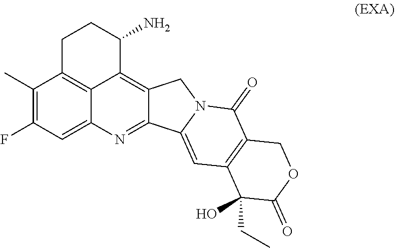 [SMILES: CC[C@@]1(O)C(=O)OCc2c1cc1n(c2=O)Cc2c-1nc1cc(F)c(C)c3c1c2[C@@H](N)CC3]

Exatecan (chemical name: (1S,9S)-1-amino-9-ethyl-5-fluoro-2,3-dihydro-9-hydroxy-4-methyl-1H,12H-benzo[de]pyrano[3′,4′:6,7]imidazo[1,2-b]quinoline-10,13(9H,15H)-dione)) is a camptothecin derivative. Functionally, exatecan is classified as a topoisomerase inhibitor, more specifically a topoisomerase I inhibitor.

### I-C.

In one embodiment, the antibody drug conjugate comprises formula (I-C) or a tautomer, a mesomer, a racemate, an enantiomer or a diastereoisomer thereof, or a mixture thereof, or a pharmaceutically acceptable salt thereof:

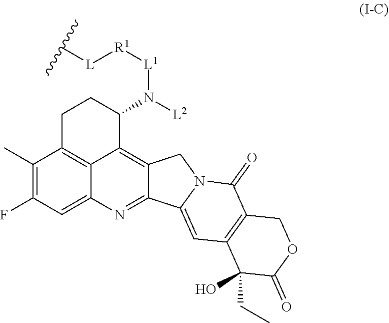

wherein, L may be -Lₐ-L_{b}-L_{c}-, and Lₐ, L_{b} and L_{c} are defined as in any formula (I-B) embodiments as described herein above;
R¹, L¹ and L² are defined as in any formula (I-A) in embodiments as described herein above.

### II-Cx, II-Cy

In one embodiment, the antibody drug conjugate comprises formula (II-Cx) or (II-Cy) or a tautomer, a mesomer, a racemate, an enantiomer or a diastereoisomer thereof, or a mixture thereof, or a pharmaceutically acceptable salt thereof

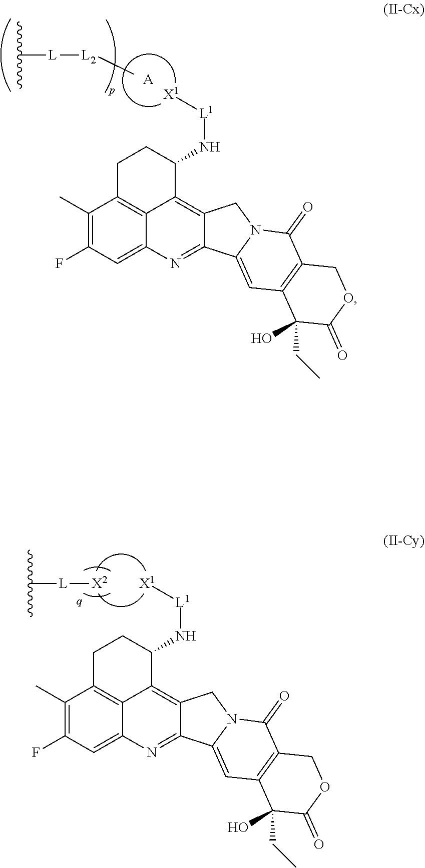

wherein, L may be -Lₐ-L_{b}-L_{c}-, and Lₐ, L_{b} and L_{c} are defined as in any formula (II-Bx) in embodiments as described herein above;
L², p, ring A, X¹ and L¹ are defined as in any formula (II-Ax) in embodiments as described herein above;
or X², q, ring A, X¹ and L¹ are defined as in any formula (II-Ay) in embodiments as described herein above.

### III-C

In one embodiment, the antibody drug conjugated comprises formula (III-C) or a tautomer, a mesomer, a racemate, an enantiomer or a diastereoisomer thereof, or a mixture thereof, or a pharmaceutically acceptable salt thereof:

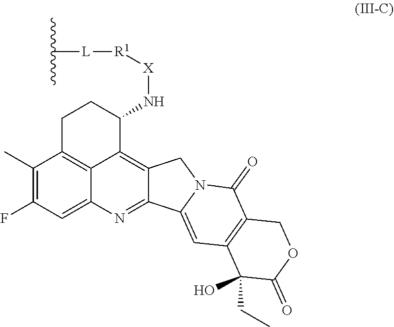

wherein, L may be -Lₐ-L_{b}-L_{c}-, and Lₐ, L_{b} and L_{c} are defined as in any formula (III-B) in embodiments as described herein above;
wherein R¹ and X are defined as in any formula (III-A) in embodiments as described herein above.

### I-D

In one embodiment, the antibody drug conjugate comprises formula (I-D) or a tautomer, a mesomer, a racemate, an enantiomer or a diastereoisomer thereof, or a mixture thereof, or a pharmaceutically acceptable salt thereof:

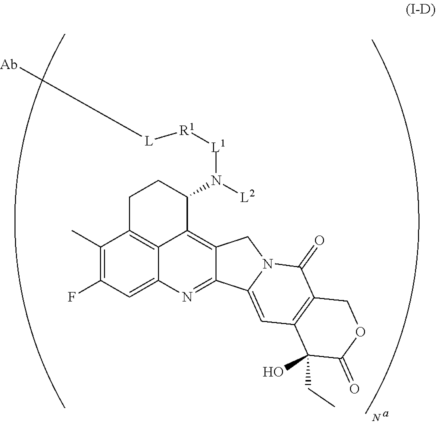

wherein Ab represents an antibody as defined herein, comprising:
   i) an immunoglobulin light chain variable region comprising or consisting of the amino acid sequence of SEQ ID NO: 3; and
   ii) an immunoglobulin heavy chain variable region comprising or consisting of the amino acid sequence of SEQ ID NO: 6;
N^{a} represents an average connection number and may be an integer or a decimal from 1 to 10;
L may be -Lₐ-L_{b}-L_{c}-, and Lₐ, L_{b} and L_{c} are defined as in any formula (I-B) in embodiments described herein above,
R¹, L¹ and L² are defined as in any formula (I-A) in embodiments described herein above.

### II-Dx, II—Dy

In one embodiment, the antibody drug conjugate comprises formula (II-Dx) or formula (II—Dy) or a tautomer, a mesomer, a racemate, an enantiomer or a diastereoisomer thereof, or a mixture thereof, or a pharmaceutically acceptable salt thereof:

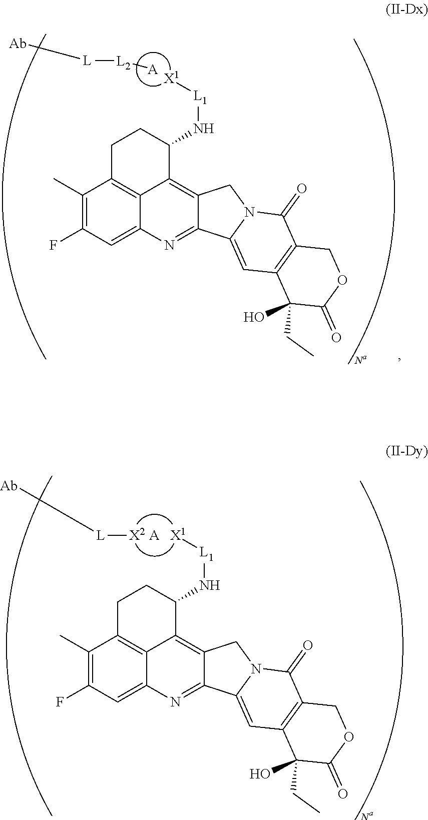

wherein Ab represents an antibody as defined herein, comprising:
   i) an immunoglobulin light chain variable region comprising or consisting of the amino acid sequence of SEQ ID NO: 3; and
   ii) an immunoglobulin heavy chain variable region comprising or consisting of the amino acid sequence of SEQ ID NO: 6;
N^{a} represents an average connection number and may be an integer or a decimal from 1 to 10,
L may be -Lₐ-L_{b}-L_{c}-, and Lₐ, L_{b} and L_{c} are defined as in any formula (II-Bx) or formula (II-By) in embodiments described herein above;
L², ring A, X¹ and L¹ are defined as in any formula (II-Ax) in embodiments as described herein above;
or X², ring A, X¹ and L¹ are defined as in any formula (II-Ay) in embodiments as described herein above.

### III-D

In one embodiment, the antibody drug conjugate comprises formula (III-D) or a tautomer, a mesomer, a racemate, an enantiomer or a diastereoisomer thereof, or a mixture thereof, or a pharmaceutically acceptable salt thereof:

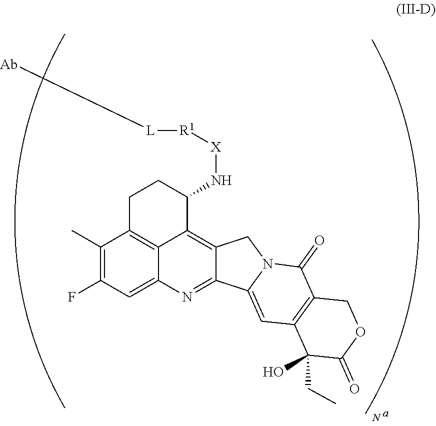

wherein Ab represents an antibody as defined herein, comprising:
   i) an immunoglobulin light chain variable region comprising or consisting of the amino acid sequence of SEQ ID NO: 3; and
   ii) an immunoglobulin heavy chain variable region comprising or consisting of the amino acid sequence of SEQ ID NO: 6;
N^{a} represents an average connection number and may be an integer or a decimal from 1 to 10,
L may be -Lₐ-L_{b}-L_{c}-, and Lₐ, L_{b} and L_{c} are defined as in any formula (III-B) in embodiments described herein above;
wherein R¹ and X are defined as in any formula (III-A) in embodiments described herein above.

In one embodiment, the ADC is according to any one of formulas (I-D), (II-Dx), (II—Dy) or (III-D) as defined herein, wherein Ab represents an antibody comprising:
i) an immunoglobulin light chain comprising or consisting of the amino acid sequence of SEQ ID NO: 1; and
ii) an immunoglobulin heavy chain comprising or consisting of the amino acid sequence of SEQ ID NO: 4.

In one embodiment, the ADC is according to any one of formulas (I-D), (II-Dx), (II—Dy) or (III-D) as defined herein, wherein Ab represents an antibody comprising:
i) an immunoglobulin light chain consisting of the amino acid sequence of SEQ ID NO: 1; and
ii) an immunoglobulin heavy chain consisting of the amino acid sequence of SEQ ID NO: 4.

In one embodiment, the antibody drug conjugate is selected from any one of the structures in Table 2, or a tautomer, a mesomer, a racemate, an enantiomer or a diastereoisomer thereof, or a mixture thereof, or a pharmaceutically acceptable salt thereof.

| | |
|---|---|
| 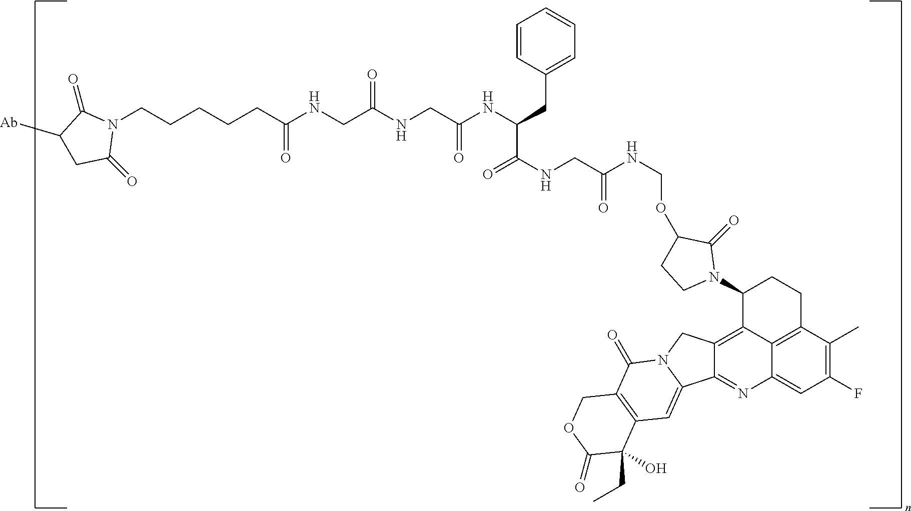 | D-I-1 |
| 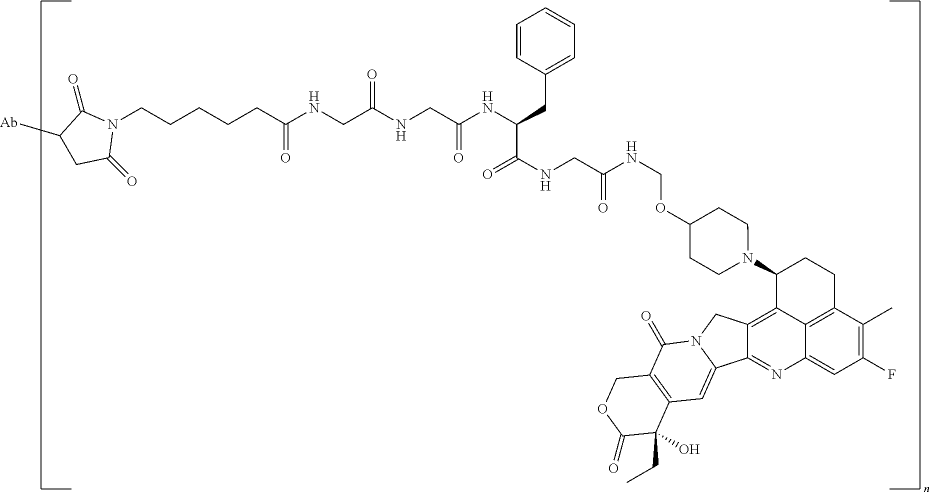 | D-I-2 |
| 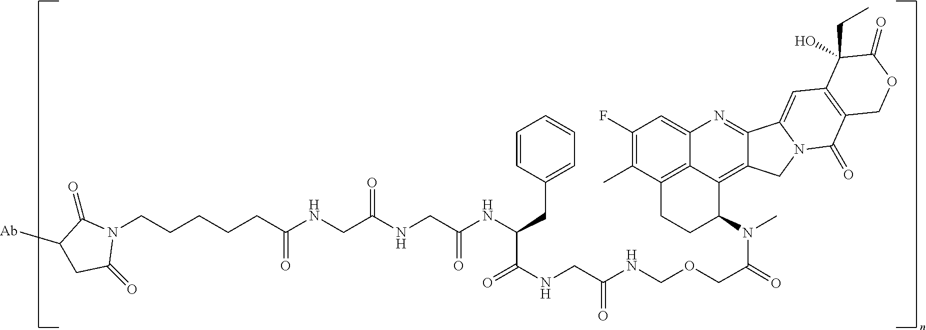 | D-I-3 |
| 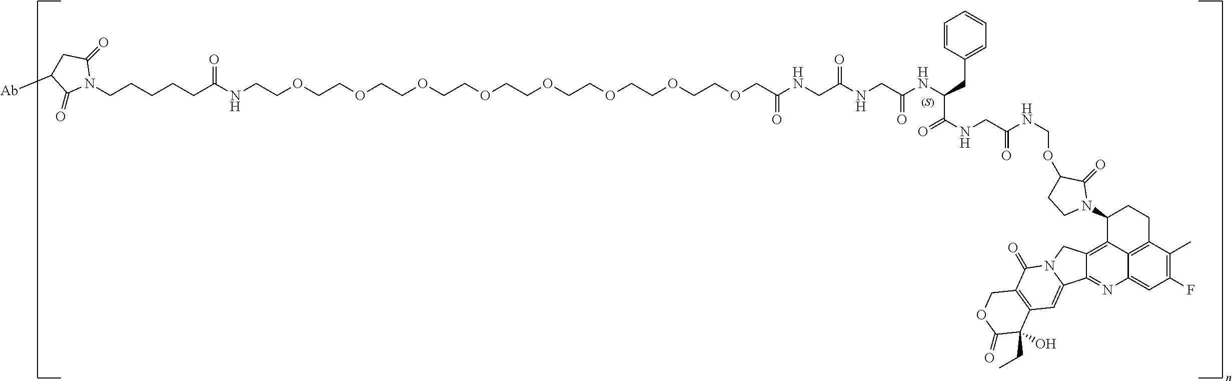 | D-I-4 |
| 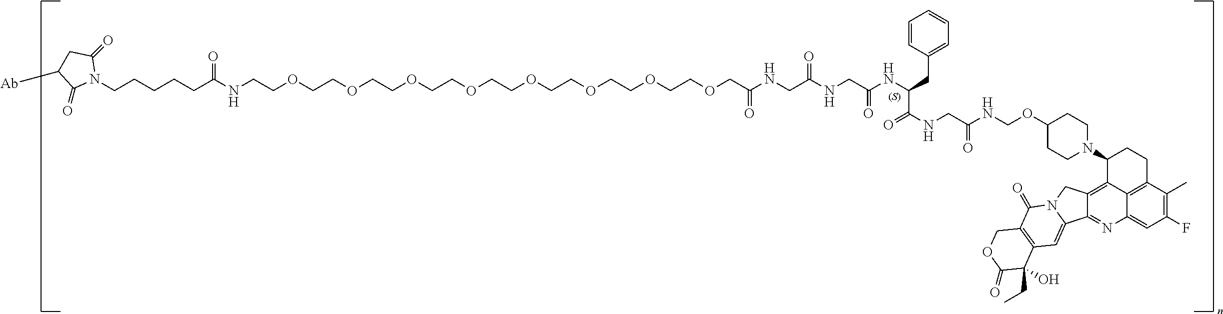 | D-I-5 |
| 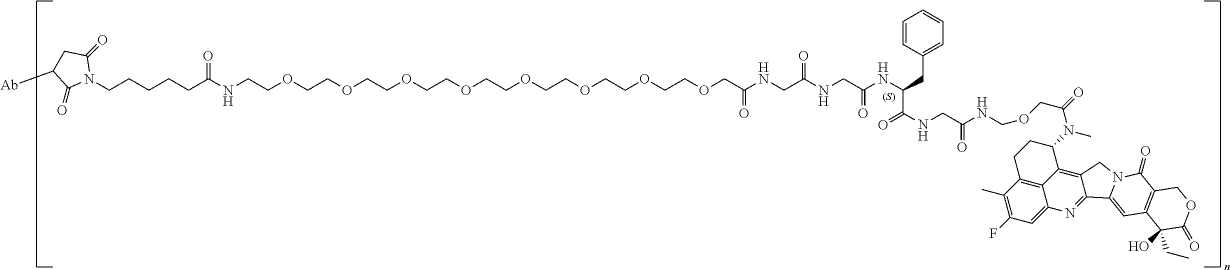 | D-I-6 |
| 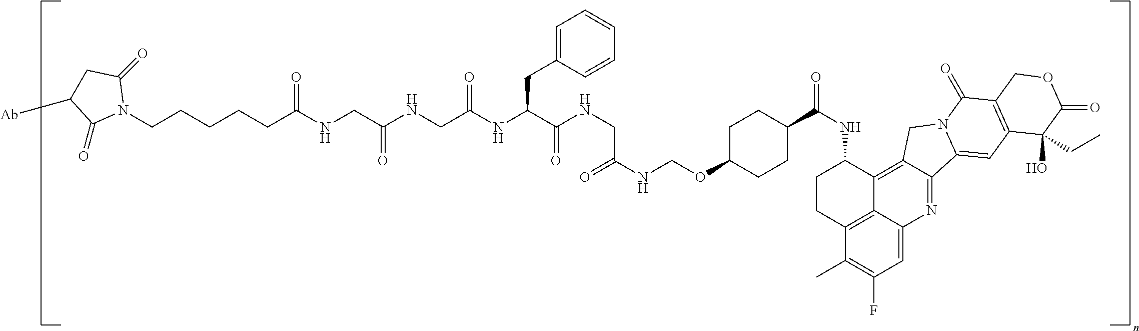 | D-II-1 |
| 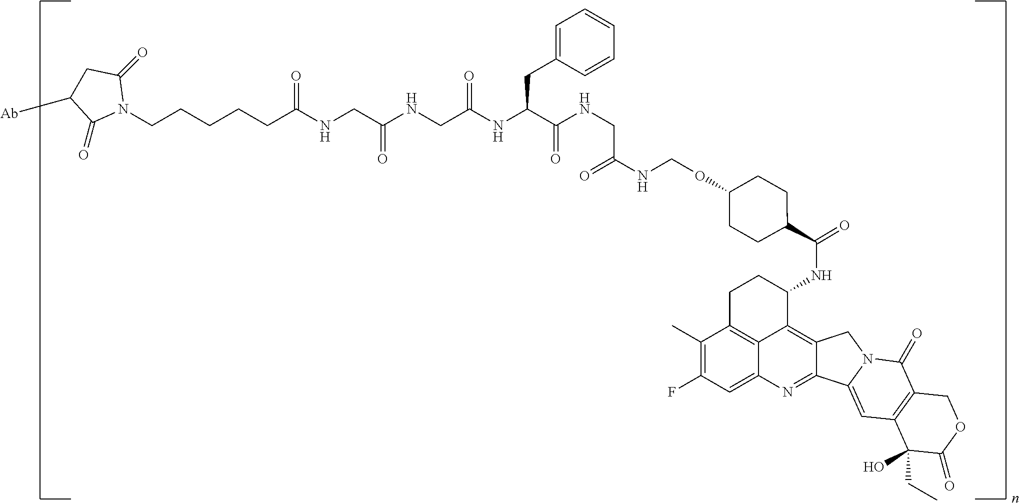 | D-II-2 |
| 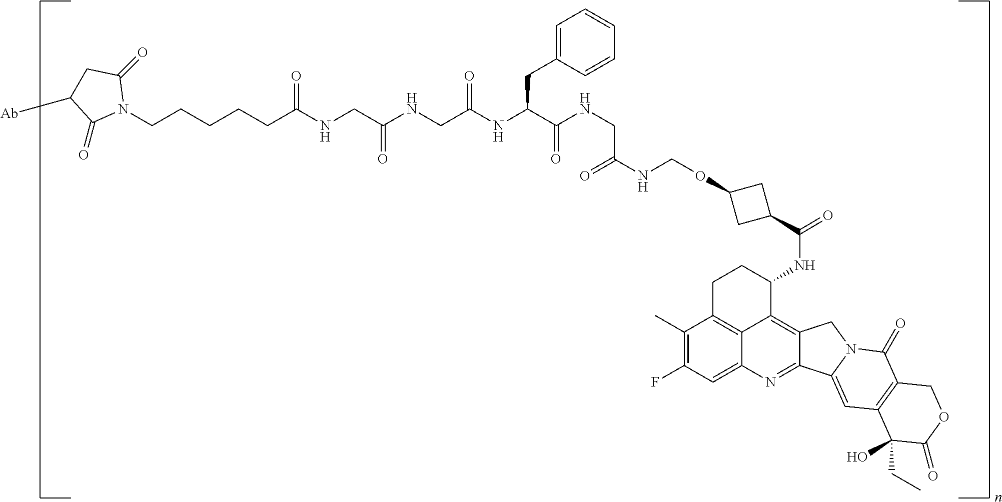 | D-II-3 |
| 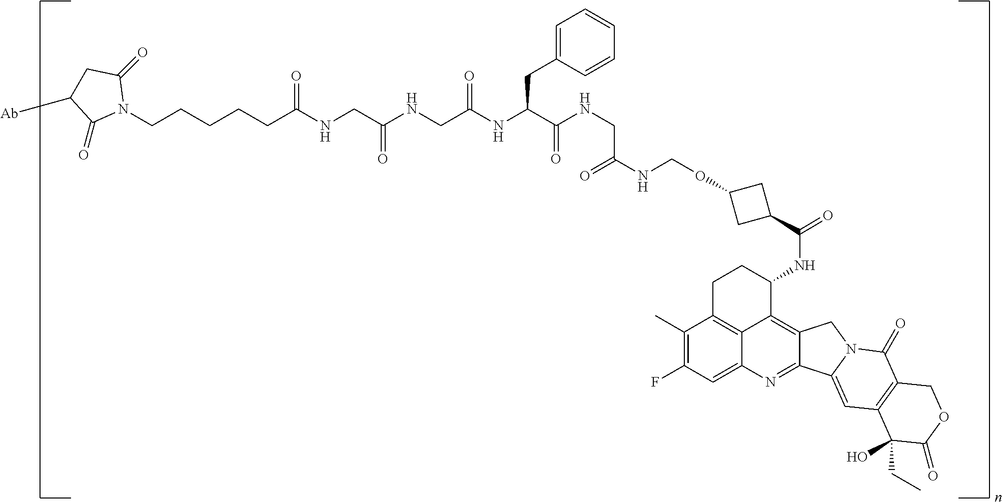 | D-II-4 |
| 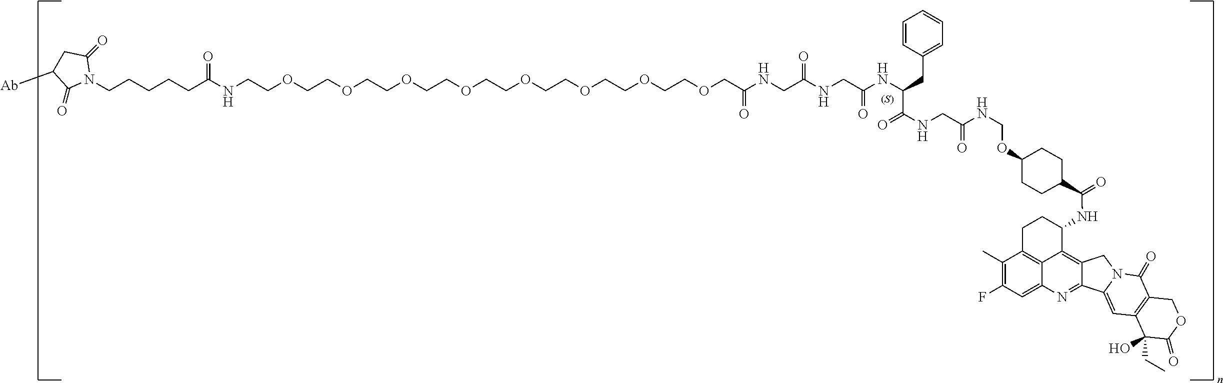 | D-II-5 |
| 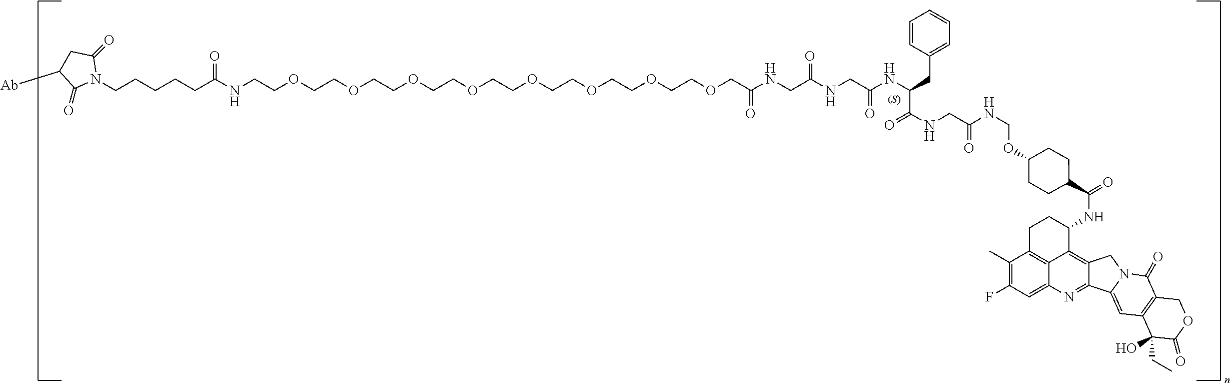 | D-II-6 |
| 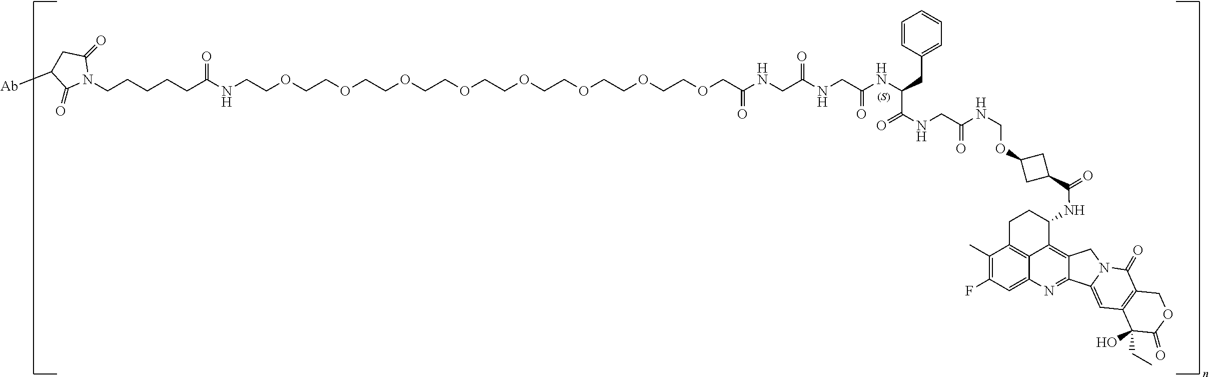 | D-II-7 |
| 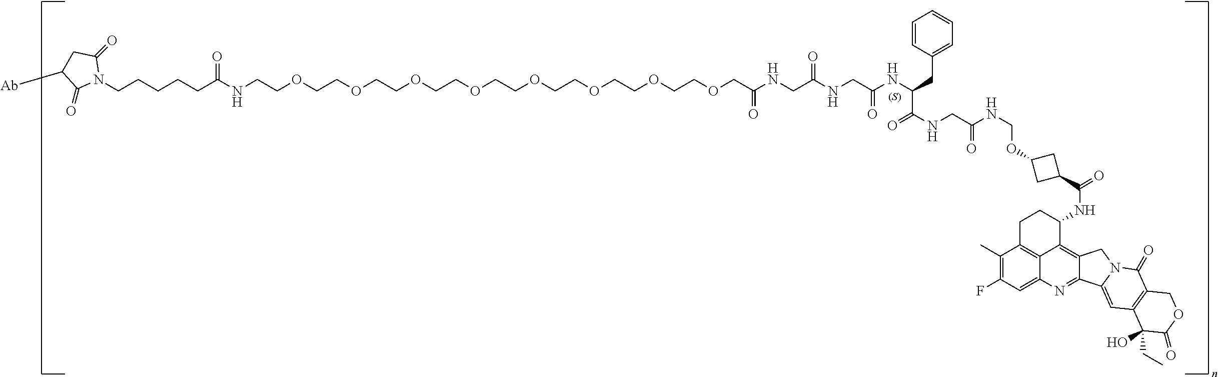 | D-II-8 |
| 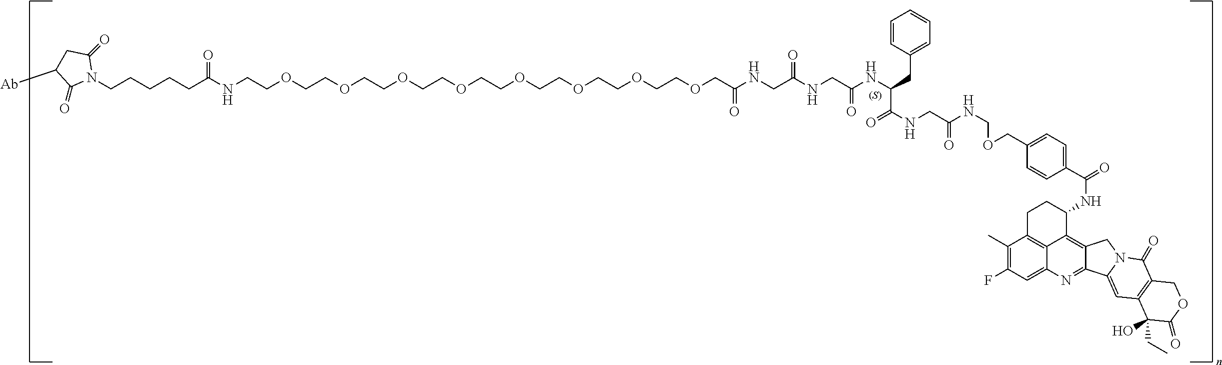 | D-II-9 |
| 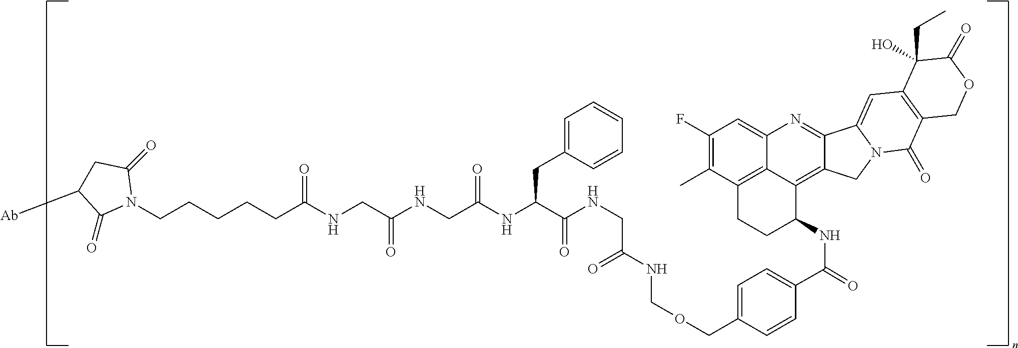 | D-II-10 |
| 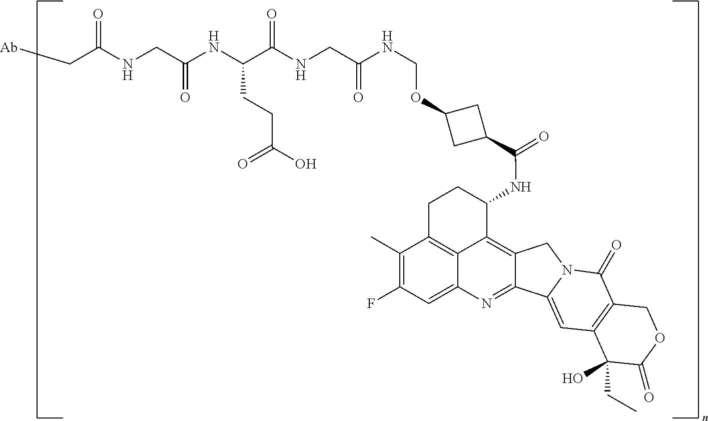 | D-II-11 |
| 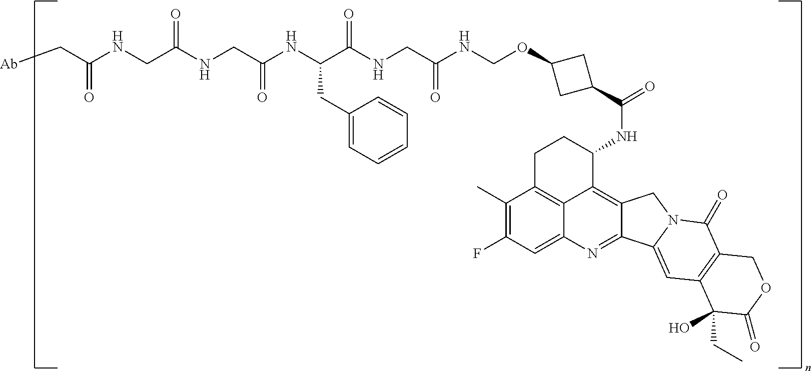 | D-II-12 |
| 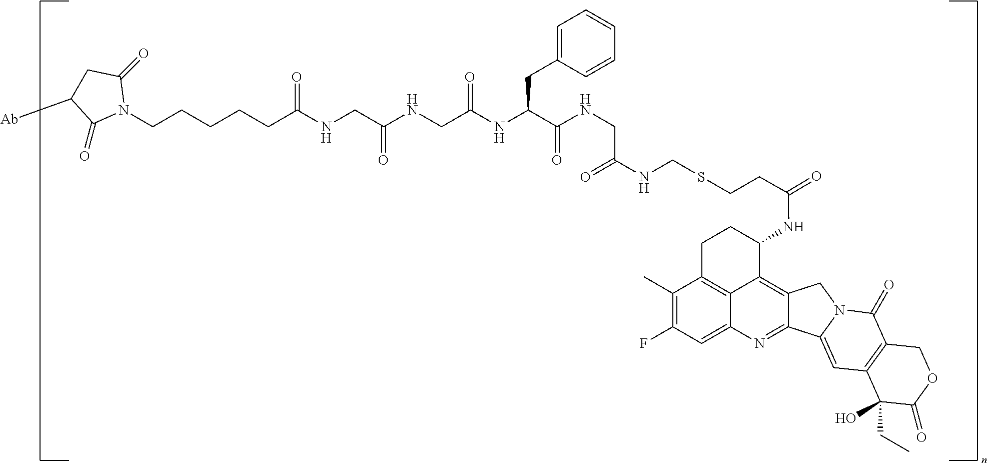 | D-III-1 |
| 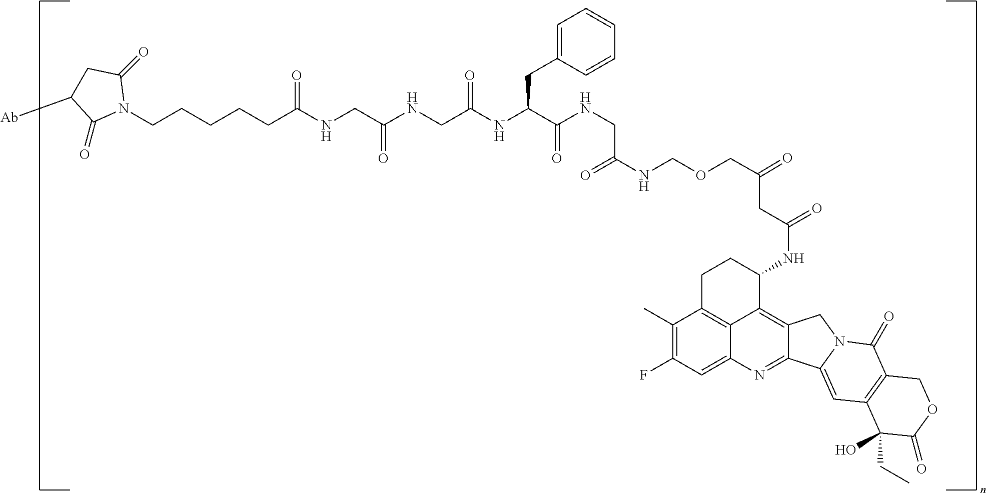 | D-III-2 |
| 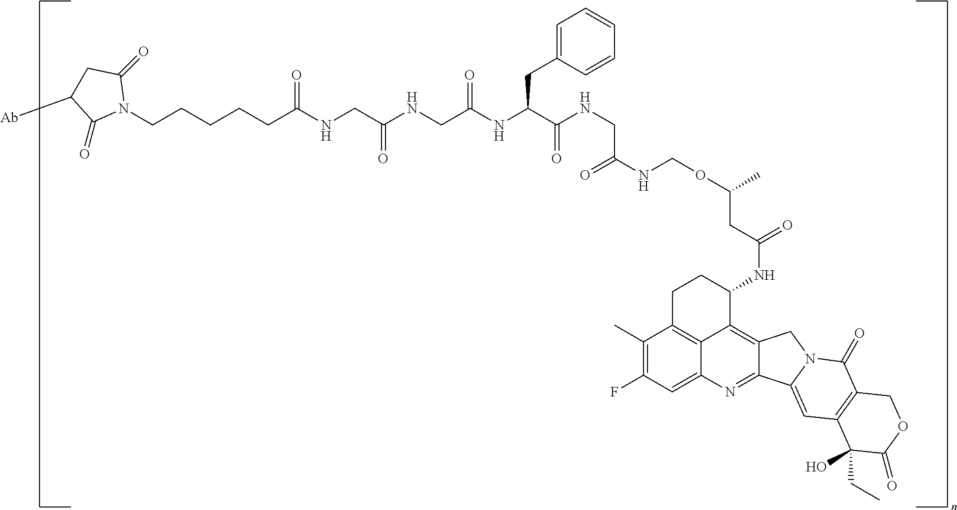 | D-III-3 |
| 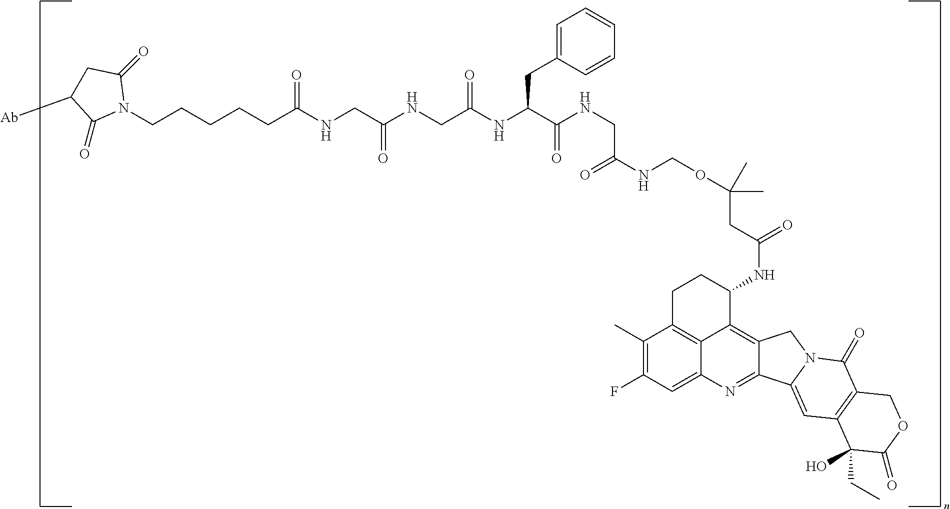 | D-III-4 |
| 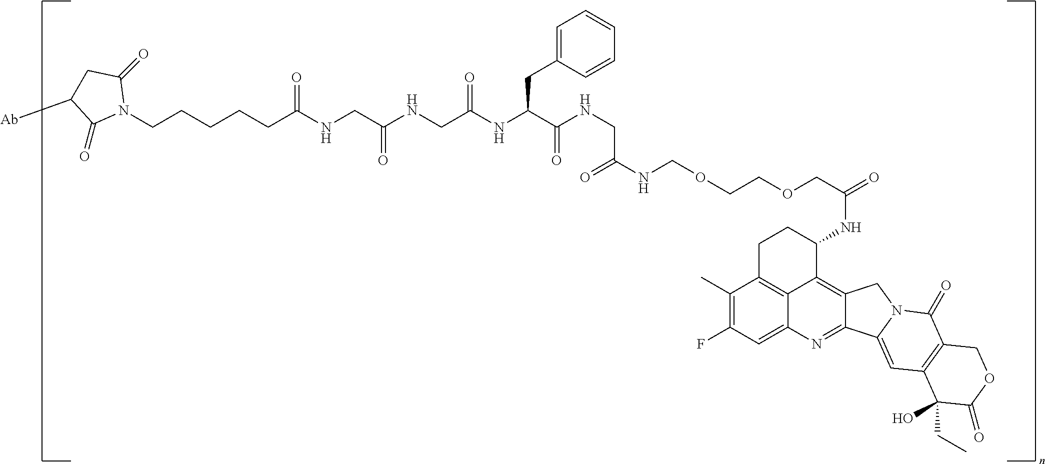 | D-III-5 |
| 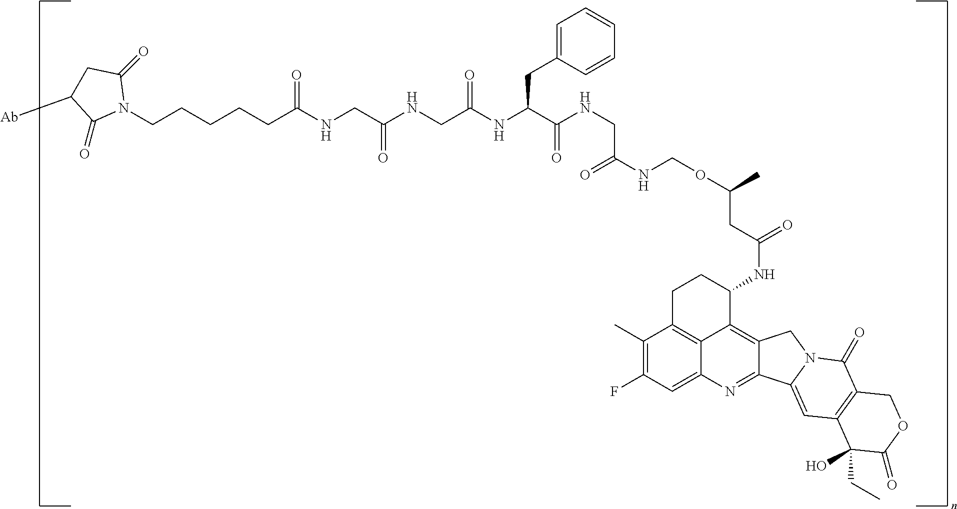 | D-III-6 |
| 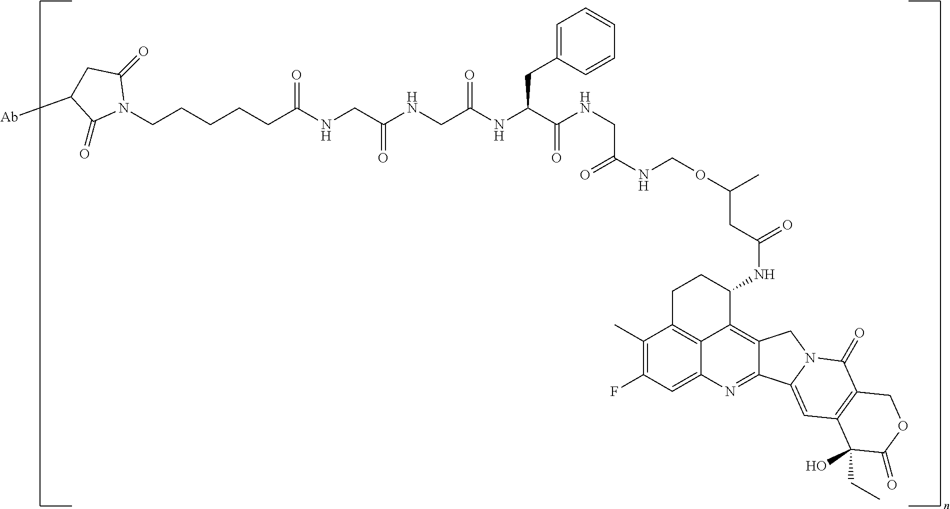 | D-III-7 |

wherein Ab represents the antibody which binds uPARAP receptor as described herein, wherein n is an integer from 1 to 10.

In one embodiment, the average connection number N^{a} may be an integer or a decimal from 2 to 8. For example, the average connection number N^{a} may be an integer or a decimal from 3 to 8. For example, the average connection number N^{a} may be an integer or a decimal from 1 to 2, 2 to 3, 3 to 4, 4 to 5, 5 to 6, 6 to 7, 7 to 8, 8 to 9, or 9 to 10.

### I-E

In one embodiment, the antibody-drug conjugate comprises an active agent with a structure according to formula (I-E), or a tautomer, a mesomer, a racemate, an enantiomer or a diastereoisomer thereof, or a mixture thereof, or a pharmaceutically acceptable salt thereof:

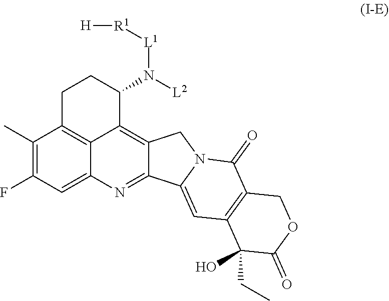

wherein R¹, L¹ and L² are defined as in any formula (I-A) in embodiments defined herein above.

### II-Ex, IIEy

In one embodiment, the antibody-drug conjugate comprises an active agent with a structure according to formula (II-Ex), or a tautomer, a mesomer, a racemate, an enantiomer or a diastereoisomer thereof, or a mixture thereof, or a pharmaceutically acceptable salt thereof:

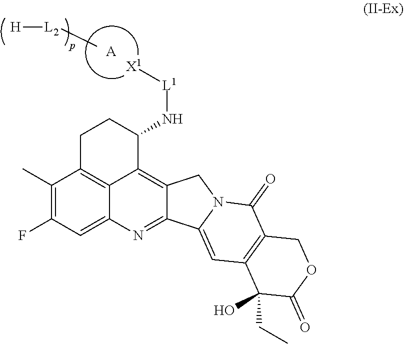

wherein L², p, ring A, X¹ and L¹ are defined as in any formula (II-Ax) in embodiments described herein above.

In one embodiment, the antibody-drug conjugate comprises an active agent with a structure according to formula (II-Ey), or a tautomer, a mesomer, a racemate, an enantiomer or a diastereoisomer thereof, or a mixture thereof, or a pharmaceutically acceptable salt thereof:

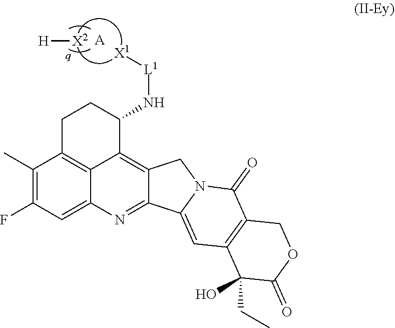

wherein X², q, ring A, X¹ and L¹ are defined as in any formula (II-Ay) in embodiments defined herein above.

### III-E

In one embodiment, the antibody-drug conjugate comprises an active agent with a structure according to formula (III-E), or a tautomer, a mesomer, a racemate, an enantiomer or a diastereoisomer thereof, or a mixture thereof, or a pharmaceutically acceptable salt thereof:

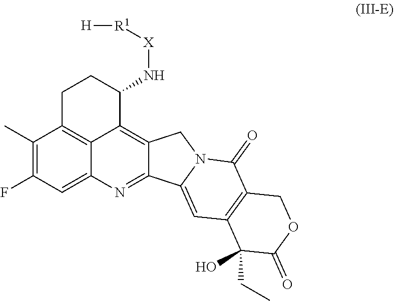

wherein R¹ and X are defined as in any formula (III-A) in embodiments defined herein above.

### A Method of Manufacture, I-F, II-Fx, II-Fy and III-F

In one aspect, the present disclosure provides for a method for manufacturing the antibody-drug conjugate as described herein, the method comprising reacting a compound according to one selected from the group consisting of: formula (I-F), formula (II-Fx), formula (II-Fy) and formula (III-F) as described herein below with the anti-uPARAP antibody as defined herein.

In one embodiment, the method for manufacturing the antibody-drug conjugate as described herein comprises reacting a compound according to formula (I-F) or a tautomer, a mesomer, a racemate, an enantiomer or a diastereoisomer thereof, or a mixture thereof, or a pharmaceutically acceptable salt thereof:

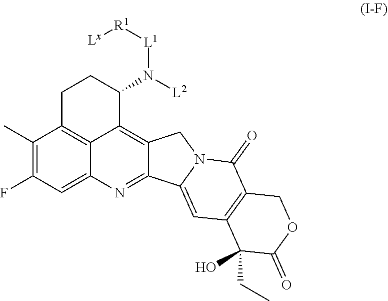

wherein, L^{x} may be -Lₐₓ-L_{b}-L_{c}-;
-Lₐₓ- may be selected from the group consisting of:

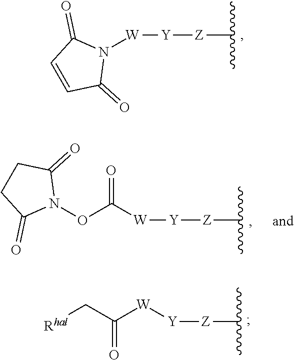

wherein R^{hal} may be iodine or bromine;
wherein W may be —(C(R^{wa})(R^{wb}))_{wn}—, Y may be —(OCH₂CH₂)_{yn}—O_{yp}—, and Z may be —(C(R^{za})(R^{zb}))_{zn};
wherein wn may be selected from the group consisting of integers≥_0, and
0 or no less than 1 methylene unit of W may be independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn may be selected from the group consisting of integers≥0, and yp may be 0 or 1;
wherein zn may be selected from the group consisting of integers≥0, and
0 or no less than 1 methylene unit of Z may be independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- may be selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or may be independently substituted with no less than 1 substituent R^{cx};
wherein each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r} or a C₁₋₆ aliphatic group which may be optionally substituted with R^{r};
wherein each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
L^{b} and L^{c} are defined as in any formula (I-B) in embodiments described herein above;
R¹, L¹ and L² are defined as in any formula (I-A) in embodiments described herein above.

In one embodiment, the method for manufacturing the antibody-drug conjugate as described herein comprises reacting a compound according to formula (II-Fx) or formula (II-Fy) or a tautomer, a mesomer, a racemate, an enantiomer or a diastereoisomer thereof, or a mixture thereof, or a pharmaceutically acceptable salt thereof:

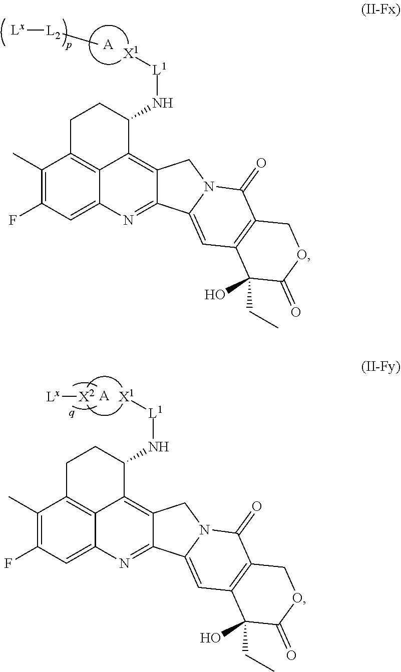

wherein, L^{x} may be -Lₐₓ-L_{b}-L_{c}-;
-Lₐₓ- may be selected from the group consisting of:

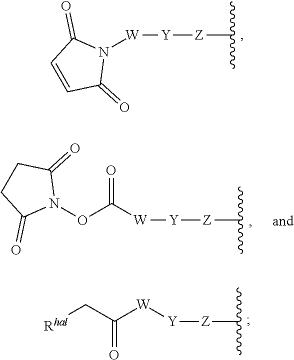

wherein R^{hal} may be iodine or bromine;
wherein W may be —(C(R^{wa})(R^{wb}))_{wn}—, Y may be —(OCH₂CH₂)_{yn}—O_{yp}—, and Z may be —(C(R^{za})(R^{zb}))_{zn};
wherein wn may be selected from the group consisting of integers≥0, and
0 or no less than 1 methylene unit of W may be independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn may be selected from the group consisting of integers≥0, and yp may be 0 or 1;
wherein zn may be selected from the group consisting of integers≥0, and
0 or no less than 1 methylene unit of Z may be independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
Cyr- may be selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or may be independently substituted with no less than 1 substituent R^{cx};
wherein each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r} or a C₁₋₆ aliphatic group which may be optionally substituted with R^{r};
wherein each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
L^{b} and L^{c} are defined as in any formula (II-Bx) or formula (II-By) in embodiments as described herein above;
wherein L², p, ring A, X¹ and L¹ are defined as in any formula (II-Ax) in embodiments as described herein above;
or X², q, ring A, X¹ and L¹ are defined as in any formula (II-Ay) in embodiments as described herein above.

In one embodiment, the method for manufacturing the antibody-drug conjugate as described herein comprises reacting a compound according to formula (III-F) or a tautomer, a mesomer, a racemate, an enantiomer or a diastereoisomer thereof, or a mixture thereof, or a pharmaceutically acceptable salt thereof:

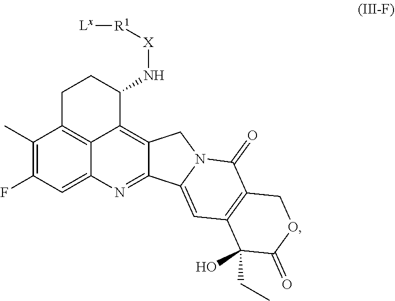

wherein, L^{x} may be -Lₐₓ-L_{b}-L_{c}-;
-Lₐₓ- may be selected from the group consisting of:

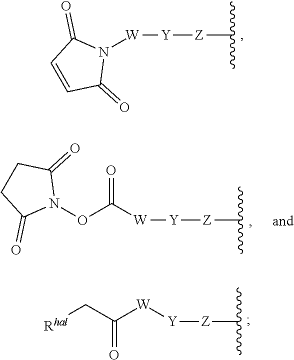

wherein R^{hal} may be iodine or bromine;
wherein W may be —(C(R^{wa})(R^{wb}))_{wn}—, Y may be —(OCH₂CH₂)_{yn}—O_{yp}—, and Z may be —(C(R^{za})(R^{zb}))_{zn};
wherein wn may be selected from the group consisting of integers≥0, and
0 or no less than 1 methylene unit of W may be independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn may be selected from the group consisting of integers≥0, and yp may be 0 or 1;
wherein zn may be selected from the group consisting of integers≥0, and
0 or no less than 1 methylene unit of Z may be independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- may be selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or may be independently substituted with no less than 1 substituent R^{cx};
wherein each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r} or a C₁₋₆ aliphatic group which may be optionally substituted with R^{r};
wherein each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
L^{b} and L^{c} are defined as in any formula (III-B) in embodiments described herein above;
wherein R¹ and X are defined as in any formula (III-A) in embodiments described herein above.

In one embodiment, Lₐₓ- may be selected from the group consisting of:

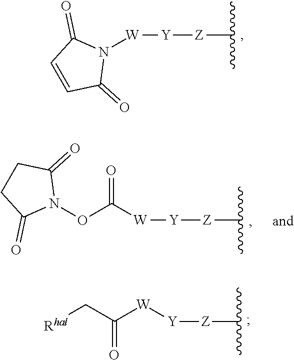

wherein R^{hal} may be iodine or bromine;
wherein W may be —(C(R^{wa})(R^{wb}))_{wn}—, Y may be —(OCH₂CH₂)_{yn}—O_{yp}—, and Z may be —(C(R^{za})(R^{zb}))_{zn}.

In another embodiment, wn may be selected from the group consisting of integers from 2 to 6, and 0 or 1 methylene unit of W may be independently replaced by -Cyr-, —N(R^{wx})C(O)—, —C(O)N(R^{wx})—, —C(O)—, —NR^{wx}— or —O—.

For example, wn may be 1, 2, 3 or 6, and 1 methylene unit of W may be independently replaced by -Cyr-, —N(R^{wx})C(O)—, —C(O)N(R^{wx})— or —C(O)—.

In another embodiment, yn may be selected from the group consisting of integers from 0 to 12, and yp may be 0 or 1.

For example, yn may be 0, 4 or 8, and yp may be 0 or 1.

In another embodiment, zn may be selected from the group consisting of integers from 0 to 10, and 0 or 1 methylene unit of Z may be independently replaced by -Cyr-, —N(R^{zx})C(O)—, —C(O)N(R^{zx})— or —C(O)—.

For example, zn may be 1, 2 or 3, and 1 methylene unit of Z may be independently replaced by -Cyr-, —N(R^{zx})C(O)—, —C(O)N(R^{zx})— or —C(O)—.

In another embodiment, —Cyr- may be selected from the group consisting of: 6-10 membered arylene and 3-10 membered saturated or partially unsaturated carbocyclylene, wherein -Cyr- is unsubstituted or may be independently substituted with 1 to 3 substituent R^{cx}.

For example, —Cyr- may be 3-10 membered saturated carbocyclylene, wherein -Cyr- is unsubstituted or may be independently substituted with 1 to 3 substituent R^{cx}.

In another embodiment, each R^{wa}, each R^{wb}, each R^{za}, each R^{zb}, each R^{wx}, each R^{zx} and each R^{cx} may each independently be hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group which may be optionally substituted with R^{r}; each R^{r}, each R^{ra} and each R^{rb} may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

For example, each R^{wa}, each R^{wb}, each R^{za}, each R^{zb}, each R^{wx}, each R^{zx} and each R^{cx} may each independently be hydrogen, halogen, —OR^{r}, or a C₁₋₆ aliphatic group which may be optionally substituted with R^{r}; each R^{r} may independently be hydrogen, halogen or a C₁₋₆ aliphatic group.

In one embodiment, Lₐₓ- may be

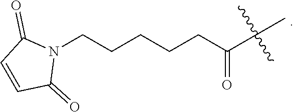 [SMILES: *C(=O)CCCCCN1C(=O)C=CC1=O]

In one embodiment, Lₐₓ-L_{b}-L_{c}- may be selected from the group consisting of:

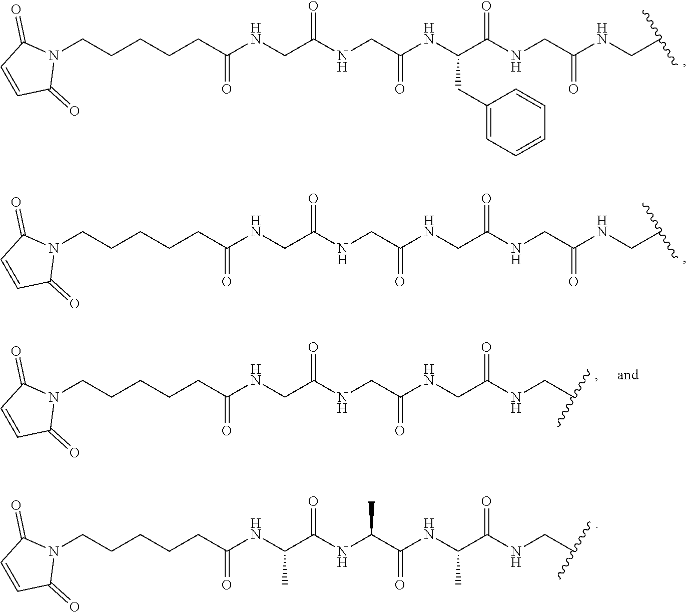 [SMILES: *CNC(=O)CNC(=O)[C@H](Cc1ccccc1)NC(=O)CNC(=O)CNC(=O)CCCCCN1C(=O)C=CC1=O ; *CNC(=O)CNC(=O)CNC(=O)CNC(=O)CNC(=O)CCCCCN1C(=O)C=CC1=O ; *CNC(=O)CNC(=O)CNC(=O)CNC(=O)CCCCCN1C(=O)C=CC1=O ; *CNC(=O)[C@H](C)NC(=O)[C@H](C)NC(=O)[C@H](C)NC(=O)CCCCCN1C(=O)C=CC1=O]

In one embodiment, the method for manufacturing the antibody-drug conjugate as described herein comprises reacting a compound according to any one of the structures in Table 3 or a tautomer, a mesomer, a racemate, an enantiomer or a diastereoisomer thereof, or a mixture thereof, or a pharmaceutically acceptable salt thereof with the anti-uPARAP antibody as defined herein.

TABLE 3

| | |
|---|---|
| L-I-1 | 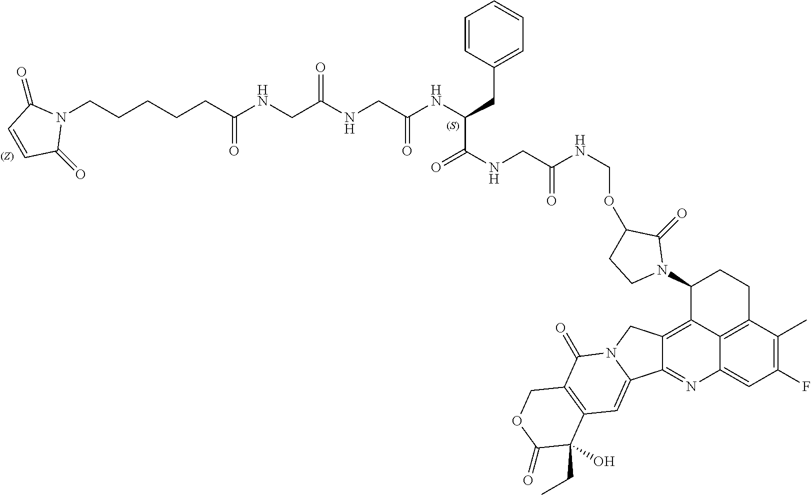 |
| L-I-2 | 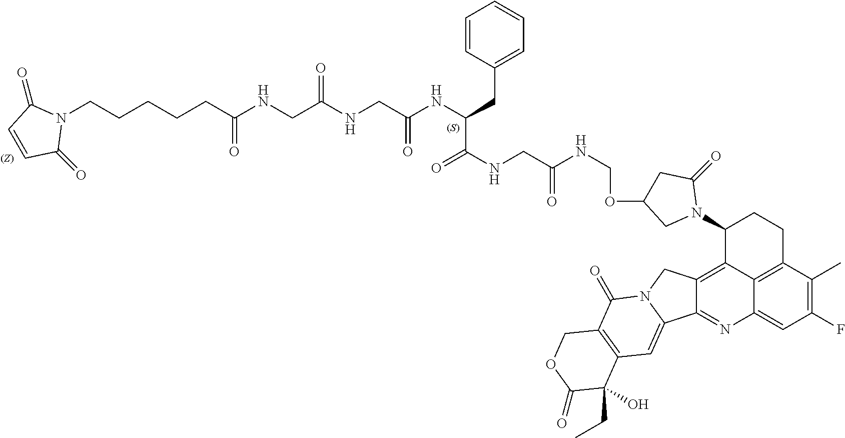 |
| L-I-3 | 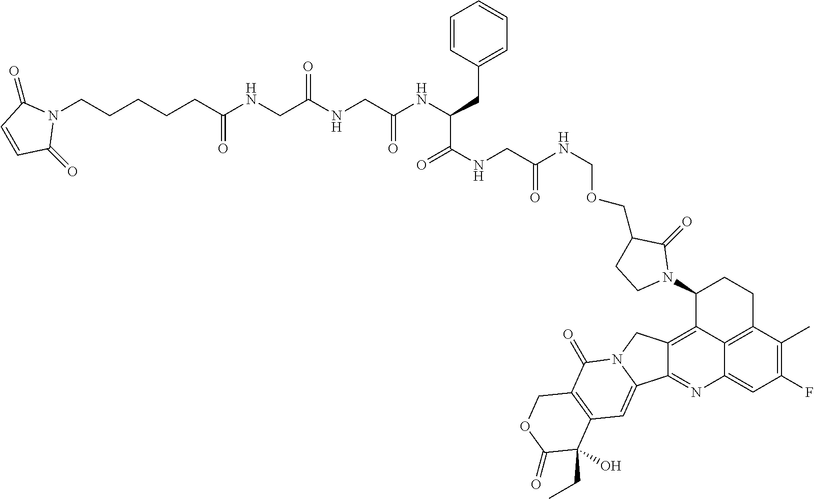 [SMILES: CC[C@@]1(O)C(=O)OCc2c1cc1n(c2=O)Cc2c-1nc1cc(F)c(C)c3c1c2[C@@H](N1CCC(COCNC(=O)CNC(=O)[C@H](Cc2ccccc2)NC(=O)CNC(=O)CNC(=O)CCCCCN2C(=O)C=CC2=O)C1=O)CC3] |
| L-I-4 | 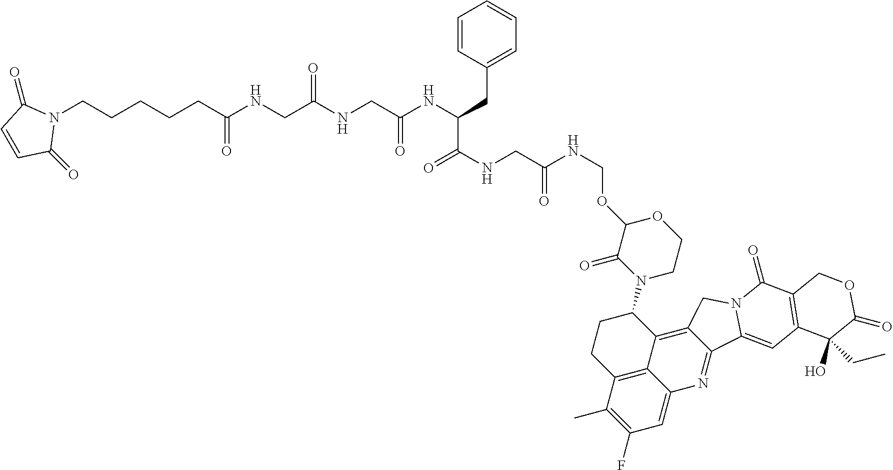 [SMILES: CC[C@@]1(O)C(=O)OCc2c1cc1n(c2=O)Cc2c-1nc1cc(F)c(C)c3c1c2[C@@H](N1CCOC(OCNC(=O)CNC(=O)[C@H](Cc2ccccc2)NC(=O)CNC(=O)CNC(=O)CCCCCN2C(=O)C=CC2=O)C1=O)CC3] |
| L-I-5 | 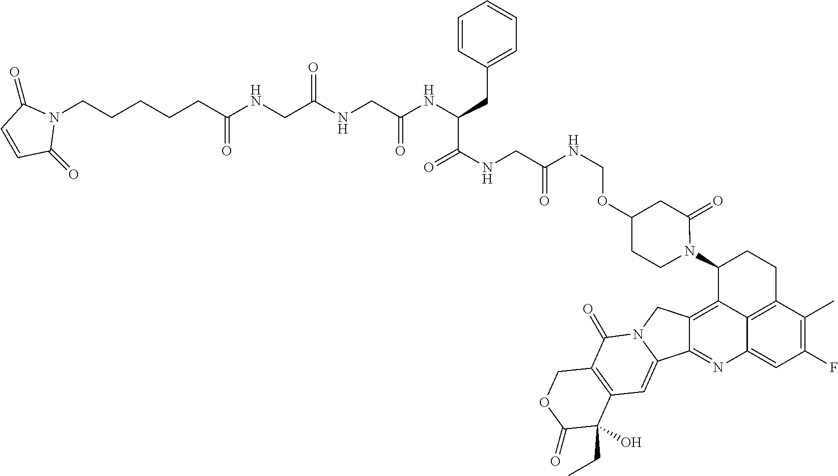 [SMILES: CC[C@@]1(O)C(=O)OCc2c1cc1n(c2=O)Cc2c-1nc1cc(F)c(C)c3c1c2[C@@H](N1CCC(OCNC(=O)CNC(=O)[C@H](Cc2ccccc2)NC(=O)CNC(=O)CNC(=O)CCCCCN2C(=O)C=CC2=O)CC1=O)CC3] |
| L-I-6 | 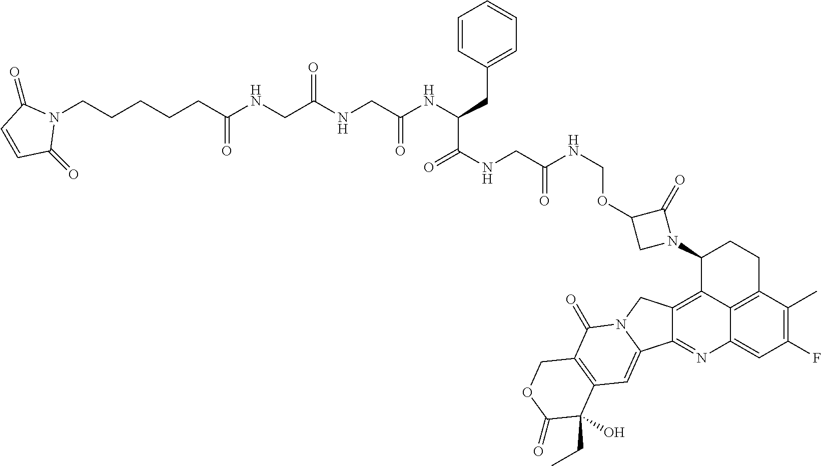 [SMILES: CC[C@@]1(O)C(=O)OCc2c1cc1n(c2=O)Cc2c-1nc1cc(F)c(C)c3c1c2[C@@H](N1CC(OCNC(=O)CNC(=O)[C@H](Cc2ccccc2)NC(=O)CNC(=O)CNC(=O)CCCCCN2C(=O)C=CC2=O)C1=O)CC3] |
| L-I-7 | 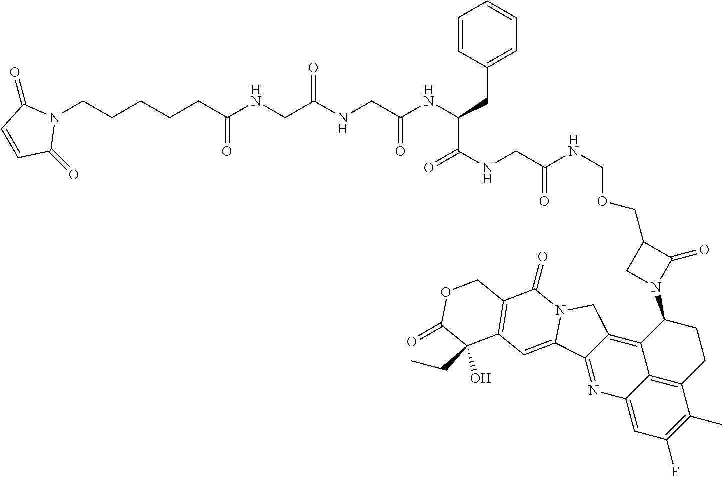 [SMILES: CC[C@@]1(O)C(=O)OCc2c1cc1n(c2=O)Cc2c-1nc1cc(F)c(C)c3c1c2[C@@H](N1CC(COCNC(=O)CNC(=O)[C@H](Cc2ccccc2)NC(=O)CNC(=O)CNC(=O)CCCCCN2C(=O)C=CC2=O)C1=O)CC3] |
| L-I-8 | 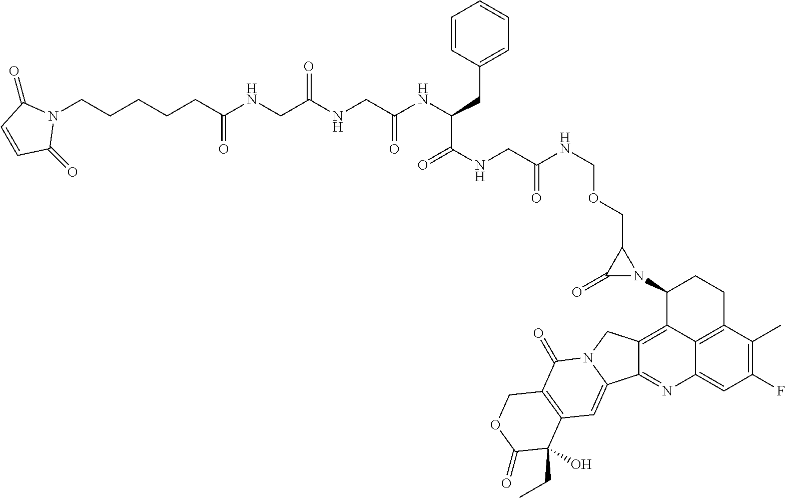 [SMILES: CC[C@@]1(O)C(=O)OCc2c1cc1n(c2=O)Cc2c-1nc1cc(F)c(C)c3c1c2[C@@H](N1C(=O)C1COCNC(=O)CNC(=O)[C@H](Cc1ccccc1)NC(=O)CNC(=O)CNC(=O)CCCCCN1C(=O)C=CC1=O)CC3] |
| L-I-9 | 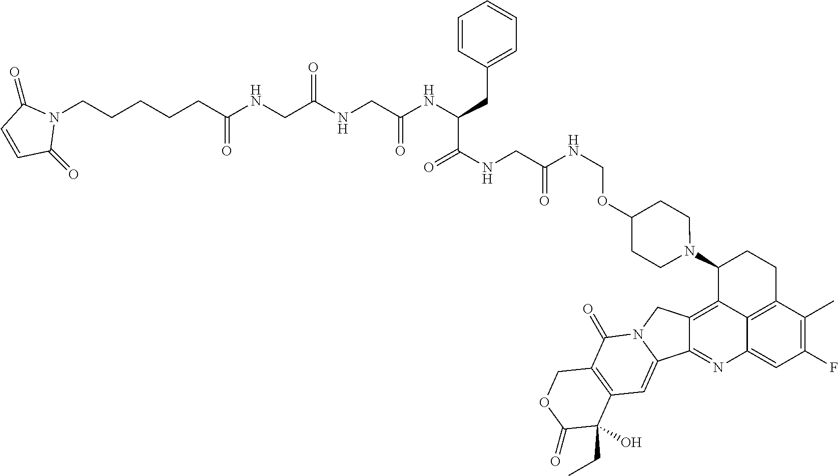 [SMILES: CC[C@@]1(O)C(=O)OCc2c1cc1n(c2=O)Cc2c-1nc1cc(F)c(C)c3c1c2[C@@H](N1CCC(OCNC(=O)CNC(=O)[C@H](Cc2ccccc2)NC(=O)CNC(=O)CNC(=O)CCCCCN2C(=O)C=CC2=O)CC1)CC3] |
| L-I-10 | 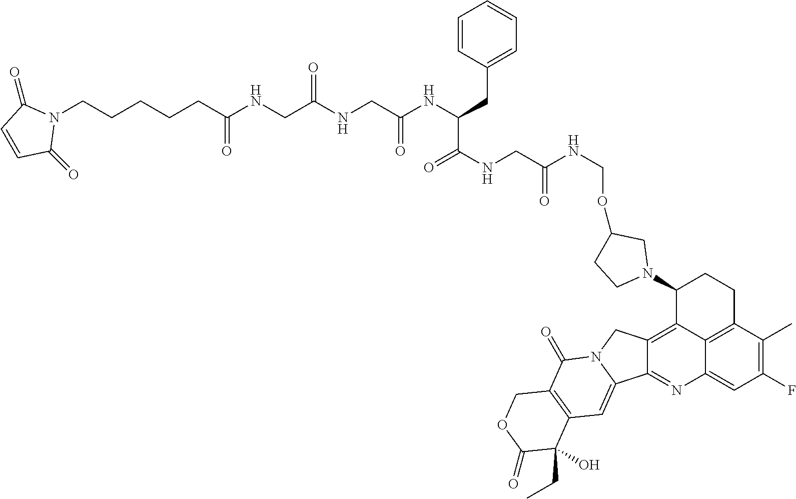 [SMILES: CC[C@@]1(O)C(=O)OCc2c1cc1n(c2=O)Cc2c-1nc1cc(F)c(C)c3c1c2[C@@H](N1CCC(OCNC(=O)CNC(=O)[C@H](Cc2ccccc2)NC(=O)CNC(=O)CNC(=O)CCCCCN2C(=O)C=CC2=O)C1)CC3] |
| L-I-11 | 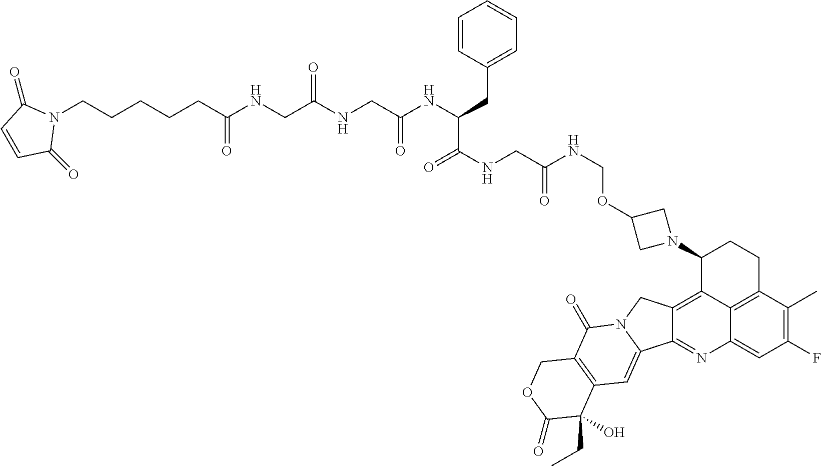 [SMILES: CC[C@@]1(O)C(=O)OCc2c1cc1n(c2=O)Cc2c-1nc1cc(F)c(C)c3c1c2[C@@H](N1CC(OCNC(=O)CNC(=O)[C@H](Cc2ccccc2)NC(=O)CNC(=O)CNC(=O)CCCCCN2C(=O)C=CC2=O)C1)CC3] |
| L-I-12 | 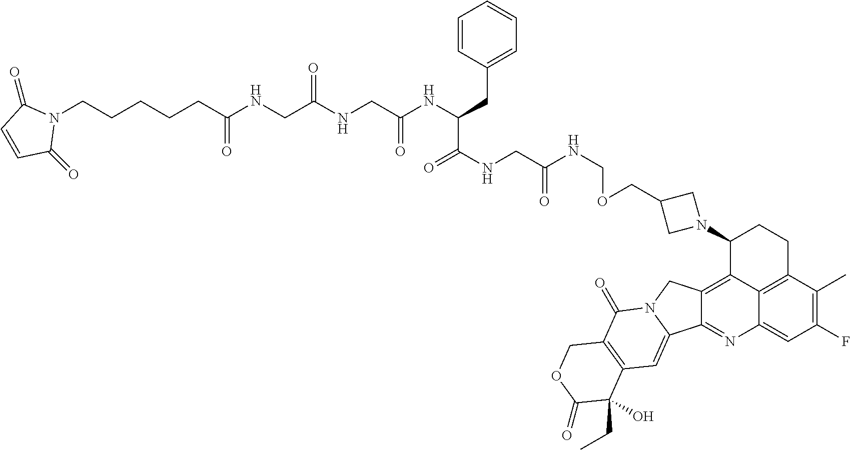 [SMILES: CC[C@@]1(O)C(=O)OCc2c1cc1n(c2=O)Cc2c-1nc1cc(F)c(C)c3c1c2[C@@H](N1CC(COCNC(=O)CNC(=O)[C@H](Cc2ccccc2)NC(=O)CNC(=O)CNC(=O)CCCCCN2C(=O)C=CC2=O)C1)CC3] |
| L-I-13 | 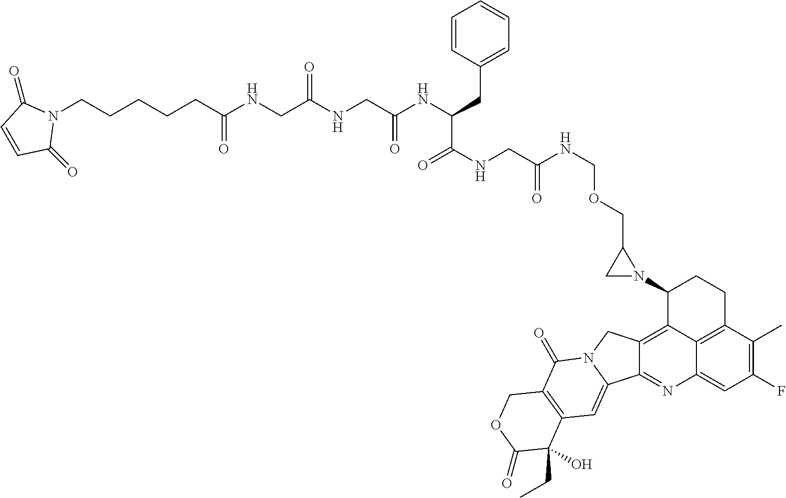 [SMILES: CC[C@@]1(O)C(=O)OCc2c1cc1n(c2=O)Cc2c-1nc1cc(F)c(C)c3c1c2[C@@H](N1CC1COCNC(=O)CNC(=O)[C@H](Cc1ccccc1)NC(=O)CNC(=O)CNC(=O)CCCCCN1C(=O)C=CC1=O)CC3] |
| L-I-14 | 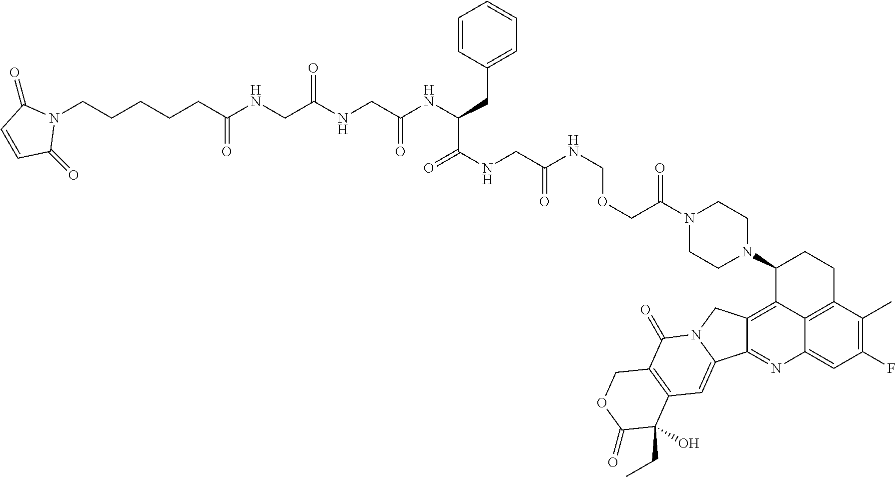 [SMILES: CC[C@@]1(O)C(=O)OCc2c1cc1n(c2=O)Cc2c-1nc1cc(F)c(C)c3c1c2[C@@H](N1CCN(C(=O)COCNC(=O)CNC(=O)[C@H](Cc2ccccc2)NC(=O)CNC(=O)CNC(=O)CCCCCN2C(=O)C=CC2=O)CC1)CC3] |
| L-I-15 | 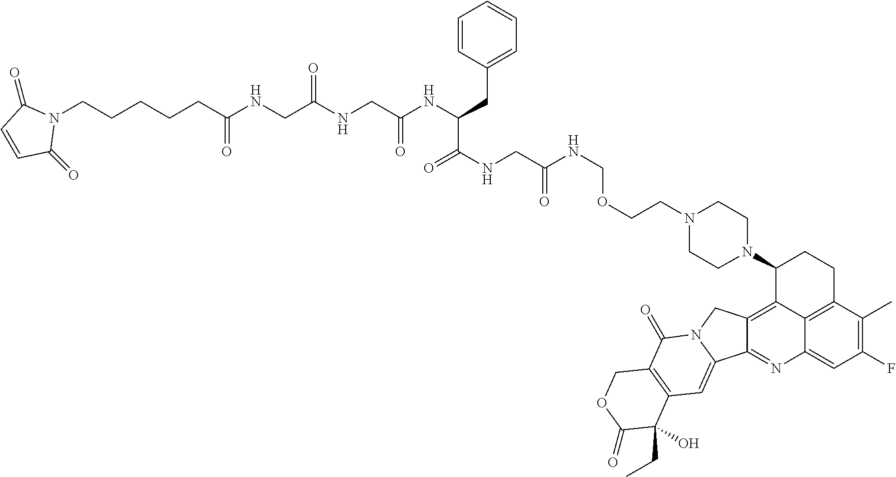 [SMILES: CC[C@@]1(O)C(=O)OCc2c1cc1n(c2=O)Cc2c-1nc1cc(F)c(C)c3c1c2[C@@H](N1CCN(CCOCNC(=O)CNC(=O)[C@H](Cc2ccccc2)NC(=O)CNC(=O)CNC(=O)CCCCCN2C(=O)C=CC2=O)CC1)CC3] |
| L-I-16 | 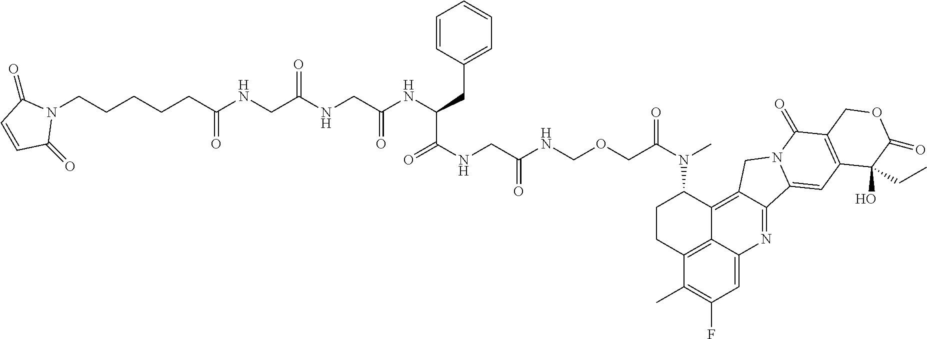 [SMILES: CC[C@@]1(O)C(=O)OCc2c1cc1n(c2=O)Cc2c-1nc1cc(F)c(C)c3c1c2[C@@H](N(C)C(=O)COCNC(=O)CNC(=O)[C@H](Cc1ccccc1)NC(=O)CNC(=O)CNC(=O)CCCCCN1C(=O)C=CC1=O)CC3] |
| L-I-17 | 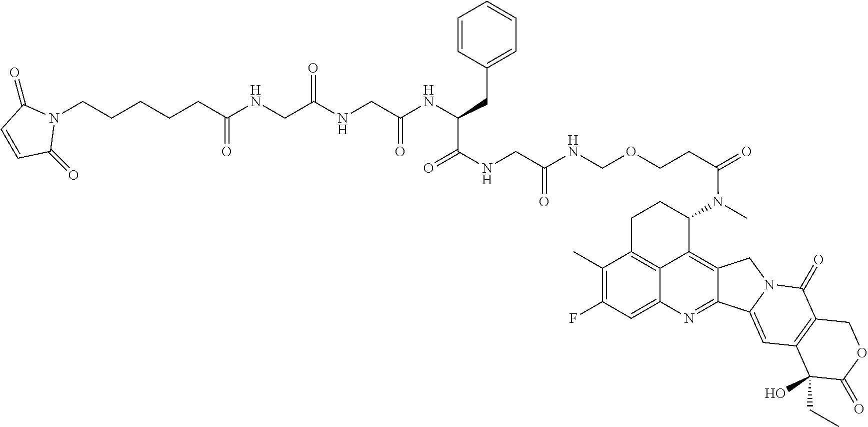 [SMILES: CC[C@@]1(O)C(=O)OCc2c1cc1n(c2=O)Cc2c-1nc1cc(F)c(C)c3c1c2[C@@H](N(C)C(=O)CCOCNC(=O)CNC(=O)[C@H](Cc1ccccc1)NC(=O)CNC(=O)CNC(=O)CCCCCN1C(=O)C=CC1=O)CC3] |
| L-I-18 | 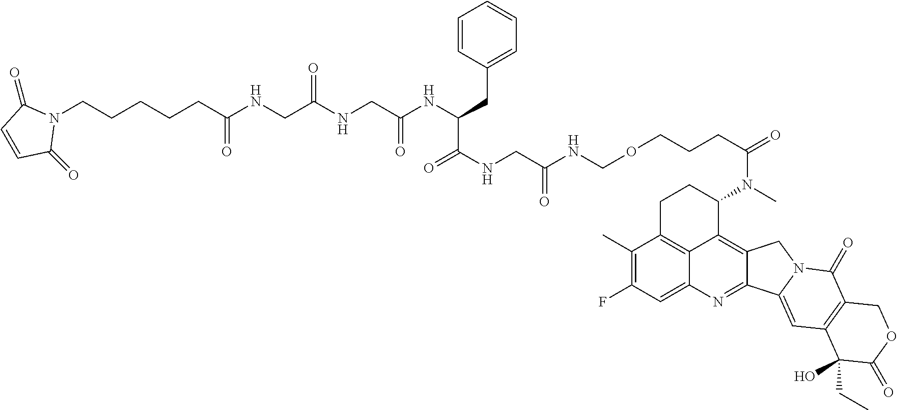 [SMILES: CC[C@@]1(O)C(=O)OCc2c1cc1n(c2=O)Cc2c-1nc1cc(F)c(C)c3c1c2[C@@H](N(C)C(=O)CCCOCNC(=O)CNC(=O)[C@H](Cc1ccccc1)NC(=O)CNC(=O)CNC(=O)CCCCCN1C(=O)C=CC1=O)CC3] |
| L-I-19 | 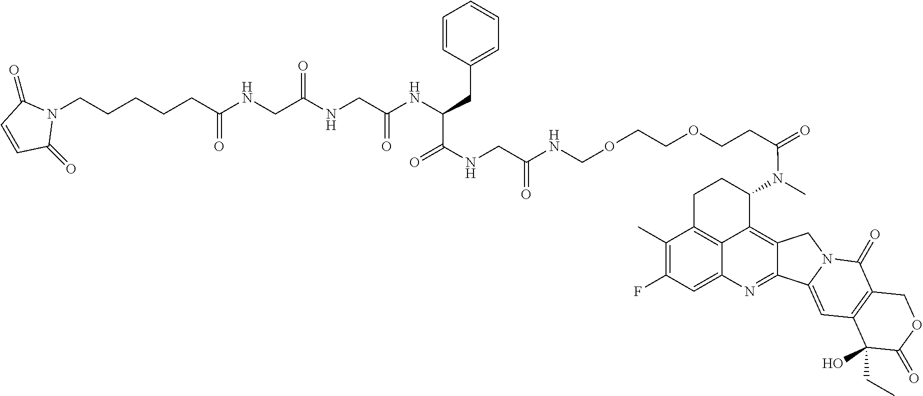 [SMILES: CC[C@@]1(O)C(=O)OCc2c1cc1n(c2=O)Cc2c-1nc1cc(F)c(C)c3c1c2[C@@H](N(C)C(=O)CCOCCOCNC(=O)CNC(=O)[C@H](Cc1ccccc1)NC(=O)CNC(=O)CNC(=O)CCCCCN1C(=O)C=CC1=O)CC3] |
| L-I-20 | 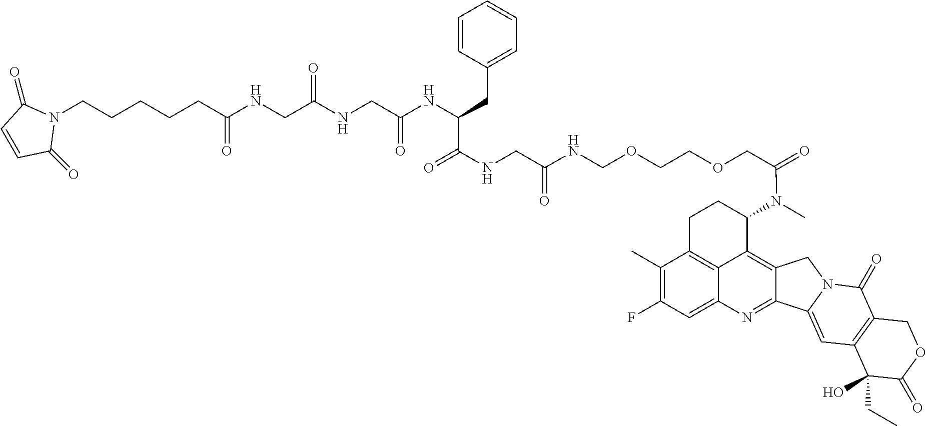 [SMILES: CC[C@@]1(O)C(=O)OCc2c1cc1n(c2=O)Cc2c-1nc1cc(F)c(C)c3c1c2[C@@H](N(C)C(=O)COCCOCNC(=O)CNC(=O)[C@H](Cc1ccccc1)NC(=O)CNC(=O)CNC(=O)CCCCCN1C(=O)C=CC1=O)CC3] |
| L-I-21 | 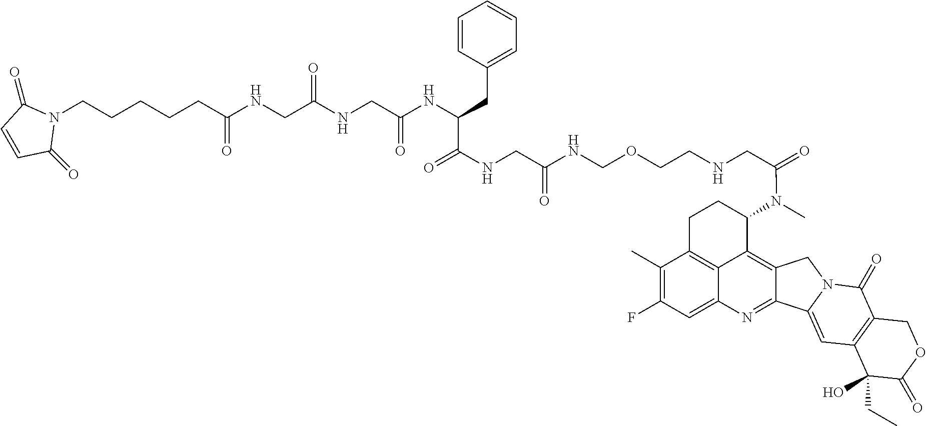 [SMILES: CC[C@@]1(O)C(=O)OCc2c1cc1n(c2=O)Cc2c-1nc1cc(F)c(C)c3c1c2[C@@H](N(C)C(=O)CNCCOCNC(=O)CNC(=O)[C@H](Cc1ccccc1)NC(=O)CNC(=O)CNC(=O)CCCCCN1C(=O)C=CC1=O)CC3] |
| L-I-22 | 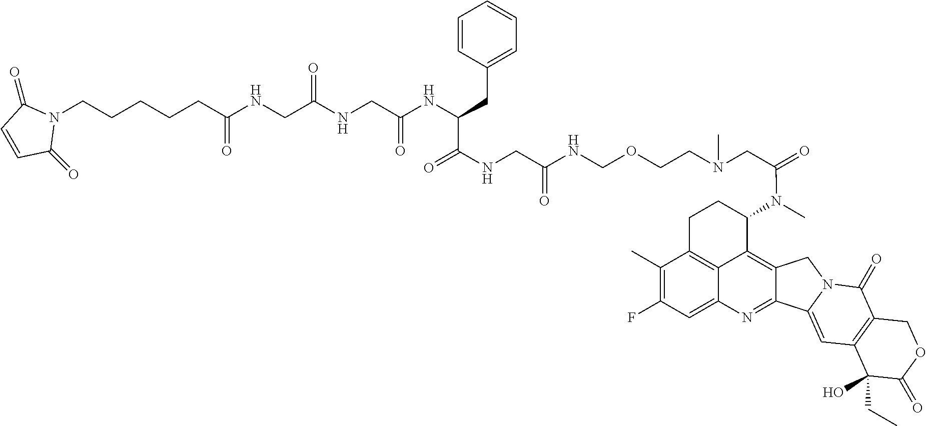 [SMILES: CC[C@@]1(O)C(=O)OCc2c1cc1n(c2=O)Cc2c-1nc1cc(F)c(C)c3c1c2[C@@H](N(C)C(=O)CN(C)CCOCNC(=O)CNC(=O)[C@H](Cc1ccccc1)NC(=O)CNC(=O)CNC(=O)CCCCCN1C(=O)C=CC1=O)CC3] |
| L-I-23 | 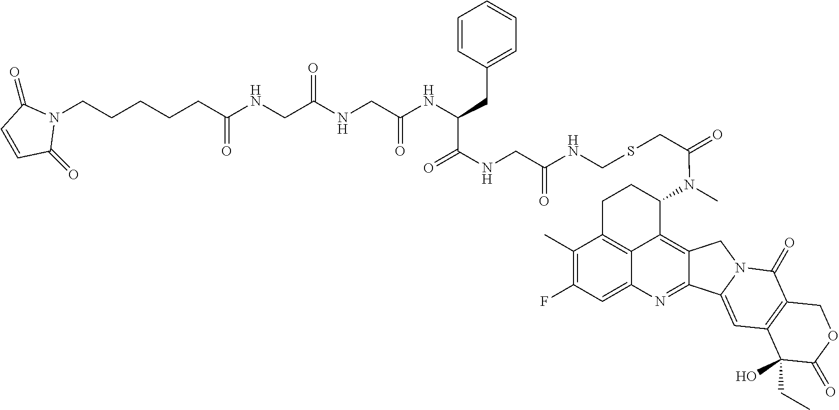 [SMILES: CC[C@@]1(O)C(=O)OCc2c1cc1n(c2=O)Cc2c-1nc1cc(F)c(C)c3c1c2[C@@H](N(C)C(=O)CSCNC(=O)CNC(=O)[C@H](Cc1ccccc1)NC(=O)CNC(=O)CNC(=O)CCCCCN1C(=O)C=CC1=O)CC3] |
| L-I-24 | 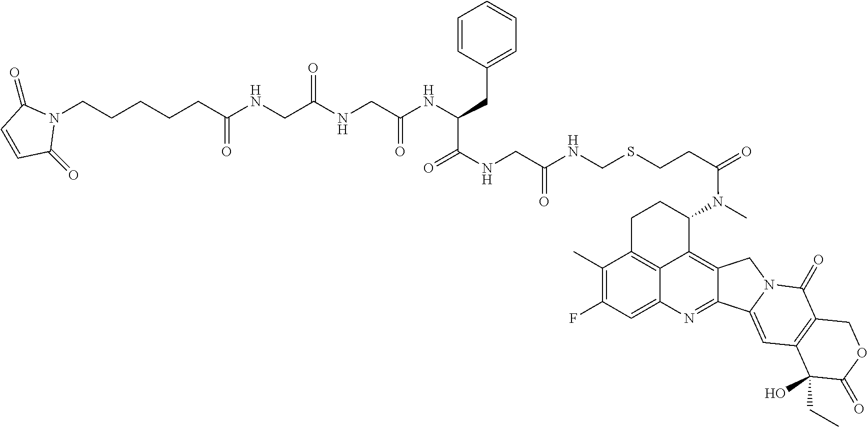 [SMILES: CC[C@@]1(O)C(=O)OCc2c1cc1n(c2=O)Cc2c-1nc1cc(F)c(C)c3c1c2[C@@H](N(C)C(=O)CCSCNC(=O)CNC(=O)[C@H](Cc1ccccc1)NC(=O)CNC(=O)CNC(=O)CCCCCN1C(=O)C=CC1=O)CC3] |
| L-I-25 | 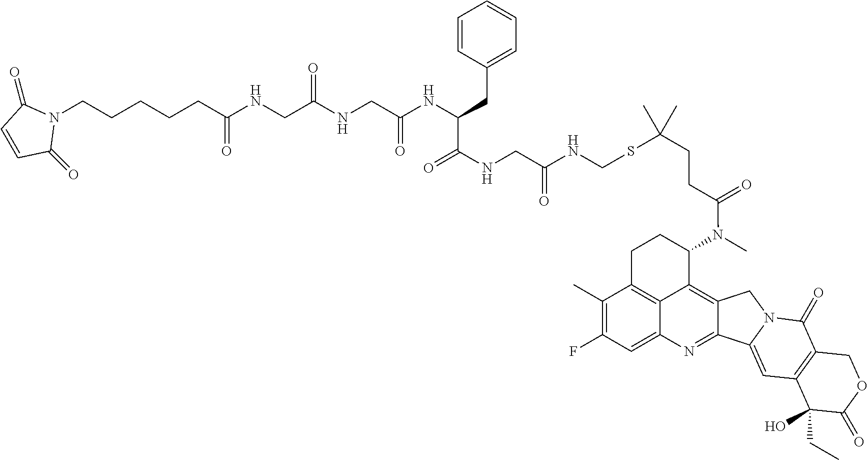 [SMILES: CC[C@@]1(O)C(=O)OCc2c1cc1n(c2=O)Cc2c-1nc1cc(F)c(C)c3c1c2[C@@H](N(C)C(=O)CCC(C)(C)SCNC(=O)CNC(=O)[C@H](Cc1ccccc1)NC(=O)CNC(=O)CNC(=O)CCCCCN1C(=O)C=CC1=O)CC3] |
| L-I-26 | 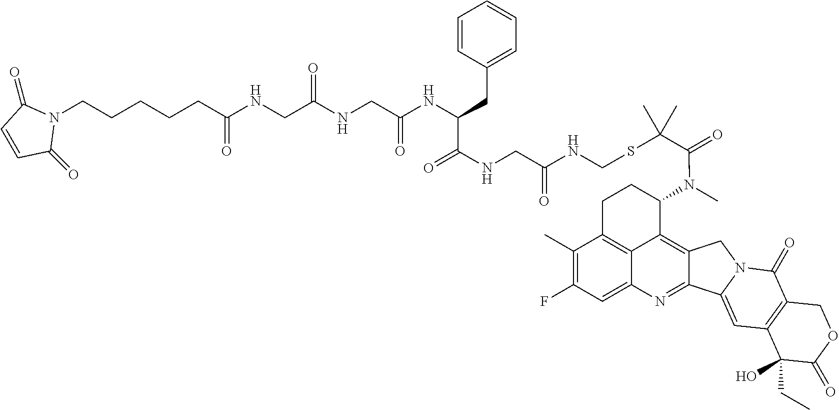 [SMILES: CC[C@@]1(O)C(=O)OCc2c1cc1n(c2=O)Cc2c-1nc1cc(F)c(C)c3c1c2[C@@H](N(C)C(=O)C(C)(C)SCNC(=O)CNC(=O)[C@H](Cc1ccccc1)NC(=O)CNC(=O)CNC(=O)CCCCCN1C(=O)C=CC1=O)CC3] |
| L-I-27 | 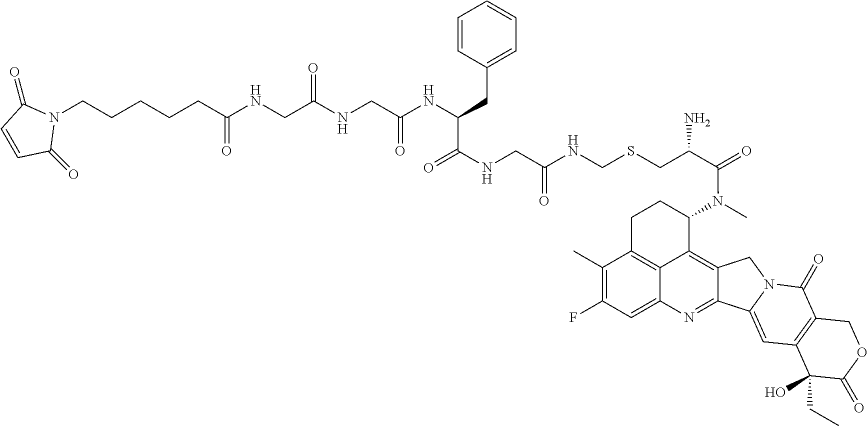 [SMILES: CC[C@@]1(O)C(=O)OCc2c1cc1n(c2=O)Cc2c-1nc1cc(F)c(C)c3c1c2[C@@H](N(C)C(=O)[C@@H](N)CSCNC(=O)CNC(=O)[C@H](Cc1ccccc1)NC(=O)CNC(=O)CNC(=O)CCCCCN1C(=O)C=CC1=O)CC3] |
| L-I-28 | 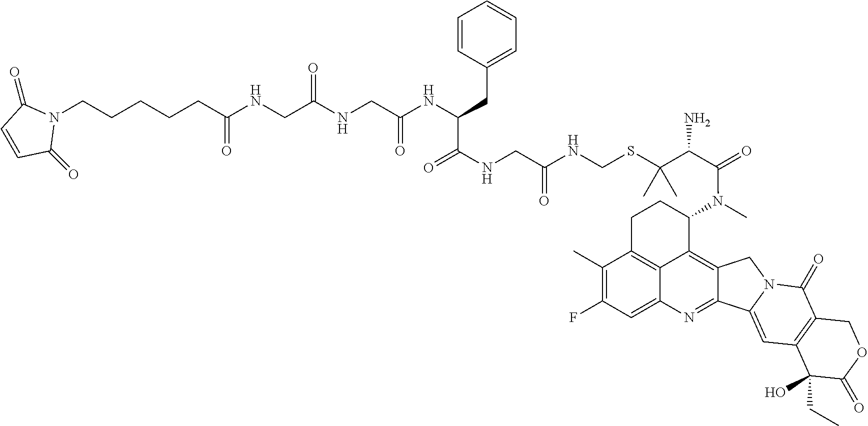 [SMILES: CC[C@@]1(O)C(=O)OCc2c1cc1n(c2=O)Cc2c-1nc1cc(F)c(C)c3c1c2[C@@H](N(C)C(=O)[C@@H](N)C(C)(C)SCNC(=O)CNC(=O)[C@H](Cc1ccccc1)NC(=O)CNC(=O)CNC(=O)CCCCCN1C(=O)C=CC1=O)CC3] |
| L-I-29 | 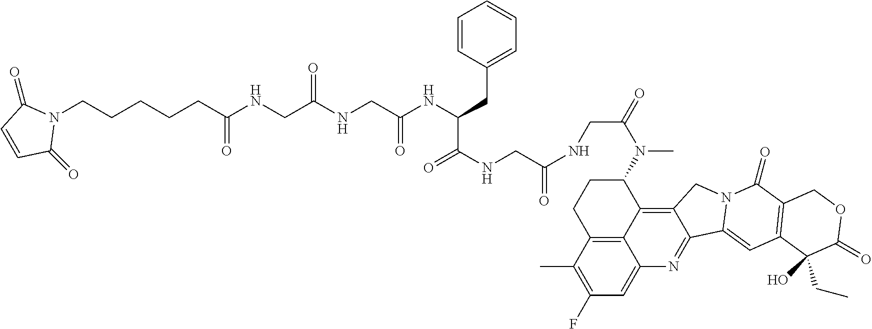 [SMILES: CC[C@@]1(O)C(=O)OCc2c1cc1n(c2=O)Cc2c-1nc1cc(F)c(C)c3c1c2[C@@H](N(C)C(=O)CNC(=O)CNC(=O)[C@H](Cc1ccccc1)NC(=O)CNC(=O)CNC(=O)CCCCCN1C(=O)C=CC1=O)CC3] |
| L-I-30 | 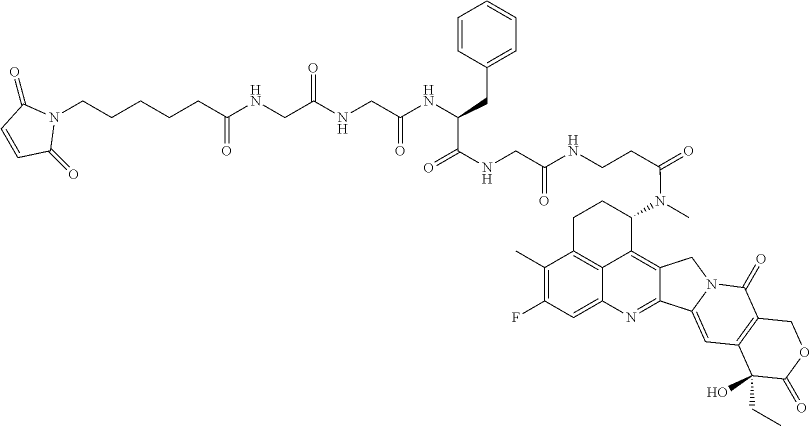 [SMILES: CC[C@@]1(O)C(=O)OCc2c1cc1n(c2=O)Cc2c-1nc1cc(F)c(C)c3c1c2[C@@H](N(C)C(=O)CCNC(=O)CNC(=O)[C@H](Cc1ccccc1)NC(=O)CNC(=O)CNC(=O)CCCCCN1C(=O)C=CC1=O)CC3] |
| L-I-31 | 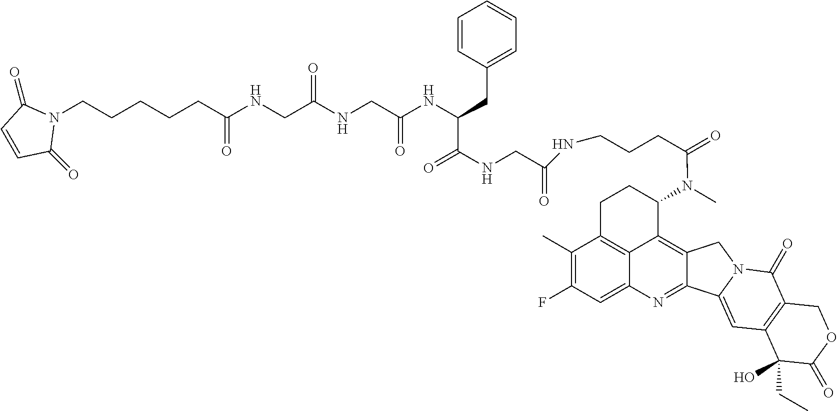 [SMILES: CC[C@@]1(O)C(=O)OCc2c1cc1n(c2=O)Cc2c-1nc1cc(F)c(C)c3c1c2[C@@H](N(C)C(=O)CCCNC(=O)CNC(=O)[C@H](Cc1ccccc1)NC(=O)CNC(=O)CNC(=O)CCCCCN1C(=O)C=CC1=O)CC3] |
| L-I-32 | 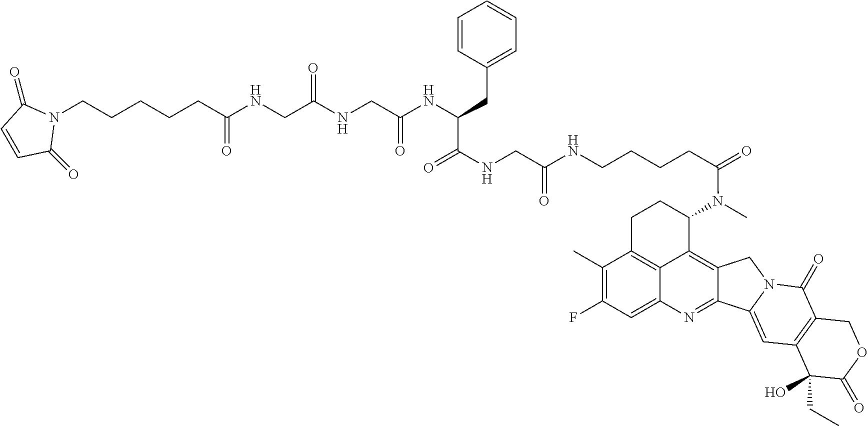 [SMILES: CC[C@@]1(O)C(=O)OCc2c1cc1n(c2=O)Cc2c-1nc1cc(F)c(C)c3c1c2[C@@H](N(C)C(=O)CCCCNC(=O)CNC(=O)[C@H](Cc1ccccc1)NC(=O)CNC(=O)CNC(=O)CCCCCN1C(=O)C=CC1=O)CC3] |
| L-I-33 | 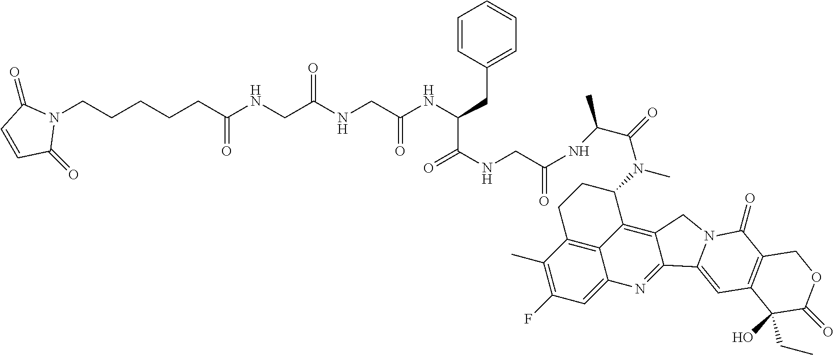 [SMILES: CC[C@@]1(O)C(=O)OCc2c1cc1n(c2=O)Cc2c-1nc1cc(F)c(C)c3c1c2[C@@H](N(C)C(=O)[C@H](C)NC(=O)CNC(=O)[C@H](Cc1ccccc1)NC(=O)CNC(=O)CNC(=O)CCCCCN1C(=O)C=CC1=O)CC3] |
| L-I-34 | 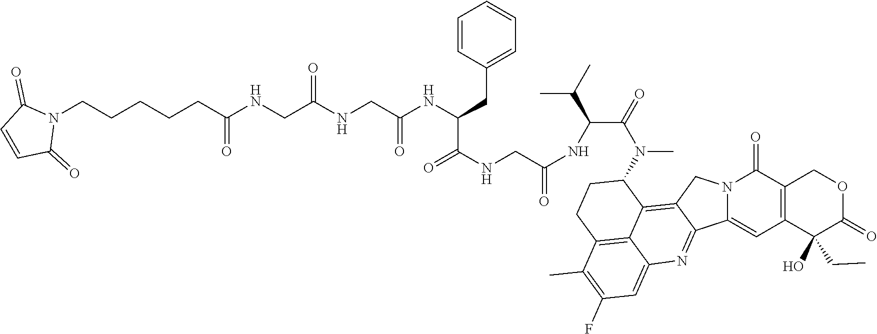 [SMILES: CC[C@@]1(O)C(=O)OCc2c1cc1n(c2=O)Cc2c-1nc1cc(F)c(C)c3c1c2[C@@H](N(C)C(=O)[C@@H](NC(=O)CNC(=O)[C@H](Cc1ccccc1)NC(=O)CNC(=O)CNC(=O)CCCCCN1C(=O)C=CC1=O)C(C)C)CC3] |
| L-I-35 | 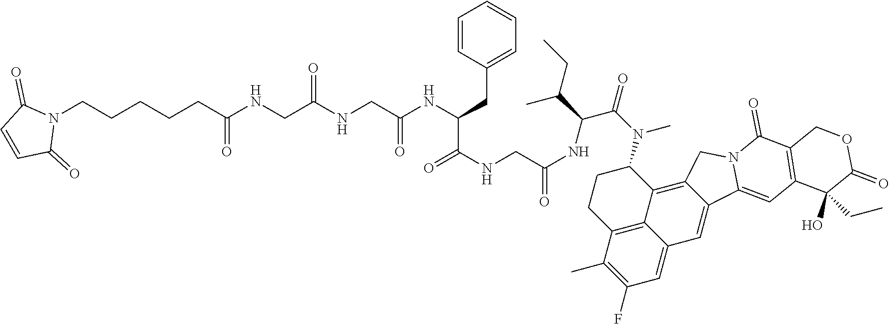 [SMILES: CCC(C)[C@H](NC(=O)CNC(=O)[C@H](Cc1ccccc1)NC(=O)CNC(=O)CNC(=O)CCCCCN1C(=O)C=CC1=O)C(=O)N(C)[C@H]1CCc2c(C)c(F)cc3cc4c(c1c23)Cn1c-4cc2c(c1=O)COC(=O)[C@]2(O)CC] |
| L-I-36 | 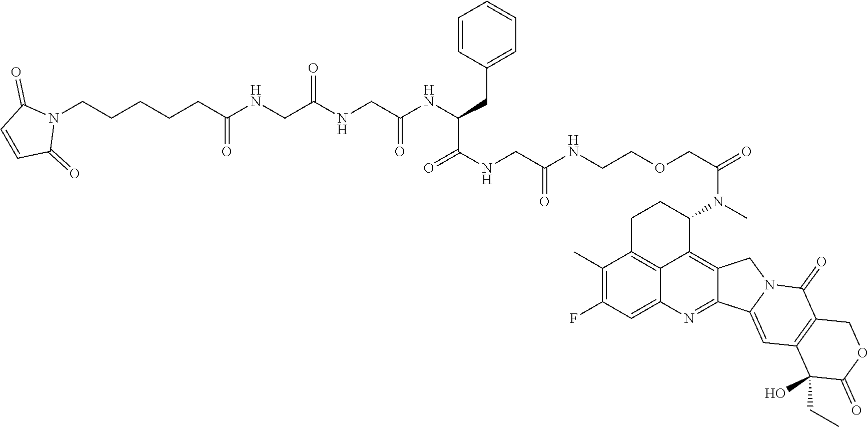 [SMILES: CC[C@@]1(O)C(=O)OCc2c1cc1n(c2=O)Cc2c-1nc1cc(F)c(C)c3c1c2[C@@H](N(C)C(=O)COCCNC(=O)CNC(=O)[C@H](Cc1ccccc1)NC(=O)CNC(=O)CNC(=O)CCCCCN1C(=O)C=CC1=O)CC3] |
| L-I-37 | 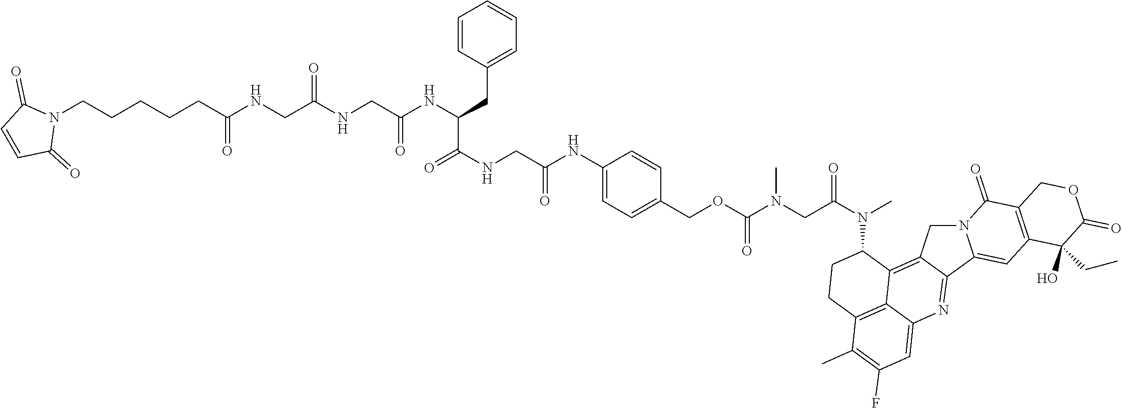 [SMILES: CC[C@@]1(O)C(=O)OCc2c1cc1n(c2=O)Cc2c-1nc1cc(F)c(C)c3c1c2[C@@H](N(C)C(=O)CN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-I-38 | 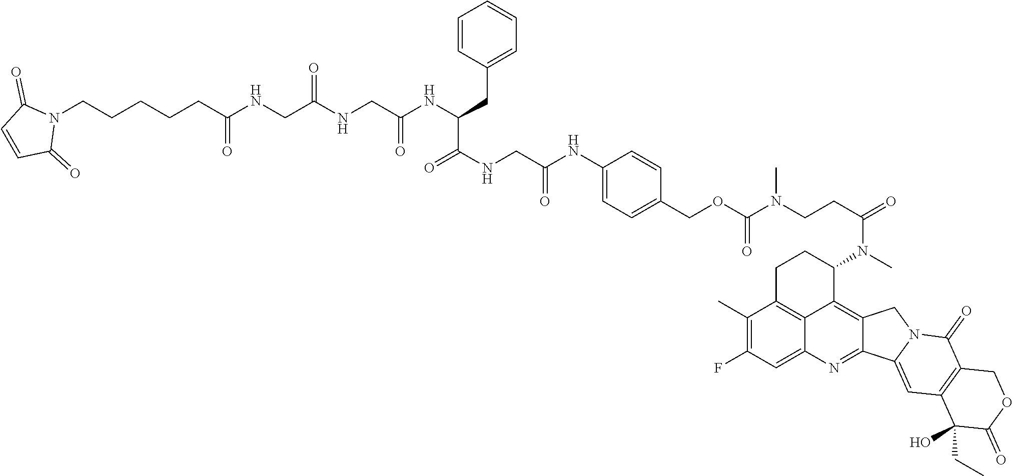 [SMILES: CC[C@@]1(O)C(=O)OCc2c1cc1n(c2=O)Cc2c-1nc1cc(F)c(C)c3c1c2[C@@H](N(C)C(=O)CCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-I-39 | 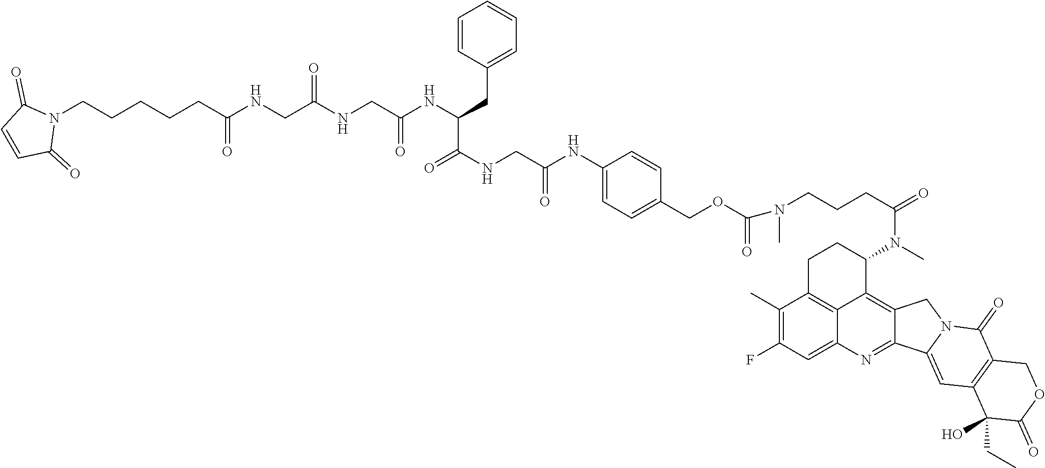 [SMILES: CC[C@@]1(O)C(=O)OCc2c1cc1n(c2=O)Cc2c-1nc1cc(F)c(C)c3c1c2[C@@H](N(C)C(=O)CCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-I-40 | 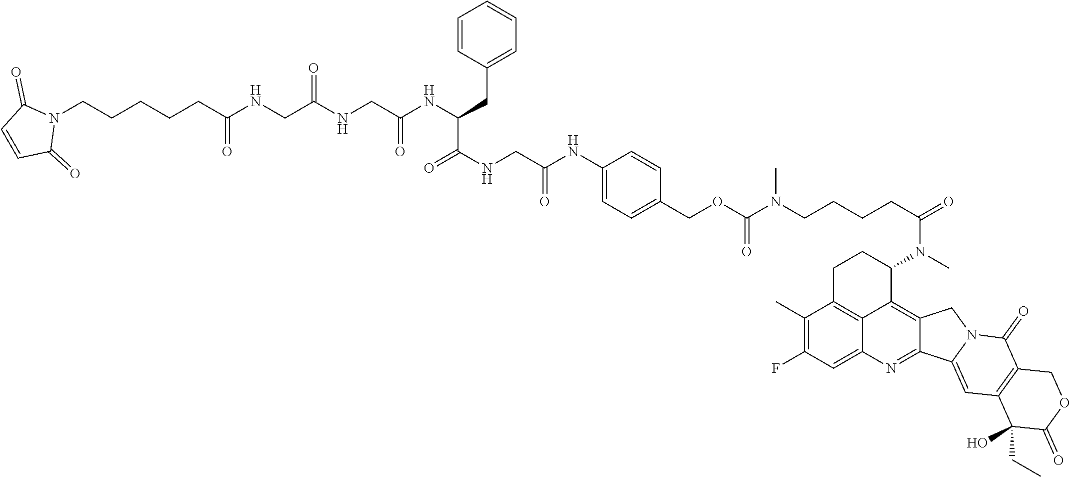 [SMILES: CC[C@@]1(O)C(=O)OCc2c1cc1n(c2=O)Cc2c-1nc1cc(F)c(C)c3c1c2[C@@H](N(C)C(=O)CCCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-I-41 | 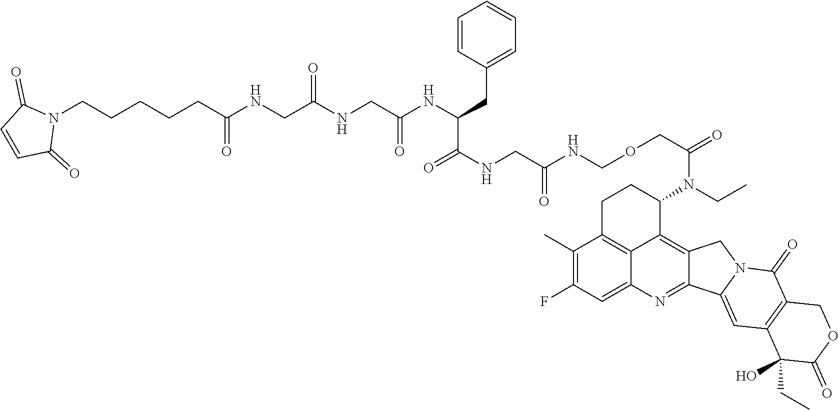 [SMILES: CCN(C(=O)COCNC(=O)CNC(=O)[C@H](Cc1ccccc1)NC(=O)CNC(=O)CNC(=O)CCCCCN1C(=O)C=CC1=O)[C@H]1CCc2c(C)c(F)cc3nc4c(c1c23)Cn1c-4cc2c(c1=O)COC(=O)[C@]2(O)CC] |
| L-I-42 | 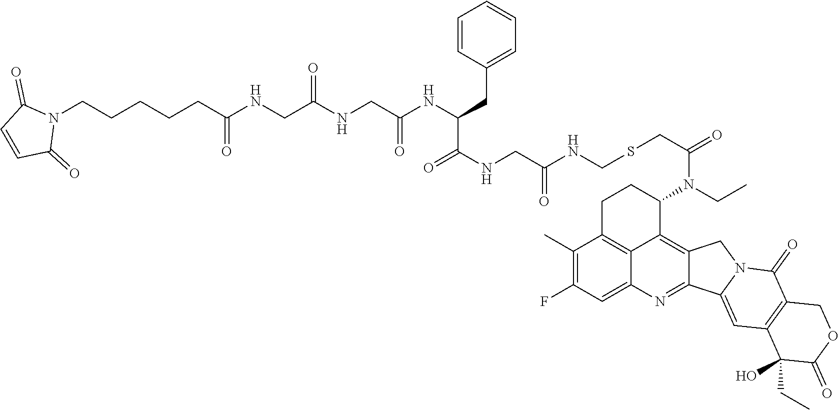 [SMILES: CCN(C(=O)CSCNC(=O)CNC(=O)[C@H](Cc1ccccc1)NC(=O)CNC(=O)CNC(=O)CCCCCN1C(=O)C=CC1=O)[C@H]1CCc2c(C)c(F)cc3nc4c(c1c23)Cn1c-4cc2c(c1=O)COC(=O)[C@]2(O)CC] |
| L-I-43 | 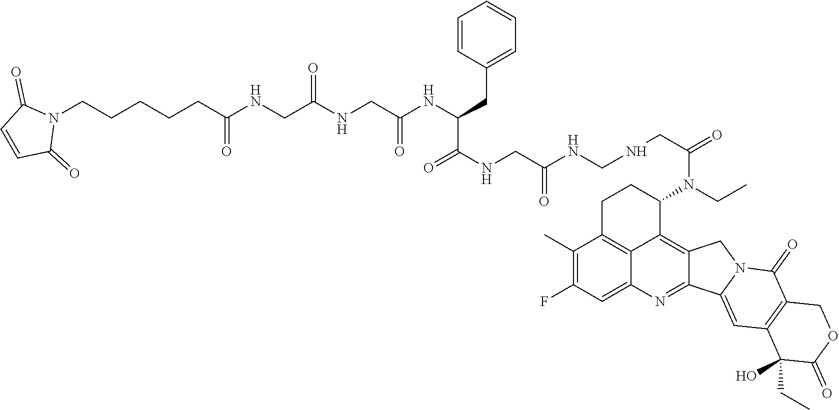 [SMILES: CCN(C(=O)CNCNC(=O)CNC(=O)[C@H](Cc1ccccc1)NC(=O)CNC(=O)CNC(=O)CCCCCN1C(=O)C=CC1=O)[C@H]1CCc2c(C)c(F)cc3nc4c(c1c23)Cn1c-4cc2c(c1=O)COC(=O)[C@]2(O)CC] |
| L-I-44 | 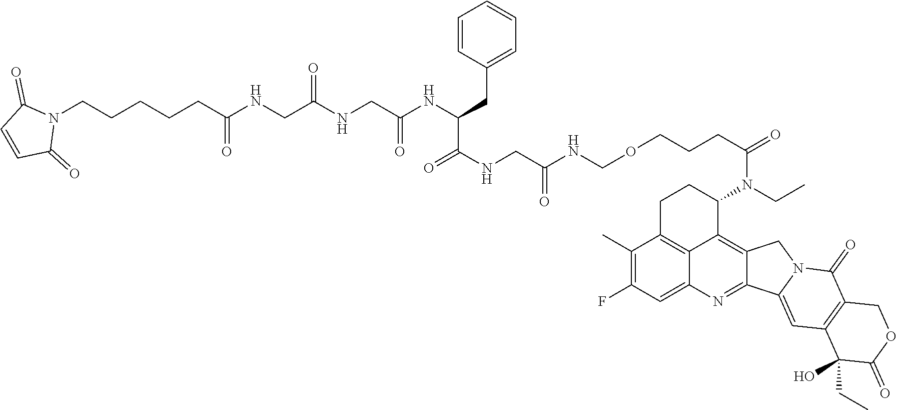 [SMILES: CCN(C(=O)CCCOCNC(=O)CNC(=O)[C@H](Cc1ccccc1)NC(=O)CNC(=O)CNC(=O)CCCCCN1C(=O)C=CC1=O)[C@H]1CCc2c(C)c(F)cc3nc4c(c1c23)Cn1c-4cc2c(c1=O)COC(=O)[C@]2(O)CC] |
| L-I-45 | 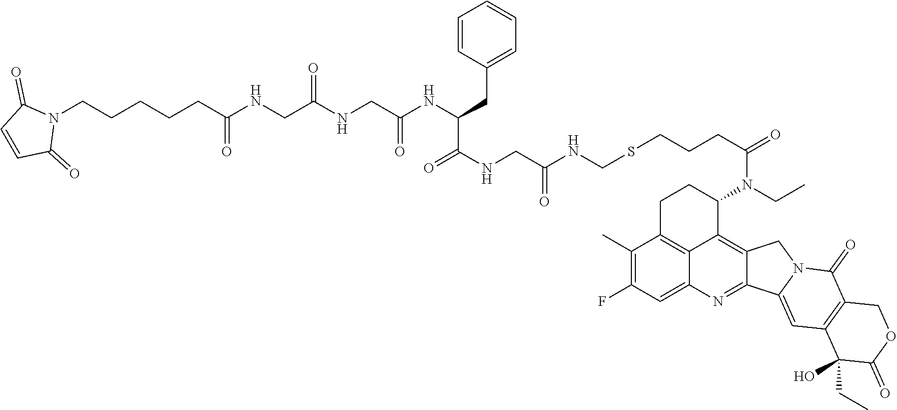 [SMILES: CCN(C(=O)CCCSCNC(=O)CNC(=O)[C@H](Cc1ccccc1)NC(=O)CNC(=O)CNC(=O)CCCCCN1C(=O)C=CC1=O)[C@H]1CCc2c(C)c(F)cc3nc4c(c1c23)Cn1c-4cc2c(c1=O)COC(=O)[C@]2(O)CC] |
| L-I-46 | 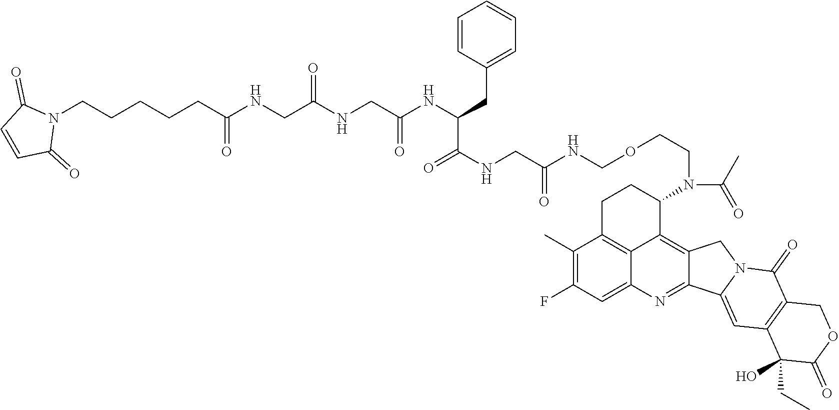 [SMILES: CC[C@@]1(O)C(=O)OCc2c1cc1n(c2=O)Cc2c-1nc1cc(F)c(C)c3c1c2[C@@H](N(CCOCNC(=O)CNC(=O)[C@H](Cc1ccccc1)NC(=O)CNC(=O)CNC(=O)CCCCCN1C(=O)C=CC1=O)C(C)=O)CC3] |
| L-I-47 | 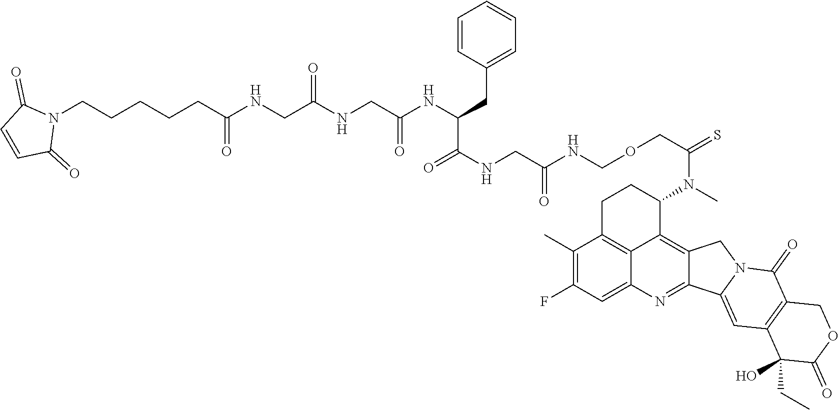 [SMILES: CC[C@@]1(O)C(=O)OCc2c1cc1n(c2=O)Cc2c-1nc1cc(F)c(C)c3c1c2[C@@H](N(C)C(=S)COCNC(=O)CNC(=O)[C@H](Cc1ccccc1)NC(=O)CNC(=O)CNC(=O)CCCCCN1C(=O)C=CC1=O)CC3] |
| L-I-48 | 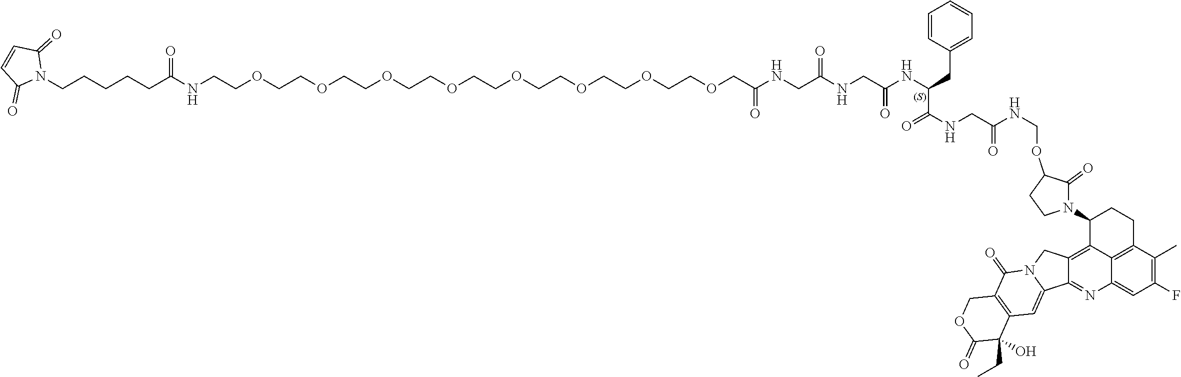 |
| L-I-49 | 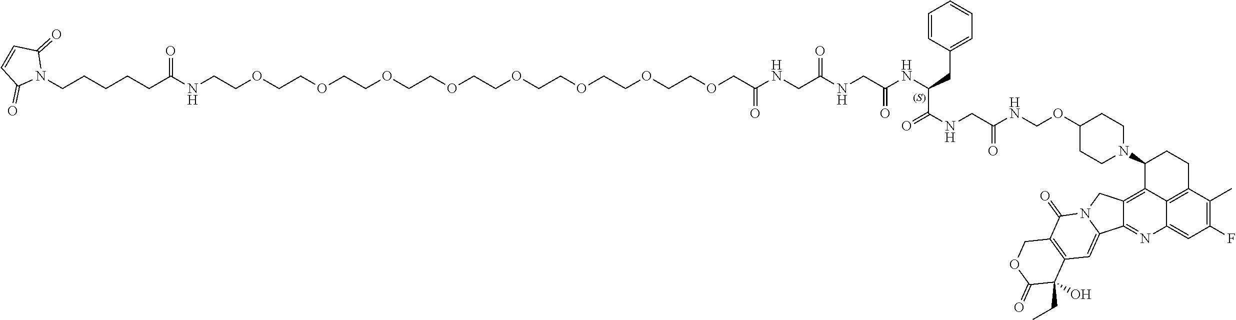 |
| L-I-50 | 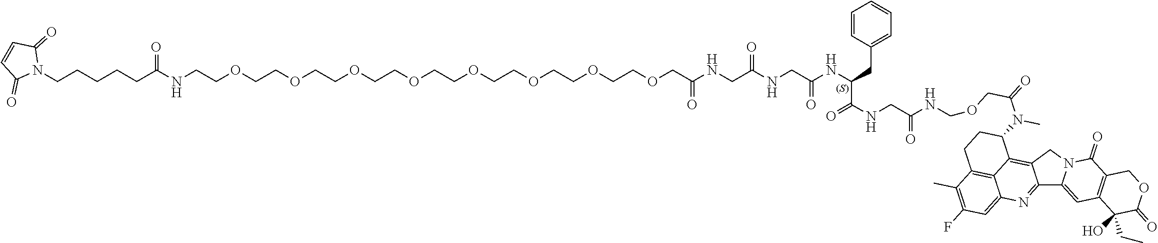 |
| L-II-1 | 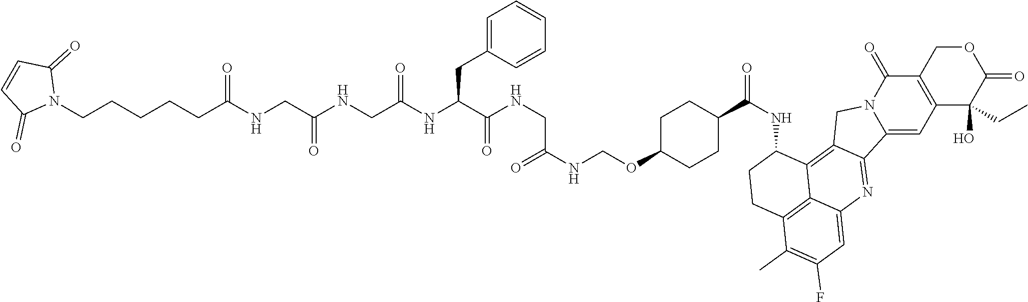 [SMILES: CC[C@@]1(O)C(=O)OCc2c1cc1n(c2=O)Cc2c-1nc1cc(F)c(C)c3c1c2[C@@H](NC(=O)[C@H]1CC[C@@H](OCNC(=O)CNC(=O)[C@H](Cc2ccccc2)NC(=O)CNC(=O)CNC(=O)CCCCCN2C(=O)C=CC2=O)CC1)CC3] |
| L-II-2 | 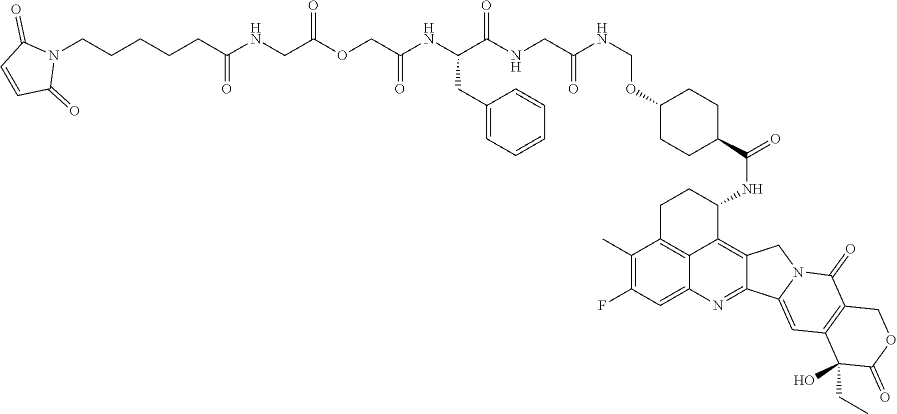 [SMILES: CC[C@@]1(O)C(=O)OCc2c1cc1n(c2=O)Cc2c-1nc1cc(F)c(C)c3c1c2[C@@H](NC(=O)[C@H]1CC[C@H](OCNC(=O)CNC(=O)[C@H](Cc2ccccc2)NC(=O)COC(=O)CNC(=O)CCCCCN2C(=O)C=CC2=O)CC1)CC3] |
| L-II-3 | 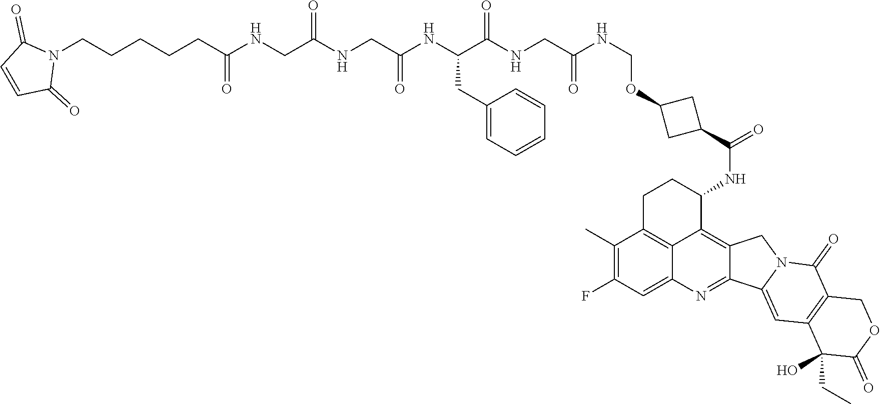 [SMILES: CC[C@@]1(O)C(=O)OCc2c1cc1n(c2=O)Cc2c-1nc1cc(F)c(C)c3c1c2[C@@H](NC(=O)[C@H]1C[C@@H](OCNC(=O)CNC(=O)[C@H](Cc2ccccc2)NC(=O)CNC(=O)CNC(=O)CCCCCN2C(=O)C=CC2=O)C1)CC3] |
| L-II-4 | 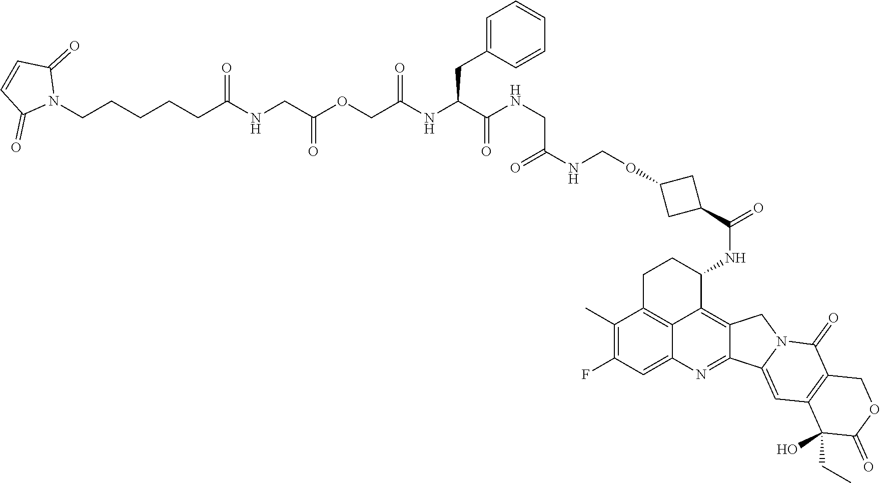 [SMILES: CC[C@@]1(O)C(=O)OCc2c1cc1n(c2=O)Cc2c-1nc1cc(F)c(C)c3c1c2[C@@H](NC(=O)[C@H]1C[C@H](OCNC(=O)CNC(=O)[C@H](Cc2ccccc2)NC(=O)COC(=O)CNC(=O)CCCCCN2C(=O)C=CC2=O)C1)CC3] |
| L-II-5 | 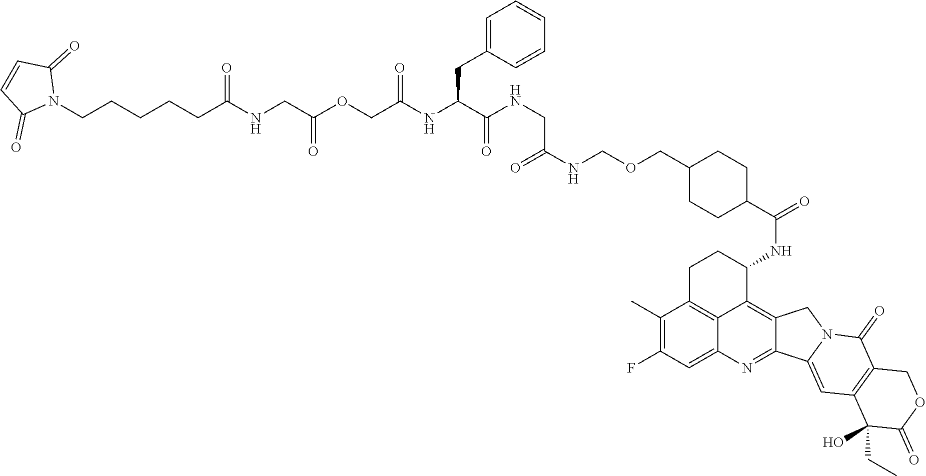 [SMILES: CC[C@@]1(O)C(=O)OCc2c1cc1n(c2=O)Cc2c-1nc1cc(F)c(C)c3c1c2[C@@H](NC(=O)C1CCC(COCNC(=O)CNC(=O)[C@H](Cc2ccccc2)NC(=O)COC(=O)CNC(=O)CCCCCN2C(=O)C=CC2=O)CC1)CC3] |
| L-II-6 | 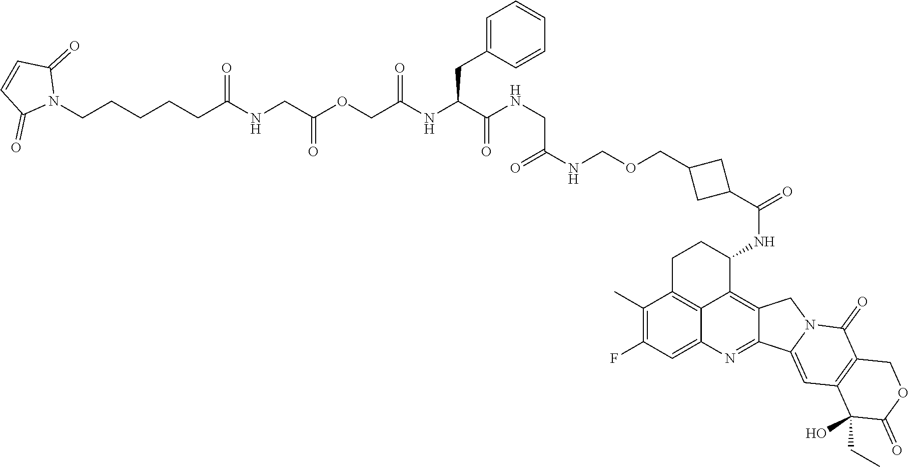 [SMILES: CC[C@@]1(O)C(=O)OCc2c1cc1n(c2=O)Cc2c-1nc1cc(F)c(C)c3c1c2[C@@H](NC(=O)C1CC(COCNC(=O)CNC(=O)[C@H](Cc2ccccc2)NC(=O)COC(=O)CNC(=O)CCCCCN2C(=O)C=CC2=O)C1)CC3] |
| L-II-7 | 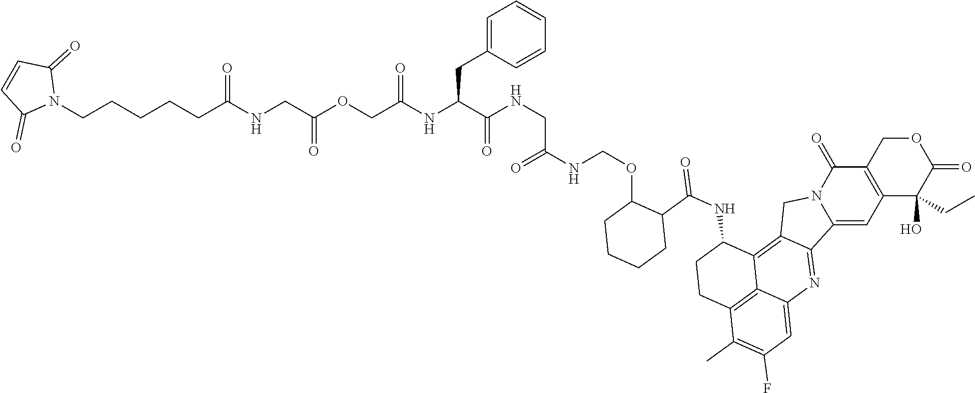 [SMILES: CC[C@@]1(O)C(=O)OCc2c1cc1n(c2=O)Cc2c-1nc1cc(F)c(C)c3c1c2[C@@H](NC(=O)C1CCCCC1OCNC(=O)CNC(=O)[C@H](Cc1ccccc1)NC(=O)COC(=O)CNC(=O)CCCCCN1C(=O)C=CC1=O)CC3] |
| L-II-8 | 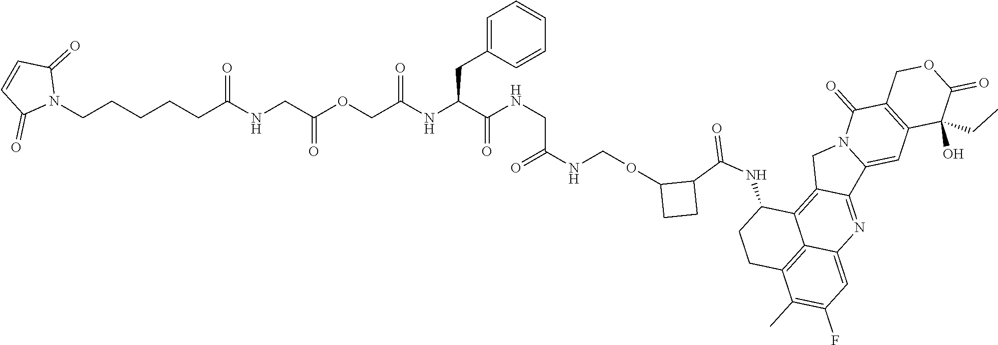 [SMILES: CC[C@@]1(O)C(=O)OCc2c1cc1n(c2=O)Cc2c-1nc1cc(F)c(C)c3c1c2[C@@H](NC(=O)C1CCC1OCNC(=O)CNC(=O)[C@H](Cc1ccccc1)NC(=O)COC(=O)CNC(=O)CCCCCN1C(=O)C=CC1=O)CC3] |
| L-II-9 | 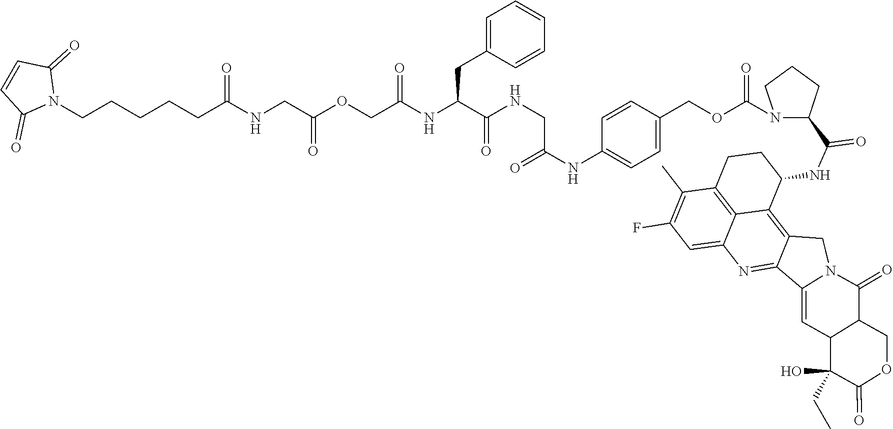 [SMILES: CC[C@@]1(O)C(=O)OCC2C(=O)N3Cc4c(nc5cc(F)c(C)c6c5c4[C@@H](NC(=O)[C@@H]4CCCN4C(=O)OCc4ccc(NC(=O)CNC(=O)[C@H](Cc5ccccc5)NC(=O)COC(=O)CNC(=O)CCCCCN5C(=O)C=CC5=O)cc4)CC6)C3=CC21] |
| L-II-10 | 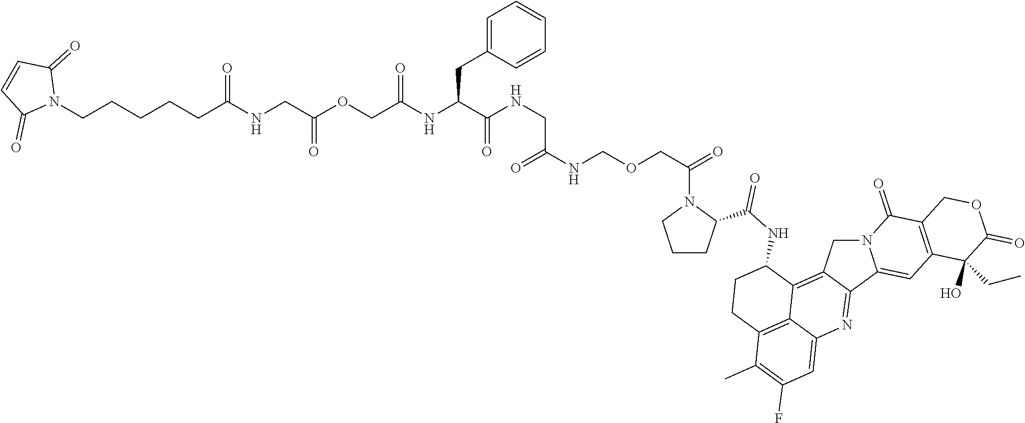 [SMILES: CC[C@@]1(O)C(=O)OCc2c1cc1n(c2=O)Cc2c-1nc1cc(F)c(C)c3c1c2[C@@H](NC(=O)[C@@H]1CCCN1C(=O)COCNC(=O)CNC(=O)[C@H](Cc1ccccc1)NC(=O)COC(=O)CNC(=O)CCCCCN1C(=O)C=CC1=O)CC3] |
| L-II-11 | 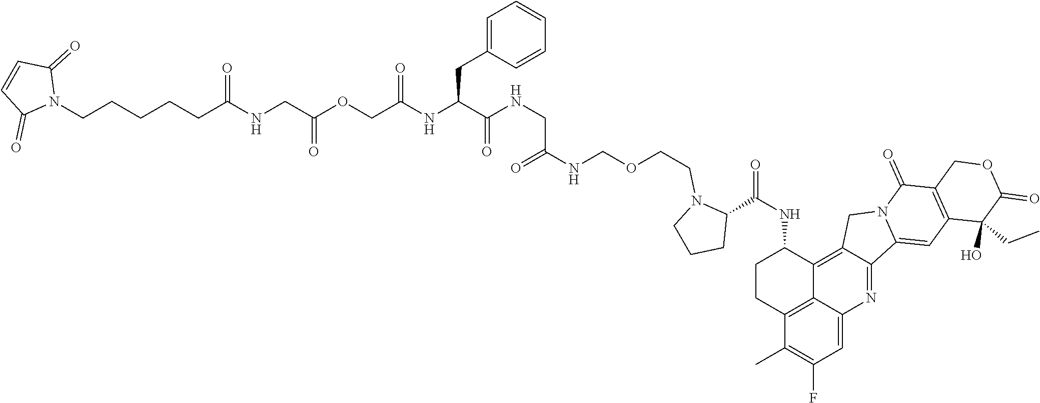 [SMILES: CC[C@@]1(O)C(=O)OCc2c1cc1n(c2=O)Cc2c-1nc1cc(F)c(C)c3c1c2[C@@H](NC(=O)[C@@H]1CCCN1CCOCNC(=O)CNC(=O)[C@H](Cc1ccccc1)NC(=O)COC(=O)CNC(=O)CCCCCN1C(=O)C=CC1=O)CC3] |
| L-II-12 | 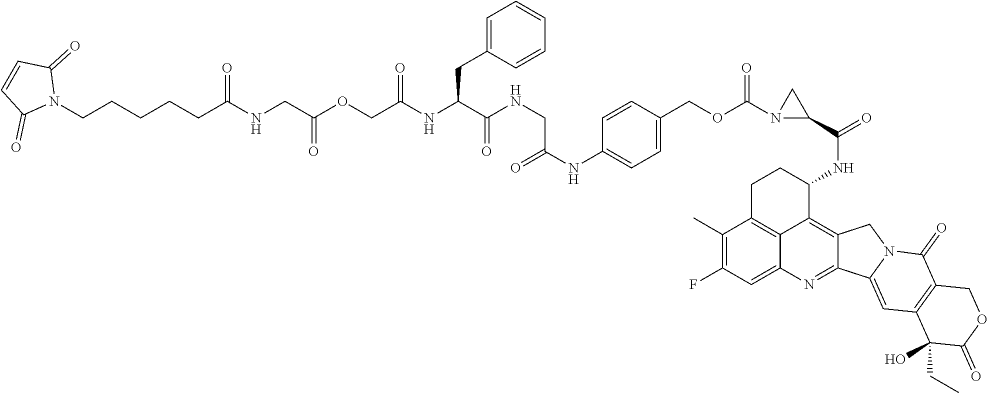 [SMILES: CC[C@@]1(O)C(=O)OCc2c1cc1n(c2=O)Cc2c-1nc1cc(F)c(C)c3c1c2[C@@H](NC(=O)[C@@H]1CN1C(=O)OCc1ccc(NC(=O)CNC(=O)[C@H](Cc2ccccc2)NC(=O)COC(=O)CNC(=O)CCCCCN2C(=O)C=CC2=O)cc1)CC3] |
| L-II-13 | 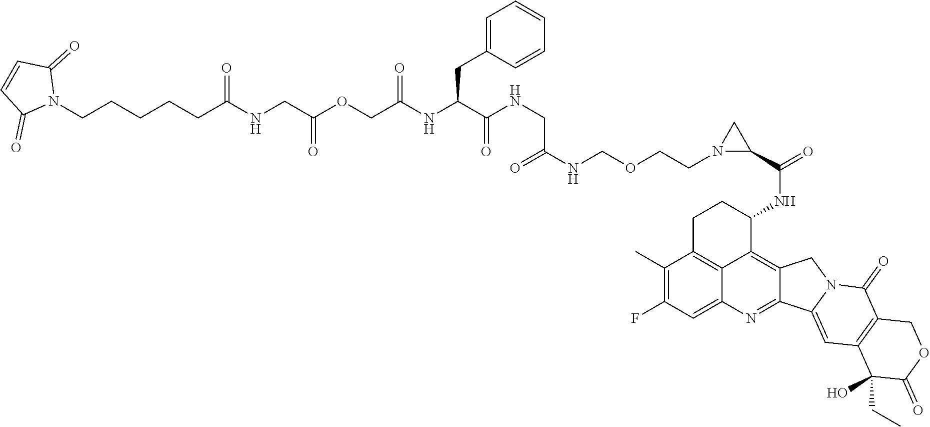 [SMILES: CC[C@@]1(O)C(=O)OCc2c1cc1n(c2=O)Cc2c-1nc1cc(F)c(C)c3c1c2[C@@H](NC(=O)[C@@H]1CN1CCOCNC(=O)CNC(=O)[C@H](Cc1ccccc1)NC(=O)COC(=O)CNC(=O)CCCCCN1C(=O)C=CC1=O)CC3] |
| L-II-14 | 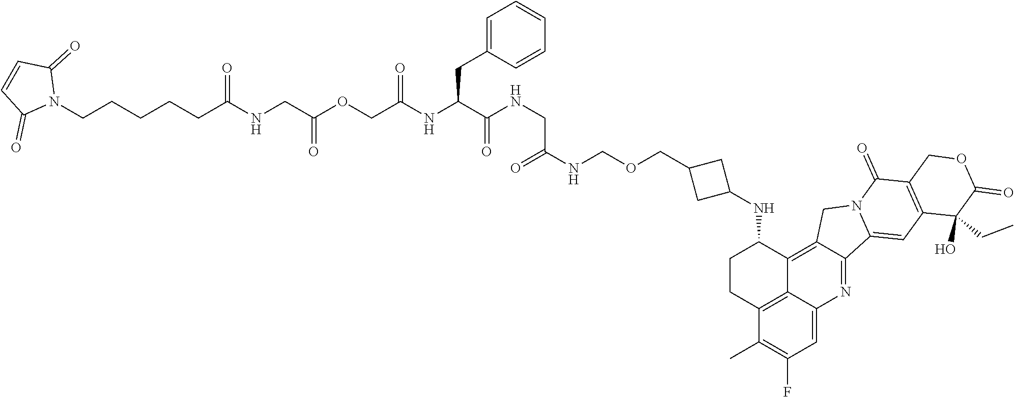 [SMILES: CC[C@@]1(O)C(=O)OCc2c1cc1n(c2=O)Cc2c-1nc1cc(F)c(C)c3c1c2[C@@H](NC1CC(COCNC(=O)CNC(=O)[C@H](Cc2ccccc2)NC(=O)COC(=O)CNC(=O)CCCCCN2C(=O)C=CC2=O)C1)CC3] |
| L-II-15 | 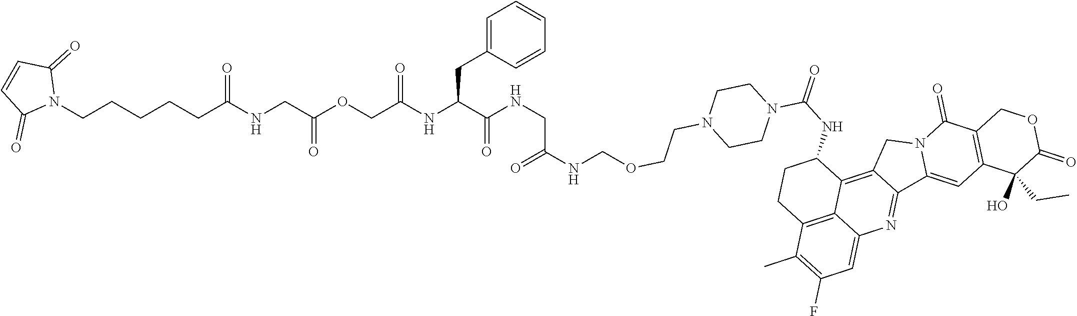 [SMILES: CC[C@@]1(O)C(=O)OCc2c1cc1n(c2=O)Cc2c-1nc1cc(F)c(C)c3c1c2[C@@H](NC(=O)N1CCN(CCOCNC(=O)CNC(=O)[C@H](Cc2ccccc2)NC(=O)COC(=O)CNC(=O)CCCCCN2C(=O)C=CC2=O)CC1)CC3] |
| L-II-16 | 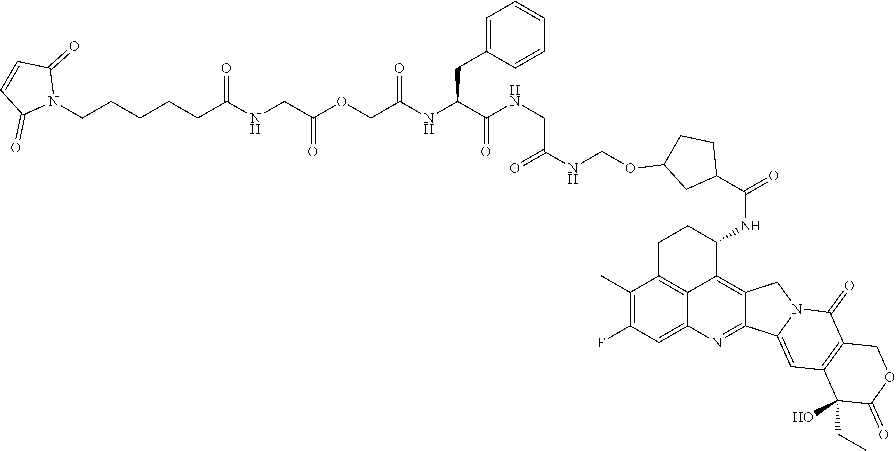 [SMILES: CC[C@@]1(O)C(=O)OCc2c1cc1n(c2=O)Cc2c-1nc1cc(F)c(C)c3c1c2[C@@H](NC(=O)C1CCC(OCNC(=O)CNC(=O)[C@H](Cc2ccccc2)NC(=O)COC(=O)CNC(=O)CCCCCN2C(=O)C=CC2=O)C1)CC3] |
| L-II-17 | 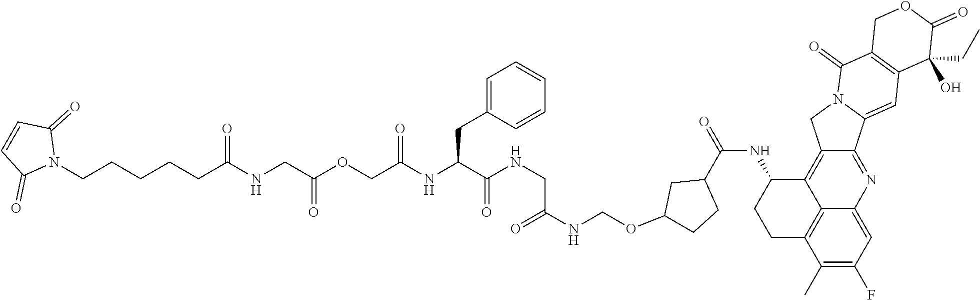 [SMILES: CC[C@@]1(O)C(=O)OCc2c1cc1n(c2=O)Cc2c-1nc1cc(F)c(C)c3c1c2[C@@H](NC(=O)C1CCC(OCNC(=O)CNC(=O)[C@H](Cc2ccccc2)NC(=O)COC(=O)CNC(=O)CCCCCN2C(=O)C=CC2=O)C1)CC3] |
| L-II-18 | 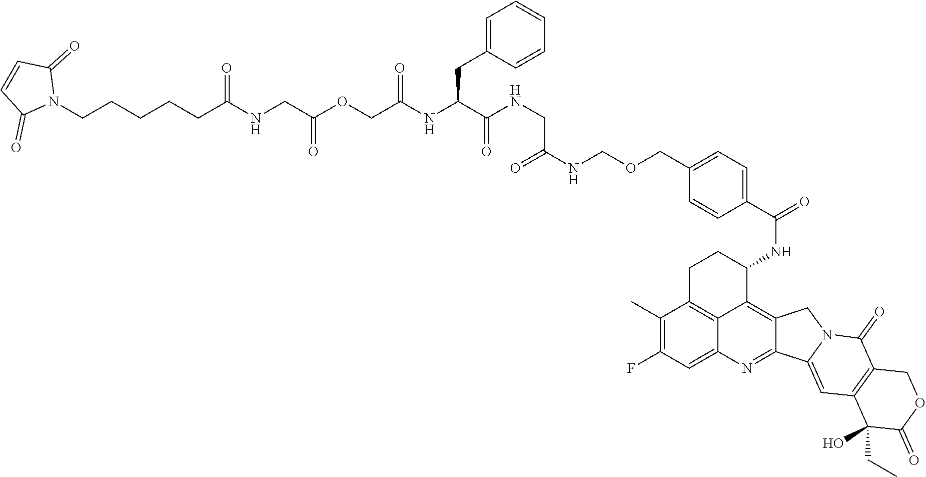 [SMILES: CC[C@@]1(O)C(=O)OCc2c1cc1n(c2=O)Cc2c-1nc1cc(F)c(C)c3c1c2[C@@H](NC(=O)c1ccc(COCNC(=O)CNC(=O)[C@H](Cc2ccccc2)NC(=O)COC(=O)CNC(=O)CCCCCN2C(=O)C=CC2=O)cc1)CC3] |
| L-II-19 | 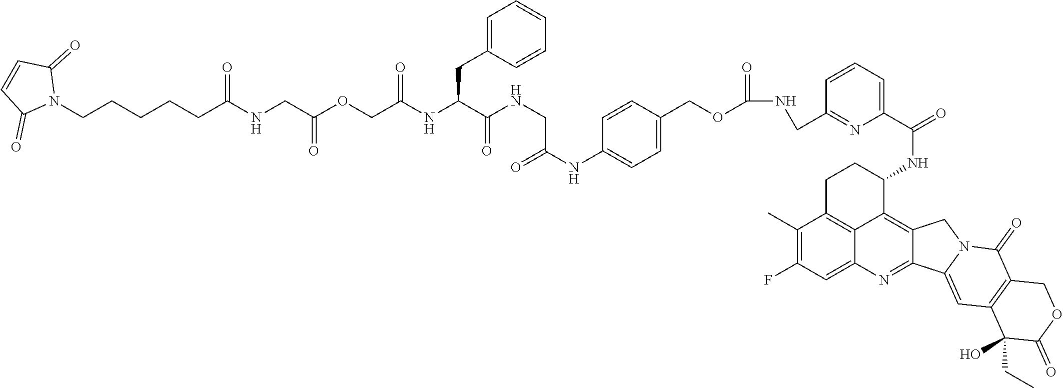 [SMILES: CC[C@@]1(O)C(=O)OCc2c1cc1n(c2=O)Cc2c-1nc1cc(F)c(C)c3c1c2[C@@H](NC(=O)c1cccc(CNC(=O)OCc2ccc(NC(=O)CNC(=O)[C@H](Cc4ccccc4)NC(=O)COC(=O)CNC(=O)CCCCCN4C(=O)C=CC4=O)cc2)n1)CC3] |
| L-II-20 | 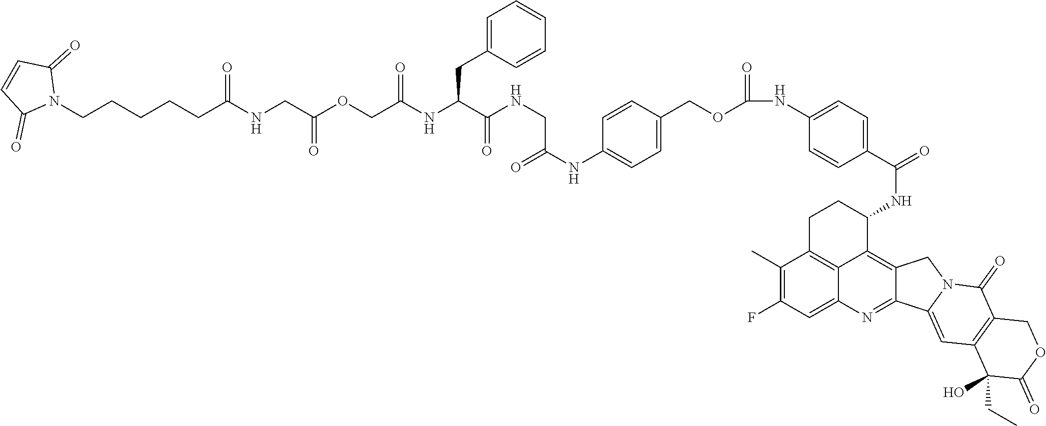 [SMILES: CC[C@@]1(O)C(=O)OCc2c1cc1n(c2=O)Cc2c-1nc1cc(F)c(C)c3c1c2[C@@H](NC(=O)c1ccc(NC(=O)OCc2ccc(NC(=O)CNC(=O)[C@H](Cc4ccccc4)NC(=O)COC(=O)CNC(=O)CCCCCN4C(=O)C=CC4=O)cc2)cc1)CC3] |
| L-II-21 | 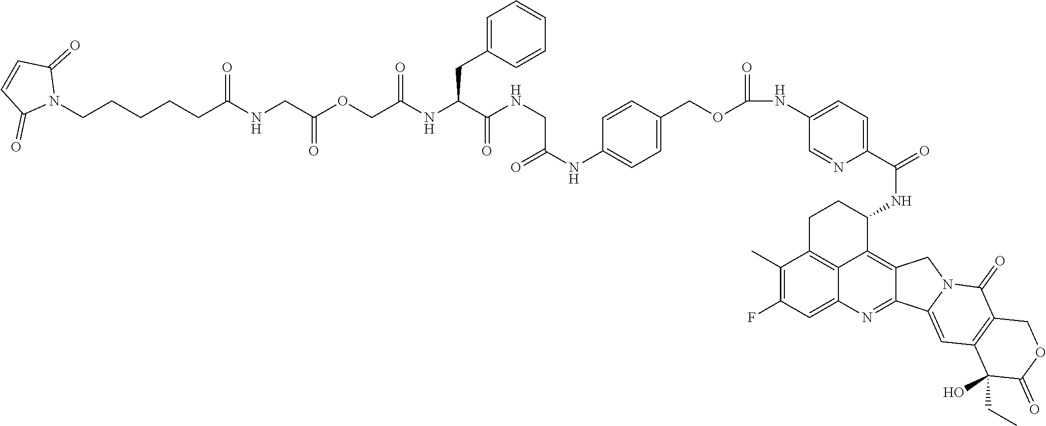 [SMILES: CC[C@@]1(O)C(=O)OCc2c1cc1n(c2=O)Cc2c-1nc1cc(F)c(C)c3c1c2[C@@H](NC(=O)c1ccc(NC(=O)OCc2ccc(NC(=O)CNC(=O)[C@H](Cc4ccccc4)NC(=O)COC(=O)CNC(=O)CCCCCN4C(=O)C=CC4=O)cc2)cn1)CC3] |
| L-II-22 | 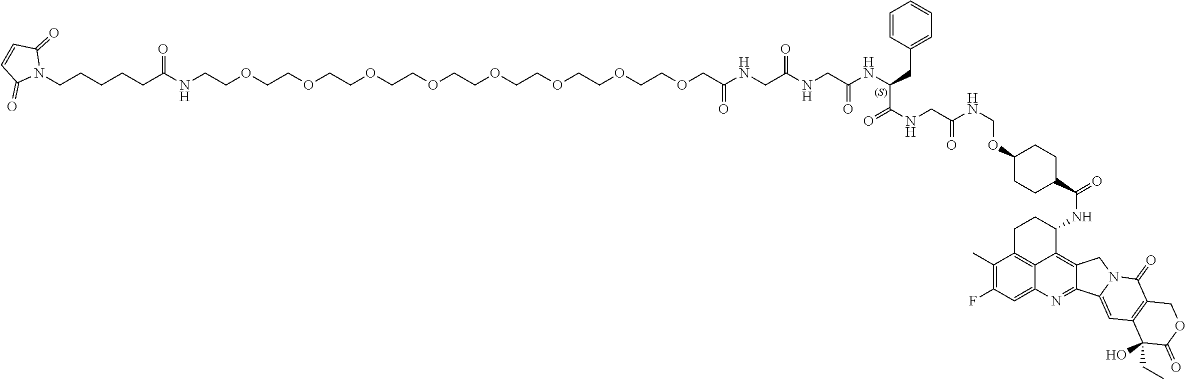 |
| L-II-23 | 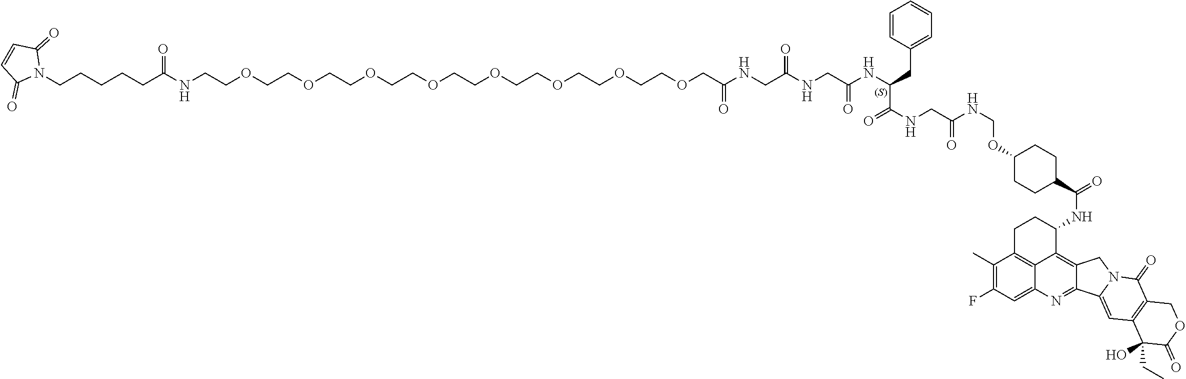 |
| L-II-24 | 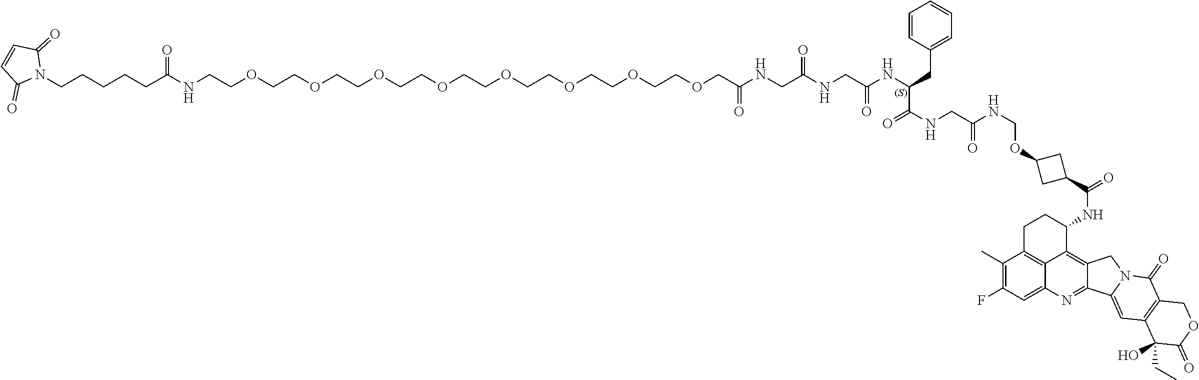 |
| L-II-25 | 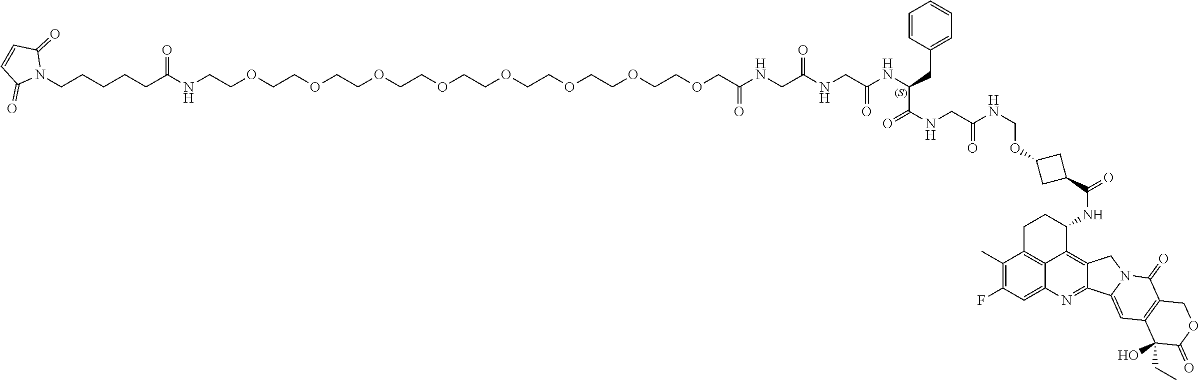 |
| L-II-26 | 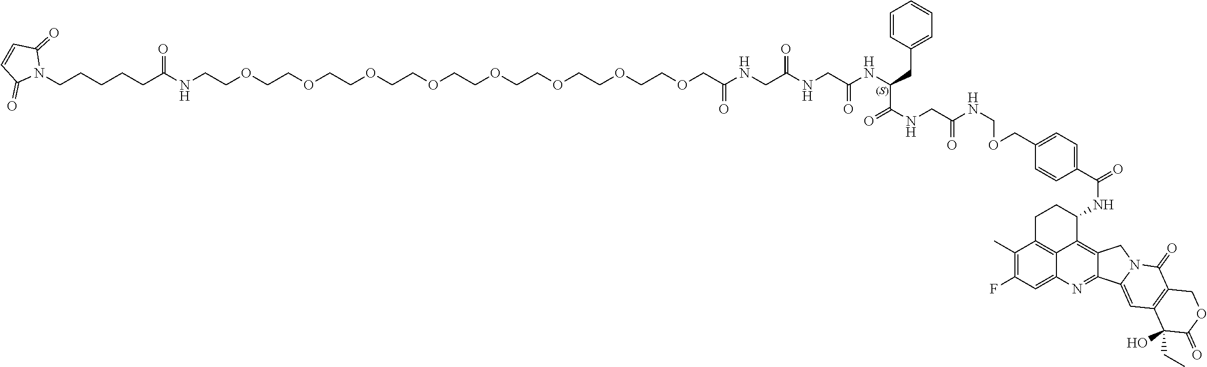 |
| L-II-27 | 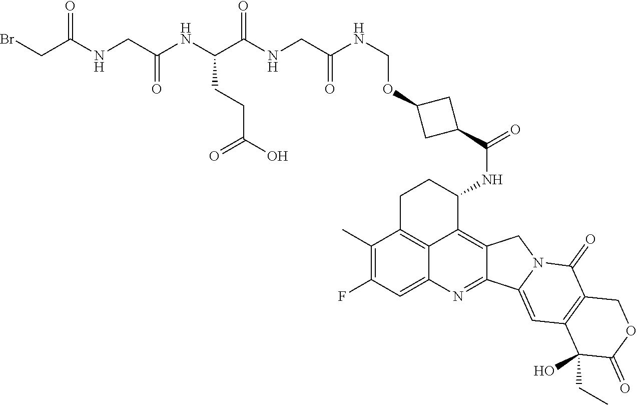 [SMILES: CC[C@@]1(O)C(=O)OCc2c1cc1n(c2=O)Cc2c-1nc1cc(F)c(C)c3c1c2[C@@H](NC(=O)[C@H]1C[C@@H](OCNC(=O)CNC(=O)[C@H](CCC(=O)O)NC(=O)CNC(=O)CBr)C1)CC3] |
| L-II-28 | 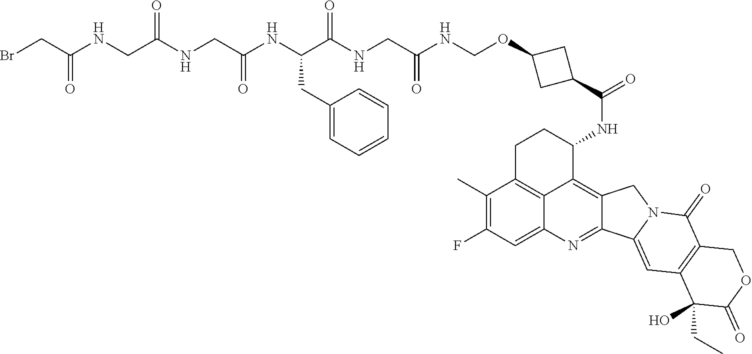 [SMILES: CC[C@@]1(O)C(=O)OCc2c1cc1n(c2=O)Cc2c-1nc1cc(F)c(C)c3c1c2[C@@H](NC(=O)[C@H]1C[C@@H](OCNC(=O)CNC(=O)[C@H](Cc2ccccc2)NC(=O)CNC(=O)CNC(=O)CBr)C1)CC3] |
| L-III-1 | 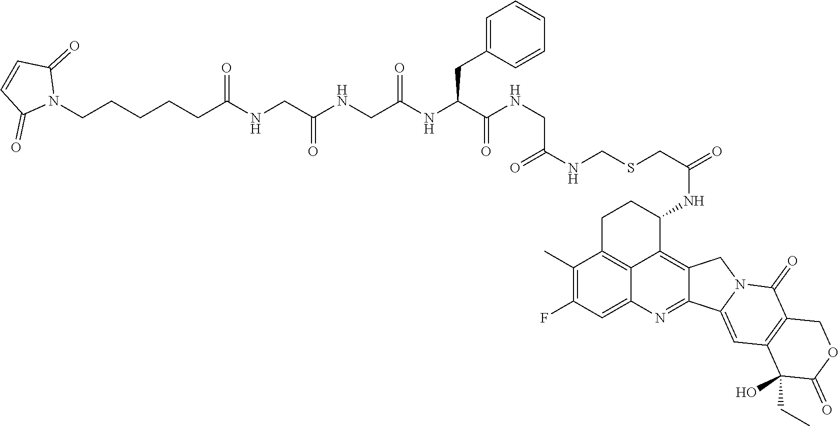 [SMILES: CC[C@@]1(O)C(=O)OCc2c1cc1n(c2=O)Cc2c-1nc1cc(F)c(C)c3c1c2[C@@H](NC(=O)CSCNC(=O)CNC(=O)[C@H](Cc1ccccc1)NC(=O)CNC(=O)CNC(=O)CCCCCN1C(=O)C=CC1=O)CC3] |
| L-III-2 | 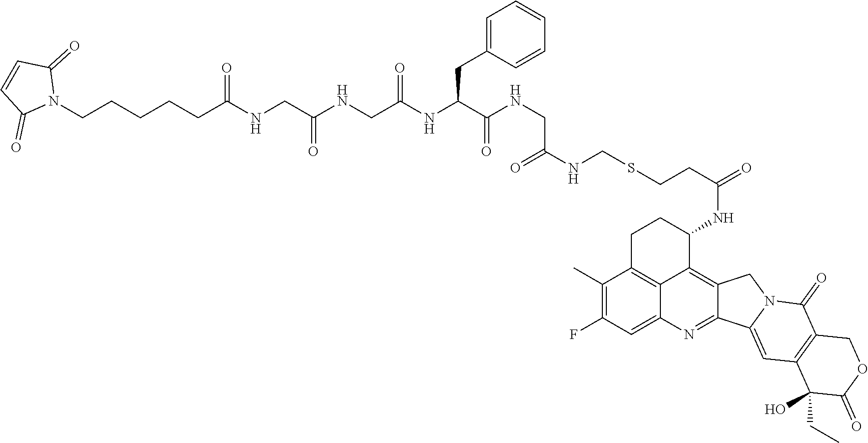 [SMILES: CC[C@@]1(O)C(=O)OCc2c1cc1n(c2=O)Cc2c-1nc1cc(F)c(C)c3c1c2[C@@H](NC(=O)CCSCNC(=O)CNC(=O)[C@H](Cc1ccccc1)NC(=O)CNC(=O)CNC(=O)CCCCCN1C(=O)C=CC1=O)CC3] |
| L-III-3 | 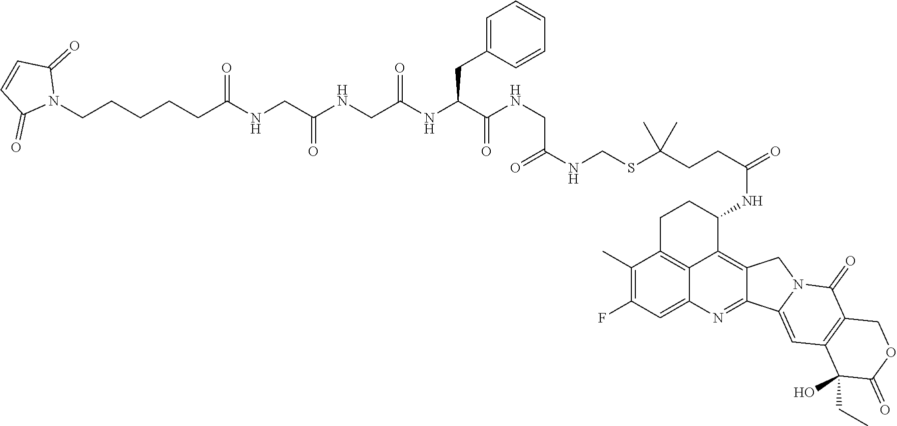 [SMILES: CC[C@@]1(O)C(=O)OCc2c1cc1n(c2=O)Cc2c-1nc1cc(F)c(C)c3c1c2[C@@H](NC(=O)CCC(C)(C)SCNC(=O)CNC(=O)[C@H](Cc1ccccc1)NC(=O)CNC(=O)CNC(=O)CCCCCN1C(=O)C=CC1=O)CC3] |
| L-III-4 | 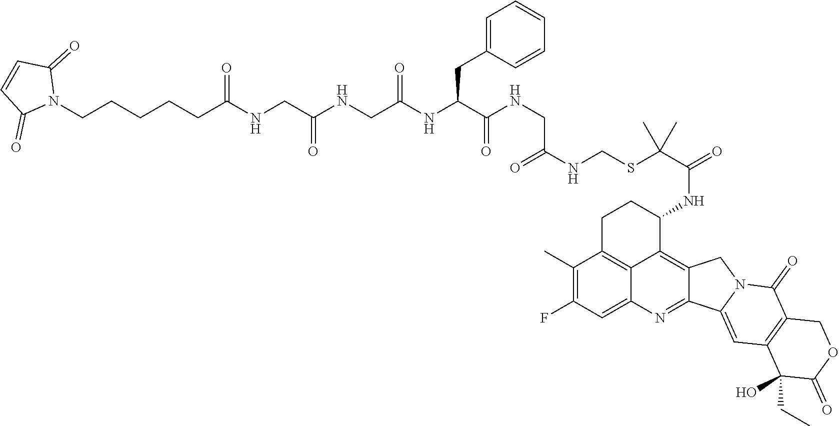 [SMILES: CC[C@@]1(O)C(=O)OCc2c1cc1n(c2=O)Cc2c-1nc1cc(F)c(C)c3c1c2[C@@H](NC(=O)C(C)(C)SCNC(=O)CNC(=O)[C@H](Cc1ccccc1)NC(=O)CNC(=O)CNC(=O)CCCCCN1C(=O)C=CC1=O)CC3] |
| L-III-5 | 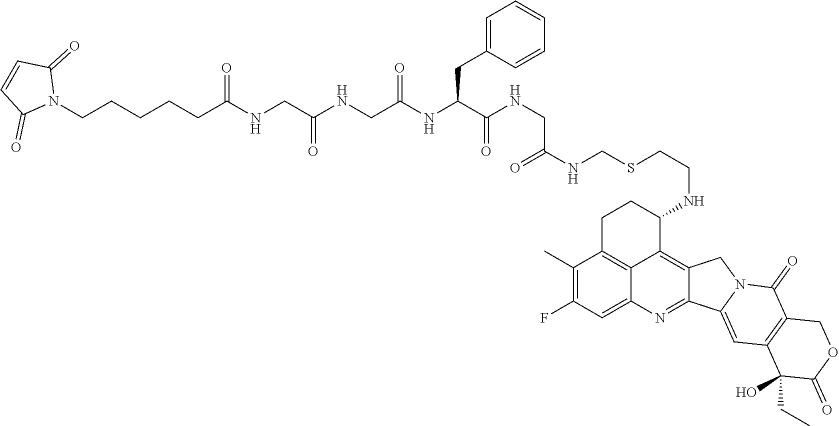 [SMILES: CC[C@@]1(O)C(=O)OCc2c1cc1n(c2=O)Cc2c-1nc1cc(F)c(C)c3c1c2[C@@H](NCCSCNC(=O)CNC(=O)[C@H](Cc1ccccc1)NC(=O)CNC(=O)CNC(=O)CCCCCN1C(=O)C=CC1=O)CC3] |
| L-III-6 | 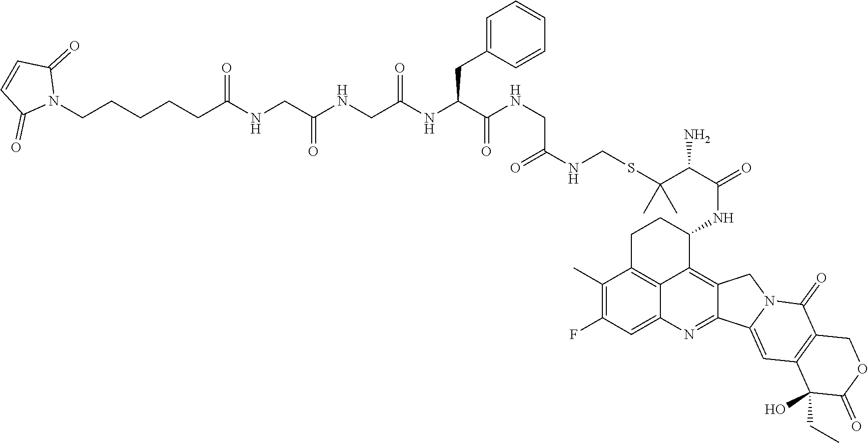 [SMILES: CC[C@@]1(O)C(=O)OCc2c1cc1n(c2=O)Cc2c-1nc1cc(F)c(C)c3c1c2[C@@H](NC(=O)[C@@H](N)C(C)(C)SCNC(=O)CNC(=O)[C@H](Cc1ccccc1)NC(=O)CNC(=O)CNC(=O)CCCCCN1C(=O)C=CC1=O)CC3] |
| L-III-7 | 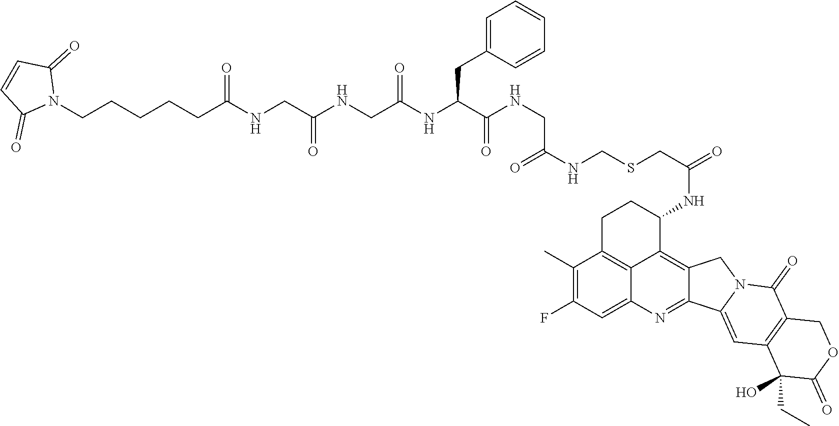 [SMILES: CC[C@@]1(O)C(=O)OCc2c1cc1n(c2=O)Cc2c-1nc1cc(F)c(C)c3c1c2[C@@H](NC(=O)CSCNC(=O)CNC(=O)[C@H](Cc1ccccc1)NC(=O)CNC(=O)CNC(=O)CCCCCN1C(=O)C=CC1=O)CC3] |
| L-III-8 | 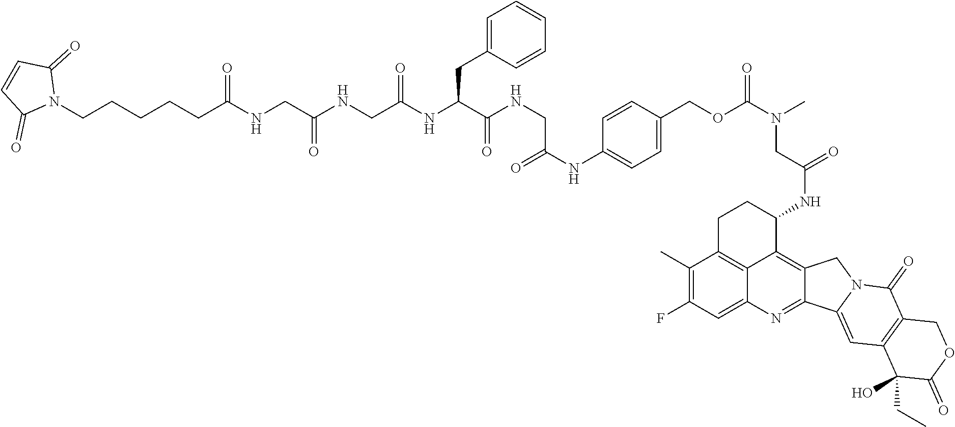 [SMILES: CC[C@@]1(O)C(=O)OCc2c1cc1n(c2=O)Cc2c-1nc1cc(F)c(C)c3c1c2[C@@H](NC(=O)CN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-9 | 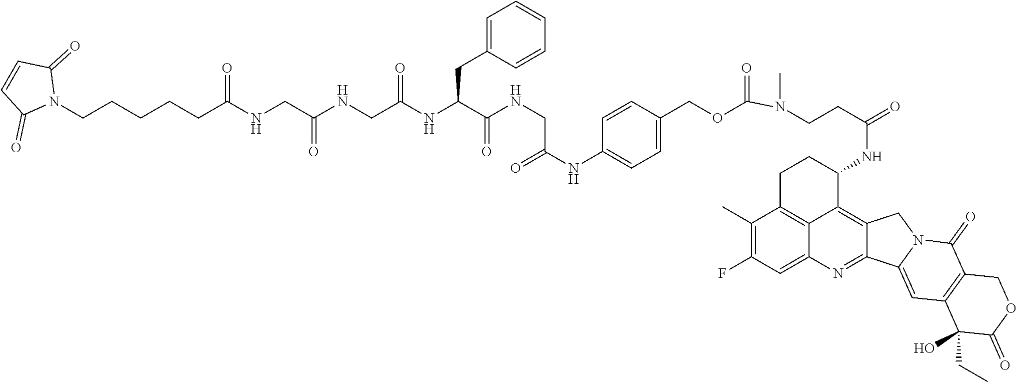 [SMILES: CC[C@@]1(O)C(=O)OCc2c1cc1n(c2=O)Cc2c-1nc1cc(F)c(C)c3c1c2[C@@H](NC(=O)CCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-10 | 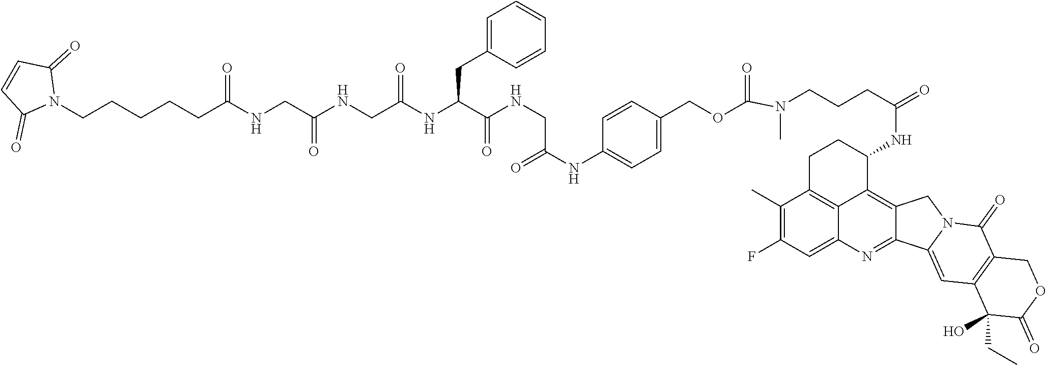 [SMILES: CC[C@@]1(O)C(=O)OCc2c1cc1n(c2=O)Cc2c-1nc1cc(F)c(C)c3c1c2[C@@H](NC(=O)CCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-11 | 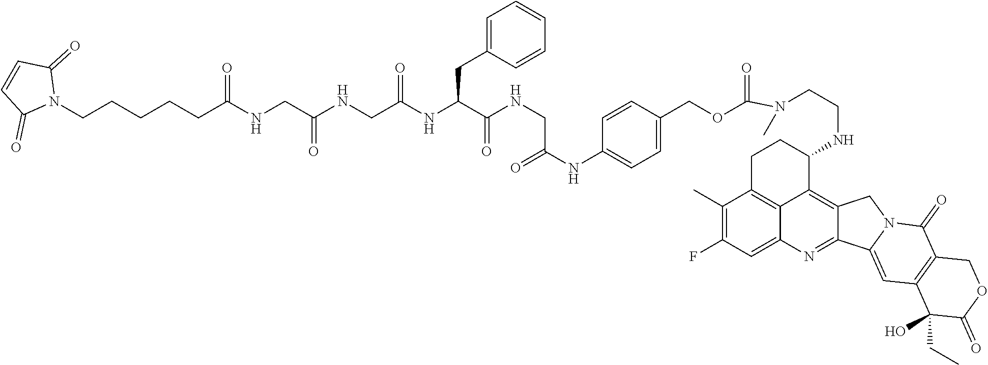 [SMILES: CC[C@@]1(O)C(=O)OCc2c1cc1n(c2=O)Cc2c-1nc1cc(F)c(C)c3c1c2[C@@H](NCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-12 | 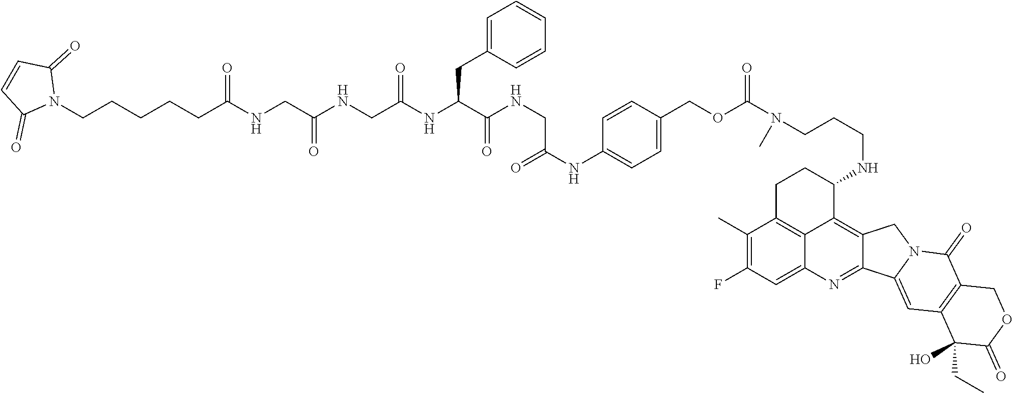 [SMILES: CC[C@@]1(O)C(=O)OCc2c1cc1n(c2=O)Cc2c-1nc1cc(F)c(C)c3c1c2[C@@H](NCCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-13 | 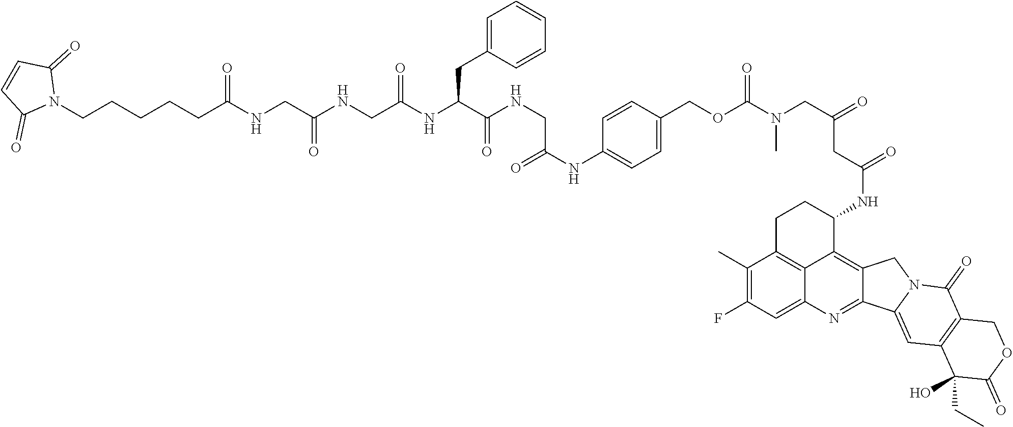 [SMILES: CC[C@@]1(O)C(=O)OCc2c1cc1n(c2=O)Cc2c-1nc1cc(F)c(C)c3c1c2[C@@H](NC(=O)CC(=O)CN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-14 | 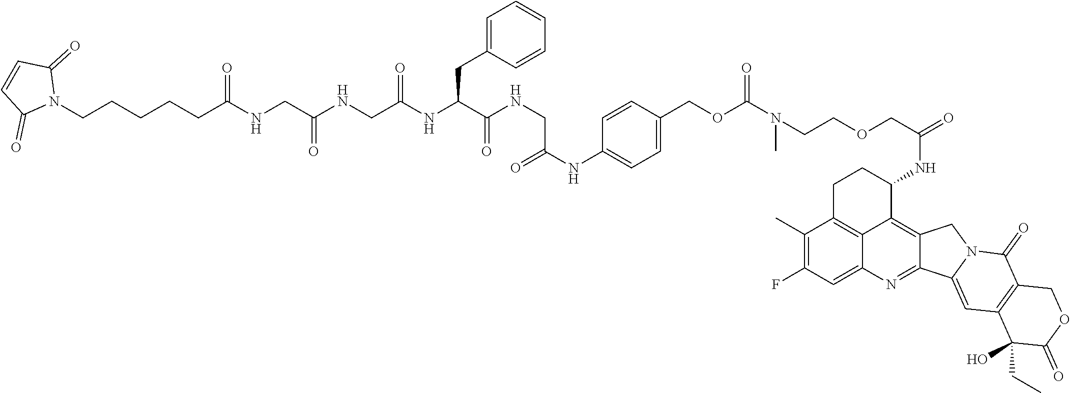 [SMILES: CC[C@@]1(O)C(=O)OCc2c1cc1n(c2=O)Cc2c-1nc1cc(F)c(C)c3c1c2[C@@H](NC(=O)COCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-15 | 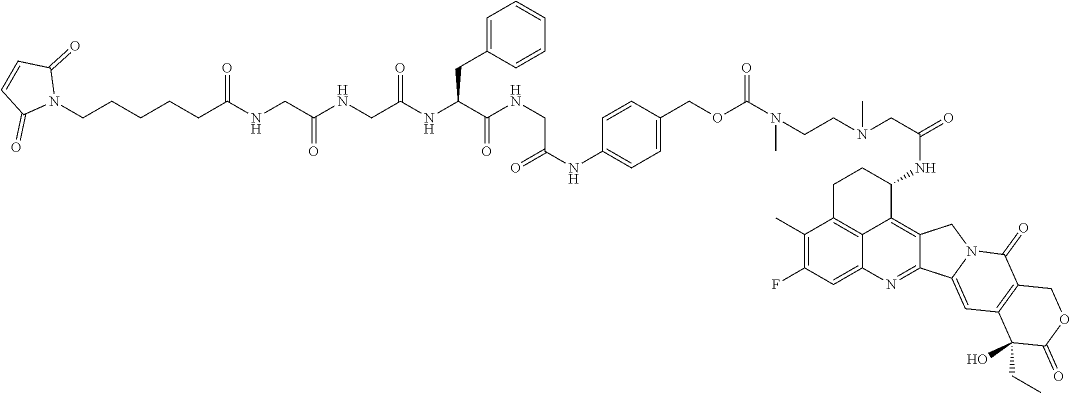 [SMILES: CC[C@@]1(O)C(=O)OCc2c1cc1n(c2=O)Cc2c-1nc1cc(F)c(C)c3c1c2[C@@H](NC(=O)CN(C)CCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-16 | 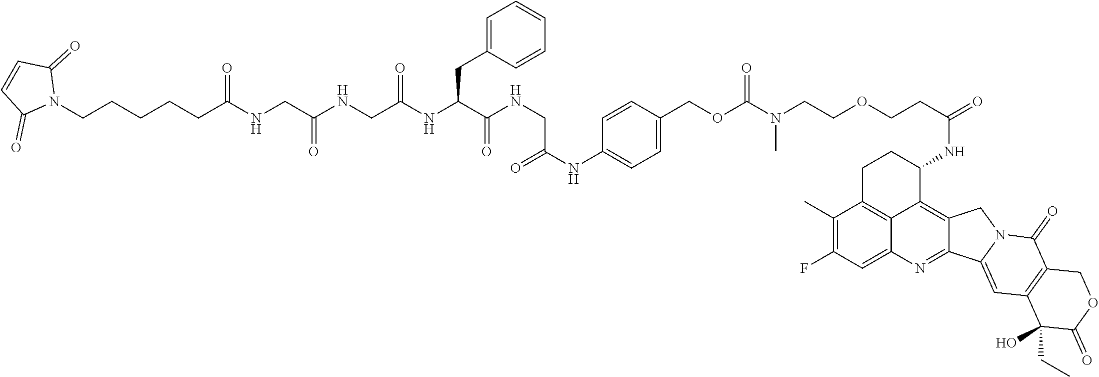 [SMILES: CC[C@@]1(O)C(=O)OCc2c1cc1n(c2=O)Cc2c-1nc1cc(F)c(C)c3c1c2[C@@H](NC(=O)CCOCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-17 | 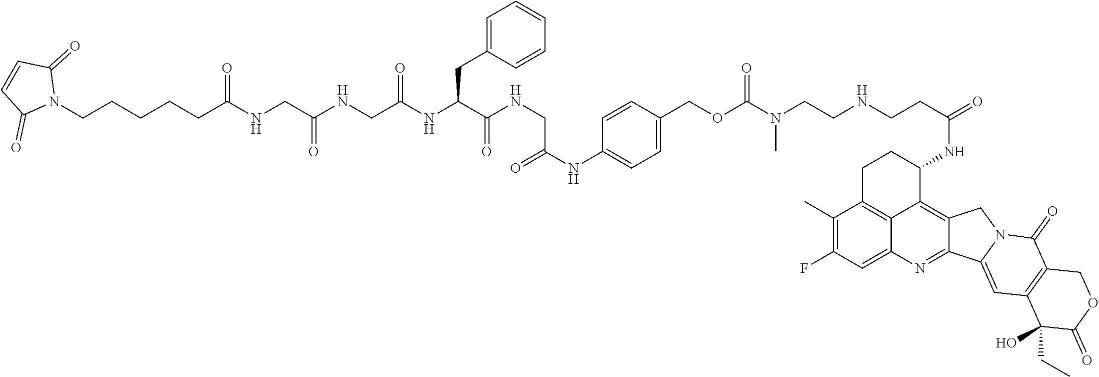 [SMILES: CC[C@@]1(O)C(=O)OCc2c1cc1n(c2=O)Cc2c-1nc1cc(F)c(C)c3c1c2[C@@H](NC(=O)CCNCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-18 | 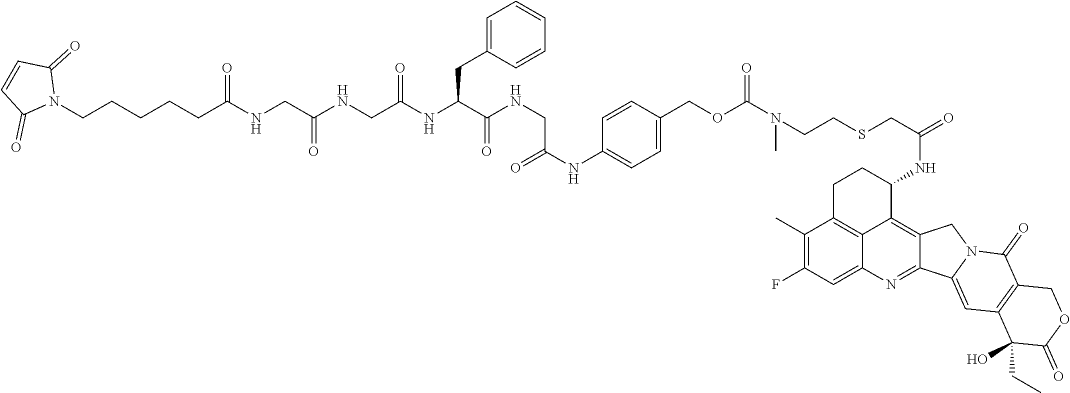 [SMILES: CC[C@@]1(O)C(=O)OCc2c1cc1n(c2=O)Cc2c-1nc1cc(F)c(C)c3c1c2[C@@H](NC(=O)CSCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-19 | 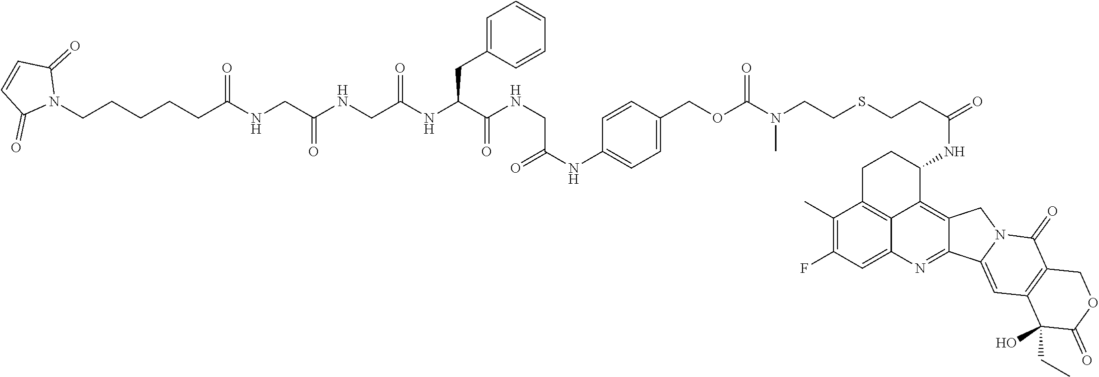 [SMILES: CC[C@@]1(O)C(=O)OCc2c1cc1n(c2=O)Cc2c-1nc1cc(F)c(C)c3c1c2[C@@H](NC(=O)CCSCCN(C)C(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-20 | 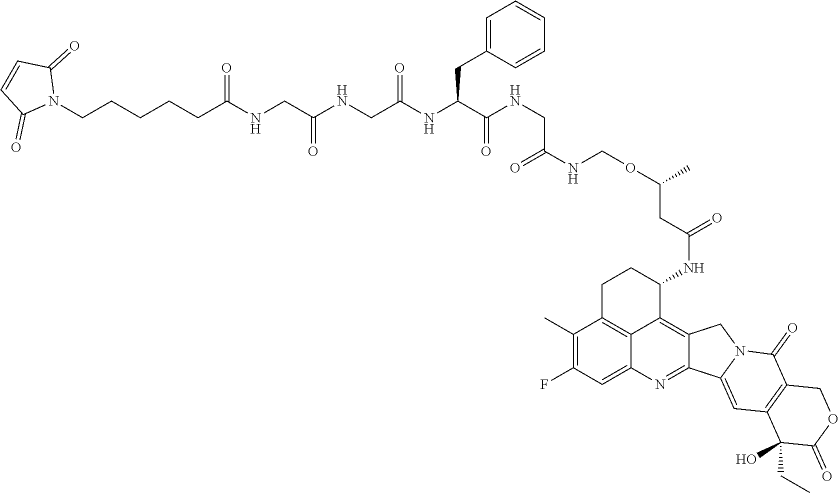 [SMILES: CC[C@@]1(O)C(=O)OCc2c1cc1n(c2=O)Cc2c-1nc1cc(F)c(C)c3c1c2[C@@H](NC(=O)C[C@@H](C)OCNC(=O)CNC(=O)[C@H](Cc1ccccc1)NC(=O)CNC(=O)CNC(=O)CCCCCN1C(=O)C=CC1=O)CC3] |
| L-III-21 | 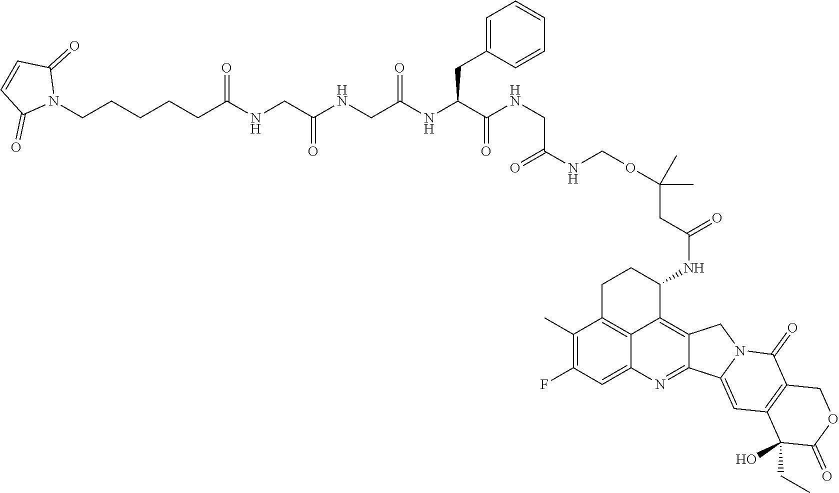 [SMILES: CC[C@@]1(O)C(=O)OCc2c1cc1n(c2=O)Cc2c-1nc1cc(F)c(C)c3c1c2[C@@H](NC(=O)CC(C)(C)OCNC(=O)CNC(=O)[C@H](Cc1ccccc1)NC(=O)CNC(=O)CNC(=O)CCCCCN1C(=O)C=CC1=O)CC3] |
| L-III-22 | 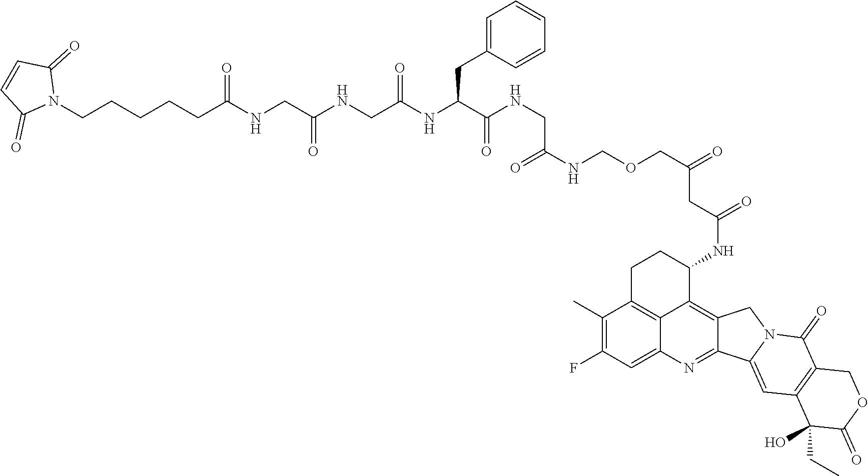 [SMILES: CC[C@@]1(O)C(=O)OCc2c1cc1n(c2=O)Cc2c-1nc1cc(F)c(C)c3c1c2[C@@H](NC(=O)CC(=O)COCNC(=O)CNC(=O)[C@H](Cc1ccccc1)NC(=O)CNC(=O)CNC(=O)CCCCCN1C(=O)C=CC1=O)CC3] |
| L-III-23 | 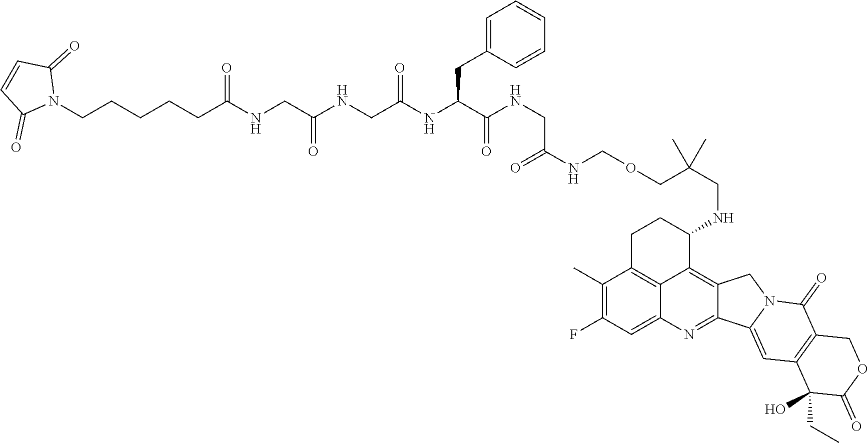 [SMILES: CC[C@@]1(O)C(=O)OCc2c1cc1n(c2=O)Cc2c-1nc1cc(F)c(C)c3c1c2[C@@H](NCC(C)(C)COCNC(=O)CNC(=O)[C@H](Cc1ccccc1)NC(=O)CNC(=O)CNC(=O)CCCCCN1C(=O)C=CC1=O)CC3] |
| L-III-24 | 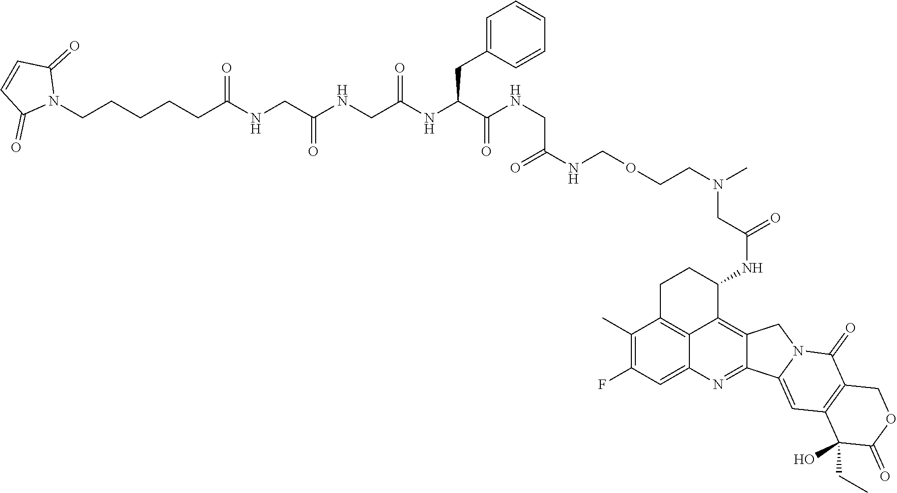 [SMILES: CC[C@@]1(O)C(=O)OCc2c1cc1n(c2=O)Cc2c-1nc1cc(F)c(C)c3c1c2[C@@H](NC(=O)CN(C)CCOCNC(=O)CNC(=O)[C@H](Cc1ccccc1)NC(=O)CNC(=O)CNC(=O)CCCCCN1C(=O)C=CC1=O)CC3] |
| L-III-25 | 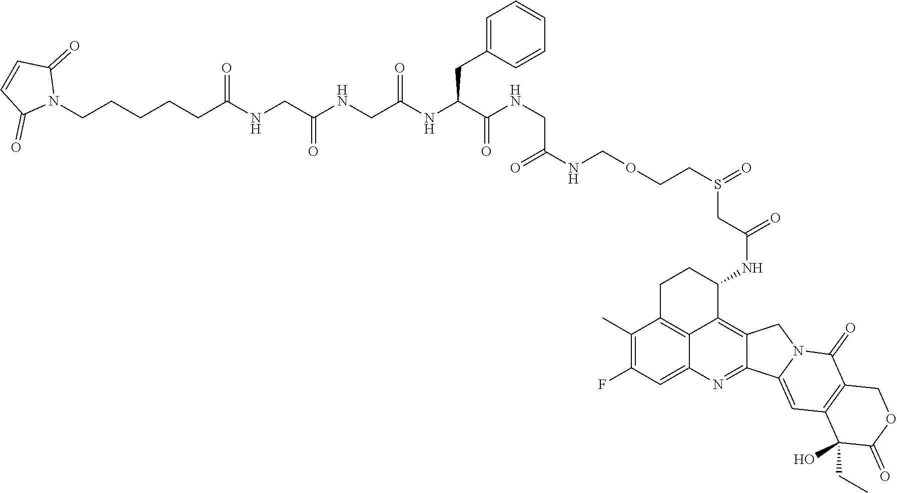 [SMILES: CC[C@@]1(O)C(=O)OCc2c1cc1n(c2=O)Cc2c-1nc1cc(F)c(C)c3c1c2[C@@H](NC(=O)CS(=O)CCOCNC(=O)CNC(=O)[C@H](Cc1ccccc1)NC(=O)CNC(=O)CNC(=O)CCCCCN1C(=O)C=CC1=O)CC3] |
| L-III-26 | 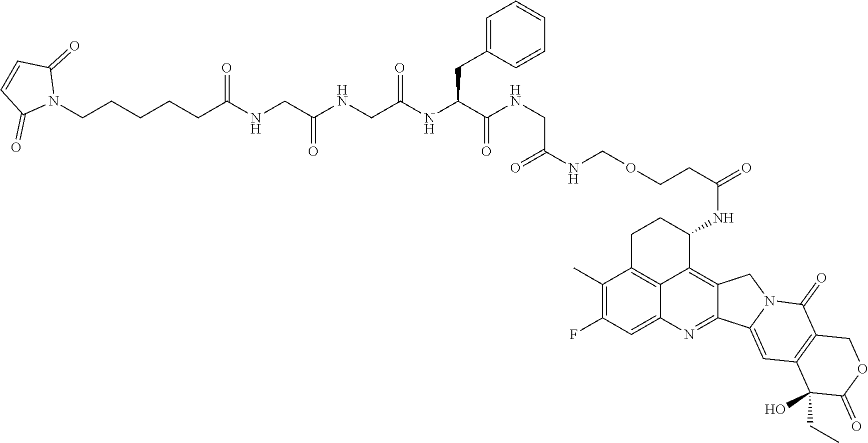 [SMILES: CC[C@@]1(O)C(=O)OCc2c1cc1n(c2=O)Cc2c-1nc1cc(F)c(C)c3c1c2[C@@H](NC(=O)CCOCNC(=O)CNC(=O)[C@H](Cc1ccccc1)NC(=O)CNC(=O)CNC(=O)CCCCCN1C(=O)C=CC1=O)CC3] |
| L-III-27 | 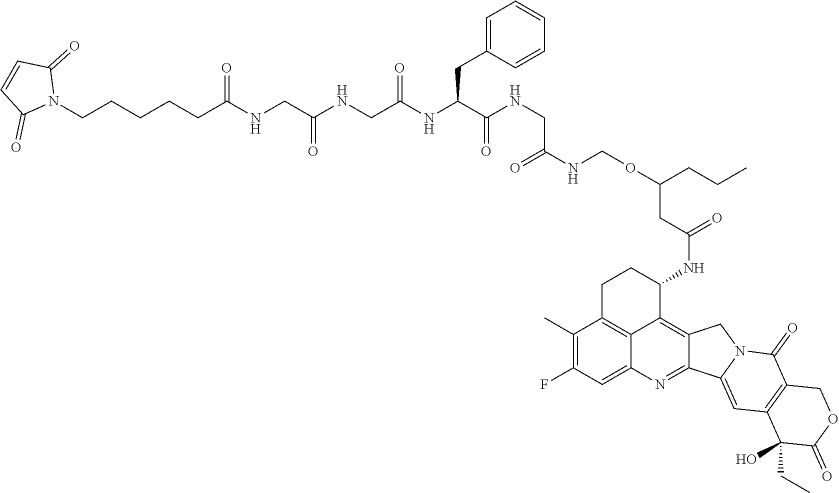 [SMILES: CCCC(CC(=O)N[C@H]1CCc2c(C)c(F)cc3nc4c(c1c23)Cn1c-4cc2c(c1=O)COC(=O)[C@]2(O)CC)OCNC(=O)CNC(=O)[C@H](Cc1ccccc1)NC(=O)CNC(=O)CNC(=O)CCCCCN1C(=O)C=CC1=O] |
| L-III-28 | 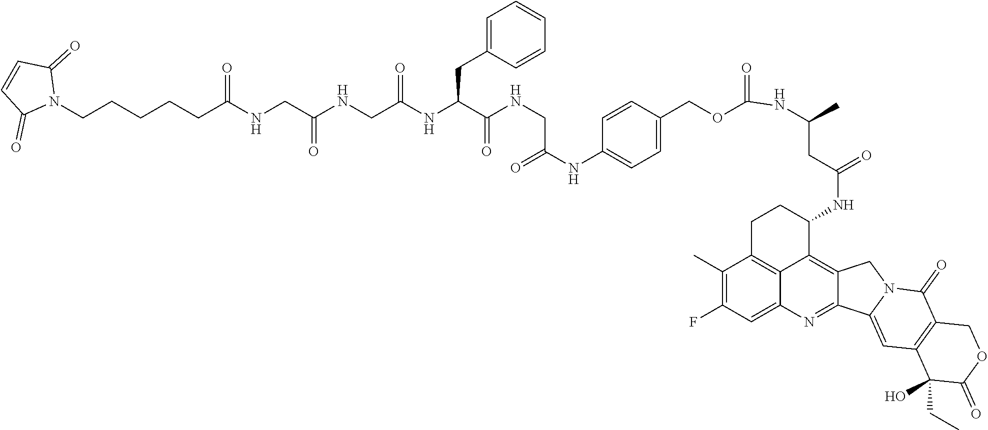 [SMILES: CC[C@@]1(O)C(=O)OCc2c1cc1n(c2=O)Cc2c-1nc1cc(F)c(C)c3c1c2[C@@H](NC(=O)C[C@H](C)NC(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-29 | 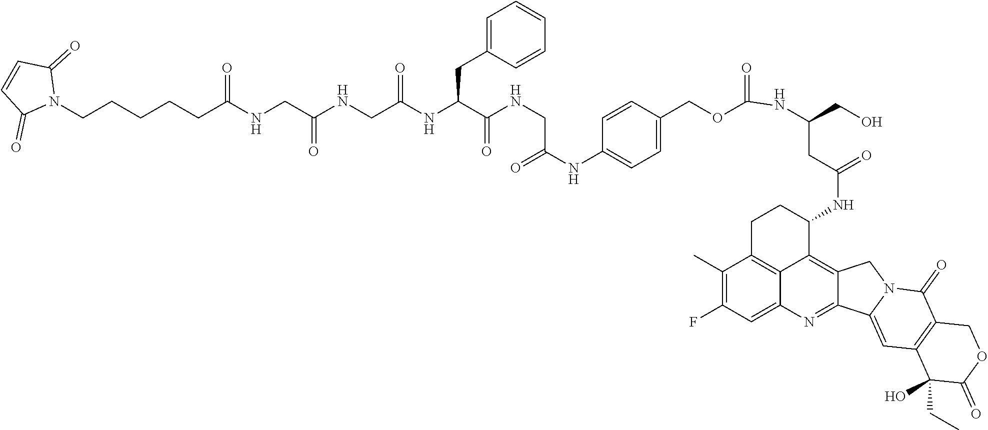 [SMILES: CC[C@@]1(O)C(=O)OCc2c1cc1n(c2=O)Cc2c-1nc1cc(F)c(C)c3c1c2[C@@H](NC(=O)C[C@H](CO)NC(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| L-III-30 | 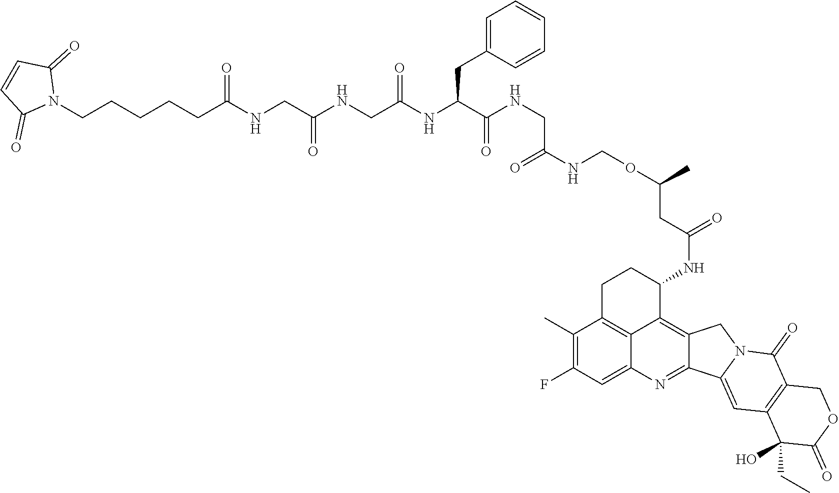 [SMILES: CC[C@@]1(O)C(=O)OCc2c1cc1n(c2=O)Cc2c-1nc1cc(F)c(C)c3c1c2[C@@H](NC(=O)C[C@H](C)OCNC(=O)CNC(=O)[C@H](Cc1ccccc1)NC(=O)CNC(=O)CNC(=O)CCCCCN1C(=O)C=CC1=O)CC3] |
| L-III-31 | |

In one embodiment of the present disclosure, the antibody-drug conjugate described herein is of general formula I-Z depicted here below

Ab-Lₐ-L_{b}-L_{c}-X (I-Z)
wherein Ab represents LC4HC3 or an antibody comprising an immunoglobulin light chain variable region comprising or consisting of amino acid sequence SEQ ID NO: 3, and an immunoglobulin heavy chain variable region comprising or consisting of amino acid sequence SEQ ID NO: 6,
Lₐ is

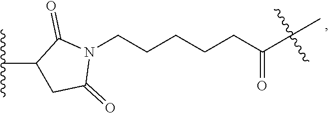 [SMILES: *C(=O)CCCCCN1C(=O)CC(*)C1=O]

L_{b} is

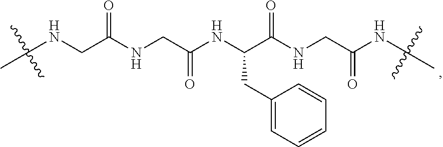 [SMILES: *NCC(=O)NCC(=O)N[C@@H](Cc1ccccc1)C(=O)NCC(=O)N*]

L_{c} is

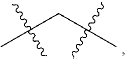 [SMILES: *C*]

and
X is selected from the group consisting of:

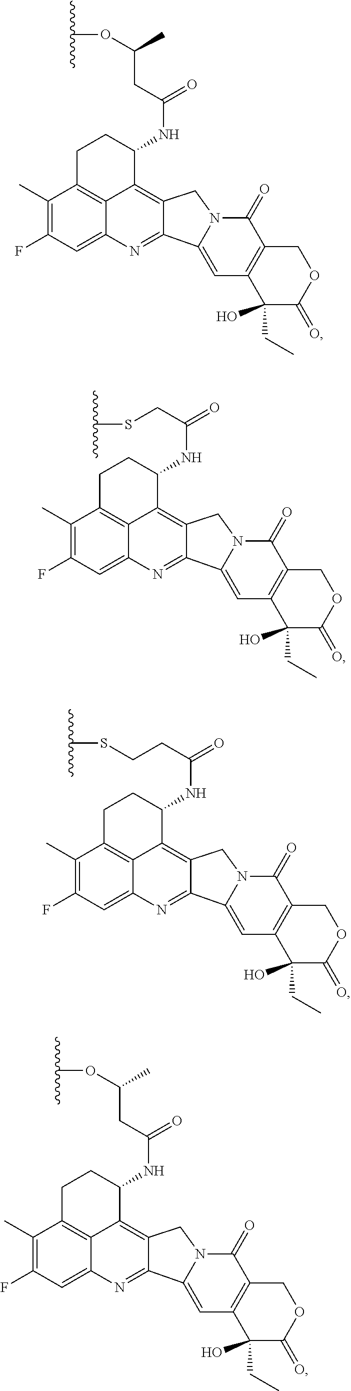 [SMILES: *O[C@@H](C)CC(=O)N[C@H]1CCc2c(C)c(F)cc3nc4c(c1c23)Cn1c-4cc2c(c1=O)COC(=O)[C@]2(O)CC ; *SCC(=O)N[C@H]1CCc2c(C)c(F)cc3nc4c(c1c23)Cn1c-4cc2c(c1=O)COC(=O)[C@]2(O)CC ; *SCCC(=O)N[C@H]1CCc2c(C)c(F)cc3nc4c(c1c23)Cn1c-4cc2c(c1=O)COC(=O)[C@]2(O)CC ; *O[C@H](C)CC(=O)N[C@H]1CCc2c(C)c(F)cc3nc4c(c1c23)Cn1c-4cc2c(c1=O)COC(=O)[C@]2(O)CC]

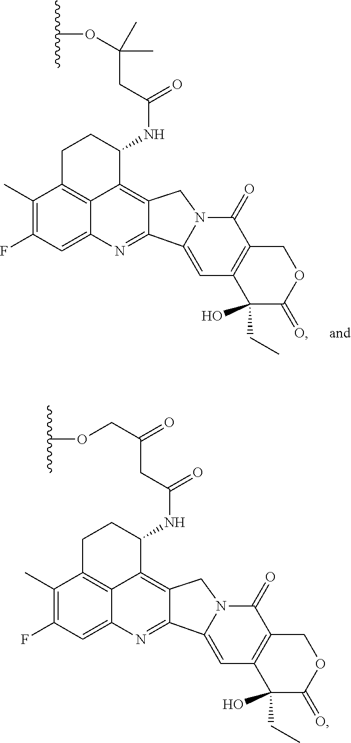 [SMILES: *OC(C)(C)CC(=O)N[C@H]1CCc2c(C)c(F)cc3nc4c(c1c23)Cn1c-4cc2c(c1=O)COC(=O)[C@]2(O)CC ; *OCC(=O)CC(=O)N[C@H]1CCc2c(C)c(F)cc3nc4c(c1c23)Cn1c-4cc2c(c1=O)COC(=O)[C@]2(O)CC]

wherein the wavy line represents a bond in formula (I-Z).

Preferably, X is selected as

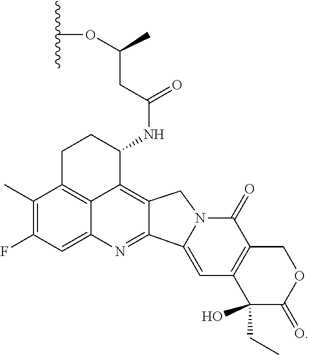 [SMILES: *O[C@@H](C)CC(=O)N[C@H]1CCc2c(C)c(F)cc3nc4c(c1c23)Cn1c-4cc2c(c1=O)COC(=O)[C@]2(O)CC]

In one embodiment of the present disclosure, the linker connecting the antibody to the active agent has the structure:

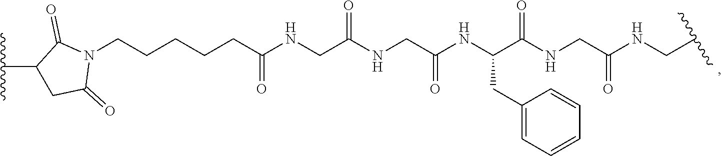 [SMILES: *CNC(=O)CNC(=O)[C@H](Cc1ccccc1)NC(=O)CNC(=O)CNC(=O)CCCCCN1C(=O)CC(*)C1=O]

and is comprising a maleimide caproic acid moiety.

That is to say in one embodiment, -Lₐ-L_{b}-L_{c}- may be:

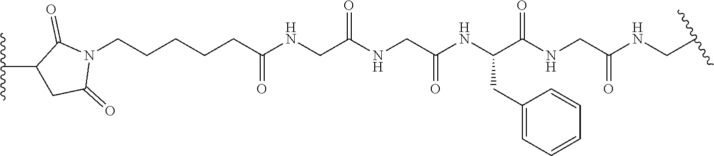 [SMILES: *CNC(=O)CNC(=O)[C@H](Cc1ccccc1)NC(=O)CNC(=O)CNC(=O)CCCCCN1C(=O)CC(*)C1=O]

In one embodiment of the present disclosure, the antibody-drug conjugate has the structure of formula (II-Z)

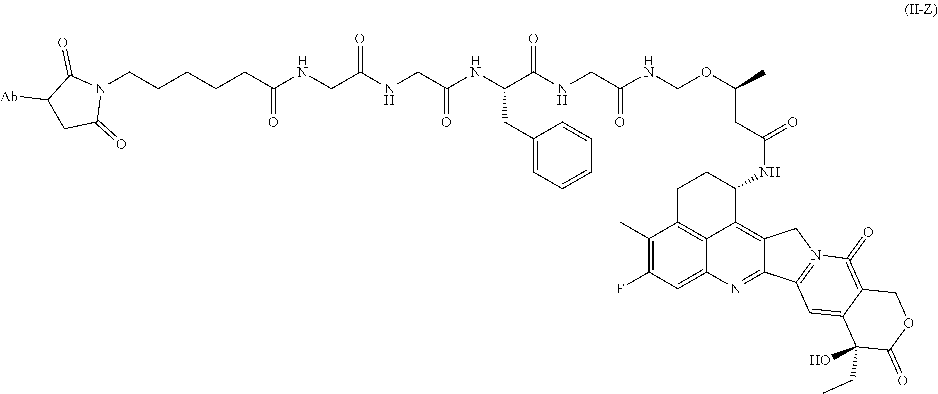

wherein Ab represents humanized antibody LC4HC3 or an antibody comprising an immunoglobulin light chain variable region comprising or consisting of amino acid sequence SEQ ID NO: 3, and an immunoglobulin heavy chain variable region comprising or consisting of amino acid sequence SEQ ID NO: 6. Preferably Ab represents humanized antibody LC4HC3. It will be readily available to those skilled in the art, that the number of cytotoxic agents linked to Ab in formulas (I-Z) and (II-Z) may be higher than 1, such as to say that the drug-antibody-ratio (DAR) is higher than 1. In one embodiment, DAR may be 2 or higher, such as 3 or higher, such as 4 or higher, such as 5 or higher, such as 6 or higher, such as 7 or higher, such as 8 or higher, such as 9 or higher, such as 10 or higher.

In one embodiment of the present disclosure, the antibody-drug conjugate of formula (II-Z) is characterized by comprising at least one, such as two, such as three, such as four, preferably four asymmetric carbon atoms of (S) configuration. Asymmetric carbon atoms are sometimes referred to also as chiral carbon atoms and are well known to persons skilled in the art of medicinal chemistry.

### Therapeutic Use

The ADCs directed against uPARAP as described herein are useful for the delivery of the active agents to cells expressing uPARAP and similar proteins and thus for the treatment of a range of diseases and disorders characterized by either expression or overexpression of said proteins.

Thus, one embodiment of the present disclosure is the antibody-drug conjugate as defined herein for use as a medicament.

One embodiment of the present disclosure is a pharmaceutical composition comprising an effective amount of the antibody-drug conjugate as defined herein, and a pharmaceutically acceptable buffer, diluent, carrier, adjuvant or excipient.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host.

As is appreciated by those skilled in the art, the amount of a compound may vary depending on its specific activity. Suitable dosage amounts may contain a predetermined quantity of active composition calculated to produce the desired therapeutic effect in association with the required diluent.

The ADCs of the present disclosure may be formulated into any type of pharmaceutical composition known in the art to be suitable for the delivery thereof.

The pharmaceutical compositions may be prepared in a manner known in the art that is sufficiently storage stable and suitable for administration to humans and/or animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the ADC. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to mean an aqueous solution containing an acid-base mixture with the purpose of stabilising pH. Pharmaceutically acceptable buffers are well known in the art.

The term “diluent” is intended to mean an aqueous or non-aqueous solution with the purpose of diluting the agent in the pharmaceutical preparation.

The term “adjuvant” is intended to mean any compound added to the formulation to increase the biological effect of the agent of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrins, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

Another embodiment of the present disclosure is a method for treatment of a disease characterised by cells in a subject expressing uPARAP, said method comprising administering to the subject the antibody-drug conjugate as defined herein.

The expression and role of uPARAP in cancer has been investigated by several research groups; cf. review by Melander et al (Melander et al., 2015, Int J Oncol 47: 1177-1188) and article by Engelholm et al (Engelholm et al., 2016, J. Pathol. 238, 120-133).

In one embodiment of the present disclosure, the method is the method as defined herein, wherein the disease characterised by cells expressing uPARAP is selected from cancer, a bone degradation disease such as osteoporosis, fibrosis, and macrophage associated diseases or disorders such as atherosclerosis, arthritis, or chronic inflammation.

In one embodiment of the present disclosure, the method is the method as defined herein, wherein the arthritis is selected from osteoarthritis, inflammatory arthritis, rheumatoid arthritis, psoriatic arthritis, lupus, Lyme disease-induced arthritis such as Lyme arthritis, gout or pseudogout, and ankylosing spondylitis.

In one embodiment of the present disclosure, the method is the method as defined herein, wherein the disease is cancer.

Examples of cancers characterized by overexpression of uPARAP include, but are not limited to, sarcoma, including osteosarcoma (Engelholm et al., 2016, J Pathol 238(1): 120-33) such as giant cell-rich osteosarcoma, osteoblastoma-like osteosarcoma and fibroblastic osteosarcoma, as well as other sarcomas, glioblastoma (Huijbers et al., 2010, PLoS One 5(3):e9808), prostate cancer and bone metastases from prostate cancer (Kogianni et al., 2009, Eur J Cancer 45(4): 685-93), breast cancer and in particular “basal like” breast cancer (Wienke et al., 2007, Cancer Res 1; 67(21): 10230-40), head- and neck cancer (Sulek et al., 2007, J Histochem Cytochem 55(4): 347-53), and mesothelioma (Çakilkaya et al., 2021, Int J Mol Sci 22(21): 11452).

In one embodiment of the present disclosure, the method is the method as defined herein, wherein the cancer is selected from sarcoma, glioblastoma, mesothelioma, colon cancer, prostate cancer, bone metastases from prostate cancer, breast cancer, head- and neck cancer, ovarian cancer, endometrial cancer, lymphoma and leukaemia.

In one embodiment of the present disclosure, the method is the method as defined herein, wherein the cancer is breast cancer, such as breast adenocarcinoma and/or breast ductal carcinoma and/or invasive ductal carcinoma of the breast.

In one embodiment of the present disclosure, the method is the method as defined herein, wherein the cancer is ovarian cancer, such as ovarian adenocarcinoma.

In one embodiment of the present disclosure, the method is the method as defined herein, wherein the cancer is endometrial cancer, such as endometrial adenocarcinoma.

In one embodiment of the present disclosure, the method is the method as defined herein, wherein the cancer is lymphoma, such as histiocytic lymphoma.

In one embodiment of the present disclosure, the method is the method as defined herein, wherein the cancer is leukaemia, such as acute lymphoblastic leukaemia (ALL), acute myeloid leukaemia (AML), chronic lymphocytic leukaemia (CLL), and chronic myeloid leukaemia (CML), or subtypes of these.

In one embodiment of the present disclosure, the method is the method as defined herein, wherein the cancer is sarcoma, such as osteosarcoma, or soft tissue sarcoma (STS), or subtypes of these.

In one embodiment of the present disclosure, the method is the method as defined herein, the soft tissue sarcoma (STS) is selected from epithelioid sarcoma, clear cell sarcoma, alveolar soft part sarcoma, extraskeletal myxoid chondrosarcoma, epithelioid hemangioendothelioma, inflammatory myofibroblastic tumor, undifferentiated embryonal sarcoma, alveolar soft part sarcoma (ASPS), angiosarcoma, chondrosarcoma, dermatofibrosarcoma protuberens (DFSP), desmoid sarcoma, Ewing's sarcoma, fibrosarcoma, myxofibrosarcoma, adult-type fibrosarcoma, gastrointerstinal stromal tumor (GIST), non-uterine leiomyosarcoma, uterine leiomyosarcoma, liposarcoma, de-differentiated liposarcoma, pleomorphic liposarcoma, myxoid round cell liposarcoma, well-differentiated liposarcoma, malignant fibro histiocytoma (MFH), malignant peripheral nerve sheath tumor (MPNST), rhabdomyosarcoma, synovial sarcoma, and/or leiomyosarcoma (LMS).

In one embodiment of the present disclosure, the method is the method as defined herein, wherein the cancer is metastatic cancer

In one embodiment of the present disclosure, the method is the method as defined herein, wherein the cancer is a solid tumour.

In one embodiment of the present disclosure, the method is the method as defined herein, wherein the cancer is glioblastoma.

In one embodiment of the present disclosure, the cancer is not a solid tumour. For instance, the ADC of the present disclosure may e.g. be used for the treatment of uPARAP-expressing leukemia, for example, from the macrophage-monocyte lineage.

In other embodiments of the present disclosure, the disease or disorder characterised by cells expressing uPARAP is not cancer.

uPARAP is involved in bone growth and homeostasis (Madsen et al., 2013, PLoS One 5; 8(8): e71261). Thus, in one embodiment the ADC of the present disclosure may be used for the treatment of a disease characterized by bone degradation, wherein the bone degradation is mediated by non-malignant cells, such as osteoporosis.

Due to its role in collagen accumulation, a role for uPARAP has also been shown in fibrosis (Madsen et al., 2012, J Pathol 227(1):94-105). Thus, in one embodiment the ADC of the present disclosure may be used for the treatment of fibrosis, for example of kidney, lung and liver.

In one embodiment of the present disclosure, the ADC of the present disclosure may be used for the treatment of diseases and disorders associated with macrophages, including atherosclerosis, arthritis, and chronic inflammation.

The ADCs of the present disclosure or pharmaceutical compositions comprising the ADCs may be administered via any suitable route known to those skilled in the art. Thus, possible routes of administration include parenteral (intravenous, subcutaneous, and intramuscular), topical, ocular, nasal, pulmonar, buccal, oral, vaginal and rectal. Also, administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques.

They are conveniently used in the form of a sterile aqueous solution which may contain other substances, for example, enough salts or glucose to make the solution isotonic with blood. The aqueous solutions should be suitably buffered if necessary.

In one embodiment of the present disclosure, the method is the method as defined herein, wherein the antibody-drug conjugate is administered parenterally, for example, intravenously, intracerebroventricularly, intraarticularly, intra-arterially, intraperitoneally, intrathecally, intraventricularly, intrasternally, intracranially, intramuscularly or subcutaneously, or by infusion techniques.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

In one embodiment of the present disclosure, the method is the method as defined herein, wherein the antibody-drug conjugate is administered intravenously.

In one embodiment of the present disclosure, the method is the method as defined herein, wherein the antibody-drug conjugate is administered subcutaneously.

In one embodiment of the present disclosure, the method is the method as defined herein, wherein the antibody-drug conjugate is administered in combination with one or more further agents, such as one or more further therapeutic agents.

In one embodiment of the present disclosure, the ADC of the present disclosure is administered in conjunction with additional reagents and/or therapeutics that may increase the functional efficiency of the ADC, such as established or novel drugs that increase lysosomal membrane permeability, thereby facilitating molecular entry from the lysosome interior to the cytoplasm, or drugs that increase the permeability of the blood-brain barrier.

In one embodiment of the present disclosure, the ADCs or the antibodies described herein may be administered in combination with a range of anti-cancer agents, such as antimetabolites, alkylating agents, anthracyclines and other cytotoxic antibiotics, vinca alkyloids, anti-microtubule/anti-mitotic agents, histone deacetylase inhibitors, kinase inhibitors, peptide antibiotics, immune checkpoint inhibitors, platinum-based antineoplastics, etoposide, taxanes, topoisomerase inhibitors, antiproliferative immunosuppressants, corticosteroids, sex hormones and hormone antagonists, cytotoxic antibiotics and other therapeutic agents.

Thus, in one embodiment of the present disclosure, the method is the method as defined herein, wherein the cell expressing uPARAP displays uPARAP overexpression.

In one embodiment of the present disclosure, the method is the method as defined herein, wherein the cell expressing uPARAP is a tumour cell.

In one embodiment of the present disclosure, the method is the method as defined herein, wherein the cell expressing uPARAP is a tumour associated cell.

Tumour associated cells include, but are not limited to, activated fibroblasts, myofibroblasts, neovasculature and infiltrating cells of the macrophage-monocyte lineage or other leukocytic cell types, as well as cells of the stromal tissue surrounding the tumour.

In one embodiment of the present disclosure, the method is the method as defined herein, wherein the antibody-drug conjugate induces cell death and/or inhibits the growth and/or proliferation of the uPARAP expressing cell.

In one embodiment of the present disclosure, the method is the method as defined herein, wherein the antibody-drug conjugate induces liberation of free cytotoxin from the uPARAP expressing cells, leading to cell death and/or inhibition of the growth and/or proliferation of neighbouring cancer cells.

In one embodiment of the present disclosure, the method is the method as defined herein, wherein the treatment is ameliorative or curative.

A further embodiment of the present disclosure is a method for inhibiting tumour progression in a subject, comprising administering to the subject the antibody-drug conjugate or the pharmaceutical composition as defined herein to said subject.

A further embodiment of the present disclosure is a method for inhibiting, lowering or eliminating metastatic capacity of a tumour in a subject, comprising administering to the subject the antibody-drug conjugate or the pharmaceutical composition as defined herein to said subject.

An even further embodiment of the present disclosure is a kit comprising the antibody-drug conjugate or the pharmaceutical compositions as defined herein, optionally further comprising means for administering said antibody-drug conjugate or pharmaceutical composition to a subject and/or instructions for use.

In one embodiment, the present disclosure relates to an antibody-drug conjugate as described herein or a pharmaceutical composition as described herein for use in the manufacture of a medicament for treatment of a disease characterised by cells expressing uPARAP, such as cancer.

In one embodiment, the present disclosure relates to an antibody-drug conjugate or a pharmaceutical composition comprising said antibody-drug conjugate for use in the manufacture of a medicament for treatment of a disease characterised by cells expressing uPARAP, such as cancer, wherein said antibody-drug conjugate is as described herein.

In one embodiment, the present disclosure relates to an antibody-drug conjugate or a pharmaceutical composition comprising said antibody-drug conjugate for use in the manufacture of a medicament for treatment of a disease characterised by cells expressing uPARAP, such as cancer, wherein said antibody-drug conjugate is as defined herein.

### EXAMPLES

### Example 1: Humanization of Murine 9b7 Antibody and Potency of ADCs Based Thereon

### Materials and Methods

Humanization of the Murine Antibody 9b7 Directed Against uPARAP

Data on the murine antibody 9b7 amino acid sequence and its CDR regions are available in the published patent application WO 2017/133745.

Humanized variants of the 9b7 antibody were constructed by a third party (Fusion Antibodies, Belfast, UK). Briefly, the murine parental antibody (clone 9b7) was sequenced, and the consensus CDR sequences were grafted into human donor sequences in silico.

For this purpose, a number of human framework sequences (see search procedure below) were used as acceptor frameworks for the CDR sequences. These acceptor sequences have all come from mature Human IgG from a human source and not from phage display or other technologies. The generated humanized variants from the Antibody 9b7 sequences are combinations of light and heavy chains, referred to as Ab 980.2 LCXHCX (Light chain X, Heavy chain X), except that LC0HC0 refers to the chimeric antibody in which the variable domains of the original murine antibody is fused to the same human IgG constant regions as used in the humanized antibodies. The mature humanized antibodies are complete IgG molecules of the IgG1 Kappa type.

For the heavy chain, online databases of Human IgG sequences were searched for comparison to the murine VH domain using BLAST search algorithms, and candidate human variable domains selected from the top 200 BLAST results. These were reduced to four candidates based on a combination of framework homology, maintaining key framework residues and canonical loop structure.

For the light chain, online databases of Human IgK sequences were searched for comparison to the murine VL domain using BLAST search algorithms, and candidate human variable domains selected from the top 200 BLAST results. These were reduced to four candidates based on a combination of framework homology, maintaining key framework residues and canonical loop structure.

Altogether, DNA sequences encoding 4 humanized light chains and 4 humanized heavy chains were thus selected. All of the resulting 16 light- and heavy chain combinations were used for protein expression in CHO cells. To enable protein expression, each of the Variable Light Chain domains was positioned in-frame with a human IgK isotype constant domain sequence, while each of the Variable Heavy Chain domains was positioned in-frame with a human IgG1 isotype constant domain sequence. The chimeric antibody, LC0HC0, in which the variable domains of the murine protein were fused to the same human IgG constant regions, was expressed for comparison.

For protein expression (performed by a third party (Fusion Antibodies, Belfast, UK)), a mammalian expression vector encoding each variant was transfected into CHO cells and batch cultures of each variant grown for up to seven days. The expressed antibodies were then subsequently purified from cell culture supernatant via affinity chromatography. The concentration and purity were determined for the purified antibody products.

The obtained sequences were cloned into the mammalian transient expression plasmid pD2610-v13 (ATUM). The humanized antibody variants were expressed using a CHO based transient expression system and the resulting antibody containing cell culture supernatants were clarified by centrifugation and filtration. The humanized variants were then purified (using state-of-the-art AKTA chromatography equipment) from cell culture supernatants via affinity chromatography. Purified antibodies were dialysed/buffer exchanged into phosphate buffered saline solution. The purity of the antibody was determined to be >95%, as judged by Sodium Dodecyl Sulphate Polyacrylamide gels

Among the resulting 16 humanized antibodies, the combination designated LC4HC3 was selected for further study based on favorable protein expression yields and antigen binding properties. Another humanized antibody designated LC3HC3 was selected for comparison with LC4HC3 of key parameters such as manufacturability, internalization and in vivo efficacy.

### SPR Analyses for Determination of Antibody-Ligand Affinity

Once suitable antibodies are obtained, they may be tested for antigen specificity, for example by surface plasmon resonance (SPR) or ELISA. When a soluble recombinant protein consisting of the three N-terminal domains of uPARAP (CysR, FN-II and CTLD-1) is immobilized in a BIAcore setup, mAb 9b7 binds to this construct.

SPR analyses were performed for determination of the affinity of the obtained antibodies towards uPARAP. These analyses were performed using a Biacore 2000 instrument (Biaffin GmbH, Kassel, Germany) using a CM5 sensor chip with an anti-human Fc capture surface for antibody binding. The analysis temperature was set at 25° C. Binding of antibody onto this surface was followed by passing soluble full-length uPARAP over the chip, and the resulting association and dissociation rates were derived from the resulting binding curves. For kinetic interaction analyses, a flowrate of 30 μL/min was used and the analysis buffer consisted of 10 mM HEPES pH 7.4, 150 mM NaCl, 3 mM EDTA, 0.05% Tween 20.

### Preparation and Evaluation of Antibody-Drug Conjugates (ADCs)

ADCs used for these studies were generated using a well-established conjugation approach. In brief, targeting antibodies were subjected to conjugation to a “vedotin” type of payload (MC-VC-PABC-MMAE) by mild reduction of interchain disulphides, followed by conjugation to a surplus of the payload via the maleimide group to a moderate average drug-to-antibody ratio (DAR) of around 4. ADCs were then purified using PD-10 desalting columns (GE Healthcare).

### Cell Lines

The U937 cell line was obtained from ATCC, and maintained in RPMI, 10% fetal bovine serum, 1% penicillin/streptomycin, in a 37 degrees Celsius incubator in a 5% CO2 atmosphere.

### In Vitro Cytotoxicity of ADCs—Cell Viability Assay

U937 cells were seeded at low density (20% confluence, 2×10³ cells per well) in a flat-bottom 96 well plate in 90 μL of medium and incubated overnight. The next day, MMAE-based ADCs of the LC4HC3 and LC0HC0 antibodies, comparably synthesized using the method described above, were prepared as a serial dilution (1:4) in PBS and added in volumes of 10 μL to each well, with a final maximum ADC concentration of 0.1 μg/mL ADC (mAb component). Cells were incubated for 96 hours, before 12 μL of CellTiter 96 AQueous One Solution Cell Proliferation Assay (MTS, Promega) was added, and incubated for an appropriate time for formation of color (around 60 minutes). The plates were then read at 490 nm, with background subtraction at 630 nm, using a plate reader, to yield the resulting viability estimates. Cells treated with PBS only were used as a nontreated control, the viability of which the ADC treated wells were normalized to.

### Results

Manufacturability and expression of LC4HC3 and LC3HC3: The humanized antibodies designated as LC4HC3 and LC3HC3 were expressed in CHO cells and purified as described above. The same procedure was implemented for both antibodies. Results are summarized below in Table 4, clearly showing that the LC4HC3 can be produced in significantly higher quantities at sufficient purity:

TABLE 4
manufacturability data for humanized
antibodies LC3HC3 and LC4HC3

| | Conc. | | Total | Estimated |
|---|---|---|---|---|
| Antibody ID | (mg/ml) | Vol. (ml) | (mg) | purity (%) |
| LC4HC3 | 3.69 | 3.02 | 11.14 | >95% |
| LC3HC3 | 2.42 | 3.06 | 7.41 | >95% |

### SPR Analyses:

LC4HC3 and LC0HC0 were analyzed by SPR as described in Materials and Methods above. In particular, the binding kinetics of LC4HC3 were compared with those of LC0HC0 (Table 5). As evident from these analyses, the lower K_{D} for the antibody LC4HC3 indicates an approx. 1.7-fold higher ligand affinity than the parental antibody, LC0HC0.

TABLE 5
result overview from kinetic interaction analysis based on SPR measurements

| | Kinetic global fit (Langmuir 1:1) | | | | |
|---|---|---|---|---|---|
| | | | | | % active |
| Measurement | kₐ (M⁻¹ s⁻¹) | k_{d} (s⁻¹) | K_{D} (M) | Rₘₐₓ | ligand |
| LC0HC0 | 6.23 × 10⁴ | 5.33 × 10⁻⁵ | 8.6 × 10⁻¹⁰ | 35.9 RU | 81% |
| LC4HC3 | 8.13 × 10⁴ | 4.04 × 10⁻⁵ | 5.0 × 10⁻¹⁰ | 36.6 RU | 123% |

### ADC In Vitro Potency Analysis:

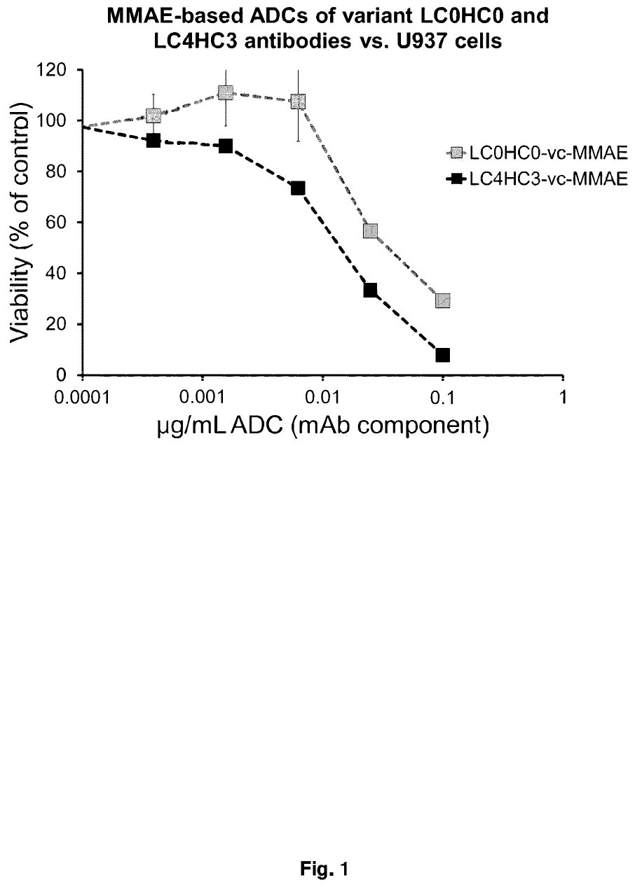

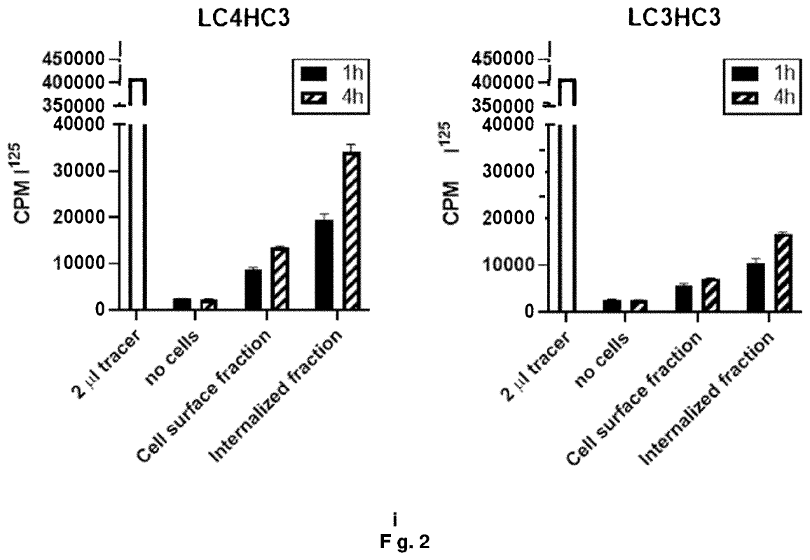

MMAE-containing ADCs including either of the antibodies LC4HC3 and LC0HC0 were prepared as described above. The in vitro cytotoxicity of these ADCs was tested against uPARAP-positive U937 cells using concentration series of the ADCs (FIG. 1). It is evident that the amount of ADC needed for cell eradication is lower for the LC4HC3-based than for the LC0HC0-based ADC, as the viability curves resulting from treatment with LC4HC3-vc-MMAE are shifted several fold towards lower concentration, compared to the curve resulting from treatment with LC0HC0-vc-MMAE.

### Conclusion

A humanized antibody, 980.2 LC4HC3, has been developed from the murine monoclonal antibody, mAb 9b7. The properties of this novel antibody can be compared directly with those resulting from the parental variable sequences by comparison with the chimeric antibody, 980.2 LC0HC0, in which the entire murine variable sequences are retained in an otherwise human IgG setting. This comparison reveals that, 1) humanized 980.2 LC4HC3 has a higher ligand affinity than 980.2 LC0HC0, and 2) an ADC based on 980.2 LC4HC3 is more efficient in terms of cytotoxicity than an otherwise equivalent ADC based on 980.2 LC0HC0.

### Example 2: Internalization of Humanized Variants of Murine 9b7 Antibody, LC3HC3 and LC4HC3

### Materials and Methods

### Antibody Labeling

Iodogen (Thermo Fischer) was dissolved at 120 μg/ml in chloroform, and used to coat the bottom of glass tubes by evaporation. In coated tubes, 200 μg/ml of humanized antibody (either LC3HC3 or LC4HC3) reacted with 588 ng/ml I-125 (Perkin Elmer) in a 0.1 M TRIS buffer at pH 7.6 for 10 minutes. The reaction was stopped by addition of a nine-fold excess of 0.1 M Tris pH 8.1 buffer containing 0.01% Tween-80. Non-bound iodine was separated from collagen over a PD-10 column, labeled antibody was eluted in a 0.1 M Tris/HCl buffer with pH 8.1, 0.01% Tween-80. Assuming all antibody is eluted in this buffer leads to a concentration of 8 μg/ml. The integrity and radioactivity of the labeled collagen was routinely confirmed by running it on SDS-PAGE, followed by Coomassie staining and phosphorimaging.

### Cell Culture and Antibody Internalization Procedure

SAOS-2 osteosarcoma cells (Finsenlab; viability 98.7%, density of 1.07×10\hat{ }6/ml) were diluted to 1×10\hat{ }5/ml and 1 ml per 24-well was seeded for experiments. Cells were allowed to adhere overnight. At least 30 minutes prior to the addition of radiolabeled antibodies the medium was replaced by internalization medium, consisting of DMEM/F12 with 1.5% FBS and 20 mM HEPES. Internalization medium without cells was seeded in separate wells as controls. It is assumed that radioactivity from these samples represents the amount of radiolabeled protein that sticks to plastic and is retrieved upon trypsin treatment. These measurements could be considered “baseline levels” and could be subtracted from measurements in samples that did contain cells. 5 μl of LC4HC3 or LC3HC3, presumed to be slightly less than 40 ng based on assumptions mentioned above, was added to each well. After 1 hour or 4 hours, media was removed by suction and the cells were washed three times with 500 μl ice cold PBS. 500 μl of Trypsin-EDTA with 50 μg/ml proteinase K was added to each well for 2 minutes. Cells were harvested, transferred to Eppendorf tubes and spun at 1000 g, 4° C., for 1 minute. Supernatants (containing cell-bound antibodies) and pellets were collected separately and analyzed on a gamma counter. 2 μl of labeled antibody stock was analyzed simultaneously to assess labeling efficiency.

### Results

The results presented in FIG. 2 clearly illustrate that the humanized antibody LC4HC3 is internalized not only significantly faster, but also to a larger extent than the humanized antibody LC3HC3.

### Conclusion

Humanized antibody LC4HC3 was internalized to the largest extent by SAOS-2 osteosarcoma cells, in a time-dependent manner. LC3HC3 was also internalized, but to a far less extent, and not as quickly as LC4HC3. The two referenced antibodies comprise the same heavy chain, and the difference in internalization can be attributed solely to the difference in amino acid sequence of the light chain.

### Example 3: In Vivo Efficacy of ADCs Based on Humanized Murine 9b7 Antibodies LC3HC3 and LC4HC3

### Materials and Methods

### Cell Culture and Preparation

U937 cells (as described above) were passaged according to standard procedures until enough cells for this experiment were acquired. Cells were spun down at 150 g for 5 min and washed 3 times in cold PBS (Gibco). The cell concentration was adjusted to 3.6×10⁶ cells/ml. This translates to approximately 3 million live cells per 100 μl, which is the intended inoculation volume.

### Xenograft Tumor Inoculation

Recipient CB17 mice were anesthetized with Zoletil (AEM), Viscotears eye drops were applied, and earmarks were made. The right flank was shaved and disinfected with 70% ethanol. A 25G needle was used to inject 100 μl of re-suspended U937 cells into the subcutaneous space (no incision or suture necessary). The mice were allowed to recover from the anesthesia in their cages. Recovery was monitored until the mice were mobile.

The mice were monitored again the next day, and tumor sizes were monitored closely until the start of treatment.

Treatments were initiated as soon as the tumors reached a proper size (approximately 80-150 mm³).

### ADC Treatment and Monitoring

Vedotin-type (MMAE) ADCs comprising the LC3HC3 or LC4HC3 humanized antibodies were prepared as previously described above. The mice were divided into groups, with 3-5 animals per group (N=3-5), the groups differing by the employed ADC or dose administered of said ADC. One cohort of mice was tested with the LC3HC3 ADC in groups receiving concentrations of 2, 4 or 6 mg/kg and another cohort was tested with the LC4HC3 ADC in groups receiving the same range of concentrations.

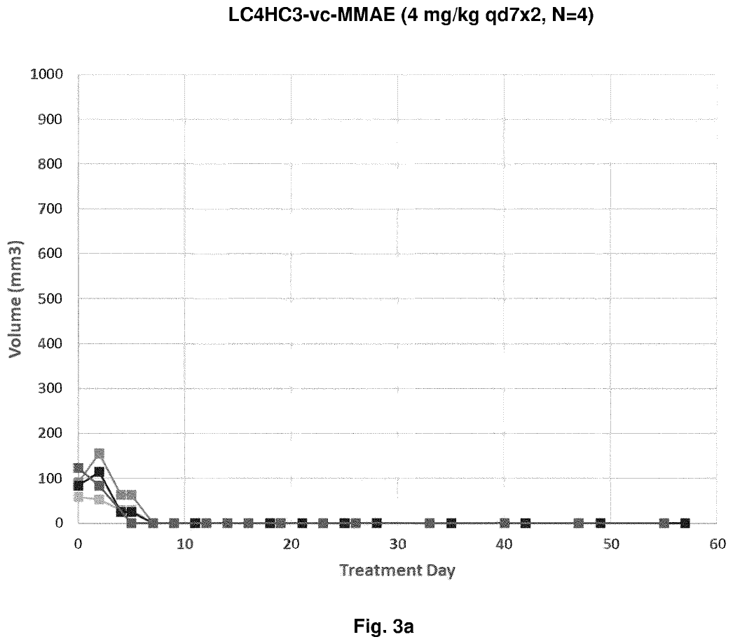

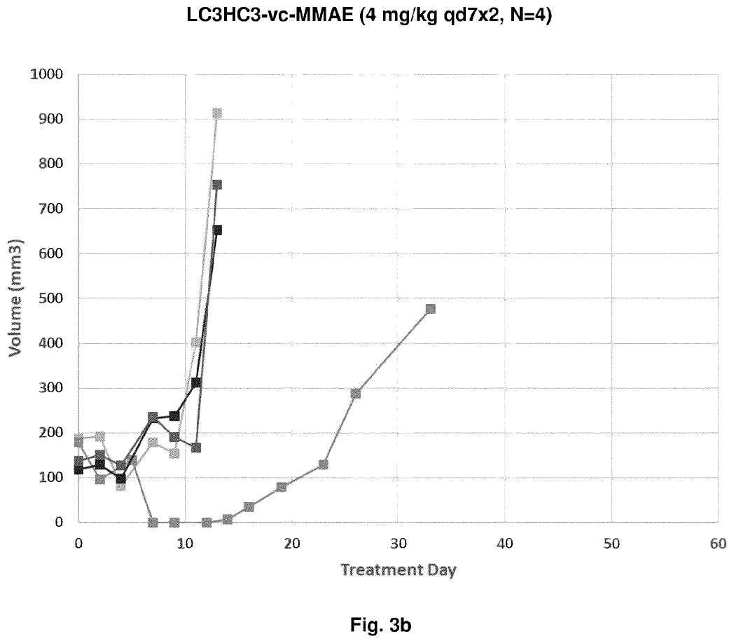

For each group, mice were administered a controlled amount of ADC intravenously (tail vein) once weekly, for two injections total (qd7×2), and tumor progression was closely monitored also post-treatment. Exemplary, FIGS. 3a and 3b show the tumor progression in groups treated with a 4 mg/kg dose of LC4HC3 and LC3HC3 ADCs respectively.

The monitoring consists of checking for overall wellbeing, and measuring the width and length dimensions of the tumor with a digital caliper. All observations and measurements are noted by hand and transferred to the digital data sheets following inspection. Animals were euthanized if the tumor size exceeded 12 mm in one dimension, if the volume of the tumor (calculated as (length×width²)/2) exceeded 1000 mm³, or if a severe impact on general wellbeing was observed. Animals were euthanized via cervical dislocation.

### Results

U937 tumor volumes following treatment with ADCs based on LC4HC3 and LC3HC3 are found in FIGS. 3a and 3b respectively. FIG. 3a shows tumor volumes for a group of four mice (N=4), each administered the LC4HC3 ADC in a 4 mg/kg once weekly for two injections total (qd7×2), and FIG. 3b shows tumor volumes for a different group of four mice (N=4), each administered the LC3HC3 ADC in a 4 mg/kg dose once weekly for two injections total (qd7×2).

### Conclusion

As evident when comparing FIGS. 3a and 3b, the treatment based on LC4HC3 ADCs completely cured all mice with no regrowth of tumor in the post-treatment period, notably in all doses tested. In contrast, the same treatment based on LC3HC3 was not able to kill all tumor cells, and in instances, aggressive tumor growth was observed in the post-treatment monitoring period.

The data shows that humanized antibody LC4HC3 as well as ADCs comprising said humanized antibody are potent antitumor agents with improved in vivo efficacy compared to another humanized antibody, LC3HC3. The two referenced antibodies comprise the same heavy chain, and the difference in efficacy can be attributed solely to the variations in amino acid sequence of the light chain.

### Example 4: Synthesis of Compounds and ADCs

The exatecan derivatives disclosed herein can be prepared as described in WO 2022/068878. Specifically compounds P-III-30, P-III-1, P-III-2, P-III-20, P-III-21, P-III-22, P-III-28 and P-III-29 may be prepared according to the reference examples given in the table below. It will be apparent to those skilled in the art that other examples from WO2022/068878 on syntheses of herein disclosed compounds is also incorporated by reference.

| Synthesized | Reference example in | Found on page # in |
|---|---|---|
| compound | WO2022/068878 | WO2022/068878 |
| P-III-1 | 1.17 | 246 |
| P-III-2 | 1.6 | 238 |
| P-III-20 | 1.7 | 239 |
| P-III-21 | 1.8 | 240 |
| P-III-22 | 1.19 | 247 |
| P-III-28 | 1.20 | 248 |
| P-III-29 | 1.22 | 249 |
| P-III-30 | 1.23 | 250 |

Antibody-drug conjugates comprising the exatecan derivatives and the humanized antibody binding uPARAP as described herein (LC4HC3) can be prepared using standard methods for conjugation of payloads to antibodies known to a person of skill in the art. For example, they can be prepared in a similar manner as in the preparation of ADCs described in the examples of WO 2022/068878.

### Example 5: Efficacy of Compounds and ADCs

### Test Example 5.1. Test for Inhibition of In Vitro Proliferation of Tumor Cells by Compounds

### Objective

To test the inhibitory activity of the pharmaceutical compounds on the in vitro proliferation of NCI-N87, JIMT-1 and MBA-MB-231 tumor cells. The cells were treated with the compounds at different concentrations in vitro, and after 6 days of culture, the proliferation of cells was detected using the CTG (CellTiter-Glo® Luminescent Cell Viability Assay, Promega, Cat. No. G7558) reagent, and the in vitro activity of the compounds was evaluated according to the IC₅₀ value.

### Procedures

In the following, the test for the inhibition of the in vitro proliferation of NCI-N₈₇ cells was taken as an example to illustrate the method of the present application for testing the inhibitory activity of the compounds of the present application on the in vitro proliferation of tumor cells. The method was also applicable to, but not limited to, the test for the inhibitory activity on the in vitro proliferation of other tumor cells.
1. Culturing of cells: NCI-N87 cells were cultured in RPMI-1640 medium containing 10% FBS.
2. Preparation of cells: NCI-N87 cells in logarithmic phase were taken, washed with PBS once, and digested with 2-3 mL of trypsin for 2-3 min. After the cells were digested completely, 10-15 mL of cell culture was added to elute the digested cells. The eluate was centrifuged at 1000 rpm for 5 min, and the supernatant was discarded. The resulting cells were resuspended in 10-20 mL of cell culture to obtain a single cell suspension.
3. Cell plating: the NCI-N87 single cell suspension was mixed well and adjusted to a viable cell density of 6×10⁴ cells/mL with cell culture. The cell suspension with the adjusted density was mixed well and added to a 96-well cell culture plate at 50 μL/well. The culture plate was incubated in an incubator for 18 h (37° C., 5% CO₂).
4. Preparation of compounds: the compounds were dissolved in DMSO to obtain stock solutions at an initial concentration of 10 mM.

There were 8 concentrations in total for the small molecule compounds: 300 nM, 100 nM, 30 nM, 10 nM, 3 nM, 1 nM, 0.3 nM, and 0.1 nM.
5. Sample adding: the prepared samples to be detected at different concentrations were added to the culture plate, and two duplicate wells were set for each sample. The culture plate was incubated in an incubator for 6 days (37° C., 5% CO₂).
6. Color developing: the 96-well cell culture plate was taken out, added with the CTG reagent at 50 μL/well, and incubated at room temperature for 10 min.
7. Plate reading: the 96-well cell culture plate was taken out, placed in a microplate reader, and measured for the chemiluminescence using the microplate reader.

### Data Analysis

Data were processed and analyzed using Microsoft Excel and Graphpad Prism 5.

TABLE 6
IC₅₀ values for the inhibition of the in vitro proliferation
of NCI-N87 and JIMT-1 cells by the small molecule
fragments of the present application.

| | NCI-N87 | JIMT-1 |
|---|---|---|
| Compound No. | IC₅₀ (nM) | IC₅₀ (nM) |
| P-II-1 | 18.47 | 8.47 |
| P-II-2 | 22.1 | 17 |
| P-II-3 | 9.558 | 4.996 |
| P-II-4 | 8.032 | 6.637 |
| P-I-1 | 97.29 | 23.7 |
| P-I-1 | 61.56 | 21.81 |
| P-III-2 | 108.4 | 108.1 |
| P-III-20 | 5.888 | 4.351 |
| P-III-21 | 4.755 | 2.874 |

Conclusion: according to the results shown in Table 6, the small molecule fragments of the present application have significant inhibitory activity on the proliferation of NCI-N87 cells and JIMT-1 cells. The compounds of the present application all have similar inhibitory activity on tumor proliferation.

### Test Example 5.2. Test for Inhibition of In Vitro Proliferation of Tumor Cells by Compounds

### Objective

To test the inhibitory activity of the pharmaceutical compounds on the in vitro proliferation of NCI-N87, JIMT-1 and MBA-MB-231 tumor cells. The cells were treated with compounds at different concentrations in vitro, and after 6 days of culture, the proliferation of cells was detected using CTG (CellTiter-Glo® Luminescent Cell Viability Assay, Promega, Cat. No. G7558) reagents, and the in vitro activity of the compounds was evaluated according to the IC₅₀ value.
1. Culturing of cells: NCI-N87/JIMT-1/MBA-MB-231 cells were cultured in RPMI-1640 medium containing 10% FBS.
2. Preparation of cells: NCI-N87/JIMT-1/MBA-MB-231 cells in logarithmic phase were taken, washed with PBS once, and digested with 2-3 mL of trypsin for 2-3 min. After the cells were digested completely, 10-15 mL of cell culture was added to elute the digested cells. The eluate was centrifuged at 1000 rpm for 5 min, and the supernatant was discarded. The resulting cells were resuspended in 10-20 mL of cell culture to obtain a single cell suspension.
3. Cell plating: the NCI-N87/JIMT-1/MBA-MB-231 single cell suspension was mixed well and adjusted to a viable cell density of 6×10⁴ cells/mL with cell culture. The cell suspension with the adjusted density was mixed well and added to a 96-well cell culture plate at 50 μL/well. The culture plate was incubated in an incubator for 18 h (37° C., 5% CO₂).
4. Preparation of compounds: the compounds were dissolved in DMSO to obtain stock solutions at an initial concentration of 10 mM.

There were 8 concentrations in total for the small molecule compounds: 300 nM, 100 nM, 30 nM, 10 nM, 3 nM, 1 nM, 0.3 nM, and 0.1 nM.
5. Sample adding: the prepared samples to be detected at different concentrations were added to the culture plate, and two duplicate wells were set for each sample. The culture plate was incubated in an incubator for 6 days (37° C., 5% CO₂).
6. Color developing: the 96-well cell culture plate was taken out, added with the CTG reagent at 50 μL/well, and incubated at room temperature for 10 min.
7. Plate reading: the 96-well cell culture plate was taken out, placed in a microplate reader, and measured for the chemiluminescence using the microplate reader.

Data analysis: data were processed and analyzed using Microsoft Excel and Graphpad Prism 5.

TABLE 7
IC₅₀ values for the inhibition of the in vitro proliferation of tumor
cells by the small molecule fragments of the present application.

| | NCI- | | |
|---|---|---|---|
| | N87 | JIMT-1 | MDA-MB- |
| | IC50 | IC50 | 231 |
| Compound No. | (nM) | (nM) | IC50 (nM) |
| Comparative | — | 23.1 | — |
| example 1 | | | |
| P-II-1 | 18.47 | 8.5 | 12.9 |
| P-II-2 | 22.1 | 17.2 | 19.3 |
| P-11-3 | 9.558 | 5.0 | 9.4 |
| P-II-4 | 8.032 | 6.6 | 7.6 |
| P-II-22 | — | 3.2 | — |
| P-II-23 | — | | 7.7 |
| P-II-24 | — | 20.8 | 8.5 |

“—”: not detected

Conclusion: according to the results shown in Table 7, the small molecule fragments of the present application have significant inhibitory activity on the proliferation of NCI-N87, JIMT-1 and MDA-MB-231 cells. The compounds of the present application all have similar inhibitory activity on tumor proliferation.

### Test Example 5.3. Test for Inhibition of In Vitro Proliferation of Tumor Cells by Compounds

### Objective

To test the inhibitory activity of the pharmaceutical compounds on the in vitro proliferation of NCI-N87, JIMT-1 and MBA-MB-231 tumor cells. The cells were treated with compounds at different concentrations in vitro, and after 6 days of culture, the proliferation of cells was detected using CTG (CellTiter-Glo® Luminescent Cell Viability Assay, Promega, Cat. No. G7558) reagents, and the in vitro activity of the compounds was evaluated according to the IC₅₀ value.
1. Culturing of cells: NCI-N87/JIMT-1/MBA-MB-231 cells were cultured in RPMI-1640 medium containing 10% FBS.
2. Preparation of cells: NCI-N87/JIMT-1/MBA-MB-231 cells in logarithmic phase were taken, washed with PBS once, and digested with 2-3 mL of trypsin for 2-3 min. After the cells were digested completely, 10-15 mL of cell culture was added to elute the digested cells. The eluate was centrifuged at 1000 rpm for 5 min, and the supernatant was discarded. The resulting cells were resuspended in 10-20 mL of cell culture to obtain a single cell suspension.
3. Cell plating: the NCI-N87/JIMT-1/MBA-MB-231 single cell suspension was mixed well and adjusted to a viable cell density of 6×10⁴ cells/mL with cell culture. The cell suspension with the adjusted density was mixed well and added to a 96-well cell culture plate at 50 μL/well. The culture plate was incubated in an incubator for 18 h (37° C., 5% CO₂).
4. Preparation of compounds: the compounds were dissolved in DMSO to obtain stock solutions at an initial concentration of 10 mM.

There were 8 concentrations in total for the small molecule compounds: 300 nM, 100 nM, 30 nM, 10 nM, 3 nM, 1 nM, 0.3 nM, and 0.1 nM.
5. Sample adding: the prepared samples to be detected at different concentrations were added to the culture plate, and two duplicate wells were set for each sample. The culture plate was incubated in an incubator for 6 days (37° C., 5% CO₂).
6. Color developing: the 96-well cell culture plate was taken out, added with the CTG reagent at 50 μL/well, and incubated at room temperature for 10 min.
7. Plate reading: the 96-well cell culture plate was taken out, placed in a microplate reader, and measured for the chemiluminescence using the microplate reader.

Data analysis: data were processed and analyzed using Microsoft Excel and Graphpad Prism 5.

TABLE 8
IC₅₀ values for the inhibition of the in vitro proliferation
of NCI-N87, JIMT-1 and MDA-MB-231 cells by the small
molecule fragments of the present application.

| | NCI-N87 | JIMT-1 | MDA-MB-231 |
|---|---|---|---|
| Compound No. | IC50 (nM) | IC50 (nM) | IC50 (nM) |
| Comparative | — | 36.2 | — |
| example 2 | | | |
| Comparative | — | 81.2 | — |
| example 3 | | | |
| Comparative | — | 32.3 | — |
| example 4 | | | |
| P-III-1 | — | — | 28.9 |
| P-III-2 | 108.4 | 108.1 | — |
| P-III-9 | — | — | 28.9 |
| P-III-20 | 5.888 | 4.351 | 6.65 |
| P-III-21 | 4.755 | 2.874 | 2.88 |
| P-III-22 | — | 12.5 | — |
| P-III-27 | 3.0 | — | — |
| P-III-28 | — | — | 1.2 |
| P-III-29 | — | 16.7 | 8.6 |
| P-III-30 | — | — | 28.5 |

“—”: not detected

Conclusion: according to the results shown in Table 8, the small molecule fragments of the present application have significant inhibitory activity on the proliferation of NCI-N87, JIMT-1 and MDA-MB-231 cells. The compounds of the present application all have similar inhibitory activity on tumor proliferation.

### Test Example 5.4. Test for Inhibition of In Vitro Proliferation of Tumor Cells by Compounds

### Objective

To test the inhibitory activity of the pharmaceutical compounds on the in vitro proliferation of NCI-N87 and Colo205 tumor cells. The cells were treated with compounds at different concentrations in vitro, and after 6 days of culture, the proliferation of cells was detected using CTG (CellTiter-Glo® Luminescent Cell Viability Assay, Promega, Cat. No. G7558) reagents, and the in vitro activity of the compounds was evaluated according to the IC₅₀ value.
1. Culturing of cells: NCI-N87/Colo205 cells were cultured in RPMI-1640 medium containing 10% FBS.
2. Preparation of cells: NCI-N87/Colo205 cells in logarithmic phase were taken, washed with PBS once, and digested with 2-3 mL of trypsin for 2-3 min. After the cells were digested completely, 10-15 mL of cell culture was added to elute the digested cells. The eluate was centrifuged at 1000 rpm for 5 min, and the supernatant was discarded. The resulting cells were resuspended in 10-20 mL of cell culture to obtain a single cell suspension.
3. Cell plating: the NCI-N87/Colo205 single cell suspension was mixed well and adjusted to a viable cell density of 6×10⁴ cells/mL with cell culture. The cell suspension with the adjusted density was mixed well and added to a 96-well cell culture plate at 50 μL/well. The culture plate was incubated in an incubator for 18 h (37° C., 5% CO₂).
4. Preparation of compounds: the compounds were dissolved in DMSO to obtain stock solutions at an initial concentration of 10 mM.

There were 8 concentrations in total for the small molecule compounds: 300 nM, 100 nM, 30 nM, 10 nM, 3 nM, 1 nM, 0.3 nM, and 0.1 nM.
5. Sample adding: the prepared samples to be detected at different concentrations were added to the culture plate, and two duplicate wells were set for each sample. The culture plate was incubated in an incubator for 6 days (37° C., 5% CO₂).
6. Color developing: the 96-well cell culture plate was taken out, added with the CTG reagent at 50 μL/well, and incubated at room temperature for 10 min.
7. Plate reading: the 96-well cell culture plate was taken out, placed in a microplate reader, and measured for the chemiluminescence using the microplate reader.

Data analysis: data were processed and analyzed using Microsoft Excel and Graphpad Prism 5.

TABLE 9
IC₅₀ values for the inhibition of the in vitro proliferation
of NCI-N87 and Colo205 cells by the small molecule
fragments of the present application.

| | NCI-N87 | Colo205 |
|---|---|---|
| Compound No. | IC50 (nM) | IC50 (nM) |
| P-III-20 | 5.9 | 20 |
| P-III-30 | 3 | 9 |

Conclusion: according to the results shown in Table 9, the small molecule fragments of the present application have significant inhibitory activity on the proliferation of NCI-N87 cells and Colo205 cells. The compounds of the present application all have similar inhibitory activity on tumor proliferation.

### Test Example 5.5. Pharmacokinetic and Toxicity Studies of ADCs at a Single Administration

### Objective

To investigate the pharmacokinetic properties of the drug in monkeys and observe the toxic manifestation of the animals after a single intravenous drip of ADC in monkeys.

### Test Method

Pharmacokinetics: after the single intravenous drip of ADC drugs at difference doses in monkeys, blood samples were collected at a plurality of continuous time points, and the concentration of the drugs in the blood was detected by a proper specific detection method.

Toxicity study: after the single intravenous drip of ADC drugs at difference doses in monkeys, the tolerance of animals and drug-related toxicity toxic manifestation were investigated in multiple aspects such as clinical observation, body weight and food intake, hematology, blood biochemistry, urine and gross anatomy.

### Test Results

After the single intravenous drip of ADCs in monkeys, the concentration of free toxins is very low, and the pharmacokinetic properties of the total antibody and ADCs are similar, suggesting that ADCs are slowly released in monkeys, have a stable conjugating mode, and can be used in a clinically planned administration frequency.

After the single intravenous drip of ADCs in monkeys, animals have good tolerance and do not show serious or intolerable drug-related toxicity, suggesting that ADCs have controllable safety, and can be further studied clinically. All ADCs of the present application have similar safety.

### Example 6: In Vivo Efficacy of ADCE-D01

ADCE-D01 is an antibody drug conjugate with a theoretical molecular weight of 156 kDa. The drug-antibody-ratio (DAR) is 8. ADCE-D01 has the following structure:

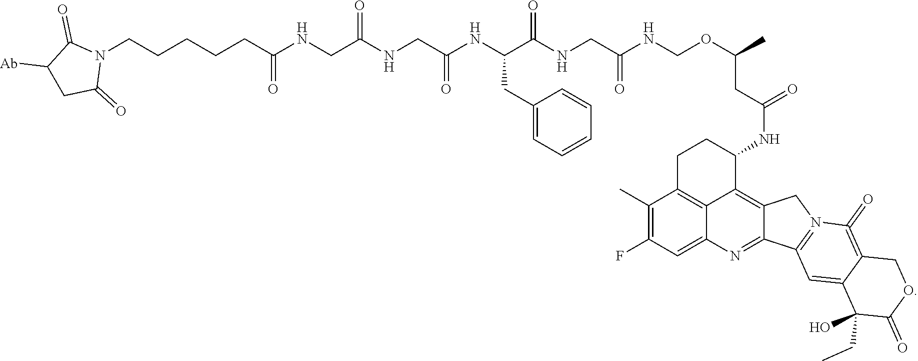

The structure is derived from linker payload combination L-III-30 described herein on p. 162, wherein maleimide conjugation to the humanised monoclonal antibody LC4HC3 (Ab in the structure) is realized by full conjugation to the two interchain heavy-light chain cysteines, and the two interchain heavy-heavy chain cysteines. ADCE-D01 thereby comprise the exatecan derivative P-III-30 denoted herein above. The conjugation of the antibody to the antibody drug conjugate drug substance (DS) is performed by well-established TCEP reduction chemistry known to those skilled in the art of antibody-drug conjugates, TCEP here referring to tris(2-carboxyethyl)phosphine. ADCE-D01 drug product (DP) is presented as powder for solution for infusion in a 20 mL vial with an aluminium seal with a plastic cap.

### In Vivo Pharmacology

In vivo efficacy of the uPARAP-directed ADC ADCE-D01 was evaluated in two cell lines.

Leiomyosarcoma is a subtype of soft tissue sarcoma that originates from smooth muscle cells. The SK-LMS-1 cell line was established from a vulva metastasis in a 43-year-caucasian-female (Fogh 1975c). CB17-SCID immune-compromised mice were inoculated subcutaneously in the right flank with human SK-LMS-1 cells. Treatment was initiated when the group mean tumour volume was approximately 120 mm³. Mice were treated with vehicle control (PBS), control ADC and ADCE-D01 at 3 and 10 mg/kg.

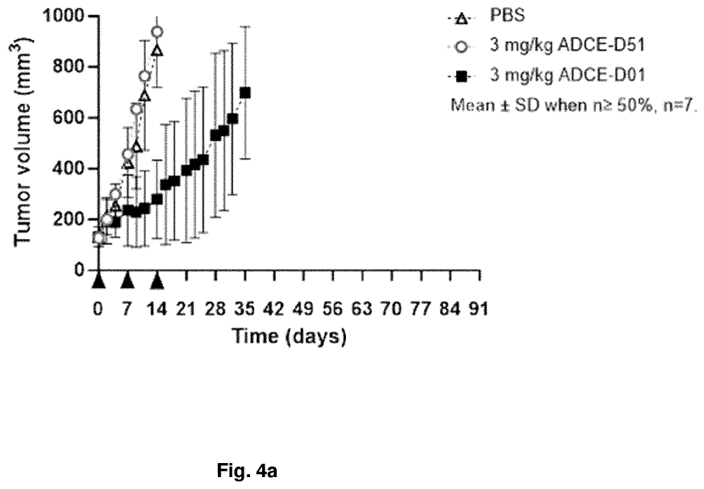

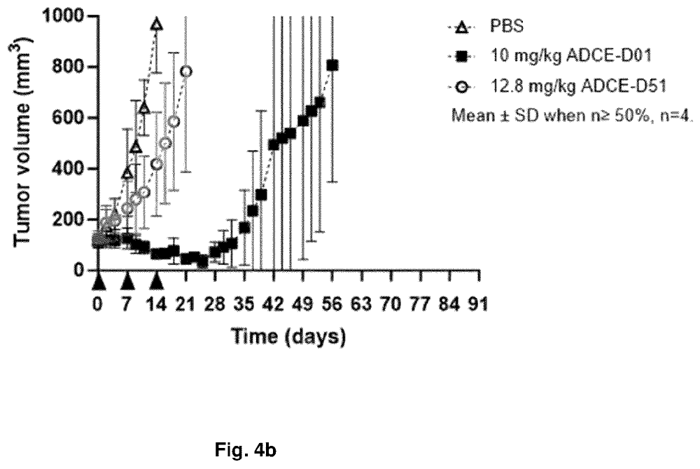

Treatments were administered intravenously (IV) via tail vein on Days 0, 7 and 14 (q1wk×3). Control ADC (ADCE-D51) was an isotype matched (IgG1) antibody directed against HIV gp120 (B12) (Parren et al. 1995) conjugated with L-III-30 (comprising payload P-III-30) at a DAR of 8. ADCE-D51 is thus identical to ADCE-D01 in all aspects aside from the antibody. Tumour size was monitored by calliper measurements 3 times per week for up to 90 days. Tumour volume versus time data is presented FIG. 4a and FIG. 4b.

Dose-dependent anti-tumour activity was observed with ADCE-D01 at 3 and 10 mg/kg when compared with PBS. Tumour growth inhibition for ADCE-D01 on Day 14 was 62% and 94% at 3 and 10 mg/kg, respectively. Tumour regression was observed at 10 mg/kg and persisted until Day 28, i.e., 2 weeks following the last dose. Tumour growth resumed after Day 28, but both the control ADC and PBS treated groups had been terminated due to excessive tumour size (800 mm³) at the time.

Rhabdomyosarcoma is a type of soft tissue sarcoma that develops from skeletal (striated) muscle cells. The RD cell line was derived directly from a human biopsy specimen of refractory RMS (McAllister et al. 1969).

CB17-SCID immune-compromised mice were inoculated subcutaneously in the right flank with human RD cells. Treatment was initiated when the group mean tumour volume was approximately 150 mm³. Mice were treated with vehicle control (PBS), control ADC and ADCE-D01 at 3 and 6 mg/kg.

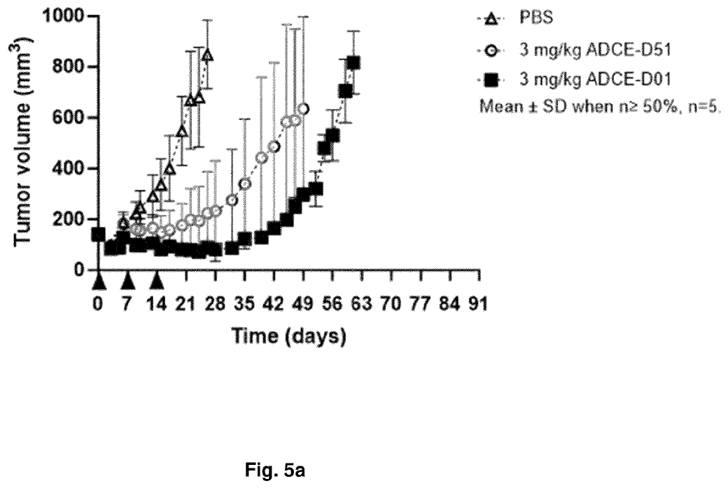

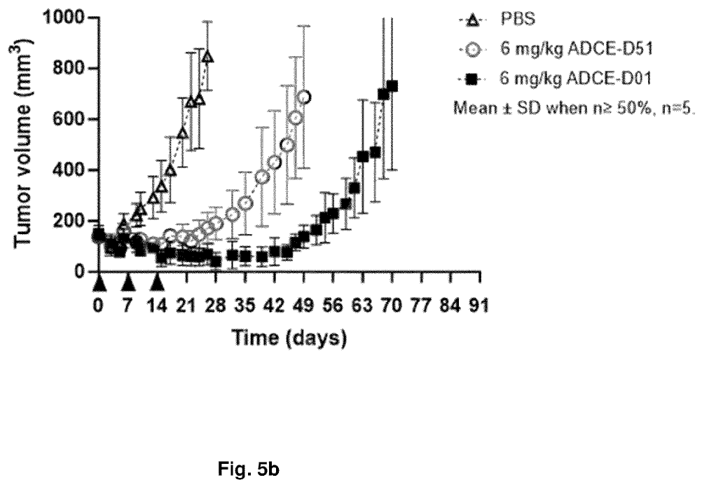

Treatments were administered IV via tail vein on Days 0, 7 and 14 (q1wk×3). Control ADC was ADCE-D51 as described above for Leiomyosarcoma. Tumour size was monitored by calliper measurements 3 times per week for up to 90 days. Tumour volume versus time data is presented in FIG. 5a and FIG. 5b.

Dose-dependent anti-tumour activity was observed with ADCE-D01 at all tested doses when compared with PBS in the RD rhabdomyosarcoma model. Tumour growth inhibition on Day 22 was 87% and 89% at 3 and 6 mg/kg, respectively. Tumour regression was observed at 6 mg/kg and persisted until Day 42 i.e., 4 weeks following the last dose. Tumour growth resumed after Day 42 but mean tumour volumes at 6 mg/kg remained smaller than both the control ADC (6 mg/kg) and PBS treated groups.

### Sequence Overview

| SEQ ID | | |
|---|---|---|
| NO | Sequence | Comment |
| 1 | EIVMTQSPDSLAVSLGERATINCKASQNVDTYVVWYQ | Complete |
| | QKPGQPPQPLIYSASSRFSGVPDRFSGSGSGTDFTLTI | light chain |
| | SSLQAEDVAIYYCQQYHNSPLTFGGGTKVEIKRTVAAP | sequence |
| | SVFIFPPSDEQLKSGTASVVQLLNNFYPREAKVQWKVD | (LC4) of |
| | NALQSGNSQESVTEQDSKDSTYSLSSTLTLSKADYEK | humanised |
| | HKVYACEVTHQGLSSPVTKSFNRGEC | LC4HC3 |
| 2 | MVSSAQFLGLLLLCFQGTRCEIVMTQSPDSLAVSLGER | Complete |
| | ATINCKASQNVDTYVVWYQQKPGQPPQPLIYSASSRFS | light chain |
| | GVPDRFSGSGSGTDFTLTISSLQAEDVAIYYCQQYHNS | sequence |
| | PLTFGGGTKVEIKRTVAAPSVFIFPPSDEQLKSGTASV | (LC4) of |
| | VCLLNNFYPREAKVQWKVDNALQSGNSQESVTEQDSKD | humanised |
| | STYSLSSTLTLSKADYEKHKVYACEVTHQGLSSPVT | LC4HC3 in |
| | KSFNRGEC | combination |
| | | with signal |
| | | peptide used |
| | | in CHO cell |
| | | expression |
| | | system; |
| | | Signal |
| | | peptide |
| | | underlined |
| 3 | EIVMTQSPDSLAVSLGERATINCKASQNVDTYVVWYQ | Light chain |
| | QKPGQPPQPLIYSASSRFSGVPDRFSGSGSGTDFTLT | (LC4) |
| | ISSLQAEDVAIYYCQQYHNSPLTFGGGTKVEIK | variable |
| | | region of |
| | | humanised |
| | | LC4HC3 |
| 4 | QVQLVQSGAEVKKPGASVKVSCKASGYIFIDYGMHWV | Complete |
| | RQAPGQRLEWMGSINTKSGVSTYAAEFKGRVTIYSDT | heavy chain |
| | SASTAYMELSSLRSEDTAVYFCARPPYYSQYGSYWGQ | sequence |
| | GTLVTVSSASTKGPSVFPLAPSSKSTSGGTAALGCLV | (HC3) of |
| | KDYFPEPVTVSWNSGALTSGVHTFPAVLQSSGLYSLS | humanised |
| | SWVTVPSSSLGTQTYICNVNHKPSNTKVDKKVEPKSC | LC4HC3 and |
| | DKTHTCPPCPAPELLGGPSVFLFPPKPKDTLMISRTP | LC3HC3 |
| | EVTCVVVDVSHEDPEVKFNWYVDGVEVHNAKTKPREE | |
| | QYNSTYRVVSVLTVLHQDWLNGKEYKCKVSNKALPAP | |
| | IEKTISKAKGQPREPQVYTLPPSRDELTKNQVSLTCL | |
| | VKGFYPSDIAVEWESNGQPENNYKTTPPVLDSDGSFF | |
| | LYSKLTVDKSRWQQGNVFSCSVMHEALHNHYTQKSLS | |
| | LSPGK | |
| 5 | MGWTLVFLFLLSVTAGVHSQVQLVQSGAEVKKPGASV | Complete |
| | KVSCKASGYIFIDYGMHWVRQAPGQRLEWMGSINTKS | heavy chain |
| | GVSTYAAEFKGRVTIYSDTSASTAYMELSSLRSEDTAV | sequence |
| | YFCARPPYYSQYGSYWGQGTLVTVSSASTKGPSVFPL | (HC3) of |
| | APSSKSTSGGTAALGCLVKDYFPEPVTVSWNSGALTS | humanised |
| | GVHTFPAVLQSSGLYSLSSWTVPSSSLGTQTYICNVN | LC4HC3 and |
| | HKPSNTKVDKKVEPKSCDKTHTCPPCPAPELLGGPSV | LC3HC3 in |
| | FLFPPKPKDTLMISRTPEVTCVVVDVSHEDPEVKFNWY | combination |
| | VDGVEVHNAKTKPREEQYNSTYRVVSVLTVLHQDWLN | with signal |
| | GKEYKCKVSNKALPAPIEKTISKAKGQPREPQVYTLPP | peptide used |
| | SRDELTKNQVSLTCLVKGFYPSDIAVEWESNGQPENN | in CHO cell |
| | YKTTPPVLDSDGSFFLYSKLTVDKSRWQQGNVF | expression |
| | SCSVMHEALHNHYTQKSLSLSPGK | system; |
| | | Signal |
| | | peptide |
| | | underlined |
| 6 | QVQLVQSGAEVKKPGASVKVSCKASGYIFIDYGMHWV | Heavy chain |
| | RQAPGQRLEWMGSINTKSGVSTYAAEFKGRVTIYSDT | (HC3) |
| | SASTAYMELSSLRSEDTAVYFCARPPYYSQYGSYWGQ | variable |
| | GTLVTVSS | region of |
| | | humanised |
| | | LC4HC3 and |
| | | LC3HC3 |
| 7 | DIVMTQSQKFMSTSVGDRVSVTCKASQNVDTYVVWY | Complete |
| | QQKPGQSPKPLIYSASSRFSGVPDRFTGTGSGTDFTLT | light chain |
| | INNVQSEDLAEYFCQQYHNSPLTFGGGTKLEIKRTVAA | sequence |
| | PSVFIFPPSDEQLKSGTASWVCLLNNFYPREAKVQWKV | (LC0) of |
| | DNALQSGNSQESVTEQDSKDSTYSLSSTLTLSKADYE | chimeric |
| | KHKVYACEVTHQGLSSPVTKSFNRGEC | LC0HC0 |
| 8 | DIVMTQSQKFMSTSVGDRVSVTCKASQNVDTYVVWY | Light chain |
| | QQKPGQSPKPLIYSASSRFSGVPDRFTGTGSGTDFTLT | (LC0) |
| | INNVQSEDLAEYFCQQYHNSPLTFGGGTKLEIK | variable |
| | | region of |
| | | chimeric |
| | | LC0HC0 |
| 9 | QVHLVQSGPELKKPGETVKISCKASGYIFIDYGMHWVK | Complete |
| | QAPGKGLKWMGSINTKSGVSTYAAEFKGRFAFSLETS | heavy chain |
| | ASTAYLQINNLKNEDTATYFCARPPYYSQYGSYWGQG | sequence |
| | TLVTVSAASTKGPSVFPLAPSSKSTSGGTAALGCLVKD | (HC0) of |
| | YFPEPVTVSWNSGALTSGVHTFPAVLQSSGLYSLSSV | chimeric |
| | VTVPSSSLGTQTYICNVNHKPSNTKVDKKVEPKSCDKT | LC0HC0 |
| | HTCPPCPAPELLGGPSVFLFPPKPKDTLMISRTPEVTC | |
| | WWVDVSHEDPEVKFNWYVDGVEVHNAKTKPREEQYN | |
| | STYRVVSVLTVLHQDWLNGKEYKCKVSNKALPAPIEKT | |
| | ISKAKGQPREPQVYTLPPSRDELTKNQVSLTCLVKGFY | |
| | PSDIAVEWESNGQPENNYKTTPPVLDSDGSFFLYSKLT | |
| | VDKSRWQQGNVFSCSVMHEALHNHYTQKSLSLSPGK | |
| 10 | QVHLVQSGPELKKPGETVKISCKASGYIFIDYGMHWVK | Heavy chain |
| | QAPGKGLKWMGSINTKSGVSTYAAEFKGRFAFSLETS | (HC0) |
| | ASTAYLQINNLKNEDTATYFCARPPYYSQYGSYWGQG | variable |
| | TLVTVSA | region of |
| | | chimeric |
| | | LC0HC0 |
| 11 | ATGGTCAGCTCTGCTCAATTTCTCGGACTCCTTCTTC | Polynucleotide |
| | TGTGCTTTCAAGGAACACGCTGCGAGATCGTGATGA | encoding |
| | CTCAGTCCCCGGACTCACTGGCAGTGTCCTTGGGC | LC4 incl. |
| | GAAAGAGCCACCATCAACTGTAAAGCCAGCCAGAAC | signal |
| | GTGGACACCTACGTGGTCTGGTACCAGCAGAAGCCT | peptide |
| | GGACAGCCACCGCAGCCGTTGATCTACTCGGCCTC | |
| | ATCAAGGTTCTCCGGGGTGCCGGACCGCTTCTCCG | |
| | GATCCGGCTCCGGCACCGATTTCACCCTGACCATCT | |
| | CCTCACTGCAAGCCGAGGACGTGGCTATCTACTATT | |
| | GCCAGCAGTACCACAACTCCCCACTGACCTTCGGTG | |
| | GCGGAACTAAGGTCGAGATTAAGCGGACCGTGGCG | |
| | GCCCCCTCTGTGTTCATTTTCCCTCCCTCGGACGAA | |
| | CAGCTGAAGTCGGGAACAGCCTCCGTCGTGTGCCT | |
| | GCTCAACAACTTCTACCCCCGGGAAGCGAAGGTCCA | |
| | GTGGAAAGTGGATAACGCACTCCAATCGGGGAACTC | |
| | CCAGGAATCCGTGACTGAGCAGGACTCGAAGGATTC | |
| | CACTTACTCCCTGTCGTCCACCCTGACTCTGAGCAA | |
| | GGCCGACTACGAGAAGCATAAGGTCTACGCCTGCG | |
| | AAGTGACCCACCAGGGTCTGAGCTCCCCTGTGACCA | |
| | AGAGCTTTAATCGGGGCGAATGTTGA | |
| 12 | ATGGGTTGGACCCTCGTCTTTCTGTTCCTTCTTTCCG | Polynucleotide |
| | TCACCGCTGGAGTGCATAGCCAGGTCCAATTGGTGC | encoding |
| | AGTCAGGCGCCGAAGTGAAAAAGCCTGGGGCGTCG | HC3 incl. |
| | GTGAAAGTGTCCTGCAAAGCCTCGGGCTACATCTTT | signal |
| | ATTGACTACGGAATGCACTGGGTCCGCCAGGCCCC | peptide |
| | GGGCCAGAGGCTGGAGTGGATGGGATCCATTAACA | |
| | CCAAGAGCGGAGTGTCAACTTACGCAGCCGAGTTCA | |
| | AGGGACGGGTGACCATCTATAGCGATACCTCTGCGT | |
| | CGACCGCCTACATGGAATTGTCATCACTCCGGTCCG | |
| | AGGACACTGCCGTGTACTTCTGCGCAAGGCCACCCT | |
| | ACTACTCGCAATACGGCAGCTACTGGGGCCAGGGA | |
| | ACACTTGTGACCGTGTCGAGCGCGTCCACCAAGGG | |
| | TCCCTCCGTGTTCCCTCTCGCGCCGTCCTCAAAGTC | |
| | TACCTCCGGTGGAACTGCCGCGCTCGGTTGTCTCGT | |
| | GAAGGACTACTTCCCGGAGCCTGTGACTGTCTCCTG | |
| | GAACTCCGGGGCCCTCACCAGCGGAGTGCACACTT | |
| | TCCCCGCCGTGCTGCAATCCTCCGGCCTGTACAGC | |
| | CTGTCCTCCGTCGTGACTGTGCCTAGCTCCTCCCTG | |
| | GGAACCCAGACCTACATCTGCAACGTGAACCACAAG | |
| | CCCTCCAACACCAAGGTCGACAAGAAGGTCGAACC | |
| | GAAGTCGTGCGACAAGACTCATACGTGCCCTCCTTG | |
| | CCCGGCCCCGGAACTGCTGGGAGGCCCATCCGTGT | |
| | TCCTGTTCCCACCCAAGCCTAAGGATACCCTGATGA | |
| | TCAGCAGAACACCGGAAGTGACCTGTGTGGTGGTG | |
| | GACGTCAGCCACGAAGATCCCGAGGTCAAGTTCAAT | |
| | TGGTACGTGGACGGGGTGGAGGTGCACAACGCAAA | |
| | GACCAAGCCCCGGGAGGAACAGTACAACTCCACCT | |
| | ATCGCGTGGTGTCGGTGCTGACGGTGCTGCACCAG | |
| | GACTGGTTGAACGGAAAGGAGTATAAGTGCAAAGTG | |
| | TCGAACAAGGCCCTGCCCGCTCCTATCGAAAAGACC | |
| | ATCTCCAAGGCCAAGGGCCAGCCGCGGGAACCCCA | |
| | GGTCTACACTCTCCCACCGAGCCGCGACGAACTGA | |
| | CTAAGAATCAAGTGTCGCTGACTTGCCTCGTCAAGG | |
| | GCTTCTACCCGTCCGACATCGCCGTGGAATGGGAG | |
| | AGCAACGGCCAGCCGGAAAACAACTACAAGACCAC | |
| | CCCTCCCGTGCTGGATTCCGACGGGTCCTTCTTCCT | |
| | GTACTCAAAACTGACCGTGGATAAGTCCAGATGGCA | |
| | GCAGGGCAATGTCTTTTCATGCTCCGTGATGCACGA | |
| | GGCTCTGCATAACCACTACACCCAGAAGTCGCTGTC | |
| | CCTGTCCCCGGGGAAGTGA | |
| 13 | MVSSAQFLGLLLLCFQGTRCDVVMTQSPDSLAVSLGE | Complete |
| | RVTINCKASQNVDTYWWYQQKPGQSPKLLIYSASSRF | light chain |
| | SGVPDRFSGSGSGTDFTLTISSLQAEDVAVYYCQQYH | sequence |
| | NSPLTFGQGTKLEIKRTVAAPSVFIFPPSDEQLKSGT | (LC3) of |
| | ASWVCLLNNFYPREAKVQWKVDNALQSGNSQESVTEQ | humanised |
| | DSKDSTYSLSSTLTLSKADYEKHKVYACEVTHQGLSS | LC3HC3 |
| | PVTKSFNRGEC | |
| 14 | GGFG | Peptide linker |
| 15 | GGGG | Peptide linker |

### REFERENCES

J. Fogh and G. Trempe, “New Human Tumor Cell Lines,” In: J. Fogh, Ed., Human Tumor Cells in Vitro, Plenum Publishing Corp., New York, 1975, pp. 115-159.
Parren P W, Ditzel H J, Gulizia R J, Binley J M, Barbas C F 3rd, Burton D R, Mosier D E. Protection against HIV-1 infection in hu-PBL-SCID mice by passive immunization with a neutralizing human monoclonal antibody against the gp120 CD4-binding site. AIDS. 1995 June; 9(6):F1-6. doi: 10.1097/00002030-199506000-00001. PMID:7662189.
McAllister, R. M., Melnyk, J., Finklestein, J. Z., Adams, E. C., Jr. and Gardner, M. B. (1969), Cultivation in vitro of cells derived from a human rhabdomyosarcoma. Cancer, 24: 520-526. https://doi.org/10.1002/1097-0142(196909)24:3<20::AID-CNCR2320240313>3.0.CO;2-M

### Items

1. An antibody-drug conjugate (ADC) comprising an antibody which binds to uPARAP comprising:
i. an immunoglobulin light chain variable region comprising or consisting of the amino acid sequence of SEQ ID NO: 3; and
ii. an immunoglobulin heavy chain variable region comprising or consisting of the amino acid sequence of SEQ ID NO: 6;
wherein the antibody-drug conjugate comprises an active agent with a structure according to one selected from the group consisting of:
a. formula (I-A) or a tautomer, a mesomer, a racemate, an enantiomer or a diastereoisomer thereof, or a mixture thereof, or a pharmaceutically acceptable salt thereof,

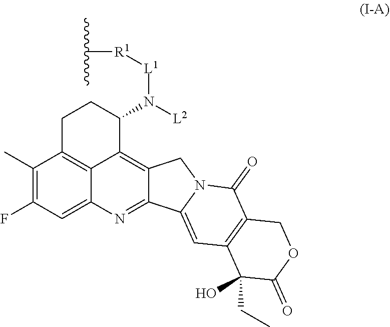

wherein, R¹ is selected from the group consisting of: —O—, —(R²)N—, —P(═O)(R²)—, —P(R²)— and —S—;
L² is —(C(R^{3a})(R^{3b}))ₘ—R,
wherein 0 or no less than 1 methylene unit of L² is independently replaced by -Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
L¹ is —(C(R^{5a})(R^{5b}))ₙ—,
wherein 0 or no less than 1 methylene unit of L¹ is independently replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
Cy- is selected from the group consisting of: 6-10 membered arylene, 5-8 membered heteroarylene, 3-10 membered heterocyclylene, and 3-10 membered saturated or partially unsaturated carbocyclylene, wherein -Cy- is unsubstituted or independently substituted with no less than 1 substituent R⁷;
wherein each R^{3a}, each R^{3b}, each R⁴, each R^{5a}, each R^{5b} and each R⁶ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R; or, R^{3a} and R^{5a}, R⁴ and R^{5a}, R^{3a} and R⁶ or R⁴ and R⁶ each independently optionally form a ring B together with an atom therebetween, wherein the ring B is selected from the group consisting of: 5-8 membered heteroarylene and 3-10 membered saturated or partially unsaturated heterocyclylene, and the ring B is unsubstituted or independently substituted with no less than 1 substituent R⁸;
wherein each R², each R⁷ and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n are each independently selected from the group consisting of integers≥1; and wherein R¹ links the structure shown in formula (I-A) to the antibody, optionally via a linker;
b. Formula (II-A) or a tautomer, a mesomer, a racemate, an enantiomer or a diastereoisomer thereof, or a mixture thereof, or a pharmaceutically acceptable salt thereof

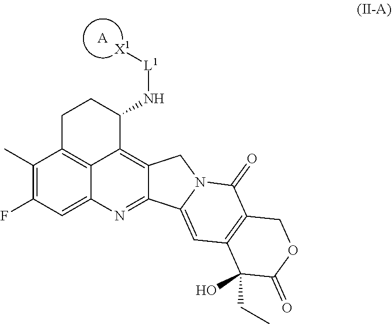

wherein, X¹ is selected from the group consisting of: N, P, and saturated or unsaturated C; when X¹ is saturated C, X¹ is substituted with Rⁿ;
wherein ring A links the structure according to formula II-A to the antibody, optionally via a linker;
when X¹ is saturated C, ring A is selected from the group consisting of: 3-10 membered saturated or partially unsaturated heterocyclyl, and 3-10 membered saturated or partially unsaturated carbocyclyl, wherein ring A is substituted with 0 or no less than 1 substituent R^{1a};
or, when X¹ is unsaturated C, ring A is selected from the group consisting of: 6-10 membered aryl, 5-8 membered heteroaryl, 3-10 membered partially unsaturated heterocyclyl, and 3-10 membered partially unsaturated carbocyclyl, wherein ring A is substituted with 0 or no less than 1 substituent R^{1b};
or, when X¹ is N or P, ring A is selected from the group consisting of: 5-8 membered heteroaryl and 3-10 membered saturated or partially unsaturated heterocyclyl, wherein ring A is substituted with 0 or no less than 1 substituent R^{1c};
when ring A is selected from the group consisting of: 6-10 membered aryl, 5-8 membered heteroaryl, and 3-10 membered saturated or partially unsaturated carbocyclyl, ring A is substituted with p L², wherein L² is not Rⁿ;
or, when ring A is 3-10 membered saturated or partially unsaturated heterocyclyl, ring A is substituted with p L², or ring A comprises q ring-forming heteroatom X², and X² is used for linking formula (II-A) to the antibody, optionally via a linker;
X² is selected from the group consisting of: N and P;
L² is —R²-L³-, and R² is used for linking formula (II-A) to the antibody, optionally via a linker;
L³ is —(C(R^{3a})(R^{3b}))ₘ—, wherein when L³ comprises a methylene unit, 0 or no less than 1 methylene unit of L³ is independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² is selected from the group consisting of: —O—, —(R^{2a})N—, —S— and —P(═O)(R^{2a})—;
L¹ is —(C(R^{5a})(R^{5b}))ₙ—, wherein when L¹ comprises a methylene unit, 0 or no less than 1 methylene unit of L¹ is independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1a}, each R^{1b}, each R^{1c}, each R^{2a}, each R^{3a}, each R^{3b}, each R⁴, each R^{5a}, each R^{5b}, each R⁶ and each Rⁿ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n are each independently selected from the group consisting of integers≥0, and p and q are each independently selected from the group consisting of integers≥1; and
c. Formula (III-A) or a tautomer, a mesomer, a racemate, an enantiomer or a diastereoisomer thereof, or a mixture thereof, or a pharmaceutically acceptable salt thereof

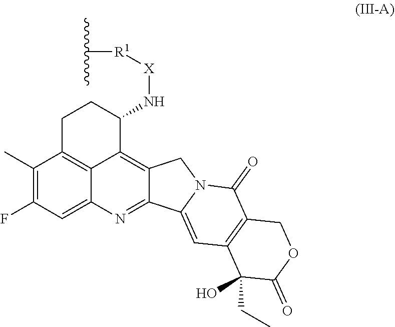

wherein R¹ is selected from the group consisting of: —O—, —(R²)N—, —P(═O)(R²)— and —S—; wherein R¹ links the structure according to formula (III-A) to the antibody, optionally via a linker;
X is selected from the group consisting of: -L¹-C(R^{1a})(R^{1b})—C(O)—, -L¹-C(R^{1a})(R^{1b})—C(S)—, -L¹-L⁰- and -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR^{B}—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group; m is selected from the group consisting of integers≥0, and n is selected from the group consisting of integers 1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

2. The antibody-drug conjugate according to item 1, wherein in formula (I-A) each R^{3a}, each R^{3b}, each R⁴, each R^{5a}, each R^{5b} and each R⁶ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R, and/or wherein in formula (I-A) R^{3a} and R^{5a}, R⁴ and R^{5a}, R^{3a} and R⁶, or R⁴ and R⁶ each independently optionally form a ring B together with an atom therebetween, wherein the ring B is selected from the group consisting of: 5-8 membered heteroarylene and 3-10 membered saturated or partially unsaturated heterocyclylene, and the ring B is unsubstituted or substituted with no less than 1 substituent R⁸.

3. The antibody-drug conjugate according to any one of items 1 to 2, wherein in formula (I-A) m is 1 or 2, optionally, m is 1, and L² is —C(R^{3a})(R^{3b})—R, or optionally, m is 2, and L² is —(C(R^{3a})(R^{3b}))₂—R, and 0 or 1 methylene units of L² are replaced by -Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—, preferably by —C(O)— or -Cy-, such as L² is —C(O)—C(R^{3a})(R^{3b})—R or —C(R^{3a})(R^{3b})-Cy-R.

4. The antibody-drug conjugate according to item 1 to 3, wherein in formula (I-A) 0 or 1 methylene units of L² are replaced by -Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—, such as 1 methylene unit of L² is replaced by —C(O)— or -Cy-.

5. The antibody-drug conjugate according to any one of items 1 to 4, wherein in formula (I-A) n is 2, 3 or 5.

6. The antibody-drug conjugate according to any one of items 1 to 5, wherein in formula (I-A) n is 2, and L¹ is —(C(R^{5a})(R^{5b}))₂—.

7. The antibody-drug conjugate according to any one of items 1 to 6, wherein in formula (I-A) L¹ is —(C(R^{5a})(R^{5b}))₂—, and 0 methylene units of L¹ are replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—; or 1 methylene unit of L¹ is replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as L¹ is —(C(R^{5a})(R^{5b}))₂—, and 1 methylene unit of L¹ is replaced by —C(O)— or —C(═S)—, such as L¹ is —C(R^{5a})(R^{5b})—C(O)— or L¹ is —C(R^{5a})(R^{5b})—C(═S)—.

8. The antibody-drug conjugate according to any one of items 1-5, wherein in formula (I-A) n is 3, and L¹ is —(C(R^{5a})(R^{5b}))₃—.

9. The antibody-drug conjugate according to item 8, wherein in formula (I-A) L¹ is —(C(R^{5a})(R^{5b}))₃—, and 0 methylene units of L¹ are replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—; or 1 methylene unit of L¹ is replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as wherein L¹ is —(C(R^{5a})(R^{5b}))₂—C(O)—.

10. The antibody-drug conjugate according to any one of items 1-5, wherein in formula (I-A) n is 5, and L¹ is —(C(R^{5a})(R^{5b}))₅—.

11. The antibody-drug conjugate according to item 10, wherein in formula (I-A) L¹ is —(C(R^{5a})(R^{5b}))₅—, and 1 methylene unit of L¹ is replaced by —NR⁶—, such as wherein L¹ is —(C(R^{5a})(R^{5b}))₂—NR⁶—(C(R^{5a})(R^{5b}))₂— or is replaced by —O—, such as wherein L¹ is —(C(R^{5a})(R^{5b}))₂—O—(C(R^{5a})(R^{5b}))₂—; or wherein in formula (I-A) L¹ is —(C(R^{5a})(R^{5b}))₅—, and 2 methylene units of L¹ are each independently replaced by —C(O)—, —NR⁶— or —O—, such as L¹ is selected from the group:
C(R^{5a})(R^{5b})—C(O)—NR₆—(C(R^{5a})(R^{5b}))₂—;
(C(R^{5a})(R^{5b}))₂—NR₆—C(O)—C(R^{5a})(R^{5b})—; and
L¹ is —(C(R^{5a})(R^{5b}))₂—O—C(R^{5a})(R^{5b})—C(O)—; or
wherein in formula (I-A) L¹ is —(C(R^{5a})(R^{5b}))₅—, and 3 methylene units of L¹ are each independently replaced by —C(O)— or —NR⁶—, such as wherein L¹ is —(C(R^{5a})(R^{5b}))₂—NR⁶—C(O)—C(O)—.

12. The antibody-drug conjugate according to any one of items 1 to 4, wherein in formula (I-A) 0 methylene units of L¹ are replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—; or wherein in formula (I-A) 1 methylene unit of L¹ is replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as is replaced by —C(O)—, —C(═S)—, —NR⁶— or —O—, more preferably by —C(O)—; or
wherein in formula (I-A) 2 methylene units of L¹ are each independently replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as each independently replaced by —C(O)—, —NR⁶— or —O—, preferably by —C(O)— or —NR⁶—; or
wherein in formula (I-A) 3 methylene units of L¹ are each independently replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as each independently replaced by —C(O)— or —NR⁶—.

13. The antibody-drug conjugate according to any one of items 1 to 2, wherein in formula (I-A) R^{3a} and R^{5a} independently optionally form a ring B together with an atom therebetween, optionally ring B is 3-10 membered saturated or partially unsaturated heterocyclylene, such as ring B is 3-6 membered saturated or partially unsaturated heterocyclylene, such as ring B is 5 membered saturated or partially unsaturated heterocyclylene; or
wherein in formula (I-A) R⁴ and R^{5a} independently optionally form a ring B together with an atom therebetween, optionally ring B is 3-10 membered saturated or partially unsaturated heterocyclylene, such as ring B is 6 membered saturated or partially unsaturated heterocyclylene.

14. The antibody-drug conjugate according to item 1, wherein in formula (I-A) L² is —C(R^{3a})(R^{3b})—R or —(C(R^{3a})(R^{3b}))₂—R, and L¹ is —(C(R^{5a})(R^{5b}))₂—, —(C(R^{5a})(R^{5b}))₃— or —(C(R^{5a})(R^{5b}))₅—.

15. The antibody-drug conjugate according to item 14, wherein in formula (I-A) L² is —(C(R^{3a})(R^{3b}))₂—R, and L¹ is —(C(R^{5a})(R^{5b}))₂—, optionally 1 methylene unit of L¹ is replaced by —C(O)—, —C(═S)—, —NR⁶— or —O—, such as L² is —(C(R^{3a})(R^{3b}))₂—R, and L¹ is —C(R^{5a})(R^{5b})—C(O)—, optionally wherein in formula (I-A) R^{3a} and R^{5a} independently optionally form a ring B together with an atom therebetween, optionally ring B is 3-10 membered saturated or partially unsaturated heterocyclylene, such as ring B is 5 membered saturated heterocyclylene; or
wherein in formula (I-A) L² is —C(R^{3a})(R^{3b})—R, and L¹ is —(C(R^{5a})(R^{5b}))₂—, optionally 1 methylene unit of L¹ is replaced by —C(O)—, —C(═S)—, —NR⁶— or —O—, such as L² is —C(R^{3a})(R^{3b})—R, and L¹ is —C(R^{5a})(R^{5b})—C(O)—.

16. The antibody-drug conjugate according to any one of items 1 to 15, wherein in formula (I-A) R¹ is selected from the group consisting of: —O—, —(R²)N— and —S—.

17. The antibody-drug conjugate according to any one of items 1 to 16, wherein in formula (I-A) R¹ is —O—; or R¹ is —(R²)N—, optionally wherein R² is hydrogen such as —HN—, or a C₁₋₆ aliphatic group.

18. The antibody-drug conjugate according to any one of items 1 to 17, wherein in formula (I-A) -Cy- is 6-10 membered arylene, such as phenylene.

19. The antibody-drug conjugate according to any one of items 1-18, wherein in formula (I-A) R⁷ is hydrogen.

20. The antibody-drug conjugate according any one of items 1 to 2, wherein in formula (I-A) R^{3a} and R^{3b} are independently hydrogen, or R^{3a} and R^{5a} independently optionally form a ring B together with an atom therebetween, optionally ring B is 3-10 membered saturated or partially unsaturated heterocyclylene, such as ring B is 5 membered saturated heterocyclylene; or wherein in formula (I-A) R⁴ is hydrogen, or R⁴ and R^{5a} independently optionally form a ring B together with an atom therebetween.

21. The antibody-drug conjugate according to item 1, wherein in formula (I-A) R⁸ is hydrogen.

22. The antibody-drug conjugate according to item 1, wherein in formula (I-A)R, R^{a} and R^{b} are each independently hydrogen.

23. The antibody-drug conjugate according to item 1, wherein formula (I-A) is according to any one of formulas (I-A-1) to (I-A-17):

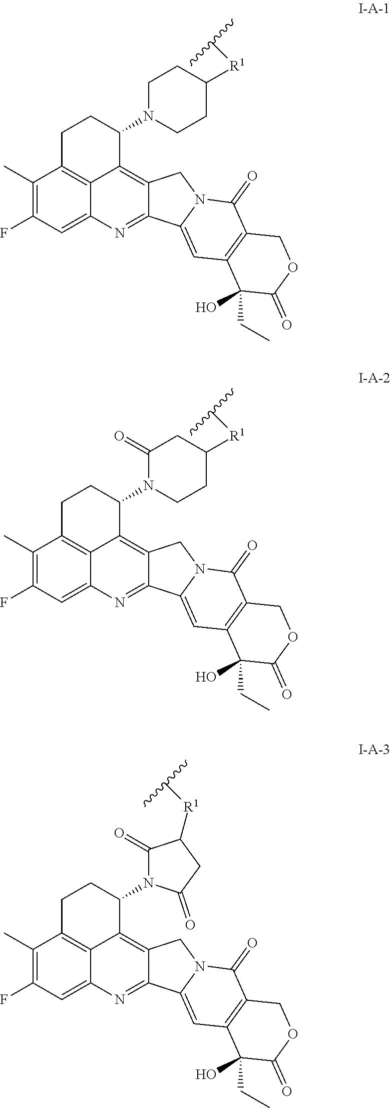

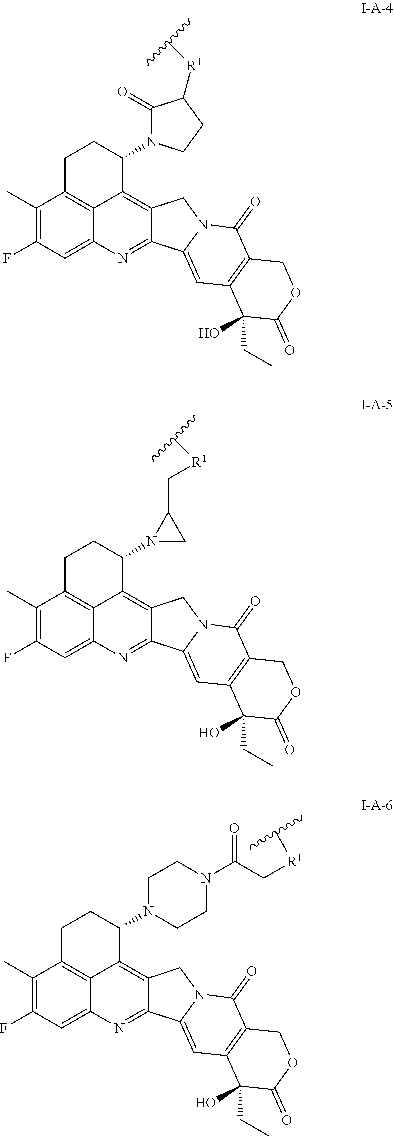

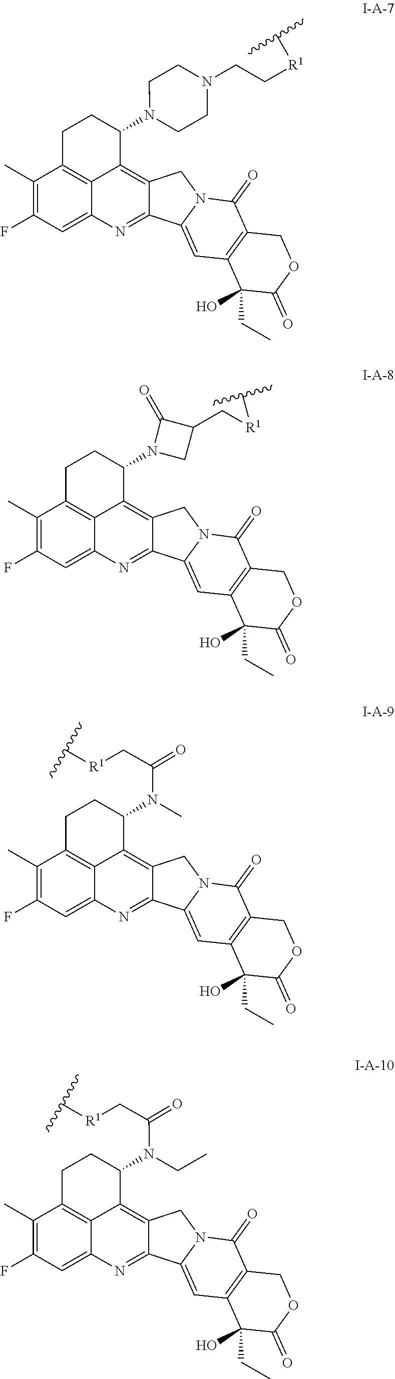

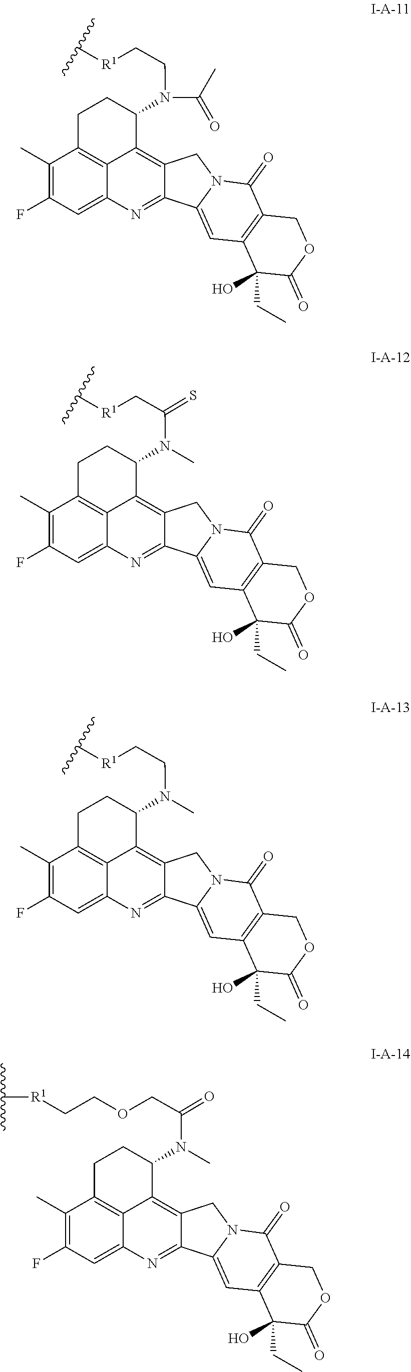

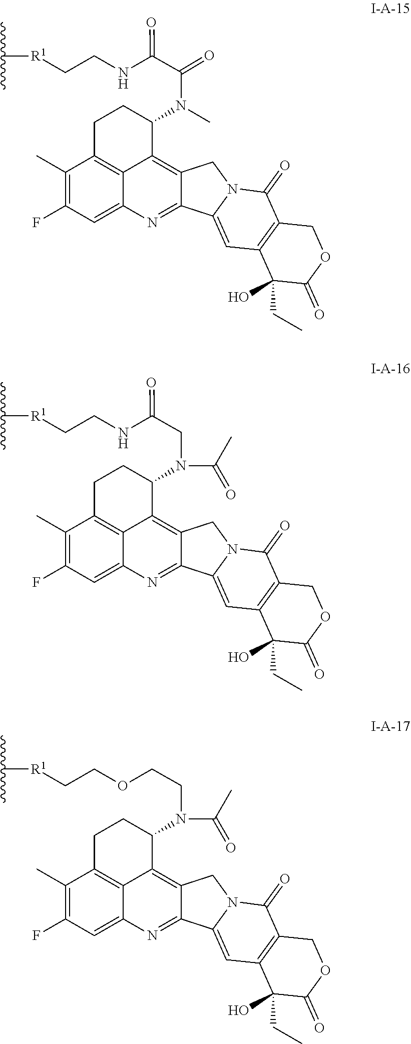

wherein, R¹ may be selected from the group consisting of: —O—, —HN—, —P(═O)H— and —S— and wherein R¹ links the structure shown in any one of formulas (I-A-1) to (I-A-17) to the antibody, optionally via a linker.

24. The antibody-drug conjugate according to item Error! Reference source not found., wherein in formula (II-A) X¹ is saturated C.

25. The antibody-drug conjugate according to any one of items 1 or 24, wherein in formula (II-A) ring A is selected from the group consisting of: 3-10 membered saturated heterocyclyl and 3-10 membered saturated carbocyclyl.

26. The antibody-drug conjugate according to any one of items 1 or 24-25, wherein in formula (II-A) ring A is 3-10 membered saturated carbocyclyl, such as 3-6 membered saturated carbocyclyl, such as 4 membered saturated carbocyclyl or 6 membered saturated carbocyclyl; or ring A is 3-10 membered saturated heterocyclyl, such as 3-6 membered saturated heterocyclyl, such as 3 membered saturated heterocyclyl.

27. The antibody-drug conjugate according to any one of items 1 or 24 to 26, wherein in formula (II-A) ring A comprises 1 heteroatom, such as comprises 1 nitrogen atom.

28. The antibody-drug conjugate according to any one of items 1 or 24 to 26, wherein in formula (II-A) ring A is 5 membered saturated heterocyclyl, optionally wherein ring A comprises 1 heteroatom, such as 1 nitrogen atom.

29. The antibody-drug conjugate according to any one of items 24 to 28, wherein in formula (II-A) ring A is substituted with 0 substituent R^{1a}.

30. The antibody-drug conjugate according to item 1, wherein in formula (II-A) X¹ is unsaturated C.

31. The antibody-drug conjugate according to item 30, wherein in formula (II-A) ring A is selected from the group consisting of: 6-10 membered aryl and 5-8 membered heteroaryl, optionally wherein said 6-10 membered aryl is phenyl.

32. The antibody-drug conjugate according to any one of items 30 to 31, wherein in formula (II-A) ring A is substituted with 0 substituent R^{1b}.

33. The antibody-drug conjugate according to item 1, wherein in formula (II-A) X¹ is N or P, preferably N.

34. The antibody-drug conjugate according to any one of items 1 and 33, wherein in formula (II-A) ring A is selected from the group consisting of: 5-8 membered heteroaryl and 3-10 membered saturated heterocyclyl.

35. The antibody-drug conjugate according to any one of items 1 and 33 to 34, wherein in formula (II-A) ring A is 3-10 membered saturated heterocyclyl, such as 3-6 membered saturated heterocyclyl, such as 6 membered saturated heterocyclyl, optionally wherein ring A independently comprises 2 heteroatoms such as 2 nitrogen atoms.

36. The antibody-drug conjugate according to any one of items 33 to 35, wherein in formula (II-A) ring A is substituted with 0 substituent R^{1c}.

37. The antibody-drug conjugate according to item 1, wherein the formula (II-A) is a structure shown as formula (II-Ax):

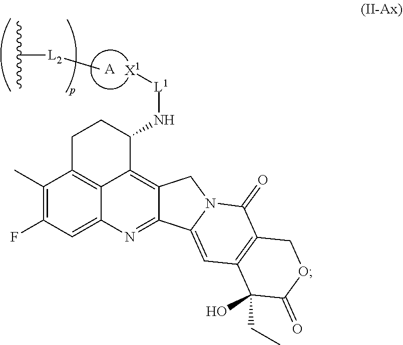

wherein, when ring A is selected from the group consisting of: 6-10 membered aryl, 5-8 membered heteroaryl, and 3-10 membered saturated or partially unsaturated carbocyclyl, ring A is substituted with p L²; or, when ring A is 3-10 membered saturated or partially unsaturated heterocyclyl, ring A is substituted with p L².

38. The antibody-drug conjugate according to item 1, wherein the formula (II-A) is a structure shown as formula (II-Ay):

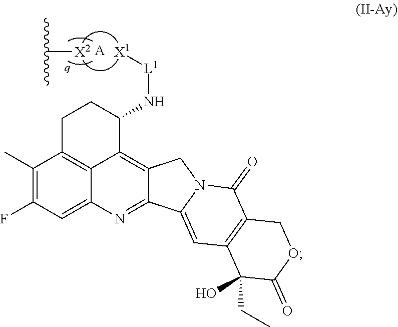

wherein ring A is 3-10 membered saturated or partially unsaturated heterocyclyl, ring A comprises q ring-forming heteroatom X², and X² is used for linking to the antibody, optionally via a linker.

39. The antibody-drug conjugate according to item 1, wherein in formula (II-A) ring A is selected from the group consisting of: 6-10 membered aryl, 5-8 membered heteroaryl, and 3-10 membered saturated carbocyclyl.

40. The antibody-drug conjugate according to any one of items 1 and 39, wherein in formula (II-A) ring A is selected from the group consisting of: phenyl and 3-6 membered saturated carbocyclyl, such as 4 membered saturated carbocyclyl or 6 membered saturated carbocyclyl, such as ring A is phenyl.

41. The antibody-drug conjugate according to any one of items 1 and 39 to 40, wherein in formula (II-A) ring A is substituted with no less than 1 L², such as 1 L².

42. The antibody-drug conjugate according to any one of items 1 and 39, wherein in formula (II-A) ring A is 3-10 membered saturated heterocyclyl, such as 3-6 membered saturated heterocyclyl, optionally 3 membered saturated heterocyclyl or 5 membered saturated heterocyclyl or 6 membered saturated heterocyclyl.

43. The antibody-drug conjugate according to item 42, wherein in formula (II-A) ring A is substituted with no less than 1 L², such as with 1 L².

44. The antibody-drug conjugate according to item 1, wherein in formula (II-A) m is 0, 1 or 2, optionally wherein m is 0, and L³ is a covalent bond; or
m is 1, and L³ is —C(R^{5a})(R^{5b})—, optionally wherein 0 methylene units of L³ are replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—; or
m is 2, and L³ is —(C(R^{3a})(R^{3b}))₂— optionally wherein 0 methylene units of L³ are replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—.

45. The antibody-drug conjugate according to any one of items 1 and 38, wherein ring A comprises no less than 1 ring-forming heteroatom X², such as 1 X² and X² is used for linking to the antibody, optionally via a linker, optionally wherein X² is N.

46. The antibody-drug conjugate according to any one of items 1 to 45, wherein n is 0 or 1, optionally, wherein n is 0, and L¹ is a covalent bond; or
optionally, wherein n is 1, and L¹ is —C(R^{5a})(R^{5b})— and optionally 1 methylene unit of L¹ is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as by —C(O)—.

47. The antibody-drug conjugate according to any one of items 1 to 46, wherein R² is selected from the group consisting of: —O—, —(R^{2a})N— and —S—.

48. The antibody-drug conjugate according to any one of items 1 to 47, wherein R² is —O—; or R² is —(R^{2A})N—, optionally wherein R^{2a} is hydrogen, such as —HN—.

49. The antibody-drug conjugate according to any one of items 1 to 29, wherein R^{1a} is hydrogen.

50. The antibody-drug conjugate according to any one of items 1 and 30 to 33, wherein R^{1b} is hydrogen.

51. The antibody-drug conjugate according to any one of items 1 and 33 to 36, wherein R^{1c} is hydrogen.

52. The antibody-drug conjugate according to item 1, wherein R^{3a} and R^{3b} are each independently hydrogen; and/or R⁴ is hydrogen; and/or R^{5a} and R^{5b} are each independently hydrogen; and/or R⁶ is hydrogen, and/or R, R^{a} and R^{b} are each independently hydrogen.

53. The antibody-drug conjugate according to item 1, wherein formula II-A is according to any one of formulas (II-A-1) to (II-A-12):

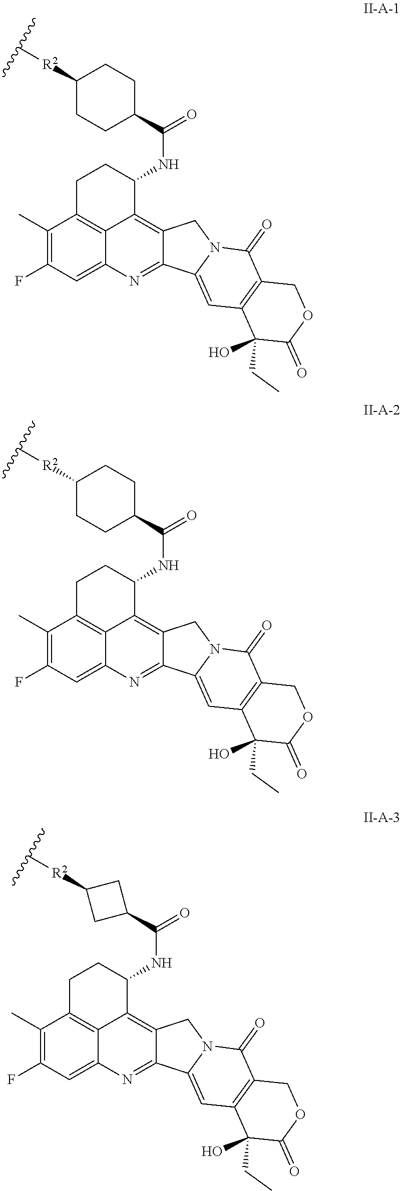

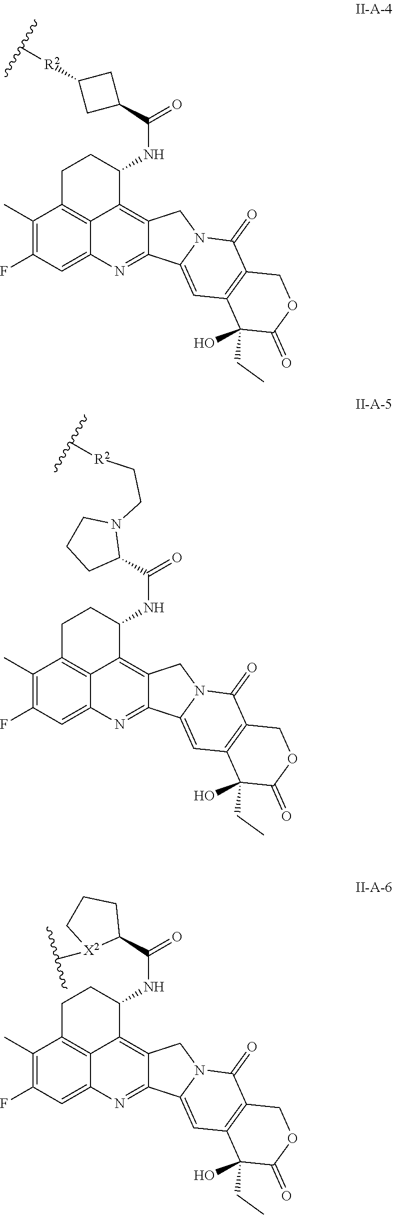

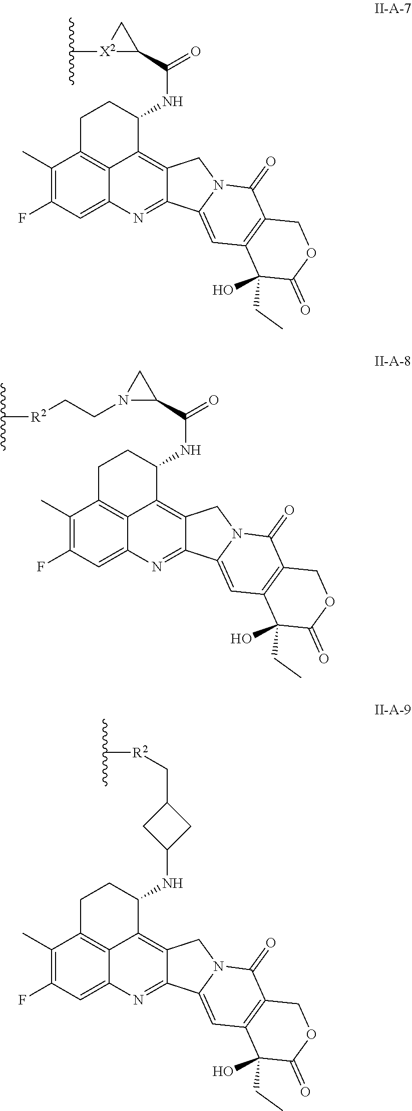

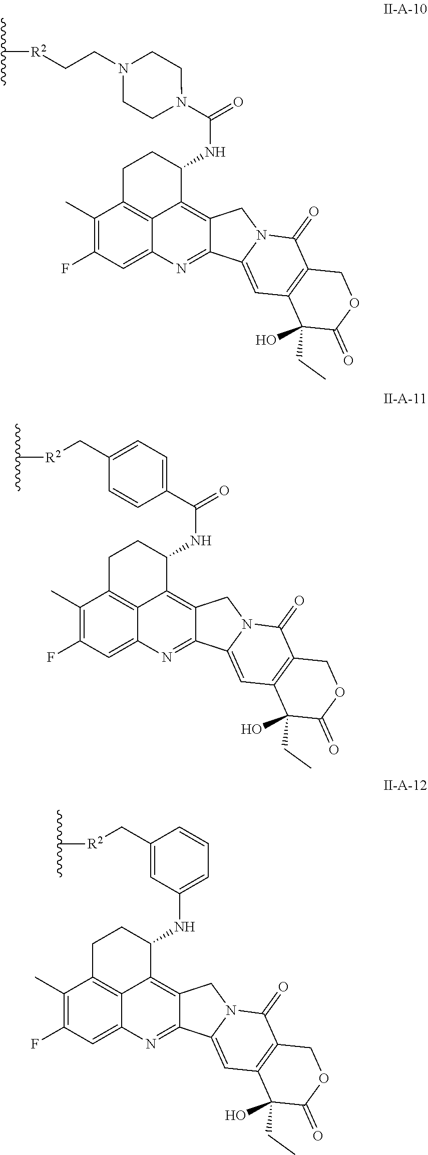

wherein, R² is selected from the group consisting of: —O—, —HN—, —P(═O)H— and —S—; X² is selected from the group consisting of N and P; wherein R² or X² links any one of the structures shown in formulas (II-A-1) to (II-A-12) to the antibody, optionally via a linker.

54. The antibody-drug conjugate according to item 1, wherein in formula (III-A) X is -L¹-C(R^{1a})(R^{1b})—C(O)—, R¹ is —S— or —(R²)N—, and R² is not hydrogen.

55. The antibody-drug conjugate according to any one of items 1 to 54, wherein in formula (III-A) L¹ is —(C(R^{3a})(R^{3b}))ₘ—, and 0 or 1 methylene unit of L¹ is replaced by —C(O)—, —C(═S)—, —C(═NR^{4b})— or —C(═N₂)—, such as 1 methylene unit of L¹ is replaced by —C(O)—.

56. The antibody-drug conjugate according to item 1, wherein in formula (III-A) X is -L¹-C(R^{1a})(R^{1b})—C(O)—, R¹ is —O— or —HN—, m is not 0, and no less than 1 methylene unit of L¹ is replaced by —C(O)—, —C(═S)—, —C(═NR^{4b})— or —C(═N₂)—, such as 1 methylene unit, optionally wherein in formula (III-A) 1 methylene unit of L¹ is replaced by —C(O)—.

57. The antibody-drug conjugate according to item 1, wherein in formula (III-A) X is -L¹-C(R^{1a})(R^{1b})—C(O)—, R¹ is —O— or —HN—, L¹ is —(C(R^{3a})(R^{3b}))ₘ—, m is not 0, 0 methylene units of L¹ are replaced by —C(O)—, —C(═S)—, —C(═NR^{4b})— or —C(═N₂)—, each R^{3a} and each R^{3b} are not both hydrogen, and R^{1a} and R^{1b} are hydrogen.

58. The antibody-drug conjugate according to item 1, wherein in formula (III-A) X is -L¹-C(R^{1a})(R^{1b})—C(O)—, and m is 0, 1 or 2.

59. The antibody-drug conjugate according to any one of items 1 and 58, wherein in formula (III-A) m is 0, and L¹ is a covalent bond, optionally further wherein in formula (III-A) R¹ is (R²)N- or —S—, and R² is not hydrogen.

60. The antibody-drug conjugate according to any one of items 1 and 58, wherein in formula (III-A) m is 1, and L¹ is —C(R^{3a})(R^{3b})—, optionally 0 methylene units of L¹ are replaced by —C(O)—, —C(═S)—, —C(═NR^{4b})— or —C(═N₂)—.

61. The antibody-drug conjugate according to any one of items 1, 58 and 60, wherein in formula (III-A) R¹ is (R²)N— or —S—, and R² is not hydrogen; optionally wherein R¹ is —S—; and/or R¹ is —S—, and R^{1a} and R^{1b} are hydrogen or R^{1a} and R^{1b} are each independently —N(R^{a})(R^{b}), such as wherein R^{1a} is —N(R^{a})(R^{b}).

62. The antibody-drug conjugate according to any one of items 1, 58 and 60 to 61, wherein in formula (III-A) R is hydrogen.

63. The antibody-drug conjugate according to any one of items 1 to 62, wherein in formula (III-A) R¹ is —(R²)N—, and R² is a C₁₋₆ aliphatic group, such as methyl.

64. The antibody-drug conjugate according to any one of items 1 to 62, wherein in formula (III-A) L¹ is —C(R^{3a})(R^{3b})—, R¹ is —O— or —HN—, 0 methylene units of L¹ are replaced by —C(O)—, —C(═S)—, —C(═NR^{4b})— or —C(═N₂)—, and R^{3a} and R^{3b} are not both hydrogen; optionally wherein R¹ is —O— and/or R^{3a} is a C₁₋₆ aliphatic group; optionally R^{3a} is a C₁₋₆ aliphatic group, and R^{3b} is hydrogen or a C₁₋₆ aliphatic group, such as wherein R^{3a} is a C₁₋₆ aliphatic group, and R^{3b} is hydrogen.

65. The antibody-drug conjugate according to any one of items 1 to 62 and 64, wherein in formula (III-A) R^{3a} is methyl, and R^{3b} is hydrogen.

66. The antibody-drug conjugate according to item 1, wherein in formula (III-A) R^{3a} is a C₁₋₆ aliphatic group, and R^{3b} is a C₁₋₆ aliphatic group, such as wherein R^{3a} is methyl, and R^{3b} is a C₁₋₆ aliphatic group, such as wherein R^{3a} is methyl, and R^{3b} is methyl.

67. The antibody-drug conjugate according to any one of items 1 and 58, wherein in formula (III-A) m is 2, and L¹ is —(C(R^{3a})(R^{3b}))₂—; optionally wherein in formula (III-A) 1 methylene unit of L¹ is replaced by —C(O)—, —C(═S)—, —C(═NR^{4b})— or —C(═N₂)—, such as replaced by —C(O)—.

68. The antibody-drug conjugate according to any one of items 1, 58 and 67, wherein in formula (III-A) R¹ is selected from the group consisting of: —O—, —(R²)N—, —P(═O)(R²)— and —S—.

69. The antibody-drug conjugate according to any one of items 1, 58 and 67 to 68, wherein in formula (III-A) R¹ is —O—; or R¹ is —S—; or R¹ is —(R²)N—, optionally wherein R² is a C₁₋₆ aliphatic group, such as methyl.

70. The antibody-drug conjugate according to item 1, wherein in formula (III-A) X is -L¹-L⁰-, R¹ is —O—, —S— or —(R²)N—, and R² is not hydrogen, optionally wherein L¹ is —C(R^{3a})(R^{3b}), and 0 or 1 methylene unit of L¹ is replaced by —C(O)—, —C(═S)—, —C(═NR^{4b})— or —C(═N₂)—, such as replaced by —C(O)—; or
wherein in formula (III-A) X is -L¹-L⁰-, R¹ is —HN—, m is not 0, and L⁰ is —C(═S)—, —C(═NR^{4a})— or —C(═N₂)—; or
wherein in formula (III-A) X is -L¹-L⁰-, R¹ is —HN—, m is not 0, L⁰ is —C(R^{2a})(R^{2b})—, L¹ is —(C(R^{3a})(R^{3b}))ₘ—, and each R^{3a} and each R^{3b} are not both hydrogen.

71. The antibody-drug conjugate according to item 1, wherein in formula (III-A) X is -L¹-L⁰-, and m is 1 or 2; optionally wherein m is 1, and L¹ is —C(R^{3a})(R^{3b})—; optionally wherein 0 methylene units of L¹ are replaced by —C(O)—, —C(═S)—, —C(═NR^{4b})— or —C(═N₂)—.

72. The antibody-drug conjugate according to any one of items 1 and 71, wherein in formula (III-A) R¹ is (R²)N— or —S—, and R² is not hydrogen; optionally wherein R¹ is —S—.

73. The antibody-drug conjugate according to any one of items 1 and 71, wherein in formula (III-A) m is 2, and L¹ is —(C(R^{3a})(R^{3b}))₂—; optionally, wherein in formula (III-A) 0 methylene units of L¹ are replaced by —C(O)—, —C(═S)—, —C(═NR^{4b})— or —C(═N₂)—.

74. The antibody-drug conjugate according to any one of items 1, 71 and 73, wherein in formula (III-A) R¹ is —O—, —S— or (R²)N—, and R² is not hydrogen; optionally wherein R¹ is —(R²)N—, and R² is not hydrogen, optionally R² is a C₁₋₆ aliphatic group, such as is methyl; or.

75. The antibody-drug conjugate according to any one of items 1, 71 and 73 to 74, wherein in formula (III-A) R¹ is —O—.

76. The antibody-drug conjugate according to any one of items 1, 71 and 73 to 75, wherein in formula (III-A) R^{3a} is a C₁₋₆ aliphatic group; and/or wherein R^{3a} is a C₁₋₆ aliphatic group, and R^{3b} is hydrogen or a C₁₋₆ aliphatic group; and/or wherein in formula (III-A) R^{3a} is a C₁₋₆ aliphatic group, and R^{3b} is a C₁₋₆ aliphatic group; and/or wherein in formula (III-A) R^{3a} is methyl, and R^{3b} is a C₁₋₆ aliphatic group; and/or wherein in formula (III-A) R^{3a} is methyl, and R^{3b} is methyl.

77. The antibody-drug conjugate according to any one of items 1, 71 and 73 to 76, wherein in formula (III-A) L¹ is —C(R^{3a})(R^{3b})—C(CH₃)₂—.

78. The antibody-drug conjugate according to item 1, wherein in formula (III-A) X is -L³-L²-, wherein in formula (III-A) L² is —C(R^{5a})(R^{5b})—, L³ is —(C(R^{7a})(R^{7b}))ₙ—, R¹ is —S— or —(R²)N—, and R² is not hydrogen.

79. The antibody-drug conjugate according to item 1, wherein in formula (III-A) X is -L³-L²-, wherein in formula (III-A) L² is —C(O)—, R¹ is —O—, L³ is —(C(R^{7a})(R^{7b}))ₙ—, and when 1 methylene unit of L³ is replaced by —NR⁸, R⁸ is not a C₁₋₆ aliphatic group substituted with —NH₂.

80. The antibody-drug conjugate according to any one of items 1 and 79, wherein in formula (III-A) 1 methylene unit of L³ is replaced by —NR⁸—, —O— or —SO—.

81. The antibody-drug conjugate according to any one of items 1 and 79 to 80, wherein in formula (III-A) R⁸ is a C₁₋₆ aliphatic group, and R⁸ is optionally substituted with hydrogen, protium, deuterium, tritium, halogen, —NO₂, —CN, —OH, —SH, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group, such as wherein R⁸ is a C₁₋₃ aliphatic group, and R⁸ is unsubstituted. 82. The antibody-drug conjugate according to item 1, wherein in formula (III-A) X is -L³-L²-, wherein in formula (III-A) when L² is —C(O)—, R¹ is —HN—, and L³ is —(C(R^{7a})(R^{7b}))ₙ—, at least 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

83. The antibody-drug conjugate according to item 1, wherein in formula (III-A) X is -L³-L²-, n is 4, and L³ is —(C(R^{7a})(R^{7b}))₄—.

84. The antibody-drug conjugate according to any one of items 1 and 83, wherein in formula (III-A) L² is —C(R^{5a})(R^{5b})—, wherein in formula (III-A) 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—, such as wherein in formula (III-A) 1 methylene unit of L² is replaced by —C(O)—, and L² is —C(O)—.

85. The antibody-drug conjugate according to any one of items 1 and 83 to 84, wherein in formula (III-A) 1 methylene unit of L³ is replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R^{a})—, —N═N—, —C═N— or —N═C—, preferably replaced by —NR⁸—, —O— or —SO—, more preferably replaced by —NR⁸—, wherein R⁸ is a C₁₋₆ aliphatic group, such as methyl.

86. The antibody-drug conjugate according to any one of items 1 and 83 to 85, wherein in formula (III-A) X is —(C(R^{7a})(R^{7b}))₂—N(CH₃)—C(R^{7a})(R^{7b})—C(O)—.

87. The antibody-drug conjugate according to any one of items 1 and 83-85, wherein in formula (III-A) 1 methylene unit of L³ is replaced by —SO—.

88. The antibody-drug conjugate according to any one of items 1 and 87, wherein in formula (III-A) X is —(C(R^{7a})(R^{7b}))₂—SO—C(R^{7a})(R^{7b})—C(O)—.

89. The antibody-drug conjugate according to item 1, wherein in formula (III-A) 1 methylene unit of L³ is replaced by —O—.

90. The antibody-drug conjugate according to any one of items 1 and 89, wherein in formula (III-A) X is —(C(R^{7a})(R^{7b}))₂—O—C(R^{7a})(R^{7b})—C(O)—.

91. The antibody-drug conjugate according to item 1, wherein in formula (III-A) R^{1a} and R^{1b} are each independently hydrogen or —N(R^{a})(R^{b}); and/or
wherein in formula (III-A) R² is a C₁₋₆ aliphatic group, such as a C₁₋₃ aliphatic group, such as R² is methyl; and/or
wherein in formula (III-A) R^{3a} and R^{3b} are each independently hydrogen or a C₁₋₆ aliphatic group, such as R^{3a} and R^{3b} are each independently a C₁₋₃ aliphatic group, such as R^{3a} and R^{3b} are methyl; and/or
wherein in formula (III-A) R^{4a} and R^{4b} are hydrogen; and/or
wherein in formula (III-A) R^{5a} and R^{5b} are hydrogen; and/or
wherein in formula (III-A) R⁶ is hydrogen; and/or
wherein in formula (III-A) R^{7a} and R^{7b} are hydrogen; and/or
wherein in formula (III-A) R⁸ is a C₁₋₆ aliphatic group, such as a C₁₋₃ aliphatic group, such as R⁸ is methyl.

92. The antibody-drug conjugate according to item 1, wherein R, R^{a} and R^{b} are hydrogen.

93. The antibody-drug conjugate according to item 1, wherein formula (III-A) is according to any one of formulas (III-A-1) to (III-A-17):

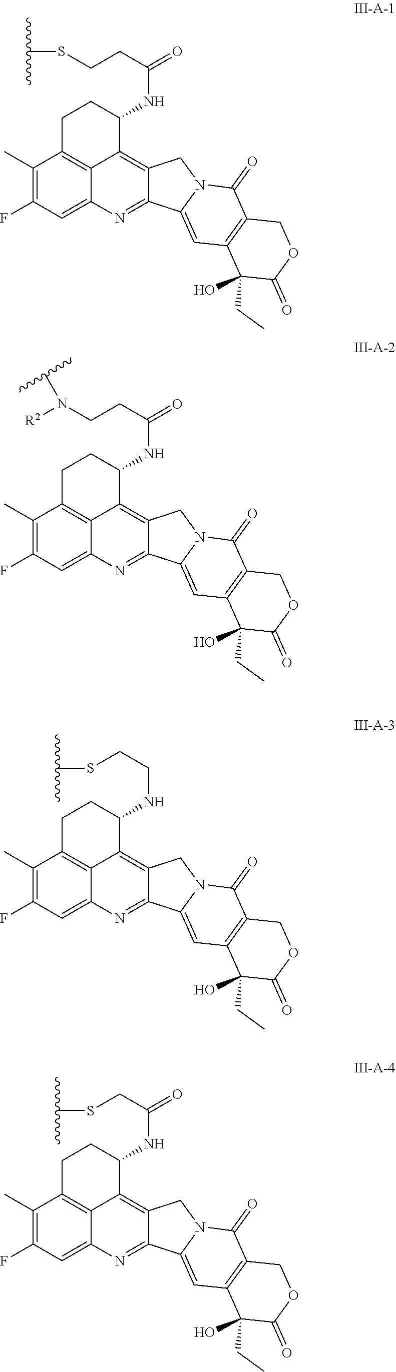

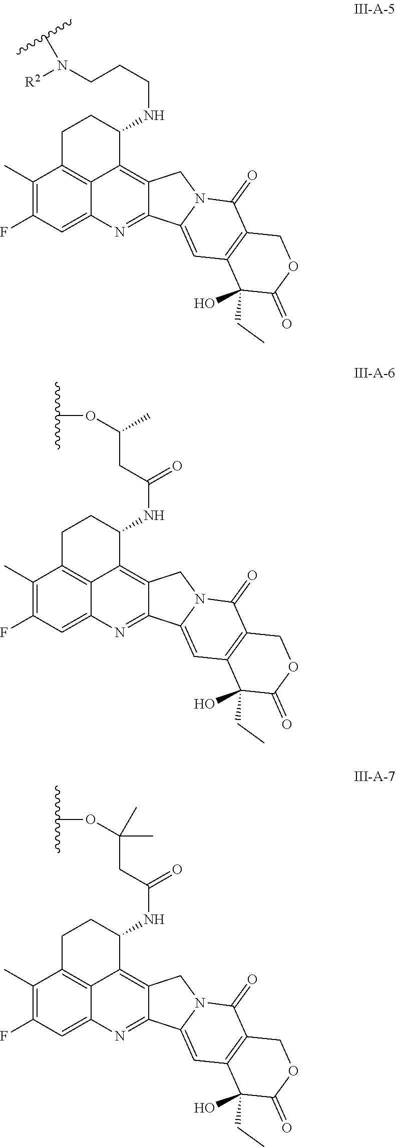

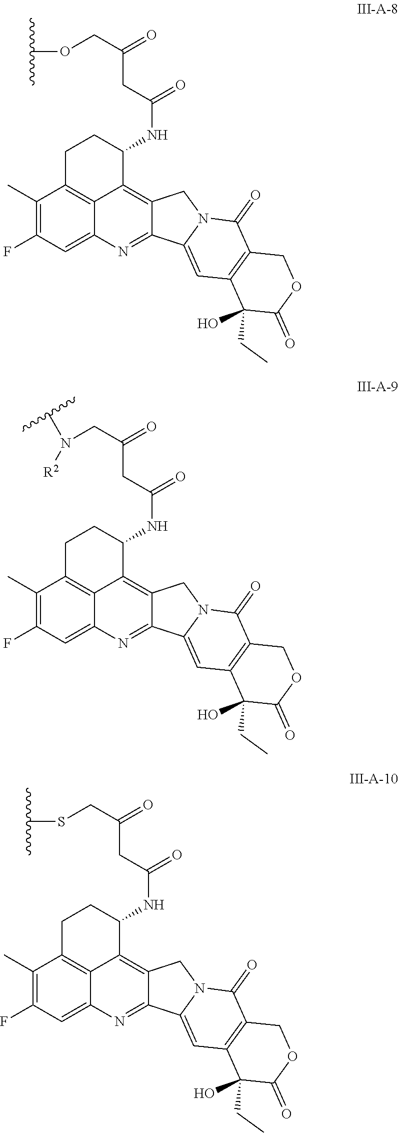

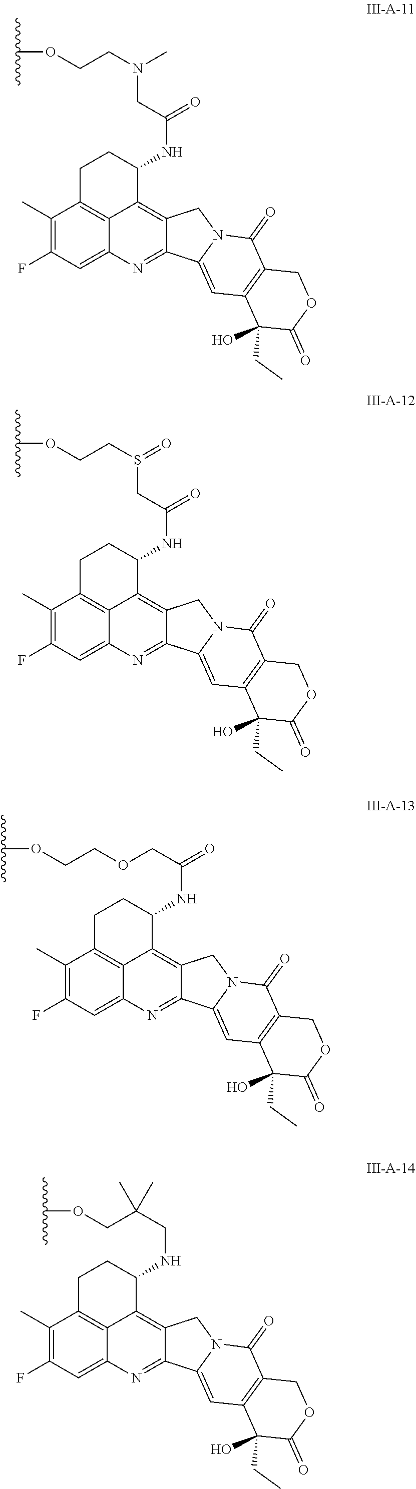 [SMILES: *OCCN(C)CC(=O)N[C@H]1CCc2c(C)c(F)cc3nc4c(c1c23)Cn1c-4cc2c(c1=O)COC(=O)[C@]2(O)CC ; *OCCS(=O)CC(=O)N[C@H]1CCc2c(C)c(F)cc3nc4c(c1c23)Cn1c-4cc2c(c1=O)COC(=O)[C@]2(O)CC ; *OCCOCC(=O)N[C@H]1CCc2c(C)c(F)cc3nc4c(c1c23)Cn1c-4cc2c(c1=O)COC(=O)[C@]2(O)CC ; *OCC(C)(C)CN[C@H]1CCc2c(C)c(F)cc3nc4c(c1c23)Cn1c-4cc2c(c1=O)COC(=O)[C@]2(O)CC]

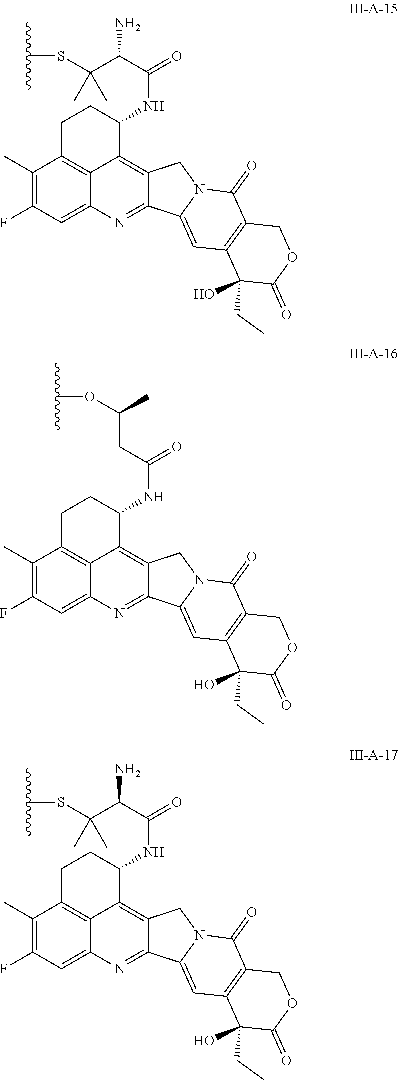 [SMILES: *SC(C)(C)[C@H](N)C(=O)N[C@H]1CCc2c(C)c(F)cc3nc4c(c1c23)Cn1c-4cc2c(c1=O)COC(=O)[C@]2(O)CC ; *O[C@@H](C)CC(=O)N[C@H]1CCc2c(C)c(F)cc3nc4c(c1c23)Cn1c-4cc2c(c1=O)COC(=O)[C@]2(O)CC ; *SC(C)(C)[C@@H](N)C(=O)N[C@H]1CCc2c(C)c(F)cc3nc4c(c1c23)Cn1c-4cc2c(c1=O)COC(=O)[C@]2(O)CC]

### III-A-17

wherein the wavy line denotes the link of the structure shown in any one of formulas (III-A-1) to (III-A-17) to the antibody, optionally via a linker.

94. The antibody-drug conjugate according to item 1, wherein the antibody-drug conjugate comprises a structure shown in formula (I-C):

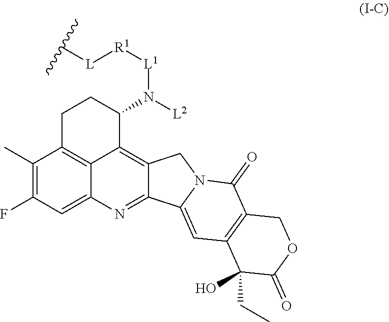

wherein L links the structure shown in formula (I-C) to the antibody, and wherein, L is -Lₐ-L_{b}-L_{c}-, each of Lₐ, L_{b}, and L_{c} having the meaning as defined in any formula (I-B) herein, and each of R¹, L¹ and L² are defined as in any formula (I-A) as described herein above.

95. The antibody-drug conjugate according to item 1, wherein the antibody-drug conjugate comprises a structure shown in formula (II-Cx) or formula (II-Cy):

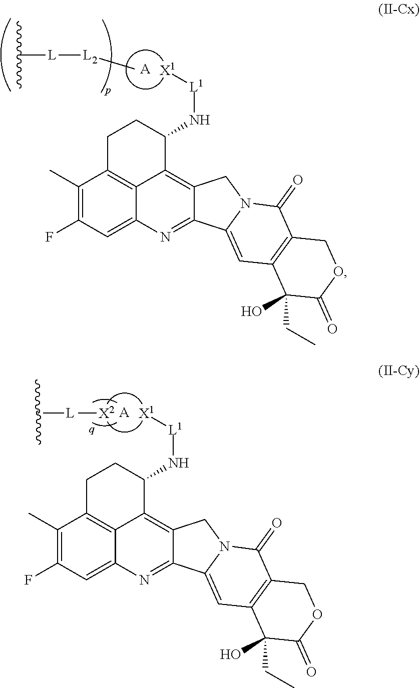

wherein, L may be -Lₐ-L_{b}-L_{c}-, and Lₐ, L_{b} and L_{c} are defined as in any formula (II-Bx) in embodiments as described herein above;
L², p, ring A, X¹ and L¹ are defined as in any formula (II-Ax) in embodiments as described herein above;
or X², q, ring A, X¹ and L¹ are defined as in any formula (II-Ay) in embodiments as described herein above.

96. The antibody-drug conjugate according to item 1, wherein the antibody-drug conjugate comprises a structure shown in formula (III-C):

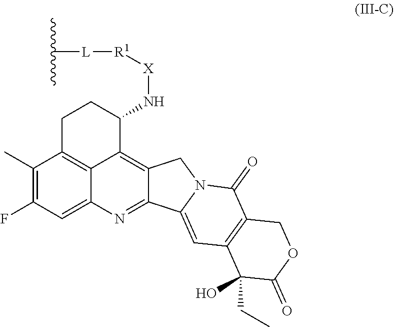

wherein, L is -Lₐ-L_{b}-L_{c}-; wherein L links the structure shown in formula (III-C) to the antibody;
Lₐ- is selected from the group consisting of:

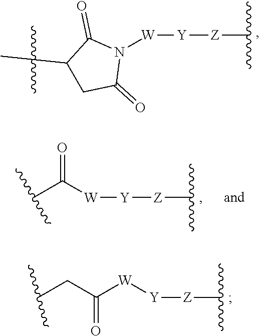

wherein W is —(C(R^{wa})(R^{wb}))_{wn}—, Y is —(OCH₂CH₂)_{yn}—O_{yp}, and Z is —(C(R^{za})(R^{zb}))_{zn};
wherein Z connects —La— to -Lb-
wherein wn is selected from the group consisting of integers≥0, and
0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers≥0, and yp is 0 or 1;
wherein zn is selected from the group consisting of integers≥0, and
0 or no less than 1 methylene unit of Z is independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
Cyr- is selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb})—SO₂N(R^{ra})(R^{rb}), —OC(O)R^{r}, —N(R)SO₂R^{r}, or a C₁₋₆ aliphatic group optionally substituted with R^{r};
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- is selected from the group consisting of:

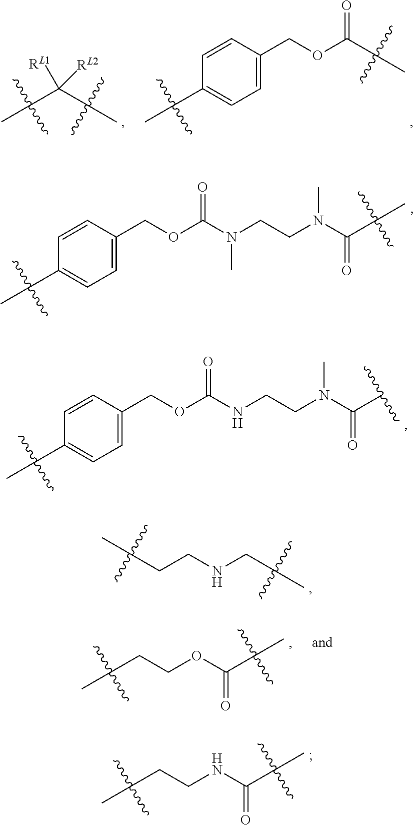

wherein R^{L1} and R^{L2} are each independently selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group;
wherein R¹ is selected from the group consisting of: —O—, —(R²)N—, —P(═O)(R²)— and —S—;
X is selected from the group consisting of: -L¹-C(R^{1a})(R^{1b})—C(O)—, -L¹-C(R^{1a})(R^{1b})—C(S)—, -L¹-L⁰- and -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is selected from the group consisting of integers≥0, and n is selected from the group consisting of integers≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

97. The antibody-drug conjugate according to item 1, wherein the antibody-drug conjugate comprises a structure shown in formula (I-D):

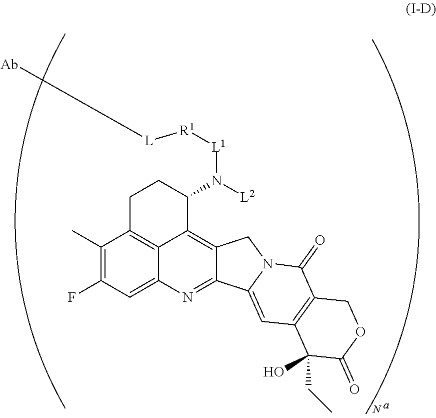

wherein, Ab is the antibody which binds to uPARAP comprising:
   i) an immunoglobulin light chain variable region comprising or consisting of the amino acid sequence of SEQ ID NO: 3; and
   ii) an immunoglobulin heavy chain variable region comprising or consisting of the amino acid sequence of SEQ ID NO: 6;
N^{a} is an integer or a decimal from 1 to 10;
L may be -Lₐ-L_{b}-L_{c}-, and Lₐ, L_{b} and L_{c} are defined as in any formula (I-B) in embodiments described herein above,
R¹, L¹ and L² are defined as in any formula (I-A) in embodiments described herein above.

98. The antibody-drug conjugate according to item 1, wherein the antibody-drug conjugate comprises a structure shown in formula (II-Dx) or formula (II—Dy):

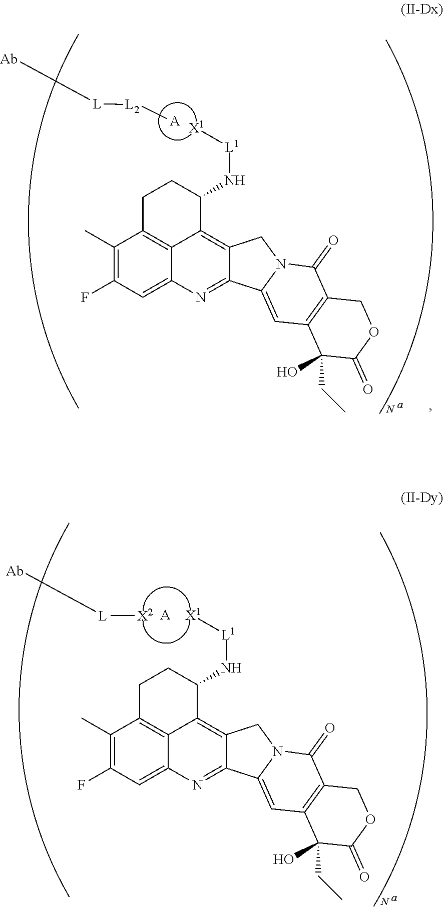

wherein, Ab is the antibody which binds to uPARAP comprising:
   i) an immunoglobulin light chain variable region comprising or consisting of the amino acid sequence of SEQ ID NO: 3; and
   ii) an immunoglobulin heavy chain variable region comprising or consisting of the amino acid sequence of SEQ ID NO: 6;
N^{a} is an integer or a decimal from 1 to 10;
L may be -Lₐ-L_{b}-L_{c}-, and Lₐ, L_{b} and L_{c} are defined as in any formula (II-Bx) or formula (II-By) in embodiments described herein above;
L², ring A, X¹ and L¹ are defined as in any formula (II-Ax) in embodiments as described herein above;
or X², ring A, X¹ and L¹ are defined as in any formula (II-Ay) in embodiments as described herein above.

99. The antibody-drug conjugate according to item 1, wherein the antibody-drug conjugate comprises a structure shown in formula (III-D):

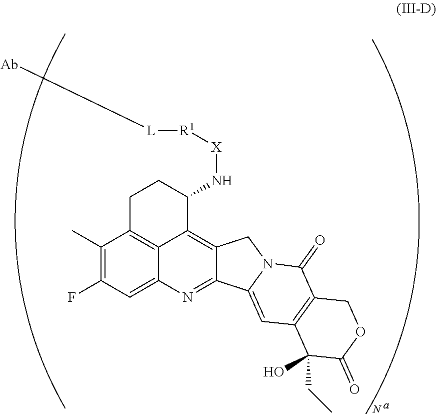

wherein, Ab is the antibody which binds to uPARAP comprising:
   i) an immunoglobulin light chain variable region comprising or consisting of the amino acid sequence of SEQ ID NO: 3; and
   ii) an immunoglobulin heavy chain variable region comprising or consisting of the amino acid sequence of SEQ ID NO: 6;
N^{a} is an integer or a decimal from 1 to 10;
L is -Lₐ-L_{b}-L_{c}-; wherein L links the structure shown in formula (III-D) to the antibody;
-Lₐ- is selected from the group consisting of:

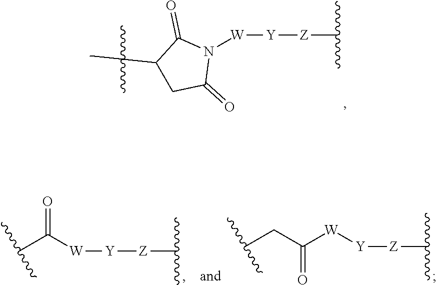

wherein W is —(C(R^{wa})(R^{wb}))_{wn}—, Y is —(OCH₂CH₂)_{yn}—O_{yp}, and Z is —(C(R^{za})(R^{zb}))_{z}n;
wherein Z connects —La— to -Lb-
wherein wn is selected from the group consisting of integers≥0, and
0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers≥0, and yp is 0 or 1;
wherein zn is selected from the group consisting of integers≥0, and
0 or no less than 1 methylene unit of Z is independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- is selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R¹;
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- is selected from the group consisting of:

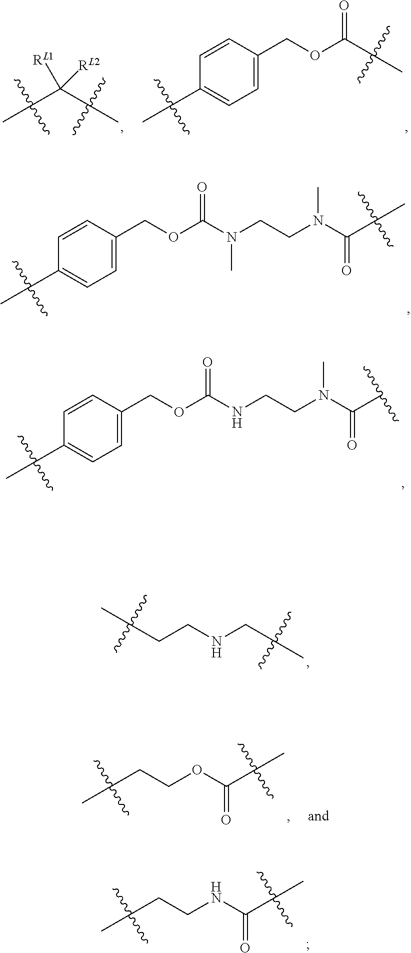

wherein R^{L1} and R^{L2} are each independently selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group;
wherein R¹ is selected from the group consisting of: —O—, —(R²)N—, —P(═O)(R²)— and —S—;
X is selected from the group consisting of: -L¹-C(R^{1a})(R^{1b})—C(O)—, -L¹-C(R^{1a})(R^{1b})—C(S)—, -L¹-L⁰- and -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is selected from the group consisting of integers≥0, and n is selected from the group consisting of integers≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

100. The antibody drug conjugate according to any one of items 1 and 94 to 99, wherein the antibody drug conjugate is according to any one of the structures shown in Table 2 or a tautomer, a mesomer, a racemate, an enantiomer or a diastereoisomer thereof, or a mixture thereof, or a pharmaceutically acceptable salt thereof.

101. An antibody-drug conjugate (ADC) comprising:
a. an antibody which binds to uPARAP comprising:
   i. an immunoglobulin light chain variable region comprising or consisting of the amino acid sequence of SEQ ID NO: 3; and
   ii. an immunoglobulin heavy chain variable region comprising or consisting of the amino acid sequence of SEQ ID NO: 6;
b. an active agent, selected from the group consisting of:
   i. formula (I-E) or a tautomer, a mesomer, a racemate, an enantiomer or a diastereoisomer thereof, or a mixture thereof, or a pharmaceutically acceptable salt thereof,

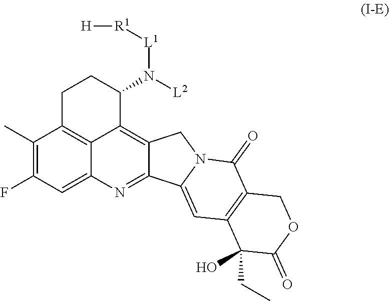

wherein R¹, L¹ and L² are defined as in any formula (I-A) in embodiments defined herein above;
ii. Formula (II-Ex) or Formula (II-Ey) or a tautomer, a mesomer, a racemate, an enantiomer or a diastereoisomer thereof, or a mixture thereof, or a pharmaceutically acceptable salt thereof

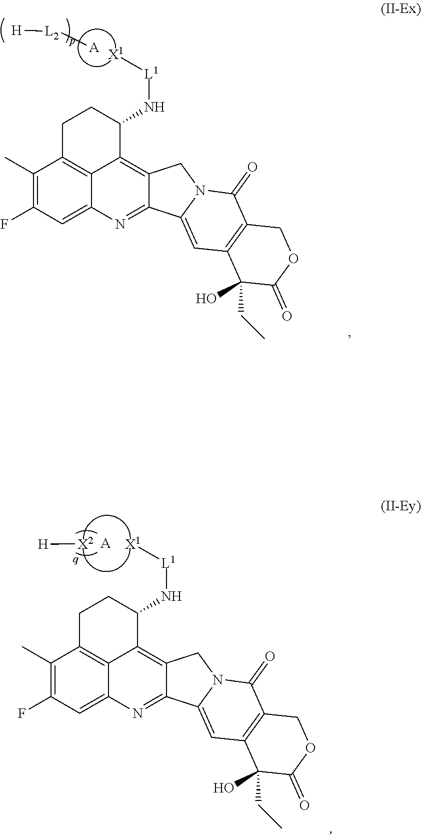

wherein for formula (II-Ex), L², p, ring A, X¹ and L¹ are defined as in any formula (II-Ax) in embodiments described herein above, and for formula (II-Ey) X², q, ring A, X¹ and L¹ are defined as in any formula (II-Ay) in embodiments defined herein above; and
iii. formula (III-E) or a tautomer, a mesomer, a racemate, an enantiomer or a diastereoisomer thereof, or a mixture thereof, or a pharmaceutically acceptable salt thereof

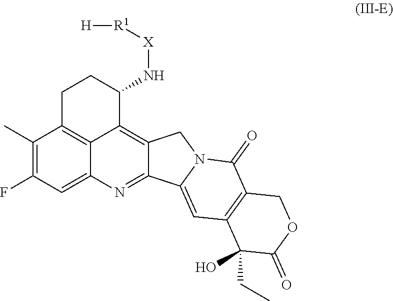

wherein R¹ is selected from the group consisting of: —O—, —(R²)N—, —P(═O)(R²)— and —S—;
X is selected from the group consisting of: -L¹-C(R^{1a})(R^{1b})—C(O)—, -L¹-C(R^{1a})(R^{1b})—C(S)—, -L¹-L⁰- and -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is selected from the group consisting of integers≥0, and n is selected from the group consisting of integers≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—;
and
c. optionally a linker which links a) to b).

102. The antibody drug conjugate according to any one of items 1 or 101, wherein the active agent is selected from any one of the structures in Table 1 or a tautomer, a mesomer, a racemate, an enantiomer or a diastereoisomer thereof, or a mixture thereof, or a pharmaceutically acceptable salt thereof.

103. A method for manufacturing the antibody-drug conjugate according to any one of the preceding items, the method comprising reacting a compound according to one selected from the group consisting of:
a. Formula (I-F) or a tautomer, a mesomer, a racemate, an enantiomer or a diastereoisomer thereof, or a mixture thereof, or a pharmaceutically acceptable salt thereof,

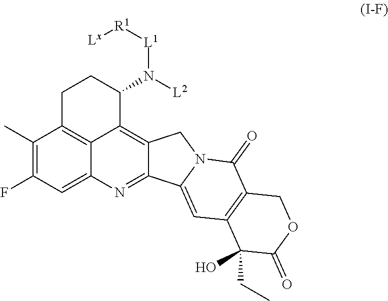

wherein, L^{x} is Lₐₓ-L_{b}-L_{c}-;
Lₐₓ- is selected from the group consisting of:

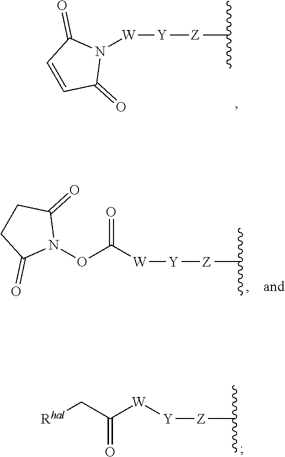

wherein R^{hal} is iodine or bromine
wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is selected from the group consisting of integers≥0, and
0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers≥0, and yp is 0 or 1;
wherein zn is selected from the group consisting of integers≥0, and
0 or no less than 1 methylene unit of Z is independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- is selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R^{r};
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
L^{b} and L^{c} are defined as in any formula (I-B) in embodiments described herein above;
R¹, L¹ and L² are defined as in any formula (I-A) in embodiments described herein above;
b. formula (II-Fx) or (II-Fy), or a tautomer, a mesomer, a racemate, an enantiomer or a diastereoisomer thereof, or a mixture thereof, or a pharmaceutically acceptable salt thereof,

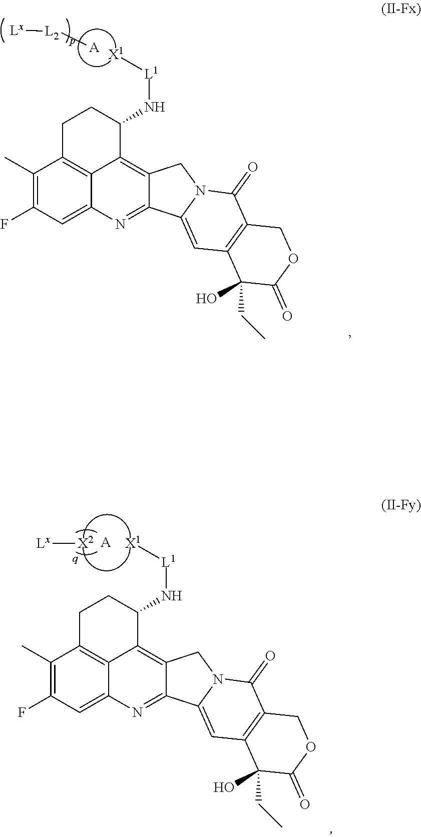

wherein, L^{x} is Lₐₓ-L_{b}-L_{c}-;
Lₐₓ- is selected from the group consisting of:

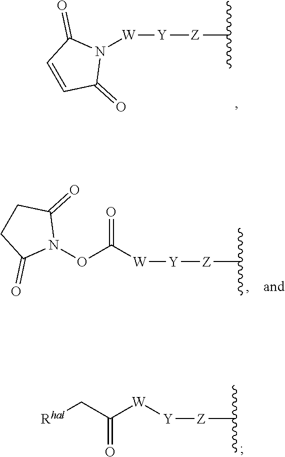

wherein R^{hal} is iodine or bromine;
wherein W is —(C(R^{wa})(R^{wb}))_{wn}—, Y is —(OCH₂CH₂)_{yn}—O_{yp}—, and Z is —(C(R^{za})(R^{zb}))_{zn};
wherein wn is selected from the group consisting of integers≥0, and
0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers≥0, and yp is 0 or 1;
wherein zn is selected from the group consisting of integers≥0, and 0 or no less than 1 methylene unit of Z is independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- is selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R^{r};
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
L^{b} and L^{c} are defined as in any formula (II-Bx) or formula (II-By) in embodiments as described herein above;
wherein L², p, ring A, X¹ and L¹ are defined as in any formula (II-Ax) in embodiments as described herein above;
or X², q, ring A, X¹ and L¹ are defined as in any formula (II-Ay) in embodiments as described herein above; and
c. formula (III-F) or a tautomer, a mesomer, a racemate, an enantiomer or a diastereoisomer thereof, or a mixture thereof, or a pharmaceutically acceptable salt thereof,

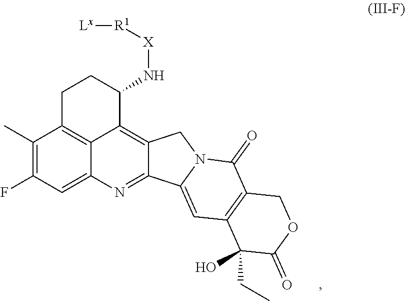

wherein, L^{x} is Lax-L_{b}-L_{c}-;
Lₐₓ- is selected from the group consisting of:

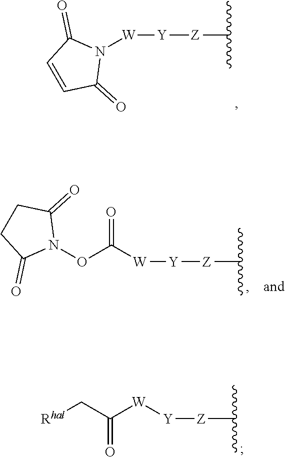

wherein R^{hal} is iodine or bromine;
wherein W is —(C(R^{wa})(R^{wb}))_{wn}—, Y is —(OCH₂CH₂)_{yn}—O_{yp}, and Z is —(C(R^{za})(R^{zb}))_{zn};
wherein wn is selected from the group consisting of integers≥0, and 0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is selected from the group consisting of integers≥0, and yp is 0 or 1;
wherein zn is selected from the group consisting of integers≥0, and 0 or no less than 1 methylene unit of Z is independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- is selected from the group consisting of: 6-10 membered arylene, 5-8 membered heteroarylene, 3-10 membered heterocyclylene, and 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR^{r}, —SR^{r}, —N(R^{ra})(R^{rb}), —C(O)R^{r}, —CO₂R^{r}, —C(O)C(O)R^{r}, —C(O)CH₂C(O)R^{r}, —S(O)R^{r}, —S(O)₂R^{r}, —C(O)N(R^{ra})(R^{rb}), —SO₂N(R^{ra})(R^{rb}), —OC(O)R^{r}, —N(R)SO₂R^{r}, or a C₁₋₆ aliphatic group optionally substituted with R^{r};
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- is selected from the group consisting of:

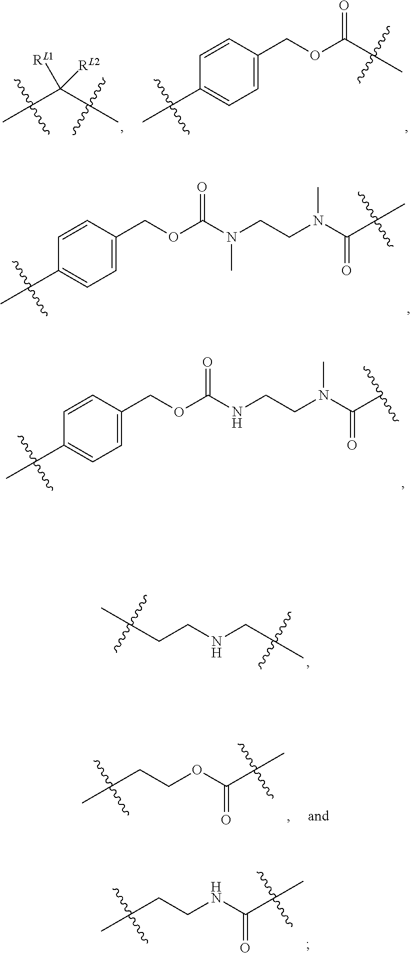

wherein R^{L1} and R^{L2} are each independently selected from the group consisting of: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H and a C₁₋₆ aliphatic group;
wherein R¹ is selected from the group consisting of: —O—, —(R²)N—, —P(═O)(R²)— and —S—;
X is selected from the group consisting of: -L¹-C(R^{1a})(R^{1b})—C(O)—, -L¹-C(R^{1a})(R^{1b})—C(S)—, -L¹-L⁰- and -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—; wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is selected from the group consisting of integers≥0, and n is selected from the group consisting of integers≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR^{B}, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—; with the antibody as defined in item 1.

104. The method according to item 103, wherein Lₐₓ- is

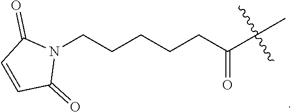 [SMILES: *C(=O)CCCCCN1C(=O)C=CC1=O]

105. The method according to any one of items 103 to 104, wherein Lₐₓ-L_{b}-L_{c}- is selected from the group consisting of:

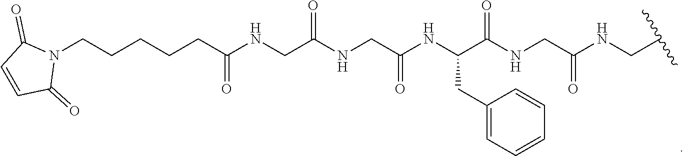 [SMILES: *CNC(=O)CNC(=O)[C@H](Cc1ccccc1)NC(=O)CNC(=O)CNC(=O)CCCCCN1C(=O)C=CC1=O]

106. The method according to any one of to items 103 to 105, wherein the compound is selected from any one of the structures in Table 3 or a tautomer, a mesomer, a racemate, an enantiomer or a diastereoisomer thereof, or a mixture thereof, or a pharmaceutically acceptable salt thereof.

107. The antibody-drug conjugate according to any one of items 1 to 106, wherein the antibody comprises:
a. an immunoglobulin light chain comprising the amino acid sequence of SEQ ID NO: 1; and
b. an immunoglobulin heavy chain comprising the amino acid sequence of SEQ ID NO: 4.

108. The antibody-drug conjugate according to any one of items 1 to 107, wherein the antibody comprises:
a. an immunoglobulin light chain consisting of the amino acid sequence of SEQ ID NO: 1; and
b. an immunoglobulin heavy chain consisting of the amino acid sequence of SEQ ID NO:4.

1-51. (canceled)

52. An antibody-drug conjugate (ADC) comprising an antibody which binds to uPARAP comprising:
i. an immunoglobulin light chain variable region comprising or consisting of the amino acid sequence of SEQ ID NO: 3; and
ii. an immunoglobulin heavy chain variable region comprising or consisting of the amino acid sequence of SEQ ID NO: 6;
wherein the antibody-drug conjugate comprises an active agent with a structure according to:
a. Formula (III-A) or a tautomer, a mesomer, a racemate, an enantiomer or a diastereoisomer thereof, or a mixture thereof, or a pharmaceutically acceptable salt thereof 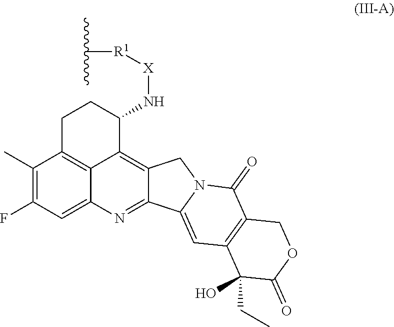
wherein R¹ is: —O—, —(R²)N—, —P(═O)(R²)— or —S—; wherein R¹ links the structure according to formula (III-A) to the antibody, optionally via a linker;
X is: -L¹-C(R^{1a})(R^{1b})—C(O)—, -L¹-C(R^{1a})(R^{1b})—C(S)—, -L¹-L⁰- or -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is an integer≥0, and n is an integer≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—;
b. formula (I-A) or a tautomer, a mesomer, a racemate, an enantiomer or a diastereoisomer thereof, or a mixture thereof, or a pharmaceutically acceptable salt thereof, 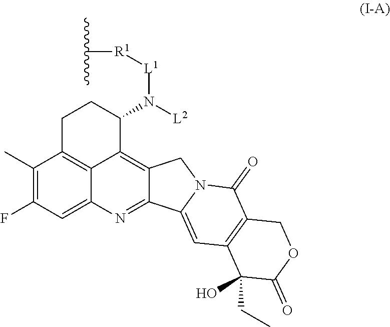
wherein, R¹ is: —O—, —(R²)N—, —P(═O)(R²)—, —P(R²)— or —S—;
L² is —(C(R^{3a})(R^{3b}))ₘ—R,
wherein 0 or no less than 1 methylene unit of L² is independently replaced by -Cy-, —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
L¹ is —(C(R^{5a})(R^{5b}))ₙ—,
wherein 0 or no less than 1 methylene unit of L¹ is independently replaced by -Cy-, —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
-Cy- is: 6-10 membered arylene, 5-8 membered heteroarylene, 3-10 membered heterocyclylene, or 3-10 membered saturated or partially unsaturated carbocyclylene, wherein -Cy- is unsubstituted or independently substituted with no less than 1 substituent R⁷;
wherein each R^{3a}, each R^{3b}, each R⁴, each R^{5a}, each R^{5b} and each R⁶ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R; or, R^{3a} and R^{5a}, R⁴ and R^{5a}, R^{3a} and R⁶ or R⁴ and R⁶ each independently optionally form a ring B together with an atom therebetween, wherein the ring B is: 5-8 membered heteroarylene or 3-10 membered saturated or partially unsaturated heterocyclylene, and the ring B is unsubstituted or independently substituted with no less than 1 substituent R⁸;
wherein each R², each R⁷ and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n are each independently integers≥1; and wherein R¹ links the structure shown in formula (I-A) to the antibody, optionally via a linker; or
c. Formula (II-A) or a tautomer, a mesomer, a racemate, an enantiomer or a diastereoisomer thereof, or a mixture thereof, or a pharmaceutically acceptable salt thereof 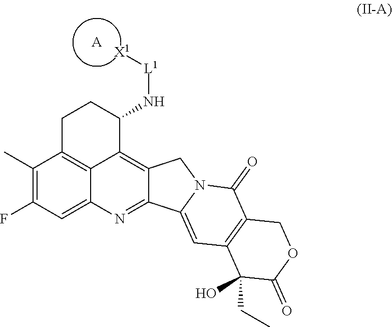
wherein, X¹ is: N, P, or saturated or unsaturated C; when X¹ is saturated C, X¹ is substituted with Rⁿ;
wherein ring A links the structure according to formula II-A to the antibody, optionally via a linker;
when X¹ is saturated C, ring A is: 3-10 membered saturated or partially unsaturated heterocyclyl, or 3-10 membered saturated or partially unsaturated carbocyclyl, wherein ring A is substituted with 0 or no less than 1 substituent R^{1a};
or, when X¹ is unsaturated C, ring A is: 6-10 membered aryl, 5-8 membered heteroaryl, 3-10 membered partially unsaturated heterocyclyl, or 3-10 membered partially unsaturated carbocyclyl, wherein ring A is substituted with 0 or no less than 1 substituent R^{1b};
or, when X¹ is N or P, ring A is: 5-8 membered heteroaryl or 3-10 membered saturated or partially unsaturated heterocyclyl, wherein ring A is substituted with 0 or no less than 1 substituent Rⁿ;
when ring A is: 6-10 membered aryl, 5-8 membered heteroaryl, or 3-10 membered saturated or partially unsaturated carbocyclyl, ring A is substituted with p L², wherein L² is not Rⁿ;
or, when ring A is 3-10 membered saturated or partially unsaturated heterocyclyl, ring A is substituted with p L², or ring A comprises q ring-forming heteroatom X², and X² is used for linking formula (II-A) to the antibody, optionally via a linker;
X² is: N or P;
L² is —R²-L³-, and R² is used for linking formula (II-A) to the antibody, optionally via a linker;
L³ is —(C(R^{3a})(R^{3b}))ₘ—, wherein when L³ comprises a methylene unit, 0 or no less than 1 methylene unit of L³ is independently replaced by —N(R⁴)C(O)—, —C(O)N(R⁴)—, —C(O)—, —OC(O)—, —C(O)O—, —NR⁴—, —O—, —S—, —SO—, —SO₂—, —P(R⁴)—, —P(═O)(R⁴)—, —N(R⁴)SO₂—, —SO₂N(R⁴)—, —C(═S)—, —C(═NR⁴)—, —N═N—, —C═N—, —N═C— or —C(═N₂)—;
R² is: —O—, —(R^{2a})N—, —S— or —P(═O)(R^{2a})—;
L¹ is —(C(R^{5a})(R^{5b}))ₙ—, wherein when L¹ comprises a methylene unit, 0 or no less than 1 methylene unit of L¹ is independently replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
wherein each R^{1a}, each R^{1b}, each R^{1c}, each R^{2a}, each R^{3a}, each R^{3b}, each R⁴, each R^{5a}, each R^{5b}, each R⁶ and each Rⁿ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m and n are each independently integers≥0, and p and q are each independently integers≥1.

53. The antibody-drug conjugate according to claim 52, wherein the antibody-drug conjugate comprises an active agent with a structure according to formula (III-A) or a tautomer, a mesomer, a racemate, an enantiomer or a diastereoisomer thereof, or a mixture thereof, or a pharmaceutically acceptable salt thereof.

54. The antibody-drug conjugate according to claim 52, wherein in formula (III-A) L¹ is —(C(R^{3a})(R^{3b}))ₘ—, and 0 or 1 methylene unit of L¹ is replaced by —C(O)—, —C(═S)—, —C(═NR^{4b})— or —C(═N₂)—.

55. The antibody-drug conjugate according to claim 52, wherein in formula (III-A) X is -L¹-C(R^{1a})(R^{1b})—C(O)—, R¹ is —O— or —HN—, L¹ is —(C(R^{3a})(R^{3b}))ₘ—, m is not 0, 0 methylene units of L¹ are replaced by —C(O)—, —C(═S)—, —C(═NR^{4b})— or —C(═N₂)—, each R^{3a} and each R^{3b} are not both hydrogen, and R^{1a} and R^{1b} are hydrogen.

56. The antibody-drug conjugate according to claim 55, wherein in formula (III-A) m is 1, and L¹ is —C(R^{3a})(R^{3b})—.

57. The antibody-drug conjugate according to claim 55, wherein in formula (III-A) R¹ is —O—.

58. The antibody-drug conjugate according to claim 52, wherein in formula (III-A) L¹ is —C(R^{3a})(R^{3b})—, R¹ is —O— or —HN—, 0 methylene units of L¹ are replaced by —C(O)—, —C(═S)—, —C(═NR^{4b})— or —C(═N₂)—, and R^{3a} and R^{3b} are not both hydrogen.

59. The antibody-drug conjugate according to claim 52, wherein in formula (III-A) R^{3a} is a C₁₋₆ aliphatic group; or R^{3a} is a C₁₋₆ aliphatic group, and R^{3b} is hydrogen or a C₁₋₆ aliphatic group; or R^{3a} is a C₁₋₆ aliphatic group, and R^{3b} is hydrogen; or R^{3a} is methyl, and R^{3b} is hydrogen.

60. The antibody-drug conjugate according to claim 52, wherein formula (III-A) is 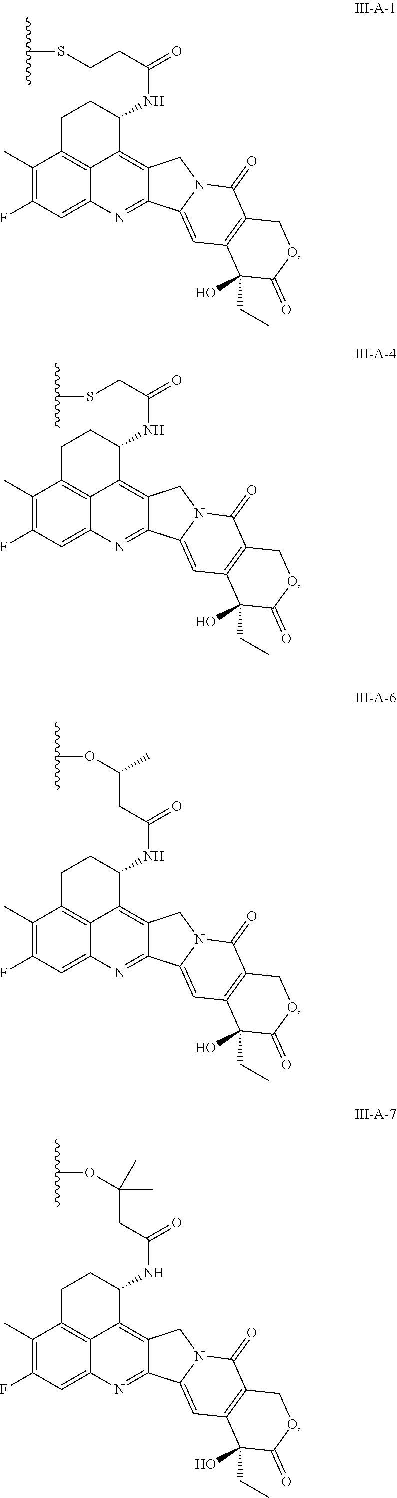 [SMILES: *SCCC(=O)N[C@H]1CCc2c(C)c(F)cc3nc4c(c1c23)Cn1c-4cc2c(c1=O)COC(=O)[C@]2(O)CC ; *SCC(=O)N[C@H]1CCc2c(C)c(F)cc3nc4c(c1c23)Cn1c-4cc2c(c1=O)COC(=O)[C@]2(O)CC ; *O[C@H](C)CC(=O)N[C@H]1CCc2c(C)c(F)cc3nc4c(c1c23)Cn1c-4cc2c(c1=O)COC(=O)[C@]2(O)CC ; *OC(C)(C)CC(=O)N[C@H]1CCc2c(C)c(F)cc3nc4c(c1c23)Cn1c-4cc2c(c1=O)COC(=O)[C@]2(O)CC] 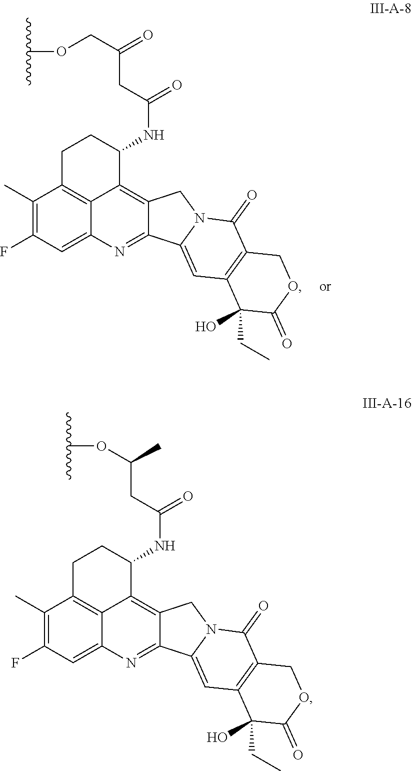 [SMILES: *OCC(=O)CC(=O)N[C@H]1CCc2c(C)c(F)cc3nc4c(c1c23)Cn1c-4cc2c(c1=O)COC(=O)[C@]2(O)CC ; *O[C@@H](C)CC(=O)N[C@H]1CCc2c(C)c(F)cc3nc4c(c1c23)Cn1c-4cc2c(c1=O)COC(=O)[C@]2(O)CC]
wherein the wavy line denotes the link of the structure shown in any one of formulas (III-A) to the antibody, optionally via a linker.

61. The antibody-drug conjugate according to claim 52, wherein formula (I-A) is according to any one of formulas (I-A-1) to (I-A-17): 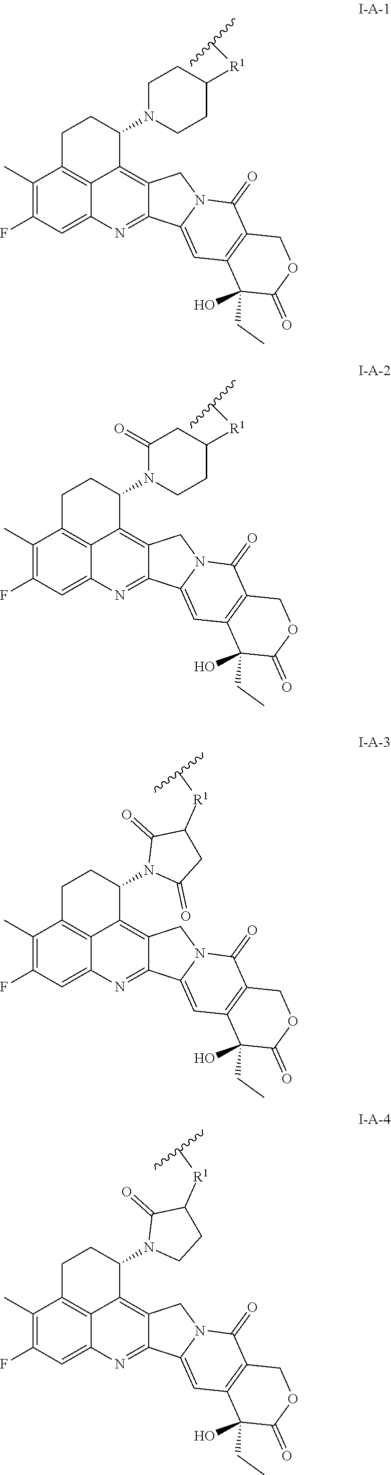 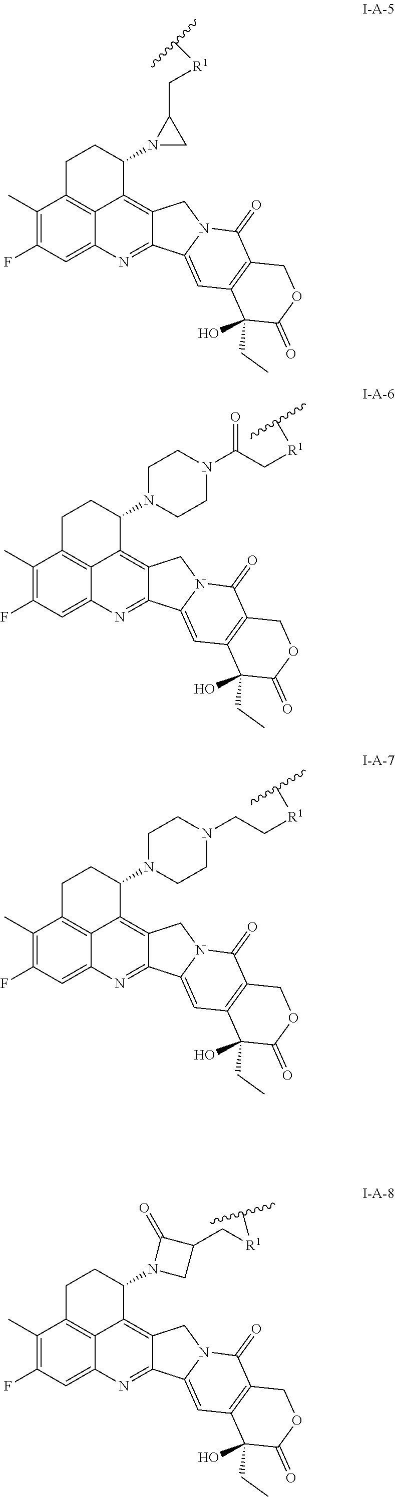 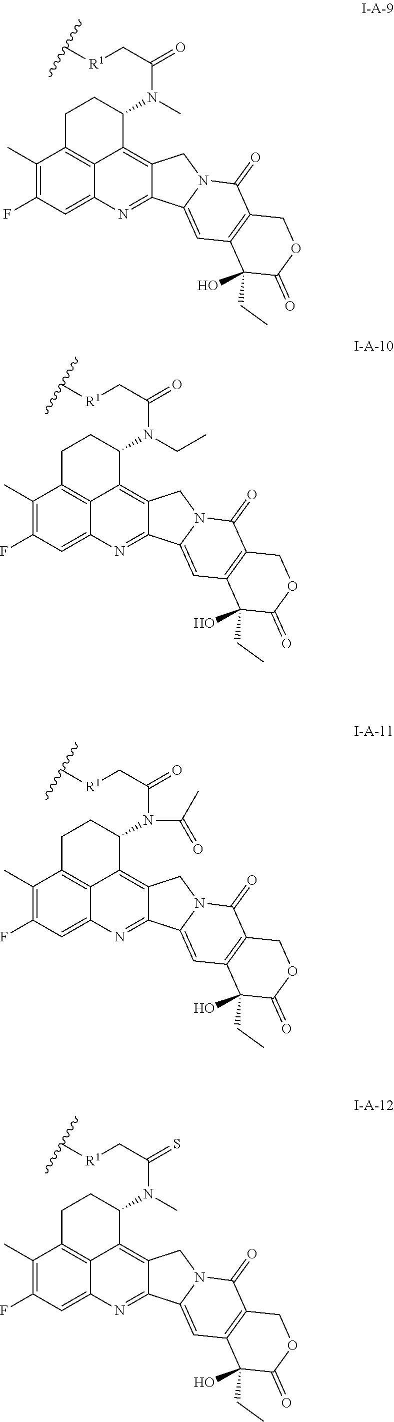 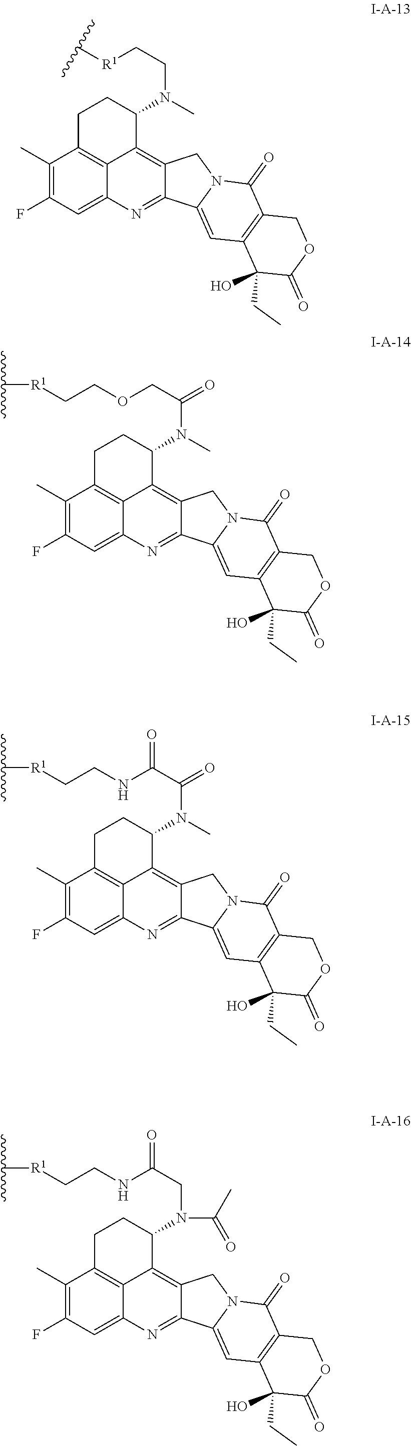 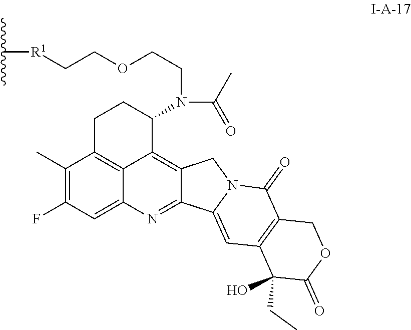
wherein, R¹ is: —O—, —HN—, —P(═O)H— or —S— and wherein R¹ links the structure shown in any one of formulas (I-A-1) to (I-A-17) to the antibody, optionally via a linker.

62. The antibody-drug conjugate according to claim 52, wherein formula (II-A) is according to any one of formulas (II-A-1) to (II-A-12): 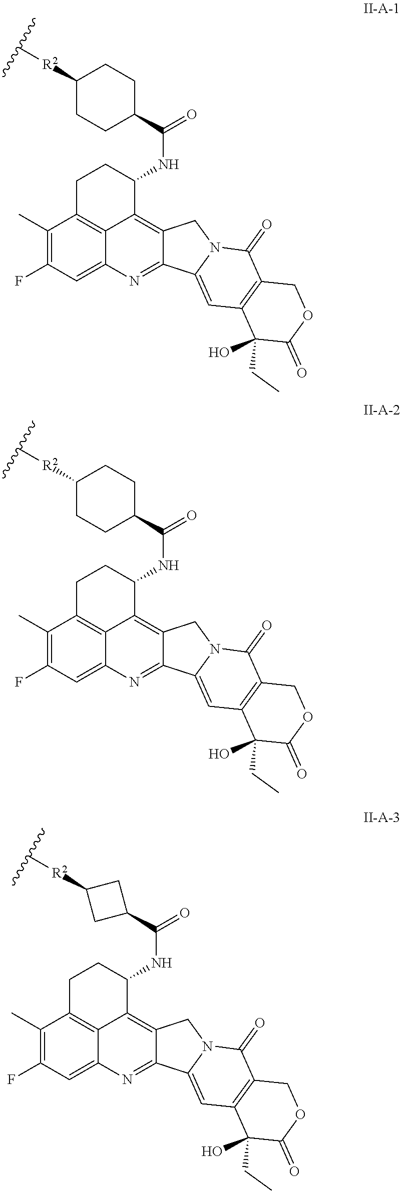 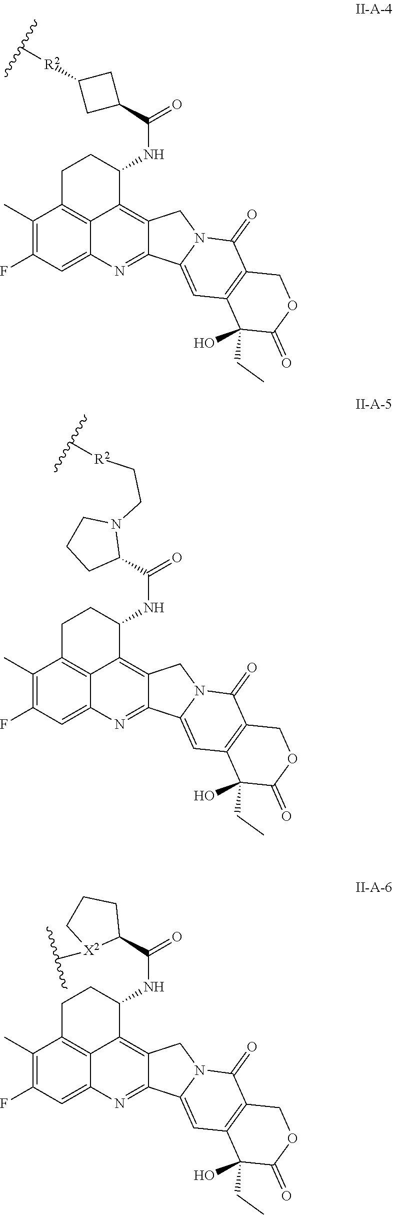 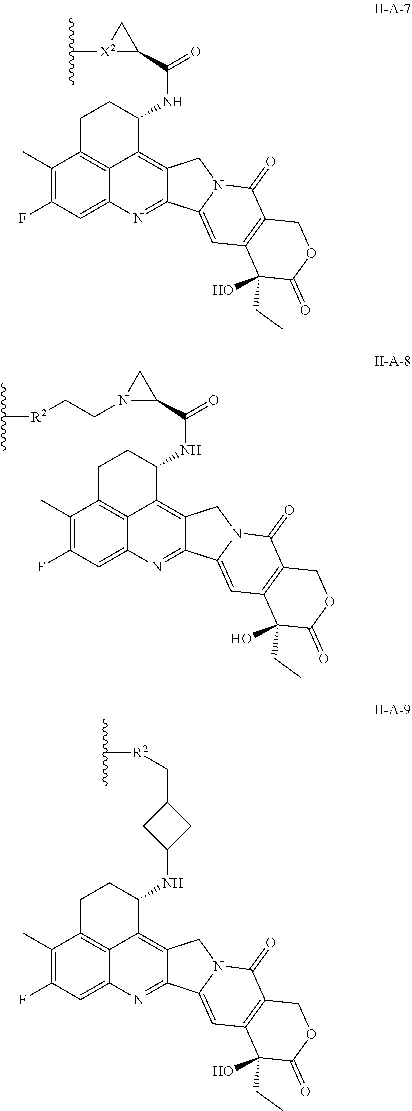 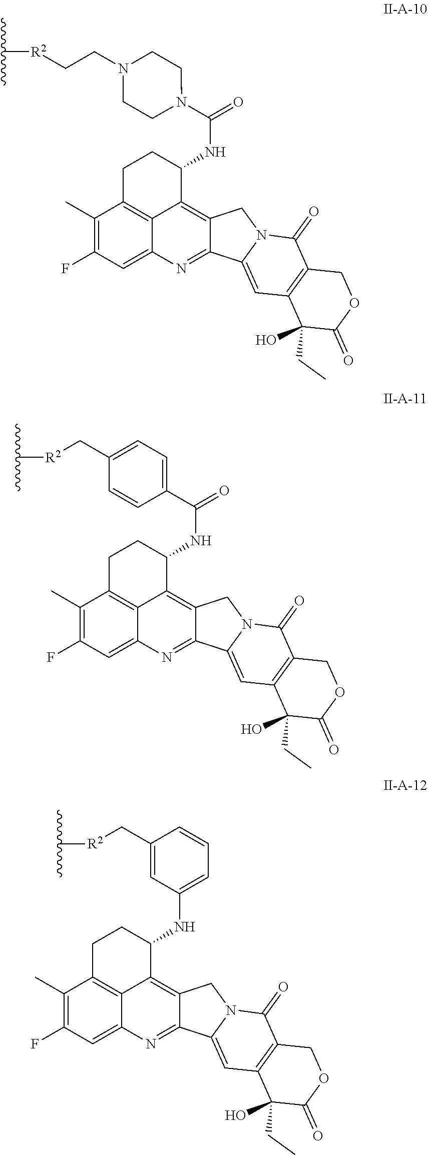
wherein, R² is: —O—, —HN—, —P(═O)H— or —S—; X² is N or P; wherein R² or X² links any one the structures shown in formulas (II-A-1) to (II-A-12) to the antibody either directly or through a linker.

63. The antibody-drug conjugate according to claim 52, wherein the antibody-drug conjugate comprises a structure shown in formula (III-C): 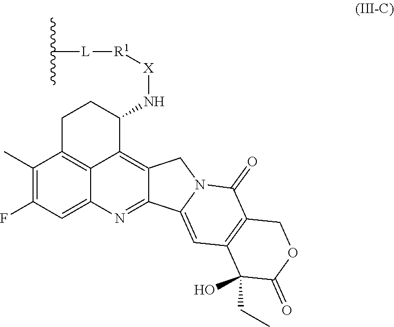
wherein, L is -Lₐ-L_{b}-L_{c}; wherein L links the structure shown in formula (III-C) to the antibody;
-Lₐ- is: 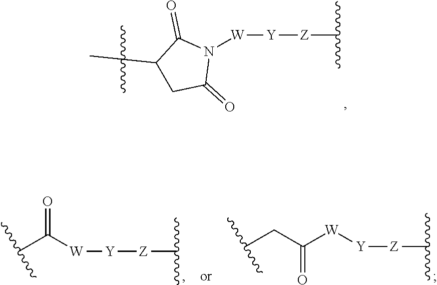
wherein W is —(C(R^{wa})(R^{b}))_{wn}—, Y is —(OCH₂CH₂)_{yn}—O_{yp}, and Z is —(C(R^{za})(R^{zb}))_{zn}; wherein
Z connects —La— to -Lb-
wherein wn is an integer≥0, and
0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is an integer≥0, and yp is 0 or 1;
wherein zn is an integer≥0, and
0 or no less than 1 methylene unit of Z is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
-Cyr- is: 6-10 membered arylene, 5-8 membered heteroarylene, 3-10 membered heterocyclylene, or 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR, —SR, —N(R^{ra})(R^{rb}), —C(O)R, —CO₂R, —C(O)C(O)R, —C(O)CH₂C(O)R, —S(O)R, —S(O)₂R, —C(O)N(R^{ra})(R^{rb}), —SO₂N(R^{ra})(R^{rb}), —OC(O)R, —N(R)SO₂R, or a C₁₋₆ aliphatic group optionally substituted with R;
wherein each R^{r}, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- is: 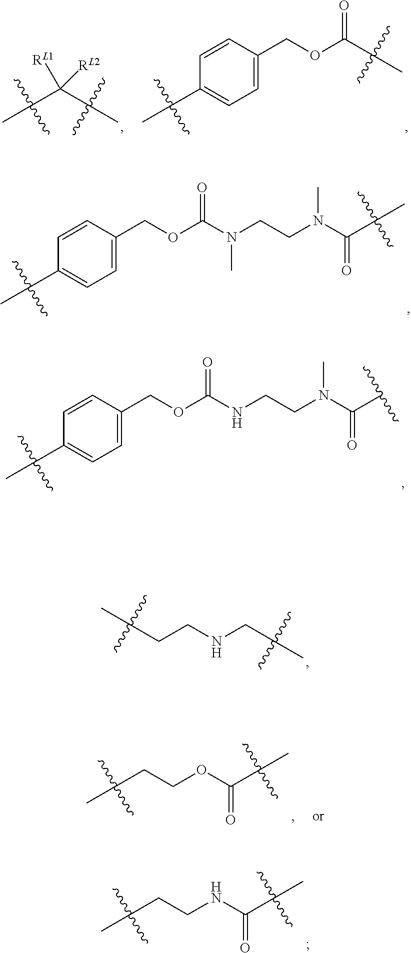
wherein R^{L1} and R^{L2} are each independently: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
wherein R¹ is: —O—, —(R²)N—, —P(═O)(R²)— or —S—;
X is: -L¹-C(R^{1a})(R^{1b})—C(O)—, -L¹-C(R^{1a})(R^{1b})—C(S)—, -L¹-L⁰- or -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R^{b}), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is an integer≥0, and n is an integer≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

64. The antibody-drug conjugate according to claim 63, wherein -Lₐ-L_{b}-L_{c}- is

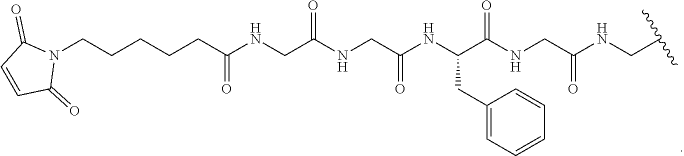 [SMILES: *CNC(=O)CNC(=O)[C@H](Cc1ccccc1)NC(=O)CNC(=O)CNC(=O)CCCCCN1C(=O)C=CC1=O]

65. The antibody-drug conjugate according to claim 52, wherein the antibody-drug conjugate comprises a structure shown in formula (III-D): 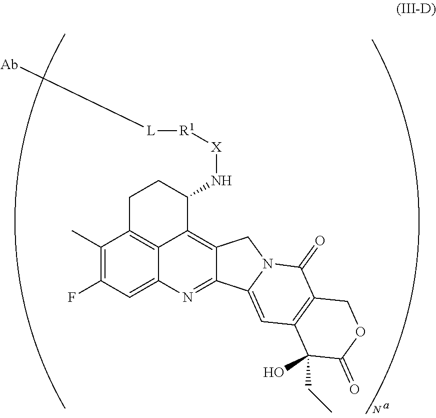
wherein, Ab is the antibody which binds to uPARAP comprising:
i) an immunoglobulin light chain variable region comprising or consisting of the amino acid sequence of SEQ ID NO: 3; and
ii) an immunoglobulin heavy chain variable region comprising or consisting of the amino acid sequence of SEQ ID NO: 6;
N^{a} is an integer or a decimal from 1 to 10;
L is -Lₐ-L_{b}-L_{c}-; wherein L links the structure shown in formula (III-D) to the antibody;
-Lₐ- is: 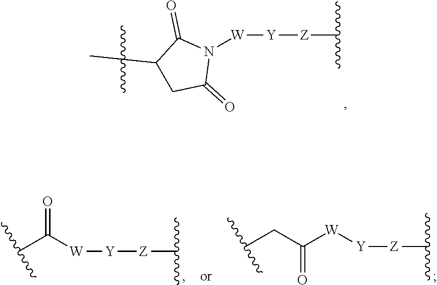
wherein W is —(C(R^{wa})(R^{wb}))_{wn}—, Y is —(OCH₂CH₂)_{yn}—O_{yp}, and Z is —(C(R^{za})(R^{zb}))_{zn}; wherein
Z connects —La— to -Lb-
wherein wn is an integer≥0, and
0 or no less than 1 methylene unit of W is independently replaced by -Cyr-, —N(R^{wx})C(O)—, —C(O)N(R^{wx})—, —C(O)—, —OC(O)—, —C(O)O—, —NR^{wx}—, —O—, —S—, —SO—, —SO₂—, —P(R^{wx})—, —P(═O)(R^{wx})—, —N(R^{wx})SO₂—, —SO₂N(R^{wx})—, —C(═S)—, —C(═NR^{wx})—, —N═N—, —C═N—, —N═C— or —C(═N₂)—;
wherein yn is an integer≥0, and yp is 0 or 1;
wherein zn is an integer≥0, and
0 or no less than 1 methylene unit of Z is independently replaced by -Cyr-, —N(R^{zx})C(O)—, —C(O)N(R^{zx})—, —C(O)—, —OC(O)—, —C(O)O—, —NR^{zx}—, —O—, —S—, —SO—, —SO₂—, —P(R^{zx})—, —P(═O)(R^{zx})—, —N(R^{zx})SO₂—, —SO₂N(R^{zx})—, —C(═S)—, —C(═NR^{zx})—, —N═N—, —C═N—, —N═C— or —C(═N₂)—;
-Cyr- is: 6-10 membered arylene, 5-8 membered heteroarylene, 3-10 membered heterocyclylene, or 3-10 membered saturated or partially unsaturated carbocyclylene, wherein -Cyr- is unsubstituted or independently substituted with no less than 1 substituent R^{cx};
wherein each R^{wa}, each R^{wb}, each R^{za}, each R^{zb}, each R^{wx}, each R^{zx} and each R^{cx} are each independently hydrogen, protium, deuterium, tritium, halogen, —NO₂, —CN, —OR, —SR, —N(R^{ra})(R^{rb}), —C(O)R, —CO₂R, —C(O)C(O)R, —C(O)CH₂C(O)R, —S(O)R, —S(O)₂R, —C(O)N(R^{ra})(R^{rb}), —SO₂N(R^{ra})(R^{rb}), —OC(O)R, —N(R)SO₂R, or a C₁₋₆ aliphatic group optionally substituted with R;
wherein each R, each R^{ra} and each R^{rb} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
-L_{b}- represents a peptide residue consisting of 2 to 7 amino acids;
-L_{c}- is: 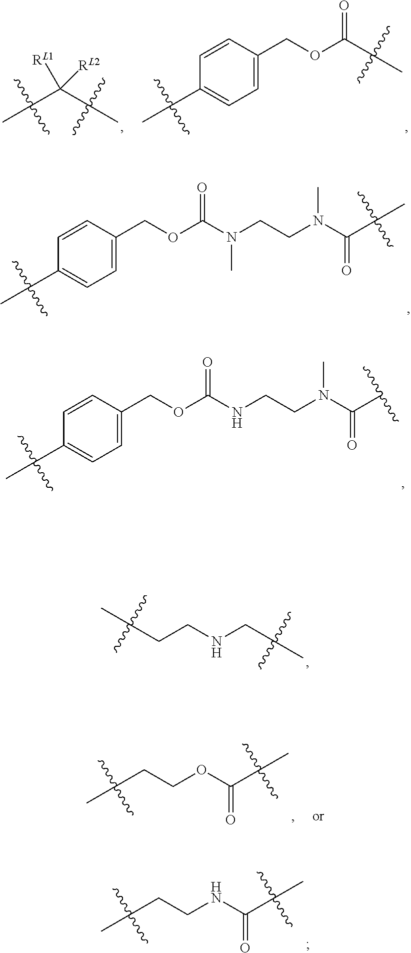
wherein R^{L1} and R^{L2} are each independently: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
wherein R¹ is: —O—, —(R²)N—, —P(═O)(R²)— or —S—;
X is: -L¹-C(R^{1a})(R^{1b})—C(O)—, -L¹-C(R^{1a})(R^{1b})—C(S)—, -L¹-L⁰- or -L³-L²-;
L¹ is —(C(R^{3a})(R^{3b}))ₘ—, wherein 0 or no less than 1 methylene unit of L¹ is independently replaced by —C(O)—, —C(═S)—, —C(═NR^{4b})— or —C(═N₂)—;
L⁰ is —C(R^{2a})(R^{2b})—, or L⁰ is —C(═S)—, —C(═NR^{4a})— or —C(═N₂)—;
L² is —C(R^{5a})(R^{5b})—, wherein 0 or 1 methylene unit of L² is replaced by —N(R⁶)C(O)—, —C(O)N(R⁶)—, —C(O)—, —OC(O)—, —C(O)O—, —NR⁶—, —O—, —S—, —SO—, —SO₂—, —P(R⁶)—, —P(═O)(R⁶)—, —N(R⁶)SO₂—, —SO₂N(R⁶)—, —C(═S)—, —C(═NR⁶)—, —N═N—, —C═N—, —N═C— or —C(═N₂)—;
L³ is —(C(R^{7a})(R^{7b}))ₙ—, wherein no less than 1 methylene unit of L³ is independently replaced by —N(R⁸)C(O)—, —C(O)N(R⁸)—, —OC(O)—, —C(O)O—, —NR⁸—, —O—, —S—, —SO—, —SO₂—, —P(R⁸)—, —P(═O)(R⁸)—, —N(R⁸)SO₂—, —SO₂N(R⁸)—, —N═N—, —C═N— or —N═C—, and 0 or no less than 1 methylene unit of L³ is also independently replaced by —C(O)—, —C(═S)—, —C(═NR⁸)— or —C(═N₂)—;
wherein each R^{1a}, each R^{1b}, each R², each R^{2a}, each R^{2b}, each R^{3a}, each R^{3b}, each R^{4a}, each R^{4b}, each R^{5a}, each R^{5b}, each R⁶, each R^{7a}, each R^{7b} and each R⁸ are each independently hydrogen, protium, deuterium, tritium, halogen, —NO₂, —CN, —OR, —SR, —N(R^{a})(R^{b}), —C(O)R, —CO₂R, —C(O)C(O)R, —C(O)CH₂C(O)R, —S(O)R, —S(O)₂R, —C(O)N(R^{a})(R^{b}), —SO₂N(R^{a})(R), —OC(O)R, —N(R)SO₂R, or a C₁₋₆ aliphatic group optionally substituted with R;
wherein each R, each R^{a} and each R^{b} are each independently hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
m is an integer≥0, and n is an integer≥1;
when R¹ is —O— or —HN— and X is -L¹-CH₂—C(O)—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —HN—, X is -L¹-L⁰-, and L⁰ is —CH₂—, no less than 1 methylene unit of L¹ is independently replaced by —C(O)—, —C(═S)—, —C(═NR^{4b})— or —C(═N₂)—, or each R^{3a} and each R^{3b} are not both hydrogen;
when R¹ is —O—, X is -L³-C(O)—, and 1 methylene unit of L³ is replaced by —NR⁸, R⁸ is not —CH₂—CH₂—NH₂;
when R¹ is —NH—, and X is -L³-C(O)—, no less than 1 methylene unit of L³ is replaced by —N(R⁸)C(O)—, —OC(O)—, —C(O)O—, —S—, —SO—, —SO₂—, —P(R⁸)—, —P(═O)(R⁸)—, —N(R⁸)SO₂—, —SO₂N(R⁸)—, —N═N—, —C═N— or —N═C—.

66. The antibody drug conjugate according to claim 65, wherein the antibody drug conjugate is according to any one of the structures D-III-1, D-III-2, D-III-3, D-III-4, D-III-5, D-III-6, or D-III-7, 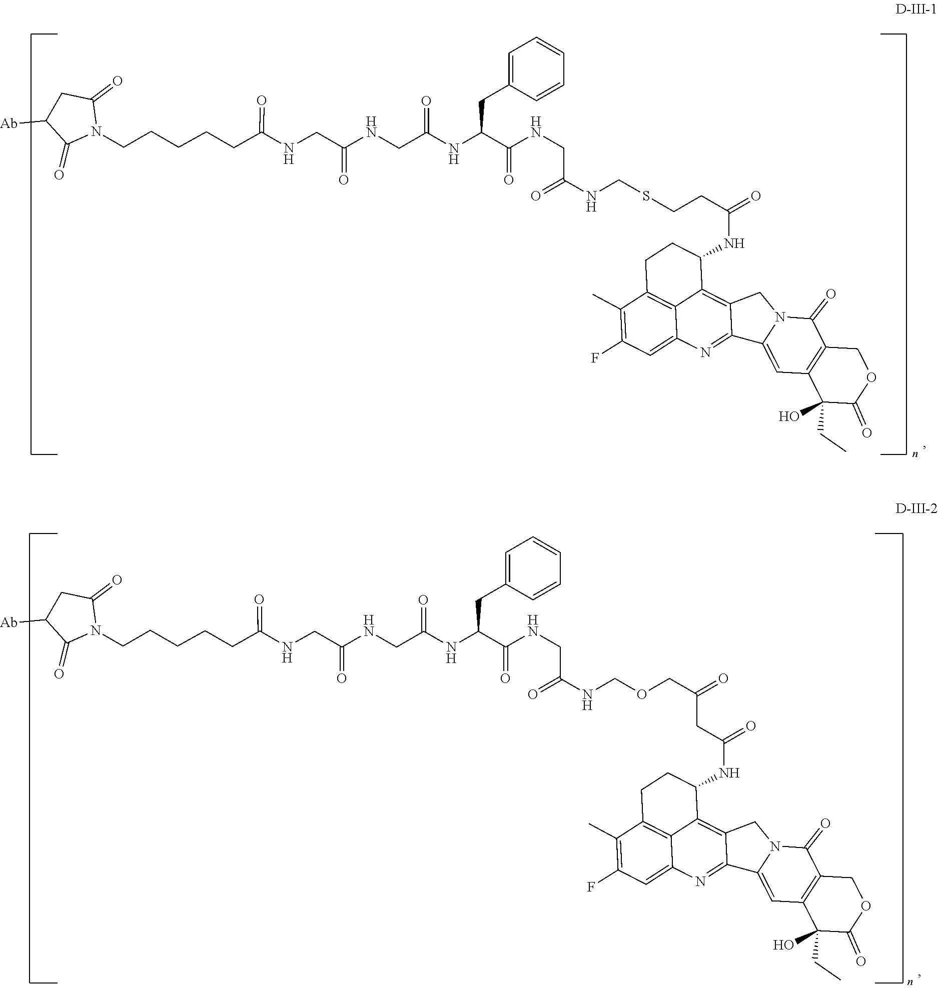 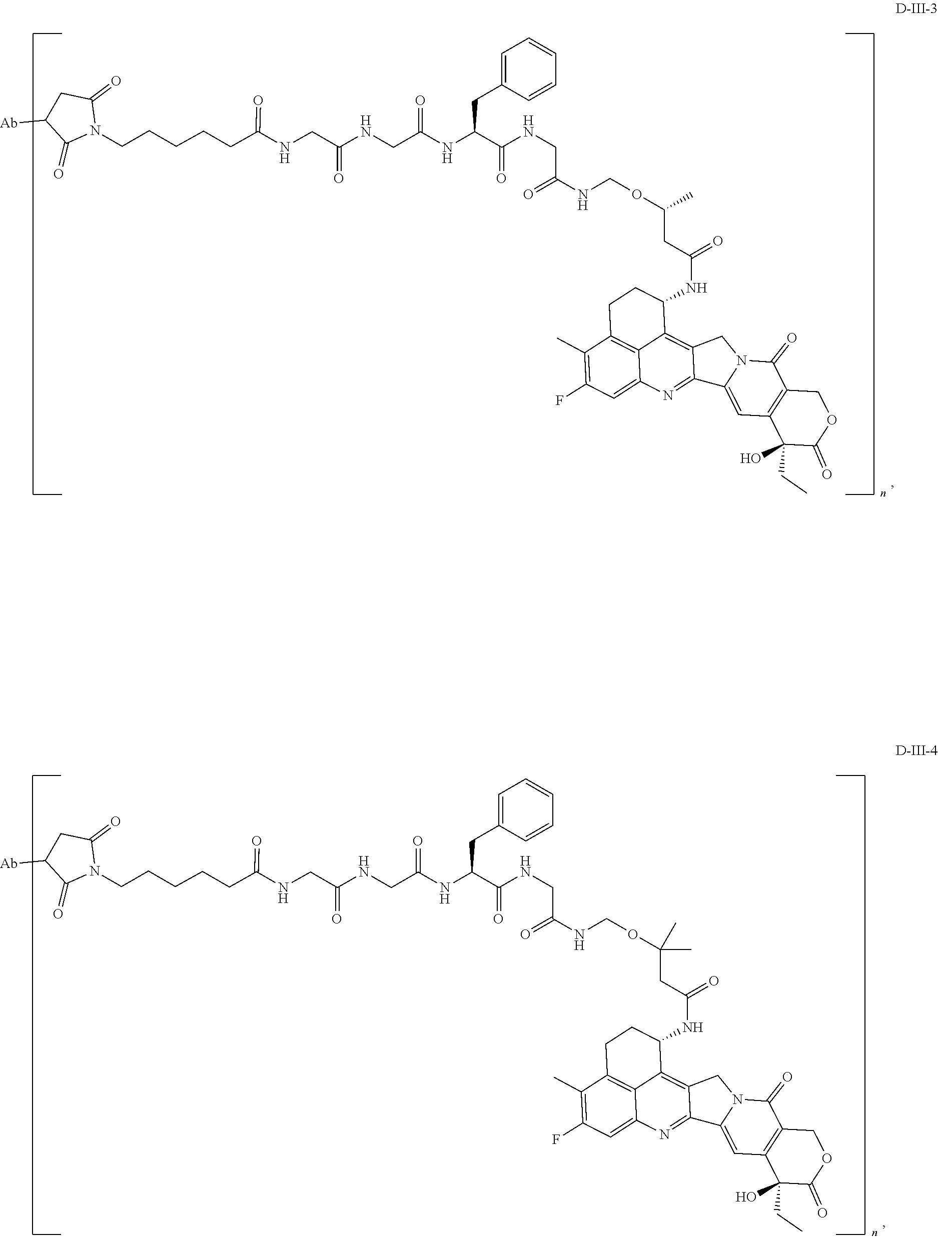 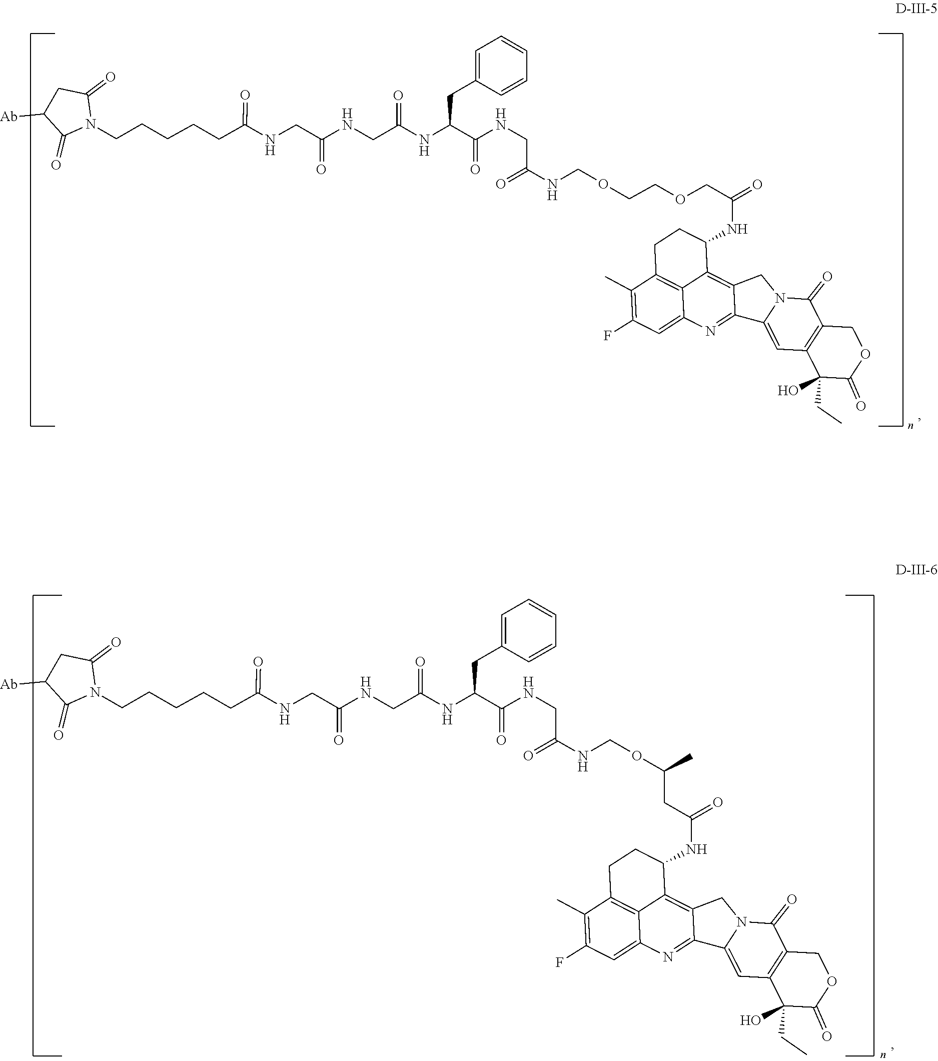 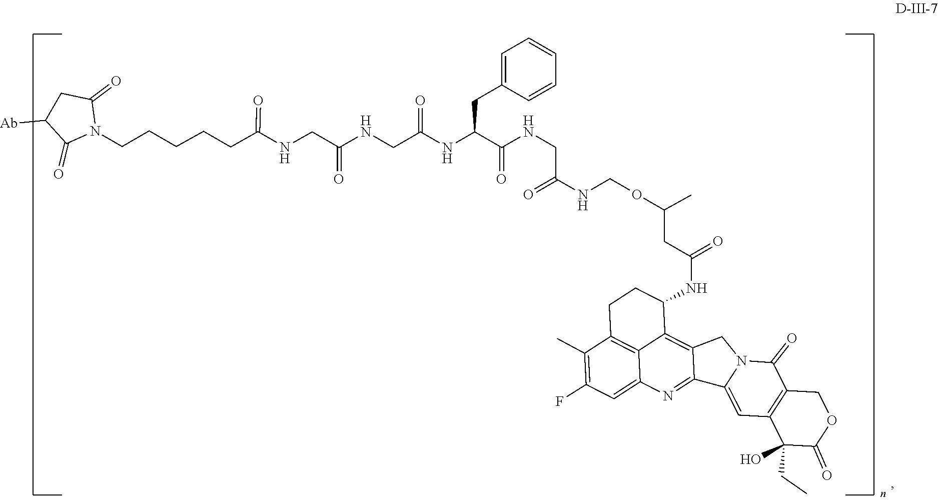
or a tautomer, a mesomer, a racemate, an enantiomer or a diastereoisomer thereof, or a mixture thereof, or a pharmaceutically acceptable salt thereof, wherein Ab represents the antibody of claim 52 which binds uPARAP, wherein n is an integer from 1 to 10.

67. The antibody-drug conjugate according to claim 65, having the structure of D-III-6, 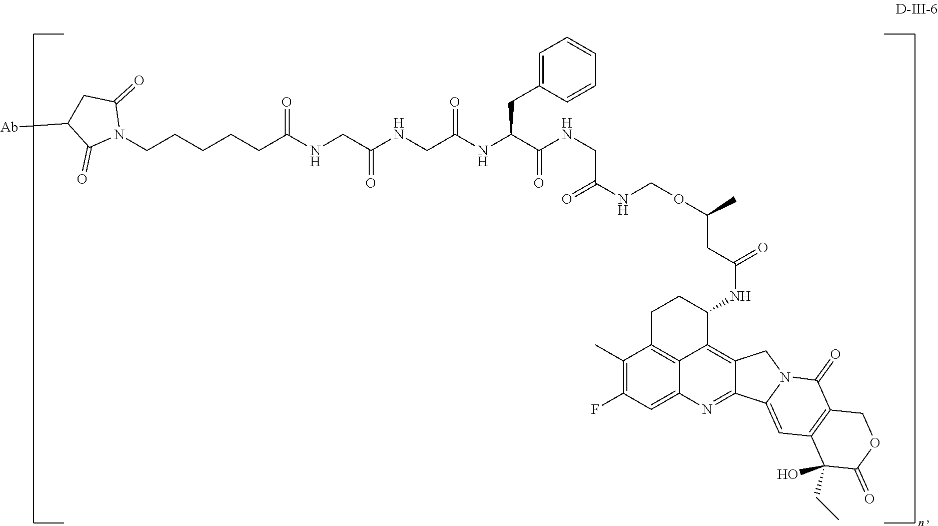
wherein n is an integer from 1 to 10 and further wherein the antibody (Ab) comprises
a. an immunoglobulin light chain variable region comprising or consisting of the amino acid sequence of SEQ ID NO: 3; and
b. an immunoglobulin heavy chain variable region comprising or consisting of the amino acid sequence of SEQ ID NO: 6.

68. The antibody-drug conjugate according to claim 67, wherein n is 8.

69. The antibody drug conjugate according to claim 52, wherein the active agent is derived from any one of the structures P-III-30, P-III-31, P-III-1, P-III-2, P-III-9, P-III-20, P-III-21, P-III-22, P-III-27, P-III-28, or P-III-29, 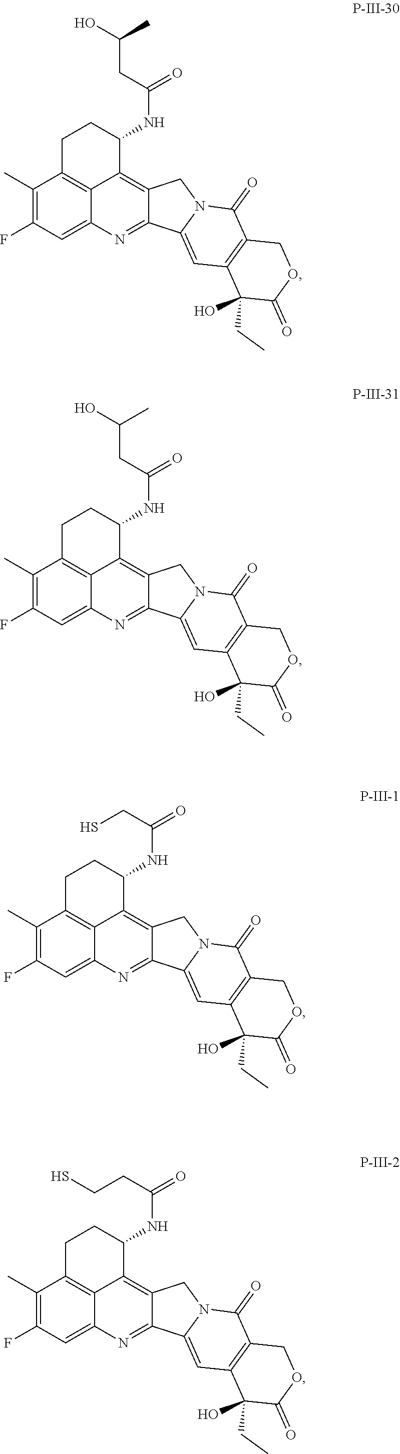 [SMILES: CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(F)c(C)c3c1c2[C@@H](NC(=O)CC(C)O)CC3 ; CC[C@@]1(O)C(=O)OCc2c1cc1n(c2=O)Cc2c-1nc1cc(F)c(C)c3c1c2[C@@H](NC(=O)CS)CC3 ; CC[C@@]1(O)C(=O)OCc2c1cc1n(c2=O)Cc2c-1nc1cc(F)c(C)c3c1c2[C@@H](NC(=O)CCS)CC3] 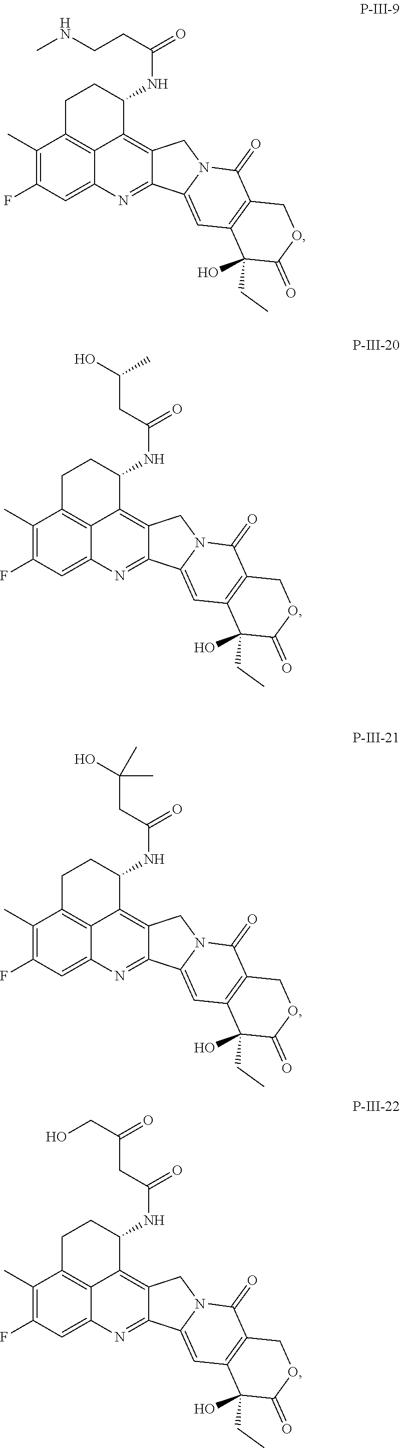 [SMILES: CC[C@@]1(O)C(=O)OCc2c1cc1n(c2=O)Cc2c-1nc1cc(F)c(C)c3c1c2[C@@H](NC(=O)CCNC)CC3 ; CC[C@@]1(O)C(=O)OCc2c1cc1n(c2=O)Cc2c-1nc1cc(F)c(C)c3c1c2[C@@H](NC(=O)C[C@@H](C)O)CC3 ; CC[C@@]1(O)C(=O)OCc2c1cc1n(c2=O)Cc2c-1nc1cc(F)c(C)c3c1c2[C@@H](NC(=O)CC(C)(C)O)CC3 ; CC[C@@]1(O)C(=O)OCc2c1cc1n(c2=O)Cc2c-1nc1cc(F)c(C)c3c1c2[C@@H](NC(=O)CC(=O)CO)CC3] 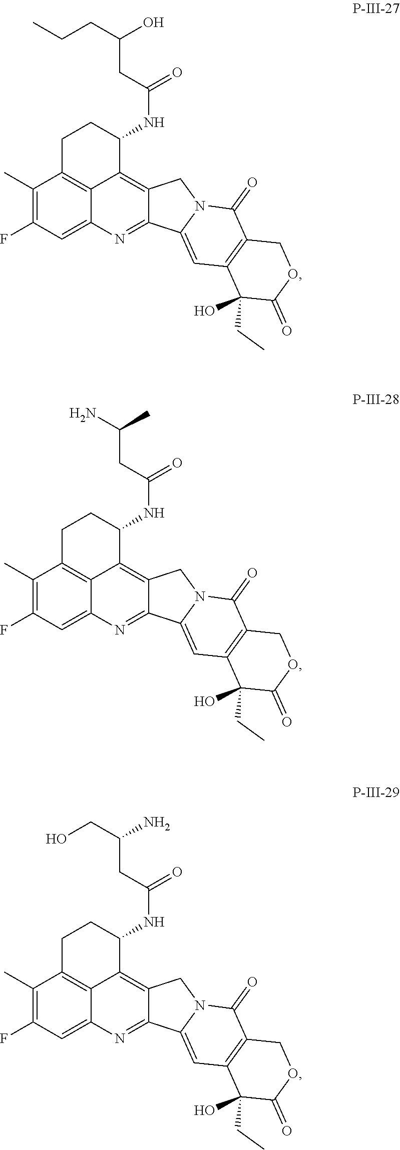 [SMILES: CCCC(O)CC(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)C[C@H](C)N)CC3 ; CC[C@@]1(O)C(=O)OCc2c1cc1n(c2=O)Cc2c-1nc1cc(F)c(C)c3c1c2[C@@H](NC(=O)C[C@@H](N)CO)CC3]
or a tautomer, a mesomer, a racemate, an enantiomer or a diastereoisomer thereof, or a mixture thereof, or a pharmaceutically acceptable salt thereof.

70. The antibody drug conjugate according to claim 52, wherein the compound is L-III-30, L-III-31, L-III-22, L-III-21, L-III-20, L-III-7, L-III-6, L-III-4, L-III-3, or L-III-2, 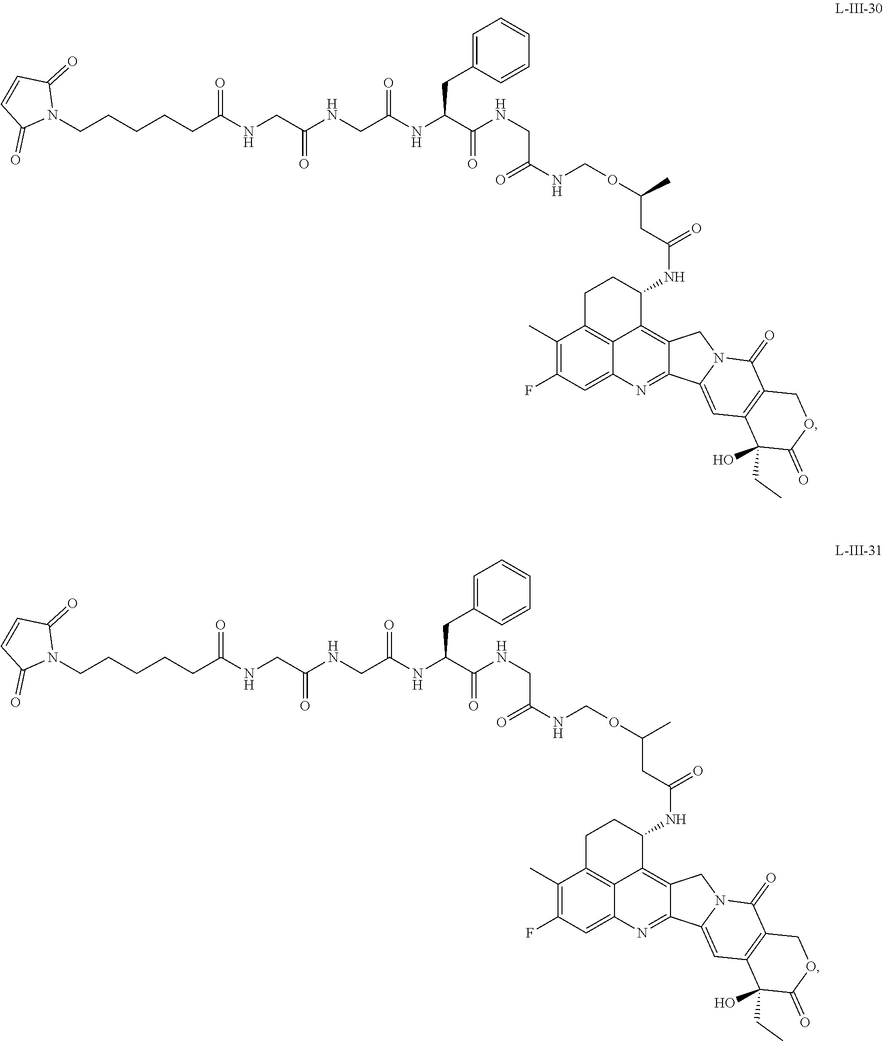 [SMILES: CC[C@@]1(O)C(=O)OCc2c1cc1n(c2=O)Cc2c-1nc1cc(F)c(C)c3c1c2[C@@H](NC(=O)C[C@H](C)OCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)CC(C)OCNC(=O)CNC(=O)[C@H](Cc1ccccc1)NC(=O)CNC(=O)CNC(=O)CCCCCN1C(=O)C=CC1=O)CC3] 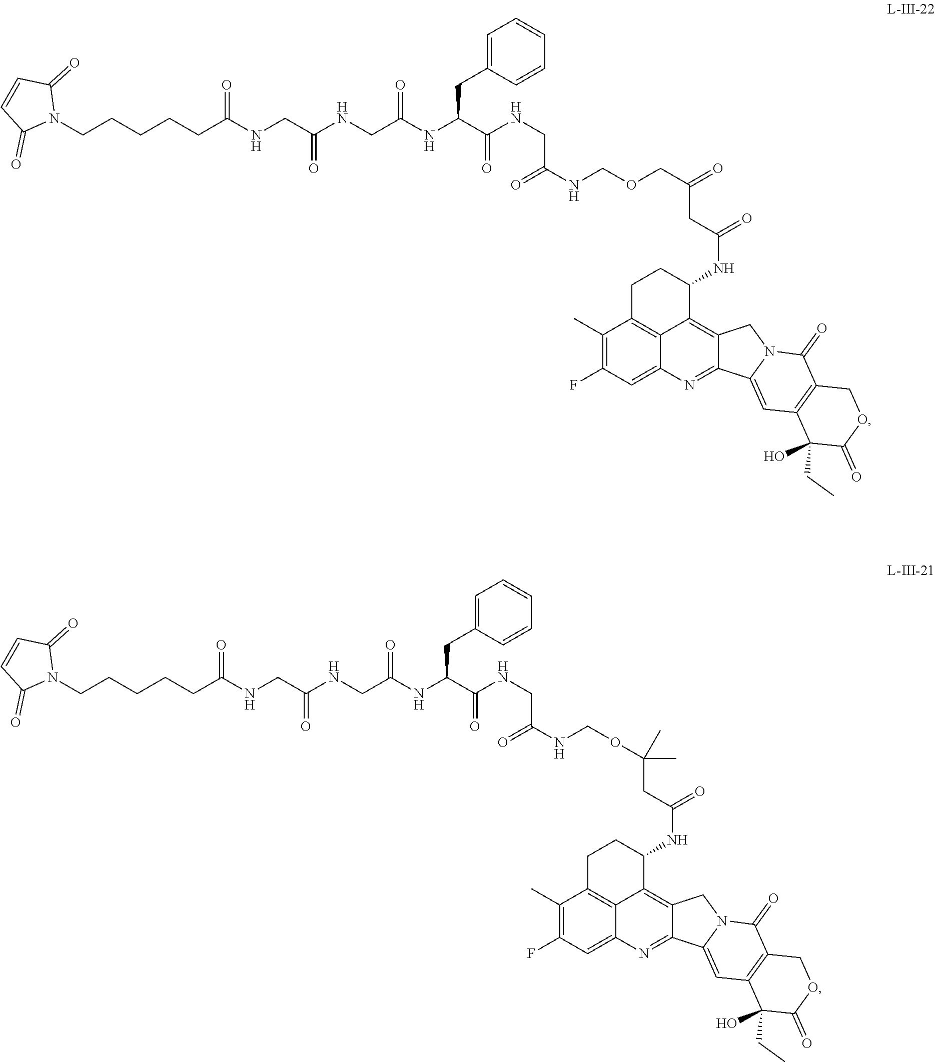 [SMILES: CC[C@@]1(O)C(=O)OCc2c1cc1n(c2=O)Cc2c-1nc1cc(F)c(C)c3c1c2[C@@H](NC(=O)CC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)CC(C)(C)OCNC(=O)CNC(=O)[C@H](Cc1ccccc1)NC(=O)CNC(=O)CNC(=O)CCCCCN1C(=O)C=CC1=O)CC3] 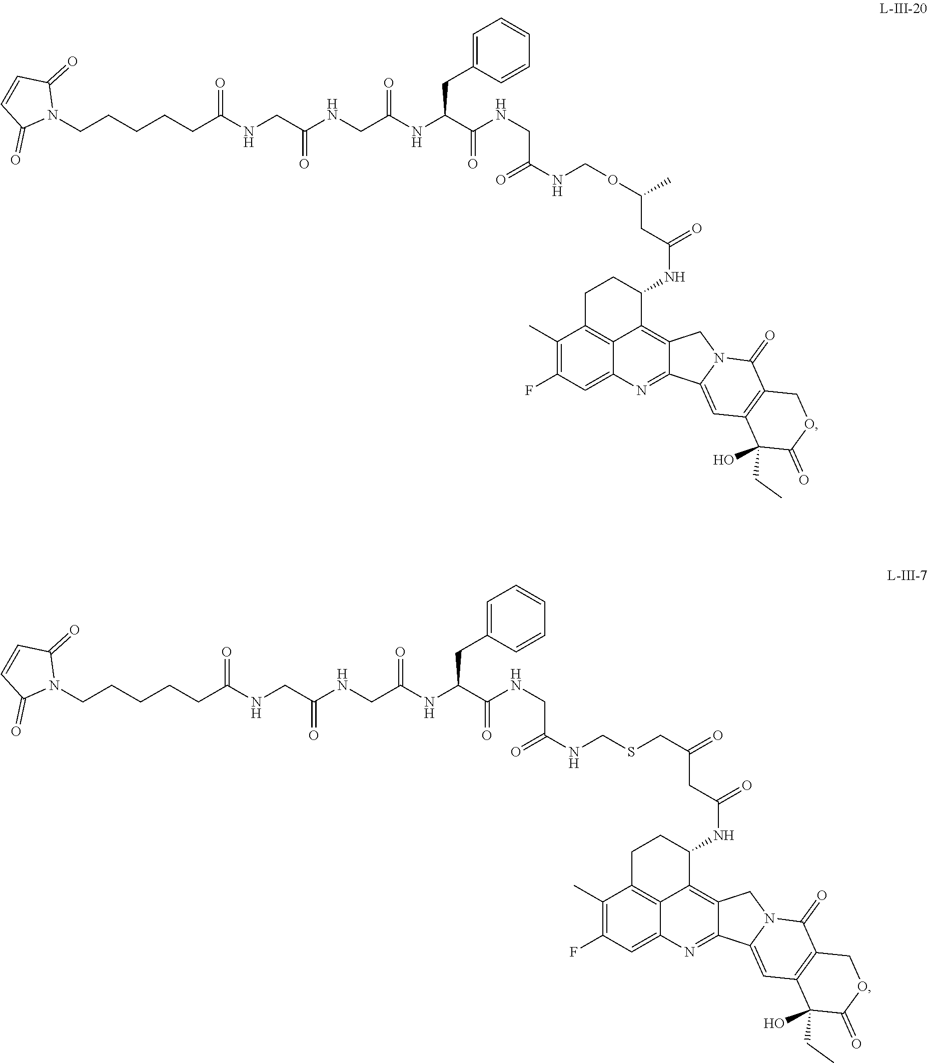 [SMILES: CC[C@@]1(O)C(=O)OCc2c1cc1n(c2=O)Cc2c-1nc1cc(F)c(C)c3c1c2[C@@H](NC(=O)C[C@@H](C)OCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)CC(=O)CSCNC(=O)CNC(=O)[C@H](Cc1ccccc1)NC(=O)CNC(=O)CNC(=O)CCCCCN1C(=O)C=CC1=O)CC3] 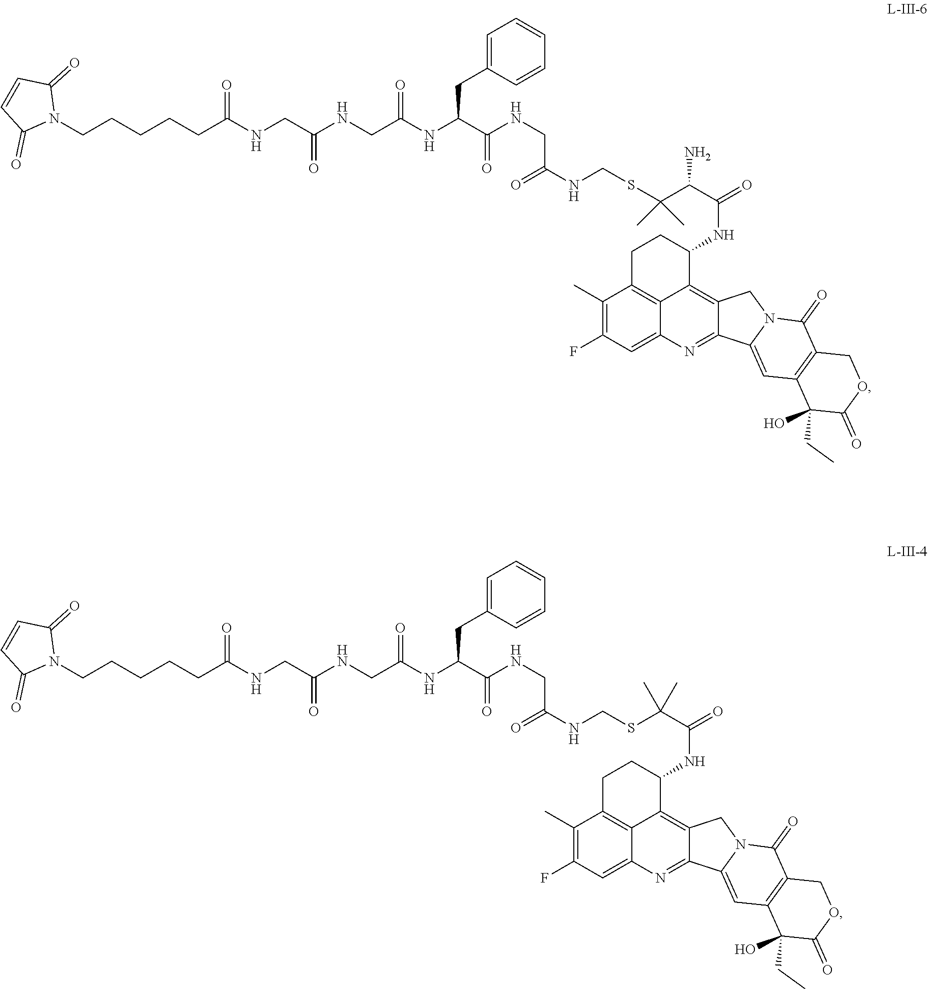 [SMILES: CC[C@@]1(O)C(=O)OCc2c1cc1n(c2=O)Cc2c-1nc1cc(F)c(C)c3c1c2[C@@H](NC(=O)[C@@H](N)C(C)(C)SCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)C(C)(C)SCNC(=O)CNC(=O)[C@H](Cc1ccccc1)NC(=O)CNC(=O)CNC(=O)CCCCCN1C(=O)C=CC1=O)CC3] 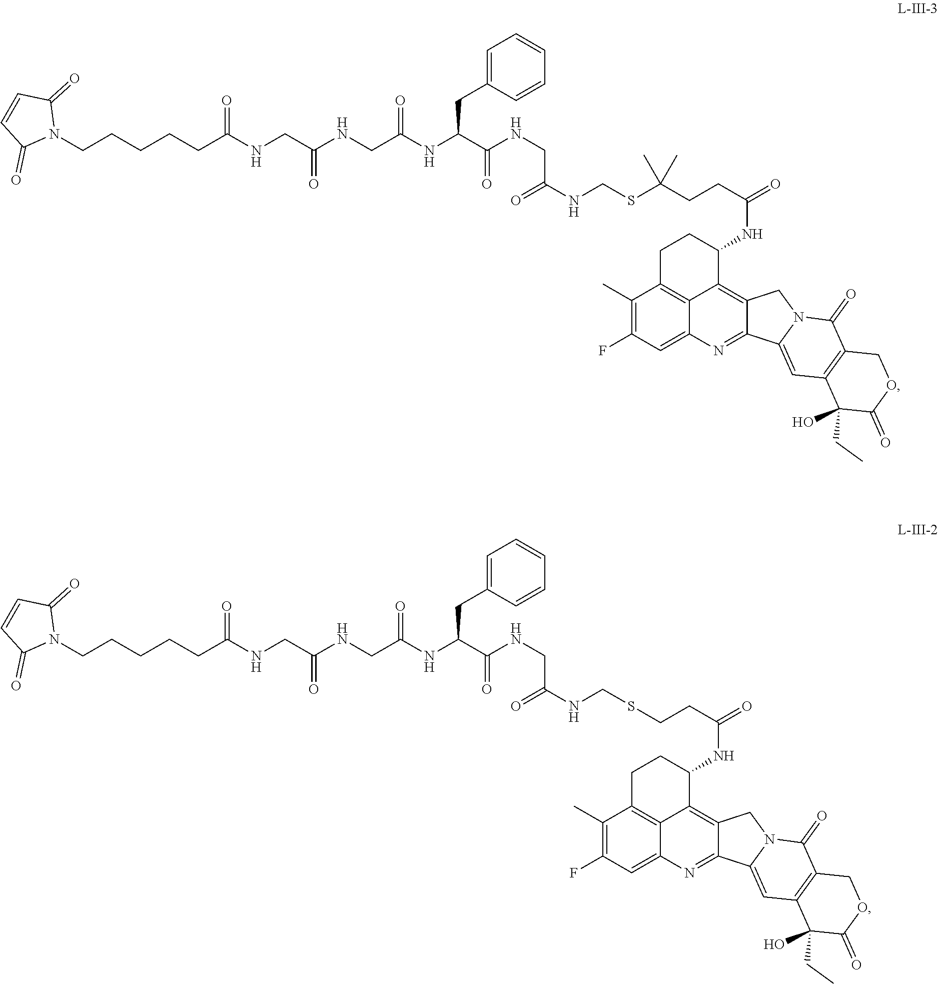 [SMILES: CC[C@@]1(O)C(=O)OCc2c1cc1n(c2=O)Cc2c-1nc1cc(F)c(C)c3c1c2[C@@H](NC(=O)CCC(C)(C)SCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)CCSCNC(=O)CNC(=O)[C@H](Cc1ccccc1)NC(=O)CNC(=O)CNC(=O)CCCCCN1C(=O)C=CC1=O)CC3]
or a tautomer, a mesomer, a racemate, an enantiomer or a diastereoisomer thereof, or a mixture thereof, or a pharmaceutically acceptable salt thereof, prior to conjugation with the antibody which binds uPARAP.

71. The antibody-drug conjugate according to claim 52, wherein the antibody comprises:
a. an immunoglobulin light chain comprising or consisting of the amino acid sequence of SEQ ID NO: 1; and
b. an immunoglobulin heavy chain comprising or consisting of the amino acid sequence of SEQ ID NO: 4.

72. The antibody-drug conjugate according to claim 52, having the structure of D-III-6, 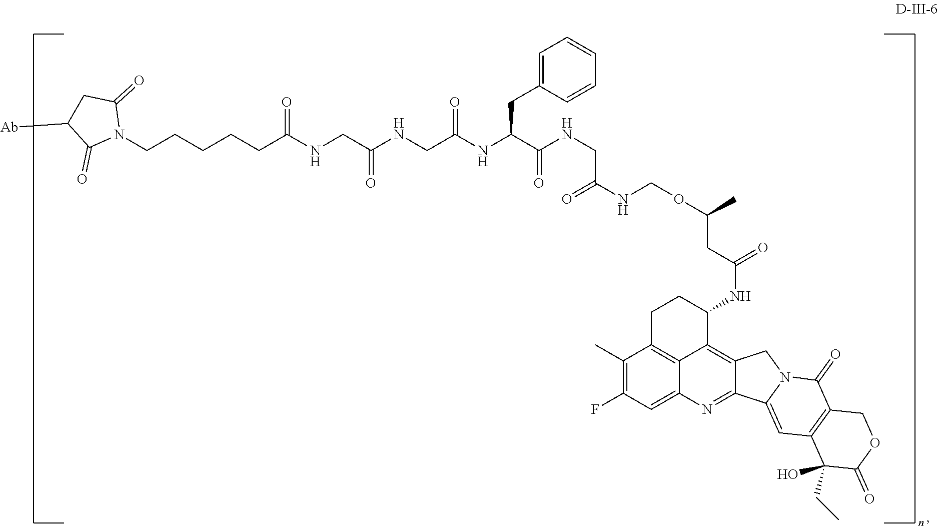
wherein n is an integer from 1 to 10 and further wherein the antibody (Ab) comprises:
a. an immunoglobulin light chain comprising or consisting of the amino acid sequence of SEQ ID NO: 1; and
b. an immunoglobulin heavy chain comprising or consisting of the amino acid sequence of SEQ ID NO: 4.

73. The antibody-drug conjugate according to claim 72, wherein n is 8.

74. A pharmaceutical composition, comprising the antibody-drug conjugate according to claim 52, and a pharmaceutically acceptable carrier.

75. A method for treatment of a disease characterised by cells expressing uPARAP in a subject comprising administering to the subject the antibody-drug conjugate according to claim 52, wherein the disease characterised by cells expressing uPARAP is cancer, a bone degradation disease, fibrosis, or macrophage associated diseases or disorders.

76. A kit comprising the antibody-drug conjugate according to claim 52.


SEQUENCE LISTING (15 sequences)

SEQ ID NO: 1 - protein, 214 aa, synthetic construct
  REGION 1..214: note = Complete light chain sequence (LC4) of humanised LC4HC3
EIVMTQSPDSLAVSLGERATINCKASQNVDTYVVWYQQKPGQPPQPLIYSASSRFSGVPDRFSGSGSGTDFTLTISSLQAEDVAIYYCQQYHNSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 234 aa, synthetic construct
  REGION 1..234: note = Complete light chain sequence (LC4) of humanised LC4HC3 in combination with signal peptide used in CHO cells expression system; Signal peptide underlined
MVSSAQFLGLLLLCFQGTRCEIVMTQSPDSLAVSLGERATINCKASQNVDTYVVWYQQKPGQPPQPLIYSASSRFSGVPDRFSGSGSGTDFTLTISSLQAEDVAIYYCQQYHNSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 107 aa, synthetic construct
  REGION 1..107: note = Light chain (LC4) variable region of humanised LC4HC3
EIVMTQSPDSLAVSLGERATINCKASQNVDTYVVWYQQKPGQPPQPLIYSASSRFSGVPDRFSGSGSGTDFTLTISSLQAEDVAIYYCQQYHNSPLTFGGGTKVEIK

SEQ ID NO: 4 - protein, 449 aa, synthetic construct
  REGION 1..449: note = Complete heavy chain sequence (HC3) of humanised LC4HC3
QVQLVQSGAEVKKPGASVKVSCKASGYIFIDYGMHWVRQAPGQRLEWMGSINTKSGVSTYAAEFKGRVTIYSDTSASTAYMELSSLRSEDTAVYFCARPPYYSQYGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 468 aa, synthetic construct
  REGION 1..468: note = Complete heavy chain sequence (HC3) of humanised LC4HC3 in combination with signal peptide used in CHO cell expression system; Signal peptide underlined
MGWTLVFLFLLSVTAGVHSQVQLVQSGAEVKKPGASVKVSCKASGYIFIDYGMHWVRQAPGQRLEWMGSINTKSGVSTYAAEFKGRVTIYSDTSASTAYMELSSLRSEDTAVYFCARPPYYSQYGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 119 aa, synthetic construct
  REGION 1..119: note = Heavy chain (HC3) variable region of humanised LC4HC3
QVQLVQSGAEVKKPGASVKVSCKASGYIFIDYGMHWVRQAPGQRLEWMGSINTKSGVSTYAAEFKGRVTIYSDTSASTAYMELSSLRSEDTAVYFCARPPYYSQYGSYWGQGTLVTVSS

SEQ ID NO: 7 - protein, 214 aa, synthetic construct
  REGION 1..214: note = Complete light chain sequence (LC0) of chimeric LC0HC0
DIVMTQSQKFMSTSVGDRVSVTCKASQNVDTYVVWYQQKPGQSPKPLIYSASSRFSGVPDRFTGTGSGTDFTLTINNVQSEDLAEYFCQQYHNSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 107 aa, synthetic construct
  REGION 1..107: note = Light chain (LC0) variable region of chimeric LC0HC0
DIVMTQSQKFMSTSVGDRVSVTCKASQNVDTYVVWYQQKPGQSPKPLIYSASSRFSGVPDRFTGTGSGTDFTLTINNVQSEDLAEYFCQQYHNSPLTFGGGTKLEIK

SEQ ID NO: 9 - protein, 449 aa, synthetic construct
  REGION 1..449: note = Complete heavy chain sequence (HC0) of chimeric LC0HC0
QVHLVQSGPELKKPGETVKISCKASGYIFIDYGMHWVKQAPGKGLKWMGSINTKSGVSTYAAEFKGRFAFSLETSASTAYLQINNLKNEDTATYFCARPPYYSQYGSYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 119 aa, synthetic construct
  REGION 1..119: note = Heavy chain (HC0) variable region of chimeric LC0HC0
QVHLVQSGPELKKPGETVKISCKASGYIFIDYGMHWVKQAPGKGLKWMGSINTKSGVSTYAAEFKGRFAFSLETSASTAYLQINNLKNEDTATYFCARPPYYSQYGSYWGQGTLVTVSA

SEQ ID NO: 11 - DNA, 705 bp, synthetic construct
  misc_feature 1..705: note = Polynucleotide encoding LC4 incl. signal peptide
  source 1..705: mol_type = other DNA
atggtcagctctgctcaatttctcggactccttcttctgtgctttcaaggaacacgctgcgagatcgtgatgactcagtccccggactcactggcagtgtccttgggcgaaagagccaccatcaactgtaaagccagccagaacgtggacacctacgtggtctggtaccagcagaagcctggacagccaccgcagccgttgatctactcggcctcatcaaggttctccggggtgccggaccgcttctccggatccggctccggcaccgatttcaccctgaccatctcctcactgcaagccgaggacgtggctatctactattgccagcagtaccacaactccccactgaccttcggtggcggaactaaggtcgagattaagcggaccgtggcggccccctctgtgttcattttccctccctcggacgaacagctgaagtcgggaacagcctccgtcgtgtgcctgctcaacaacttctacccccgggaagcgaaggtccagtggaaagtggataacgcactccaatcggggaactcccaggaatccgtgactgagcaggactcgaaggattccacttactccctgtcgtccaccctgactctgagcaaggccgactacgagaagcataaggtctacgcctgcgaagtgacccaccagggtctgagctcccctgtgaccaagagctttaatcggggcgaatgttga

SEQ ID NO: 12 - DNA, 1407 bp, synthetic construct
  misc_feature 1..1407: note = Polynucleotide encoding HC3 incl. signal peptide
  source 1..1407: mol_type = other DNA
atgggttggaccctcgtctttctgttccttctttccgtcaccgctggagtgcatagccaggtccaattggtgcagtcaggcgccgaagtgaaaaagcctggggcgtcggtgaaagtgtcctgcaaagcctcgggctacatctttattgactacggaatgcactgggtccgccaggccccgggccagaggctggagtggatgggatccattaacaccaagagcggagtgtcaacttacgcagccgagttcaagggacgggtgaccatctatagcgatacctctgcgtcgaccgcctacatggaattgtcatcactccggtccgaggacactgccgtgtacttctgcgcaaggccaccctactactcgcaatacggcagctactggggccagggaacacttgtgaccgtgtcgagcgcgtccaccaagggtccctccgtgttccctctcgcgccgtcctcaaagtctacctccggtggaactgccgcgctcggttgtctcgtgaaggactacttcccggagcctgtgactgtctcctggaactccggggccctcaccagcggagtgcacactttccccgccgtgctgcaatcctccggcctgtacagcctgtcctccgtcgtgactgtgcctagctcctccctgggaacccagacctacatctgcaacgtgaaccacaagccctccaacaccaaggtcgacaagaaggtcgaaccgaagtcgtgcgacaagactcatacgtgccctccttgcccggccccggaactgctgggaggcccatccgtgttcctgttcccacccaagcctaaggataccctgatgatcagcagaacaccggaagtgacctgtgtggtggtggacgtcagccacgaagatcccgaggtcaagttcaattggtacgtggacggggtggaggtgcacaacgcaaagaccaagccccgggaggaacagtacaactccacctatcgcgtggtgtcggtgctgacggtgctgcaccaggactggttgaacggaaaggagtataagtgcaaagtgtcgaacaaggccctgcccgctcctatcgaaaagaccatctccaaggccaagggccagccgcgggaaccccaggtctacactctcccaccgagccgcgacgaactgactaagaatcaagtgtcgctgacttgcctcgtcaagggcttctacccgtccgacatcgccgtggaatgggagagcaacggccagccggaaaacaactacaagaccacccctcccgtgctggattccgacgggtccttcttcctgtactcaaaactgaccgtggataagtccagatggcagcagggcaatgtcttttcatgctccgtgatgcacgaggctctgcataaccactacacccagaagtcgctgtccctgtccccggggaagtga

SEQ ID NO: 13 - protein, 234 aa, synthetic construct
  REGION 1..234: note = Complete light chain sequence (LC3) of humanised LC3HC3
MVSSAQFLGLLLLCFQGTRCDVVMTQSPDSLAVSLGERVTINCKASQNVDTYVVWYQQKPGQSPKLLIYSASSRFSGVPDRFSGSGSGTDFTLTISSLQAEDVAVYYCQQYHNSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 4 aa, synthetic construct
GGFG

SEQ ID NO: 15 - protein, 4 aa, synthetic construct
GGGG